SEQUENCE LISTING (83 sequences)

SEQ ID NO: 1 - protein, 5 aa, synthetic construct
  REGION 1..5: note = Synthetic: HCDR1 of anti-C1s antibody
DDYIH

SEQ ID NO: 2 - protein, 17 aa, synthetic construct
  REGION 1..17: note = Synthetic: HCDR2 of anti-C1s antibody
RIDPADGHTKYAPKFQV

SEQ ID NO: 3 - protein, 10 aa, synthetic construct
  REGION 1..10: note = Synthetic: HCDR3 of anti-C1s antibody
YGYGREVFDY

SEQ ID NO: 4 - protein, 15 aa, synthetic construct
  REGION 1..15: note = Synthetic: LCDR1 of anti-C1s antibody
KASQSVDYDGDSYMN

SEQ ID NO: 5 - protein, 7 aa, synthetic construct
  REGION 1..7: note = Synthetic: LCDR2 of anti-C1s antibody
DASNLES

SEQ ID NO: 6 - protein, 9 aa, synthetic construct
  REGION 1..9: note = Synthetic: LCDR3 of anti-C1s antibody
QQSNEDPWT

SEQ ID NO: 7 - protein, 60 aa, synthetic construct
  REGION 1..60: note = Synthetic: VH of anti-C1s antibody
QVQLVQSGAEVKKPGASVKLSCTASGFNIKDDYIHWVKQAPGQGLEWIGRIDPADGHTKY

SEQ ID NO: 8 - protein, 60 aa, synthetic construct
  REGION 1..60: note = Synthetic: VL of anti-C1s antibody
DIVLTQSPDSLAVSLGERATISCKASQSVDYDGDSYMNWYQQKPGQPPKILIYDASNLES

SEQ ID NO: 9 - protein, 245 aa, synthetic construct
  REGION 1..245: note = Synthetic: alpha-C1s scFv
QVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSSGGGGSGGGGSGGGGSDIVLTQSPDSLAVSLGERATISCKASQSVDYDGDSYMNWYQQKPGQPPKILIYDASNLESGIPARFSGSGSGTDFTLTISSLEPEDFAIYYCQQSNEDPWTFGGGTKVEIK

SEQ ID NO: 10 - protein, 217 aa, synthetic construct
  REGION 1..217: note = Synthetic: heavy chain of anti-C1s Fab
QVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 11 - protein, 218 aa, synthetic construct
  REGION 1..218: note = Synthetic: light chain of anti-C1s Fab
DIVLTQSPDSLAVSLGERATISCKASQSVDYDGDSYMNWYQQKPGQPPKILIYDASNLESGIPARFSGSGSGTDFTLTISSLEPEDFAIYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 470 aa, synthetic construct
  REGION 1..470: note = Synthetic: alpha-C1s scFab
DIVLTQSPDSLAVSLGERATISCKASQSVDYDGDSYMNWYQQKPGQPPKILIYDASNLESGIPARFSGSGSGTDFTLTISSLEPEDFAIYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSQVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 13 - protein, 5 aa, synthetic construct
  REGION 1..5: note = Synthetic: HCDR1 of anti-Bb antibody
NYAMS

SEQ ID NO: 14 - protein, 17 aa, synthetic construct
  REGION 1..17: note = Synthetic: HCDR2 of anti-Bb antibody
TISNRGSYTYYPDSVKG

SEQ ID NO: 15 - protein, 6 aa, synthetic construct
  REGION 1..6: note = Synthetic: HCDR3 of anti-Bb antibody
ERPMDY

SEQ ID NO: 16 - protein, 11 aa, synthetic construct
  REGION 1..11: note = Synthetic: LCDR1 of anti-Bb antibody
KASQDVGTAVA

SEQ ID NO: 17 - protein, 7 aa, synthetic construct
  REGION 1..7: note = Synthetic: LCDR2 of anti-Bb antibody
WASTRHT

SEQ ID NO: 18 - protein, 9 aa, synthetic construct
  REGION 1..9: note = Synthetic: LCDR3 of anti-Bb antibody
HQHSSNPLT

SEQ ID NO: 19 - protein, 115 aa, synthetic construct
  REGION 1..115: note = Synthetic: VH of anti-Bb antibody
EVQLVESGGGLVKPGGSLRLSCAASGFTFSNYAMSWVRQAPGKRLEWVATISNRGSYTYYPDSVKGRFTISRDNAKNSLYLQMNSLRAEDTALYYCARERPMDYWGQGTLVTVSS

SEQ ID NO: 20 - protein, 107 aa, synthetic construct
  REGION 1..107: note = Synthetic: VL of anti-Bb antibody
DIQMTQSPSTLSASVGDRVTITCKASQDVGTAVAWYQQKPGKAPKLLIYWASTRHTGVPDRFSGSGSGTDFTLTISSLQAEDFAVYFCHQHSSNPLTFGQGTKLEIK

SEQ ID NO: 21 - protein, 237 aa, synthetic construct
  REGION 1..237: note = Synthetic: alpha-Bb scFv
EVQLVESGGGLVKPGGSLRLSCAASGFTFSNYAMSWVRQAPGKRLEWVATISNRGSYTYYPDSVKGRFTISRDNAKNSLYLQMNSLRAEDTALYYCARERPMDYWGQGTLVTVSSGGGGSGGGGSGGGGSDIQMTQSPSTLSASVGDRVTITCKASQDVGTAVAWYQQKPGKAPKLLIYWASTRHTGVPDRFSGSGSGTDFTLTISSLQAEDFAVYFCHQHSSNPLTFGQGTKLEIK

SEQ ID NO: 22 - protein, 213 aa, synthetic construct
  REGION 1..213: note = Synthetic: heavy chain of anti-Bb Fab
EVQLVESGGGLVKPGGSLRLSCAASGFTFSNYAMSWVRQAPGKRLEWVATISNRGSYTYYPDSVKGRFTISRDNAKNSLYLQMNSLRAEDTALYYCARERPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 23 - protein, 214 aa, synthetic construct
  REGION 1..214: note = Synthetic: light chain of anti-Bb Fab
DIQMTQSPSTLSASVGDRVTITCKASQDVGTAVAWYQQKPGKAPKLLIYWASTRHTGVPDRFSGSGSGTDFTLTISSLQAEDFAVYFCHQHSSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 462 aa, synthetic construct
  REGION 1..462: note = Synthetic: alpha-Bb scFab
DIQMTQSPSTLSASVGDRVTITCKASQDVGTAVAWYQQKPGKAPKLLIYWASTRHTGVPDRFSGSGSGTDFTLTISSLQAEDFAVYFCHQHSSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSEVQLVESGGGLVKPGGSLRLSCAASGFTFSNYAMSWVRQAPGKRLEWVATISNRGSYTYYPDSVKGRFTISRDNAKNSLYLQMNSLRAEDTALYYCARERPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 25 - DNA, 2904 bp, synthetic construct
  misc_feature 1..2904: note = Synthetic: [alpha-C1s scFab(G4S)3 alpha-Bb scFab] nucleic acid sequence (construct #7, FIG. 2A)
  source 1..2904: mol_type = other DNA
atggaagcccccgcccagctgctgttcctgctgctgctgtggctgcctgacaccactggcgatatcgtgctgacacagagccctgatagcctggctgttagcctgggcgaacgcgccacaatcagctgcaaggccagccagtctgtggattatgatggtgacagctacatgaactggtaccagcagaagcccggacagcctcctaagatcctgatctacgacgccagcaacctggaatccggcattcctgcccggttcagcggctccggcagcggcaccgacttcaccctgaccatctccagcctggaacccgaggatttcgccatctactactgtcagcagagcaatgaggacccatggaccttcggcggcggtaccaaggtcgagatcaagagaacagtggccgctcctagcgtgttcatcttccctccatcagacgagcagctgaagagcggaaccgcttctgtggtgtgtctgctcaacaatttctaccctagagaagccaaggtgcagtggaaggtggacaacgctctccagagcggcaacagccaggagagcgtgaccgagcaagatagcaaggacagcacctactctttaagctctacactgacgctgtccaaggctgactacgagaagcacaaggtgtacgcctgtgaagtgacccaccagggcctgagcagccctgtgacaaagagcttcaacagaggcgagtgcggcggcggaggcagcggcggcggaggctctggcggtggcggaagcggcgggggaggctctggcggcggcggcagtggcggcggcggcagcggaggaggaggatcgcaagtgcaactggtccagtctggcgccgaggtgaaaaagcctggagccagcgtgaaactgtcatgcaccgcctccgggtttaacatcaaagatgactacatccactgggtgaaacaggctccaggacagggcctggagtggatcggcagaatcgaccctgcggatggccacaccaagtacgccccaaagttccaggtgaaggtgacaatcacagctgacaccagcaccagcacagcctacctggaactgagcagcctaagaagcgaggacaccgccgtgtactactgcgcccggtacggctacggccgggaagtgttcgactactggggtcagggcaccaccgtgacggtgagtagcgcctctacaaaaggcccttccgtgttccccctggccccttgcagccggagcaccagcgagagcaccgccgccttgggctgtctggtgaaagactatttcccagagcctgtcacagtgtcttggaactccggagccctcacctctggagtgcacacatttcccgccgtgctgcagagcagcggcttgtactctctgagcagcgtggtgacagtgccctctagcagcctgggcacaaagacctacacctgcaacgtggaccacaagccttctaacaccaaggtggataagagagtgggtggcggaggaagcggcggcggaggaagcggcggcggcgggtccgatattcagatgacccagagcccttctacccttagtgcctctgttggagaccgggtgaccatcacctgtaaagcctcccaggacgtgggaacagcagttgcttggtatcagcaaaagcccggcaaggcccctaagttgctgatctactgggcctccacaagacacaccggcgtgcctgatagattcagcggtagcggcagcggcaccgattttaccctgacaatcagctctctgcaggccgaggactttgccgtgtacttctgccaccagcattctagcaatcctctgacttttggccagggcaccaagctggaaatcaagcggacagtagccgctccttctgtatttatcttcccaccttctgacgagcagctgaagtctggtaccgcaagcgtggtgtgcctgctgaacaacttctaccccagagaggccaaagtgcaatggaaggtggacaacgccctgcagagtggcaatagccaggagtctgtcactgagcaggactccaaggatagcacctacagcctgtcttctacactcaccctgtccaaggccgactacgagaagcacaaggtgtacgcctgcgaggtgacacaccagggcctgtcttcccctgtgaccaaaagcttcaaccggggcgagtgcgggggcggcggaagcggtggcggcgggtccggcggcggcggcagcggcggcggcggcagcggaggcggcggcagtggtgggggcggctcgggcggcggaggctctgaggtgcagctggtggaaagtggcggaggcctggtgaagcccggcggcagcctgagactgagttgcgccgcgagcggattcactttctccaactacgccatgtcttgggtgagacaggcccctggcaaaagactggaatgggtcgctaccatcagcaacagaggtagctacacatactaccctgatagcgtgaaaggcaggttcaccatcagcagggacaacgccaagaacagcctgtatctgcagatgaacagcctgcgggccgaagatacagccctttattactgcgcgagagagagacccatggactactggggccagggaacactggtgaccgtttcaagcgcctctaccaagggcccctctgtgtttcctctggccccttgttctcggagcacctccgagagcaccgctgctctgggatgcctcgtgaaggactatttccccgaacccgtgaccgtgtcctggaacagcggcgccctgacaagcggggtccacaccttccccgccgtcctgcagagttctggactgtacagcctgagcagcgtcgtcacagtgccttcaagcagcctgggcaccaagacctacacctgcaacgtggaccataagccttccaataccaaggtggacaagagagtttga

SEQ ID NO: 26 - protein, 967 aa, synthetic construct
  REGION 1..967: note = Synthetic: [alpha-C1s scFab(G4S)3 - alpha-Bb scFab] amino acid (construct #7, FIG. 2A)
MEAPAQLLFLLLLWLPDTTGDIVLTQSPDSLAVSLGERATISCKASQSVDYDGDSYMNWYQQKPGQPPKILIYDASNLESGIPARFSGSGSGTDFTLTISSLEPEDFAIYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSQVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSGGGGSDIQMTQSPSTLSASVGDRVTITCKASQDVGTAVAWYQQKPGKAPKLLIYWASTRHTGVPDRFSGSGSGTDFTLTISSLQAEDFAVYFCHQHSSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSEVQLVESGGGLVKPGGSLRLSCAASGFTFSNYAMSWVRQAPGKRLEWVATISNRGSYTYYPDSVKGRFTISRDNAKNSLYLQMNSLRAEDTALYYCARERPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 27 - DNA, 2904 bp, synthetic construct
  misc_feature 1..2904: note = Synthetic: [alpha-C1s scFab(G4S)3 alpha-Bb scFab-CM] nucleic acid sequence (construct #11, FIG. 2C)
  source 1..2904: mol_type = other DNA
atggaagcccccgcccagctgctgttcctgctgctgctgtggctgcctgacaccactggcgatatcgtgctgacacagagccctgatagcctggctgttagcctgggcgaacgcgccacaatcagctgcaaggccagccagtctgtggattatgatggtgacagctacatgaactggtaccaggagaagcccggacagcctcctaagatcctgatctacgacgccagcaacctggaatccggcattcctgcccggttcagcggctccggcagcggcaccgacttcaccctgaccatctccagcctggaacccgaggatttcgccatctactactgtcagcagagcaatgaggacccatggaccttcggcggcggtaccaaggtcgagatcaagagaacagtggccgctcctagcgtgttcatcttccctccatcagacgagcagctgaagagcggaaccgcttctgtggtgtgtctgctcaacaatttctaccctagagaagccaaggtgcagtggaaggtggacaacgctctccagagcggcaacagccaggagagcgtgaccgagcaagatagcaaggacagcacctactctttaagctctacactgacgctgtccaaggctgactacgagaagcacaaggtgtacgcctgtgaagtgacccaccagggcctgagcagccctgtgacaaagagcttcaacagaggcgagtgcggcggcggaggcagcggcggcggaggctctggcggtggcggaagcggcgggggaggctctggcggcggcggcagtggcggcggcggcagcggaggaggaggatcgcaagtgcaactggtccagtctggcgccgaggtgaaaaagcctggagccagcgtgaaactgtcatgcaccgcctccgggtttaacatcaaagatgactacatccactgggtgaaaaaggctccaggacagggcctggagtggatcggcagaatcgaccctgcggatggccacaccaagtacgccccaaagttccaggtgaaggtgacaatcacagctgacaccagcaccagcacagcctacctggaactgagcagcctaagaagcgaggacaccgccgtgtactactgcgcccggtacggctacggccgggaagtgttcgactactggggtcagggcaccaccgtgacggtgagtagcgcctctacaaaaggcccttccgtgttccccctggccccttgcagccggagcaccagcgagagcaccgccgccttgggctgtctggtgaaagactatttcccagagcctgtcacagtgtcttggaactccggagccctcacctctggagtgcacacatttcccgccgtgctgcagagcagcggcttgtactctctgagcagcgtggtgacagtgccctctagcagcctgggcacaaagacctacacctgcaacgtggaccacaagccttctaacaccaaggtggataagagagtgggtggcggaggaagcggcggcggaggaagcggcggcggcgggtccgatattcagatgacccagagcccttctacccttagtgcctctgttggagaccgggtgaccatcacctgtaaagcctcccaggacgtgggaacagcagttgcttggtatcagaaaaagcccggcaaggcccctaagttgctgatctactgggcctccacaagacacaccggcgtgcctgatagattcagcggtagcggcagcggcaccgattttaccctgacaatcagctctctgcaggccgaggactttgccgtgtacttctgccaccagcattctagcaatcctctgacttttggccagggcaccaagctggaaatcaagcggacagtagccgctcctgctgtatttatcttcccaccttctgacgagcagctgaagtctggtaccgcaagcgtggtgtgcctgctgaagaacttctaccccagagaggccaaagtgcaatggaaggtggacaacgccctgcagagtggcaatagccaggagtctgtcactgagcaggactccaaggatagcacctacagcctgtcttctacactcaccctgtccaaggccgactacgagaagcacaaggtgtacgcctgcgaggtgacacaccagggcctgtcttcccctgtgaccaaaagcttcaaccggggcgagtgcgggggcggcggaagcggtggcggcgggtccggcggcggcggcagcggcggcggcggcagcggaggcggcggcagtggtgggggcggctcgggcggcggaggctctgaggtgcagctggtggaaagtggcggaggcctggtgaagcccggcggcagcctgagactgagttgcgccgcgagcggattcactttctccaactacgccatgtcttgggtgagagaggcccctggcaaaagactggaatgggtcgctaccatcagcaacagaggtagctacacatactaccctgatagcgtgaaaggcaggttcaccatcagcagggacaacgccaagaacagcctgtatctgcagatgaacagcctgcgggccgaagatacagccctttattactgcgcgagagagagacccatggactactggggccagggaacactggtgaccgtttcaagcgcctctaccaagggcccctctgtgtttcctctggccccttgttctcggagcacctccgagagcaccgctgctctgggatgcctcgtgaaggactatttccccgaacccgtgaccgtgtcctggaacagcggcgccctgacaagcggggtccacaccttccccgccgtcctgcagagttctggactgtacagcctgagcagcgtcgtcgaagtgccttcaagcagcctgggcaccaagacctacacctgcaacgtggaccataagccttccaataccaaggtggacaagagagtttga

SEQ ID NO: 28 - protein, 967 aa, synthetic construct
  REGION 1..967: note = Synthetic: [alpha-C1s scFab(G4S)3 alpha-Bb scFab-CM] amino acid sequence (construct #11, FIG. 2C)
MEAPAQLLFLLLLWLPDTTGDIVLTQSPDSLAVSLGERATISCKASQSVDYDGDSYMNWYQEKPGQPPKILIYDASNLESGIPARFSGSGSGTDFTLTISSLEPEDFAIYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSQVQLVQSGAEVKKPGASVKLSCTASGFNIKDDYIHWVKKAPGQGLEWIGRIDPADGHTKYAPKFQVKVTITADTSTSTAYLELSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSGGGGSDIQMTQSPSTLSASVGDRVTITCKASQDVGTAVAWYQKKPGKAPKLLIYWASTRHTGVPDRFSGSGSGTDFTLTISSLQAEDFAVYFCHQHSSNPLTFGQGTKLEIKRTVAAPAVFIFPPSDEQLKSGTASVVCLLKNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSEVQLVESGGGLVKPGGSLRLSCAASGFTFSNYAMSWVREAPGKRLEWVATISNRGSYTYYPDSVKGRFTISRDNAKNSLYLQMNSLRAEDTALYYCARERPMDYWGQGTLVTVSSASTKGPSVFPLAPCSRSTSESTAALGCLVKDYFPEPVTVSWNSGALTSGVHTFPAVLQSSGLYSLSSVVEVPSSSLGTKTYTCNVDHKPSNTKVDKRV

SEQ ID NO: 29 - DNA, 2907 bp, synthetic construct
  misc_feature 1..2907: note = Synthetic: [alpha-Bb scFab(G4S)3 alpha-C1s scFab] nucleic acid sequence (construct #8, FIG. 2A)
  source 1..2907: mol_type = other DNA
atggaagcccccgcccagctgcttttcctgctgctgctgtggctgcctgataccaccggcgatatccagatgacccagagccctagcaccttgagcgcctctgtgggcgacagagtgaccatcacctgcaaggccagccaggacgtgggcacagccgtggcttggtatcagcaaaaacctggcaaggcccctaagctgctgatttactgggccagcaccagacacacaggcgtgcctgaccggtttagcggcagtggcagcgggacagattttaccctgaccatcagctctctgcaggccgaggacttcgctgtgtacttctgccaccagcacagcagcaaccccctgacctttggccagggcaccaagctggagatcaagcggaccgtggccgcacccagtgtgtttatcttcccccccagcgatgagcagctgaagagcggcacagccagcgtggtgtgtctgctgaacaacttctaccctagagaggctaaggtgcagtggaaggtggataatgctctgcagagcggaaatagccaggagtctgtgaccgagcaggacagcaaggactccacatacagcctctcctccaccctgacactgtccaaggccgattacgagaagcacaaagtgtacgcctgcgaggtgacacaccagggccttagcagccctgtcaccaaatctttcaacagaggagagtgcggcggcggcggctccggcggcggcggatctggaggcggaggcagcggaggcgggggaagcggcggaggcggcagcggcggcggaggttccggcggaggcggctcagaggtgcaactcgtggaaagcggtggcggcctggttaagcccggcggcagcctgcgcctgtcatgcgctgcaagcggcttcaccttttcaaattacgccatgagctgggtgcggcaggctcctggaaaacggctggaatgggtggctacaatctctaaccggggctcttacacctactaccccgatagcgtgaaaggcagattcacaatcagccgggacaacgccaagaactcactgtacctgcagatgaactccctgcgggccgaggacacagctctgtactactgtgccagagaaagacccatggactactggggacagggcacactggttacagtctcctctgcctccacgaagggccccagcgtgttccctctggctccttgtagcagaagcacttctgaatctaccgctgccctgggctgcctggtgaaggactacttccctgagcctgtgaccgttagctggaacagcggagccctgacaagcggagtgcatacattccctgccgtgctgcagagcagcggcctctacagcctgtcctcggtggtgaccgtcccctcaagcagcctgggcaccaagacctacacttgcaacgtggaccataagcctagcaacacaaaggtggacaagagagtcggaggcggaggtggctccggcggcggtggctctggcggaggcggcagcgacatcgtgctgacccaaagccctgacagcctggccgtgtccctgggagagcgggccacgatctcctgcaaggcctcccaatccgtggactatgatggcgatagctacatgaactggtaccagcagaagcctggccagcctccaaagatcctgatttacgacgcctctaatctggaatccggcatccctgctagattcagcggaagcggtagcggcaccgacttcaccctgacaatcagcagtctggagccagaggacttcgccatctactactgtcagcagtctaacgaggatccttggaccttcggcggcggcaccaaggtggaaatcaagagaaccgtggccgcccctagcgtcttcatcttccctcctagtgatgagcagctgaaaagcggcacagccagcgtggtgtgcctcctgaacaacttctacccgcgcgaagccaaagtgcagtggaaggtggacaacgccctgcagagcggcaacagccaggagtccgtgacagagcaagatagcaaggacagcacctactccctgtcgtctacacttaccctgtctaaagccgactatgagaagcacaaggtatacgcctgtgaagtgacccaccaggggctgtcctctccagtaaccaagtccttcaacagaggcgaatgcggcggaggcggatctggcggcggcggctccggcggcggcggcagcggcggcggcggcagcgggggcggaggcagcggcggcggaggaagcggaggcggaggcagccaggtgcagctggtgcagtcaggcgctgaggtgaaaaagcctggcgccagcgtcaagctgtcttgcaccgcttctggctttaacatcaaggacgactacatccactgggtcaagcaggcccccgggcaagggctggagtggatcggcagaatcgaccctgccgacggccacaccaagtacgcccctaagttccaggtgaaggtgacaatcacagctgataccagcacgagcaccgcctacctggaactgtcatccctcagatctgaagatacagccgtttactactgcgcaaggtacgggtacgggcgggaagtgttcgactattggggccagggcacaaccgtgaccgtgagcagcgcctctaccaaaggccctagcgtgttccccctggctccttgcagcagatctacaagcgagagcacagccgccctgggatgtctggttaaagattatttcccagaacctgtgacagtgagctggaacagcggcgccctgaccagcggcgtgcacaccttcccagccgtgctgcagtcatccggtctgtatagcctgagcagcgtggttaccgtgcccagctctagcctgggcaccaaaacctacacctgcaatgtggaccacaagccaagcaataccaaggttgataagagagtctga

SEQ ID NO: 30 - protein, 968 aa, synthetic construct
  REGION 1..968: note = Synthetic: [alpha-Bb scFab(G4S)3 alpha-C1s scFab] amino acid sequence (construct #8, FIG. 2A)
MEAPAQLLFLLLLWLPDTTGDIQMTQSPSTLSASVGDRVTITCKASQDVGTAVAWYQQKPGKAPKLLIYWASTRHTGVPDRFSGSGSGTDFTLTISSLQAEDFAVYFCHQHSSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSEVQLVESGGGLVKPGGSLRLSCAASGFTFSNYAMSWVRQAPGKRLEWVATISNRGSYTYYPDSVKGRFTISRDNAKNSLYLQMNSLRAEDTALYYCARERPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGGSGGGGSGGGGSDIVLTQSPDSLAVSLGERATISCKASQSVDYDGDSYMNWYQQKPGQPPKILIYDASNLESGIPARFSGSGSGTDFTLTISSLEPEDFAIYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSQVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 31 - DNA, 2907 bp, synthetic construct
  misc_feature 1..2907: note = Synthetic: [alpha-Bb scFab(G4S)3 - alpha-C1s scFab-CM] nucleic acid sequence (construct #12, FIG. 2C)
  source 1..2907: mol_type = other DNA
atggaagcccctgcccagctgctgttcctgctgctactgtggctgcctgataccaccggcgatatccagatgacgcagagtcccagcaccctgagcgcctctgtgggcgaccgggtgaccatcacctgtaaagcctcccaggacgtgggcacagctgttgcttggtatcagaaaaagcctggcaaggcccctaagctgctgatctactgggccagcacaagacacacaggagtgcctgacagattcagcggcagcggctctgggactgatttcaccttgacaatcagctctctgcaggccgaggactttgccgtgtacttctgccaccaacacagttctaaccccctgaccttcggccaaggaaccaagctggaaatcaagcggaccgtggccgctcctgccgtgttcatcttccctccaagcgatgagcagctgaaaagcggcaccgcgtccgtcgtgtgcctgctgaagaacttctacccgagagaagcgaaggtgcagtggaaagtcgacaacgccctgcagagcggaaatagccaggagagcgtgaccgaacaagactctaaggacagcacctactcgctgtcctccacgctgactctgtctaaggccgactatgagaagcacaaggtgtacgcctgcgaggtgacccaccagggcctgagcagccccgttaccaagagcttcaacagaggagaatgcggcggaggtggcagcggcggcggcgggagcggcggcggcggctcaggcggagggggaagtggcggcggcggcagcggcggcggaggcagcggcggtggcggctctgaggtgcaactggtggaatctgggggcggactggtgaagcctggcggcagtctgagactgagctgtgccgcttccggattcacctttagcaattacgccatgagctgggtgcgggaggcccctggaaagcggctggaatgggttgctacaatcagcaatagaggcagctacacatactaccccgacagtgtcaaaggccggtttacaatcagccgcgacaacgccaaaaacagcctgtacctgcagatgaactccctgcgggctgaggatacagccctctactactgtgccagagaacgtccaatggactattggggccaaggcacactggtgaccgtgagcagcgcgtctaccaagggcccttctgttttccctctggccccctgcagcagaagcacgagcgagagcaccgctgccctgggctgtctggtgaaggattatttccctgagcctgtgaccgtgtcttggaatagcggagccctgaccagcggagtgcatacattccctgctgtgctgcagtctagtgggctgtacagcctgtcttccgttgtggaagtccctagcagcagcctgggcaccaagacctacacctgcaacgtggatcataagccaagcaacaccaaggtggataagagagtgggcggtggcggaggctcgggcggcggcggcagcggcggcggcggcagcgacatcgtgctgacccagtctccagattctctggccgtgtcactgggagagagagccaccattagctgcaaggcctctcagagcgtagactacgacggcgactcctacatgaactggtaccaggaaaagcctggccagcctcctaagatcttgatctacgatgcctccaatctggagagcgggatccccgctagattcagcgggtctggaagtggaaccgacttcacactgaccatctctagcctggagcccgaggactttgccatctactactgccagcagagcaacgaggacccctggacattcggcggcggcacaaaggttgagatcaagagaaccgttgccgctcctagcgtgtttatcttccctccctctgacgagcagctgaagagcggcacagcctccgtggtgtgcctgctgaacaacttctaccccagagaggccaaggtccagtggaaggtcgacaatgcccttcagagcggcaacagccaggagtccgtgaccgagcaggatagcaaggactctacctacagcctgtcctctacgctgaccctgagcaaagccgattacgaaaagcacaaagtgtacgcctgtgaagtgacacaccagggcctgtctagccctgtgacaaagagctttaaccggggcgagtgcggcggcggtggaagcggaggtggaggttcaggaggcggcggaagcggaggcggaggcagtgggggcggcggctccggcggcggcggcagcggaggcggcggttcccaagtgcagctcgtgcagagcggcgccgaggtgaaaaagcccggagccagcgtgaagctgtcttgcaccgcctccggattcaacatcaaagacgactacatccactgggtcaagaaagccccagggcaggggctggagtggatcggcaggatcgaccctgctgatggccacaccaaatacgccccaaagttccaggtgaaagtgacaattaccgcagatacctccaccagcaccgcttatctggaactgagctctctgcggagcgaggacacagccgtgtactactgcgccagatacggctacggcagagaagtgttcgactactggggccagggcaccacagtgacagtgagctctgccagcacaaagggccccagcgtgtttcctctggccccttgcagcagaagcaccagcgagagcaccgccgccctgggctgcctggtgaaggactacttccctgaacccgtgaccgtctcctggaacagtggcgccttgacctctggcgtgcacaccttccccgccgtgctgcagagctccggcctgtacagcctgtctagcgtggtgaccgtgcctagctcgagcctgggcacaaagacatatacctgtaacgtggaccacaagcccagcaacacgaaggtggacaagcgagtgtga

SEQ ID NO: 32 - protein, 968 aa, synthetic construct
  REGION 1..968: note = Synthetic: [alpha-Bb scFab(G4S)3 - alpha-C1s scFab-CM] amino acid sequence (construct #12, FIG. 2C)
MEAPAQLLFLLLLWLPDTTGDIQMTQSPSTLSASVGDRVTITCKASQDVGTAVAWYQKKPGKAPKLLIYWASTRHTGVPDRFSGSGSGTDFTLTISSLQAEDFAVYFCHQHSSNPLTFGQGTKLEIKRTVAAPAVFIFPPSDEQLKSGTASVVCLLKNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSEVQLVESGGGLVKPGGSLRLSCAASGFTFSNYAMSWVREAPGKRLEWVATISNRGSYTYYPDSVKGRFTISRDNAKNSLYLQMNSLRAEDTALYYCARERPMDYWGQGTLVTVSSASTKGPSVFPLAPCSRSTSESTAALGCLVKDYFPEPVTVSWNSGALTSGVHTFPAVLQSSGLYSLSSVVEVPSSSLGTKTYTCNVDHKPSNTKVDKRVGGGGGSGGGGSGGGGSDIVLTQSPDSLAVSLGERATISCKASQSVDYDGDSYMNWYQEKPGQPPKILIYDASNLESGIPARFSGSGSGTDFTLTISSLEPEDFAIYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSQVQLVQSGAEVKKPGASVKLSCTASGFNIKDDYIHWVKKAPGQGLEWIGRIDPADGHTKYAPKFQVKVTITADTSTSTAYLELSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 33 - DNA, 2214 bp, synthetic construct
  misc_feature 1..2214: note = Synthetic: [alpha-C1s scFab(G4S)2 alpha-Bb scFv] nucleic acid sequence (construct #13, FIG. 2D)
  source 1..2214: mol_type = other DNA
atggaagccccagcccagctgctgttcctgctgctgttgtggctgcccgatacaacaggcgacatcgtgctgacccagagccccgactctctggccgtgtccctgggagaaagagccacaatctcctgtaaagcctctcagagcgtggactacgacggcgattcttacatgaactggtaccaacagaaacctggacagcctcctaaaatcctgatctacgacgcctcaaacctggaaagcggcatccctgccagattctcaggctccggtagcggcaccgacttcacactgaccatcagcagcctggaacctgaggacttcgccatctactattgtcagcaaagcaacgaggacccttggaccttcggaggcggcacaaaggtggaaatcaagcggaccgtggcagcaccttctgtcttcatcttccccccatccgatgagcagctgaagagcggcacagctagtgtggtgtgcctgctgaacaacttctacccaagagaagccaaggtgcagtggaaggtggataacgccctgcagtctggtaatagccaggagagcgtgaccgagcaggattctaaggacagcacatacagtctgtctagcacactcaccctgagcaaggccgactacgagaagcacaaggtgtacgcctgcgaggtgacccaccagggcctgtcttctccggtgaccaagtctttcaaccggggcgagtgcggcggcggcggaagcggcggcggcggcagcggcggcgggggcagcggcggcggtgggtctggcggcggcggatcaggcggaggcggcagcggcggaggcggatcccaagtgcagttagttcaaagcggcgctgaggtgaaaaagcctggcgcttctgtgaagctgagctgcaccgccagcggttttaacatcaaggacgactacatccactgggtgaagcaggcccctggccagggactggagtggatcggcagaatcgaccccgctgacggccacaccaaatacgcccctaagttccaggtgaaagtgaccatcaccgctgatacctccacaagcaccgcctacctggaactgtccagcctgagaagcgaggataccgccgtctactactgtgccagatacggctacggcagagaggtgttcgactactggggacaaggcaccaccgtgacagtgtcttctgctagcacgaaaggccctagcgtgtttcctctggctccatgtagcagaagcaccagcgaaagcaccgccgccctgggctgcctggtgaaagactactttcctgagccagtgaccgtgtcctggaactccggagccctcacgtccggcgtgcacacattccccgccgtgctgcagtcatccggcctgtacagcctgagctccgttgtgaccgtgccttcttccagcctgggcacaaagacctacacatgcaacgtggaccacaagcccagcaataccaaggtggacaagagagtgggcggcggcggaagcggcggcggcggcagcgaggtgcagctggtggaatctggcggtggccttgtgaagcctggaggcagcctacggctgagctgcgccgctagcggcttcacctttagcaattacgccatgagctgggtgcggcaggctcctggaaagcggctggagtgggttgcaacaatcagcaatagaggcagctacacctactaccctgactctgttaagggcagatttacaatcagccgcgacaacgccaagaacagcctgtatctgcaaatgaacagcctgagggccgaggacaccgccctgtactactgcgccagagagcggcctatggactattggggacagggcaccctggtcaccgtcagcagcggagggggcggtagcggcggtggaggctctggcggaggaggcagcgacatacagatgacccagagccctagcacactgagcgcctccgttggcgaccgggtgacaattacctgcaaggccagccaggatgtgggcacagccgtggcctggtatcagcagaagcccggcaaggcccctaagctgctgatctactgggccagcaccagacatacaggcgtccccgacagattctctggatcaggcagcggcaccgatttcaccctgactatcagcagcctgcaggccgaagatttcgccgtgtacttctgccaccagcacagctctaaccccctgaccttcggccagggcacaaagcttgaaatcaagtga

SEQ ID NO: 34 - protein, 737 aa, synthetic construct
  REGION 1..737: note = Synthetic: [alpha-C1s scFab(G4S)2 alpha-Bb scFv] amino acid sequence (construct #13, FIG. 2D)
MEAPAQLLFLLLLWLPDTTGDIVLTQSPDSLAVSLGERATISCKASQSVDYDGDSYMNWYQQKPGQPPKILIYDASNLESGIPARFSGSGSGTDFTLTISSLEPEDFAIYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSQVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSEVQLVESGGGLVKPGGSLRLSCAASGFTFSNYAMSWVRQAPGKRLEWVATISNRGSYTYYPDSVKGRFTISRDNAKNSLYLQMNSLRAEDTALYYCARERPMDYWGQGTLVTVSSGGGGSGGGGSGGGGSDIQMTQSPSTLSASVGDRVTITCKASQDVGTAVAWYQQKPGKAPKLLIYWASTRHTGVPDRFSGSGSGTDFTLTISSLQAEDFAVYFCHQHSSNPLTFGQGTKLEIK

SEQ ID NO: 35 - DNA, 2214 bp, synthetic construct
  misc_feature 1..2214: note = Synthetic: [alpha-C1s scFab(G4S)2 alpha-Bb scFv-CM] nucleic acid sequence (construct #14, FIG. 2D)
  source 1..2214: mol_type = other DNA
atggaagcccccgcccagctgctgttcctgctgctcctgtggctgcctgataccaccggcgatatcgtcctgacccagagccctgatagcctggccgtttcactgggcgagcgggccacaatctcctgcaaggcctctcagtctgttgactacgacggcgacagctacatgaactggtaccaggagaaacccggccaacctccaaagatcctgatctacgacgcctctaatctggagagcggcatccccgcccggttcagcgggtccggcagcggcaccgactttaccctgaccatctctagcctggagcctgaggacttcgccatctactactgtcagcagagcaacgaggatccttggacctttggcggcggcacaaaggtggaaatcaagcggaccgtcgccgctccatccgtgtttatcttccctccttccgacgagcagctcaagagcggtaccgccagcgtggtgtgcctgctgaacaacttctaccccagagaggccaaggtgcagtggaaggtagacaacgccttgcagagcggcaactctcaagagagcgtgacagagcaggactctaaggacagcacatacagcctaagctccaccctgaccctcagcaaggccgactacgagaagcacaaggtgtacgcctgtgaagttacacaccagggcctgagcagtccggtgaccaagtccttcaacagaggcgaatgcggcggaggaggctctggcggcggcggcagcggcggaggcggcagcggcggcggaggctctggcggcggtggcagcggaggcggcggaagcggcggaggtggcagccaggtgcagctggtgcagagcggtgctgaagtgaagaaacccggcgcttccgtgaaactgagctgcaccgccagcggatttaacatcaaggacgactacattcactgggtgaaaaaggcccctggccagggcctggaatggatcgggagaatcgaccccgccgatggccataccaagtacgctcctaagttccaggtgaaagtgaccatcaccgctgatacaagcacctctacagcctacctggagctgagctccctgcggtctgaggacaccgccgtgtactactgcgccagatacggctacggcagagaggtgttcgactactggggacagggcactacagtcaccgtgtctagtgctagcacgaagggccctagcgtgttccctctggctccatgtagcagaagcaccagcgaaagcacagctgctctgggctgcctggtgaaagactacttccccgagcctgtgaccgtcagctggaactccggcgccctgaccagcggagtgcacacctttcctgctgtgctgcaatcctctggcctgtactctctgagctctgttgtgacagtgccttctagcagcctgggaaccaagacctacacctgcaacgtggaccacaagcccagcaacaccaaggtggataagcgcgtgggcggcggcggatctggcggaggcggcagcgaggtgcagctggtggaaagcggcggcggcctggtgaagcctggcggctcactgagactgagctgtgccgccagcggcttcaccttctccaactacgccatgagctgggtgcgggaagccccaggaaagcgcctggagtgggtcgccaccatcagcaatagaggctcgtatacatattaccctgattccgtcaaaggcagattcaccatctctagagataatgccaagaacagcctgtacctgcagatgaactccctcagagccgaggatacagccctgtattactgcgccagagaacggcctatggactactggggccaaggcactctggtgacagtgagcagcggcggcggtggttccggcggcggaggctctggaggaggcggcagcgacatccagatgacccagagccctagcaccctgtccgccagcgtgggagatagagtgaccattacctgtaaagcgagccaggatgtgggcaccgccgtggcctggtatcagaagaagcctggcaaggcccctaagctgctgatctactgggcctctacccggcacacaggcgtgcccgacagattctccggctccggttctggaacagacttcacactgaccatcagctctcttcaggccgaggacttcgccgtgtacttctgccaccagcacagctctaatcctctgacattcggccaaggcacaaagctggaaatcaagtga

SEQ ID NO: 36 - protein, 737 aa, synthetic construct
  REGION 1..737: note = Synthetic: [alpha-C1s scFab(G4S)2 alpha-Bb scFv-CM] amino acid sequence (construct #14, FIG. 2D)
MEAPAQLLFLLLLWLPDTTGDIVLTQSPDSLAVSLGERATISCKASQSVDYDGDSYMNWYQEKPGQPPKILIYDASNLESGIPARFSGSGSGTDFTLTISSLEPEDFAIYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSQVQLVQSGAEVKKPGASVKLSCTASGFNIKDDYIHWVKKAPGQGLEWIGRIDPADGHTKYAPKFQVKVTITADTSTSTAYLELSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSEVQLVESGGGLVKPGGSLRLSCAASGFTFSNYAMSWVREAPGKRLEWVATISNRGSYTYYPDSVKGRFTISRDNAKNSLYLQMNSLRAEDTALYYCARERPMDYWGQGTLVTVSSGGGGSGGGGSGGGGSDIQMTQSPSTLSASVGDRVTITCKASQDVGTAVAWYQKKPGKAPKLLIYWASTRHTGVPDRFSGSGSGTDFTLTISSLQAEDFAVYFCHQHSSNPLTFGQGTKLEIK

SEQ ID NO: 37 - DNA, 3869 bp, synthetic construct
  misc_feature 1..3869: note = Synthetic: [alpha-C1s scFab - bidirectional promoter - alpha-Bb scFab] nucleic acid sequence (construct #9, FIG. 2B)
  source 1..3869: mol_type = other DNA
tcacacccgcttatccaccttggtgttgctgggcttgtggtccacgttgcaggtgtaggtctttgtgcccaggctagagctaggcactgtcacgacagaggacagagagtacaggccgctgctctgcagcacggcggggaaggtgtgcaccccgcttgtcagggctccgctgttccaggacacggtcacaggctcagggaaataatccttgaccaggcagcccagagcagccgtgctctctgaggtacttctgctacaaggagccagtgggaacacgctagggccctttgtgctggcggacgacacggtcactgttgtgccctgtccccagtagtcgaacacttctctgccgtagccgtatctggcgcagtagtacacagcggtgtcctcggatctaaggctgctcagttccagataagctgtagaggtgctggtatcggcggtgatggtgactttcacctggaacttaggggcgtactttgtgtggccgtcggcagggtcgattctgccgatccactccagtccctggccgggggcctgcttcacccagtggatgtaatcgtccttgatattgaagccgctggcggtgcagctcagcttaacactagcgccaggctttttcacctcggctccgctctgcaccagctgcacctgggatccgccgccgccgctgccgcctccgccgctgccgcctccgccgcttccgcctcccccagagccgccgccaccgctgcctcctccgccggagccgccgccgccgcactcgccccggttgaagcttttggtcacaggagaggacaggccctgatgtgtcacttcacaggcgtacaccttgtgcttctcgtagtcggccttgctcaaggtcagggtgctggacaggctgtatgttgagtccttgctgtcctgctcggtcacgctctcttggctgttgccgctttgcagggcgttgtcaactttccattggacctttgcctctctggggtagaagttattcagcaggcacaccacagaggcggttccgctcttcagctgctcgtcgcttggagggaagataaagacagaaggggcggccacggtgcgcttgatttccaccttggtgccgcctccaaaggtccaggggtcctcgttgctctgctggcagtagtagatggcaaaatcctcgggttccagagaagaaattgtcagggtgaaatcagtgccagagccgctgccgctgaatctggcggggatgccgctttccagattgctggcgtcgtagatcaggatttttggaggctggccgggtttctgctggtaccagttcatgtagctgtcgccgtcatagtccacgctctgagaggctttacagctgattgtggcccgttcgccgaggctcacggccaggctatcagggctctgcgtcagcacgatatcgccggtggtgtcaggcagccacaggagcagcaggaacagcagctgggcaggggcttccatggtgggctctggcgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgccaactagcccaccatggaagcccccgctcagctgctgttcctgctgctgctgtggctgcctgacaccaccggcgacatccagatgacacagagccctagcaccctgagcgcctccgtgggggacagagtgacaatcacatgtaaagcctcccaggacgtgggcactgccgtggcctggtaccagcaaaaaccgggaaaagcccctaagctgctgatctactgggccagcaccagacacaccggcgtccccgatagattcagcggctctggcagcggaactgatttcaccctgaccatttcttctctgcaggccgaggacttcgccgtgtacttttgccaccagcacagcagcaaccctctgaccttcggacagggcacaaagctggaaatcaagcggacagtggctgctccttctgtgttcatctttccacctagcgacgagcagctgaagagcggcaccgcctctgtggtgtgcctgctgaacaacttctaccccagagaagccaaagtgcagtggaaggtggacaacgccctgcaatctggcaacagccaggagagcgtgacggaacaagatagcaaggacagcacctactccctgagcagcacactgaccttgtccaaggcagattacgagaagcacaaggtgtacgcctgcgaggtgacccaccagggactgagcagcccagtgaccaagagcttcaacagaggagagtgcggcggcggcggaagcggaggcggaggcagcggcggcggcggcagtggaggcggcggctctggcggagggggcagtggcggtggcggatccggcggcggcggcagcgaggtgcagcttgtggaatccggcggcggcctggtgaagcccggcggtagcctgagactgtcttgtgccgcctctggcttcacctttagcaattacgccatgagctgggtgcggcaggctcccggcaaaagactggaatgggtcgccaccatcagcaaccggggatcatatacctactaccctgatagcgtgaaaggcaggttcacaatcagccgggacaatgccaagaacagcctgtacctgcagatgaactcactgcgggccgaggacaccgccctgtattactgcgccagagagagacctatggactactggggccagggcaccctggtgaccgtttcctccgccagcaccaagggccctagcgtgttccctctggccccatgcagcagaagcacatctgagagcaccgccgctctgggctgcctggtgaaggactacttccccgagcctgtgacagtgagctggaactccggcgccctgaccagcggcgtgcacacatttccagctgtgctgcagtctagcggcctgtacagcctgagcagcgttgtgacagtgccttctagcagcctcggcaccaagacctacacctgtaacgtggatcataagccttctaataccaaggttgacaagagagtgtga

SEQ ID NO: 38 - DNA, 2955 bp, synthetic construct
  misc_feature 1..2955: note = Synthetic: [alpha-C1s F2A Fab(G4S)3 alpha-Bb scFab] nucleic acid sequence (construct #17, FIG. 2F)
  source 1..2955: mol_type = other DNA
atggaagccccagctcagctgctgttcctgctgctgctgtggctgcccgacaccaccggcgacatcgtgctgacccagagccctgatagcctggccgtttctctgggagaacgggcaaccattagctgcaaggccagccagtctgtggactacgacggcgacagctacatgaattggtatcagcagaagcctggccaacctcccaagatcctgatctacgatgccagcaacctggaatccggaatccccgcccgcttcagcggcagcggctcaggcaccgacttcaccctgacaatctcctcgctggaacccgaggatttcgctatctactactgtcagcagtctaacgaggatccttggaccttcggcggcggcacaaaggtcgagatcaagagaacagttgccgccccttctgtgtttatcttccctccctctgacgagcagctgaagagcggcactgccagcgtcgtgtgcctgctgaacaacttctacccacgtgaggccaaagtccaatggaaagtggataacgccctgcagagcggcaactctcaggagtctgtgacagagcaggacagcaaagatagcacctactctctgtctagcaccctgaccctgagcaaggccgattacgagaagcacaaagtgtacgcctgcgaggtgacccaccagggcctgagcagccccgtgacaaagtccttcaacaggggcgagtgtcggaagagacggagcggcagcggcgccccagtcaagcagaccctgaacttcgacctgcttaagctggccggcgatgtagaaagcaatcctggccccatggaagcccctgcccagctgctgttcctgctgctgctgtggctgcctgacaccacaggacaagtgcaactagtgcagtcaggcgccgaggtaaaaaagcctggcgccagcgtgaaactgtcttgcaccgcctccggcttcaatatcaaggacgactacatacactgggtgaagcaggctcccggccagggcctggaatggatcggccgcatcgaccctgctgacggccacaccaagtatgcccctaagttccaggtcaaagtgaccatcaccgctgataccagcacaagtacagcctacctggaactgagcagcctgcggagcgaggacacagccgtgtactactgcgcccggtacggctatggcagagaggtgttcgactactggggacagggcaccaccgtgacagtgtctagcgcctctacaaagggccctagcgtgttcccgctggccccctgcagcagaagcacatctgaaagcacagcagctctcggctgcctcgtcaaggactactttcctgagccggtgacagttagctggaacagcggcgccctgactagcggcgtgcatacattccctgccgtgctgcagtcctccggcctctacagcctgtccagcgtggtgacagtcccttcttccagtctgggtacgaaaacctacacctgcaacgtggaccacaagccctccaatacgaaagtggacaagagagtgggcgggggaggctctggcggaggtggctctggcgggggcggaagcgacatccagatgacacaatcccctagcaccctgagcgccagcgtgggagatagagtgacgatcacctgtaaagcctcacaggacgtgggcaccgccgtggcctggtaccagcagaaacctggaaaggcccctaagctgctgatctactgggcctccaccagacacaccggcgtgcctgacagattcagcggctctggcagcggcacagactttaccctgacaatcagcagcctgcaggctgaagatttcgccgtgtacttctgccaccaacacagcagcaaccccctgacatttggccaaggcaccaagctggagatcaagagaaccgttgctgcccctagcgtgttcatcttcccgcctagcgacgagcagctgaagagcggcaccgcctctgtggtttgcctgctgaacaacttctaccccagagaagccaaagtgcagtggaaggtggacaacgccctgcagagtggaaactctcaagagagcgtgaccgaacaggatagcaaagacagcacctatagcttgtctagcacactgaccctgtctaaggctgactacgagaagcacaaggtgtacgcatgcgaggtcacccatcagggactgagcagccccgtgaccaagtcttttaaccggggcgagtgcggcggaggaggcagtggcggcgggggatccggcggcggcggcagcggcggaggcggatccggcggcggcggtagcggcggtggcggcagcggtggagggggaagcgaggtgcagctcgtcgagtccggaggaggccttgtgaagcctggcggcagcctgagactgagctgcgccgccagcggattcaccttcagcaattacgccatgagctgggtgcggcaggcccctggcaagagactggaatgggtggccaccatcagcaacagaggcagctacacctactaccccgactccgtgaagggcagatttaccatcagccgggacaacgccaagaacagcctgtacctgcagatgaactccctgagagccgaggacaccgccctgtactactgtgccagggaaagacctatggactactggggccagggaacactggtgaccgtatcttccgcctcaaccaaaggcccctcggtgtttccactggctccttgctccagatccacctccgagagcaccgccgccctgggctgtctggtgaaggattacttcccagaacctgtgaccgtgagctggaatagcggcgctctcacctctggagtgcacaccttccctgccgtgctgcagagcagcggcctgtatagcttgtccagtgtggtgaccgtgcctagctccagcctgggcactaagacatatacatgtaacgtggaccacaagcctagcaacaccaaggtggataagagagtgtga

SEQ ID NO: 39 - protein, 984 aa, synthetic construct
  REGION 1..984: note = Synthetic: [alpha-C1s F2A Fab(G4S)3 alpha-Bb scFab] amino acid sequence (construct #17, FIG. 2F)
MEAPAQLLFLLLLWLPDTTGDIVLTQSPDSLAVSLGERATISCKASQSVDYDGDSYMNWYQQKPGQPPKILIYDASNLESGIPARFSGSGSGTDFTLTISSLEPEDFAIYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECRKRRSGSGAPVKQTLNFDLLKLAGDVESNPGPMEAPAQLLFLLLLWLPDTTGQVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSGGGGSDIQMTQSPSTLSASVGDRVTITCKASQDVGTAVAWYQQKPGKAPKLLIYWASTRHTGVPDRFSGSGSGTDFTLTISSLQAEDFAVYFCHQHSSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSEVQLVESGGGLVKPGGSLRLSCAASGFTFSNYAMSWVRQAPGKRLEWVATISNRGSYTYYPDSVKGRFTISRDNAKNSLYLQMNSLRAEDTALYYCARERPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 40 - DNA, 2934 bp, synthetic construct
  misc_feature 1..2934: note = Synthetic: [alpha-C1s GT2A Fab(G4S)3 alpha-Bb scFab] nucleic acid sequence (construct #18, FIG. 2F)
  source 1..2934: mol_type = other DNA
atggaagccccagcccagctgctgtttctgctgctgttgtggctgcccgatactaccggcgatatcgtgctgacccagagccctgatagcctggctgtgtctctgggggagcgggctaccatctcttgtaaagccagccaaagcgtggactacgacggcgactcctacatgaactggtaccagcagaaacctggccagcctccaaagatcctgatctacgacgccagcaacctggaaagcggcatccctgctcggttcagcggatcaggctcgggcacagactttacactgacaattagctctctggaacctgaagattttgctatctactattgccagcagagcaacgaggatccttggacctttggcggcggaacaaaggtggaaatcaagcggacagtcgctgcccctagtgtgttcatcttcccaccttccgatgagcagctcaagtctggaacagcctctgtggtctgcctgctgaacaacttctacccccgggaggctaaagtgcagtggaaggtggataacgccctgcagtctggcaactcgcaggagagcgttacagagcaggactctaaggacagtacctacagcctgtcatcaaccctgaccctgagcaaggccgactatgaaaagcacaaggtctacgcctgcgaggtgacacaccagggcctgagctctcctgtgactaagtccttcaatagaggagagtgcagacggaagcgcggcagcggagaaggcagaggctccctgctaacctgtggagacgtggaggaaaaccccggccccatggaagcccctgctcagctgctgttcctgctgctgctgtggctgccggatacaaccggacaagtgcagctggtgcaatctggcgccgaagtgaaaaagcccggcgcttctgtgaagctgtcttgcaccgcctctggattcaacatcaaggacgactacatccactgggtgaagcaggcccctggccagggcctggagtggatcggcagaatcgaccccgctgatggccacacaaaatacgcccctaagttccaggtgaaggtgaccatcaccgctgacacctcgacaagtaccgcctacctggagctgagctctctgagatccgaggacacagcagtgtactactgcgccagatacggctacggcagagaggttttcgactactggggccagggcaccaccgtgaccgtgtccagcgccagcacaaagggcccttctgtcttccctctggcgccttgtagccggagcacaagcgagagcactgccgctcttggctgcctggtgaaggactactttcctgaacctgttacagtgagctggaacagcggcgccctgacatctggcgtgcacacctttccagccgtgctgcagtcctccggcctgtacagtctgagcagcgtggtgaccgtgcctagcagctctctgggcaccaagacatatacctgcaatgtggaccacaaacctagcaacaccaaggtggacaagagagtgggcggcggcgggagtggaggtggaggcagcggaggtggtggcagcgacatccagatgacacagagccctagcactctgagcgccagcgtgggcgatagagtgaccattacctgcaaggcctcccaggacgtgggaaccgccgtggcctggtatcagcaaaagccaggcaaggcccccaagcttctgatctactgggccagcacaagacacaccggcgtccccgacaggttcagcggcagtggctcaggcaccgacttcaccctaactatcagctctctgcaagctgaagacttcgccgtgtacttctgccaccagcacagctccaaccccttgaccttcggccaaggcacaaagctggaaatcaaacggacagtcgccgcacctagcgtgttcatcttcccaccttctgacgagcagctgaagagcggcaccgcgtccgtggtgtgtctgctcaacaacttctacccaagagaggccaaggtgcagtggaaggttgacaatgccctgcagagcgggaatagccaggagagcgtgaccgagcaggacagcaaggactctacctacagcctcagttctaccctgaccctgtccaaggccgattacgagaagcacaaggtgtacgcctgtgaagtgacccatcagggcctgagcagtcctgtgactaaaagcttcaacagaggcgaatgcggcggcggaggctccggcggaggcggcagcggcggaggcggatctggcggcggtggctccggaggcggcggcagcggcggcggcggctctggcggcggcggctctgaggtgcaactggttgaaagcggaggcggcctggtgaagcccggaggctccctgcggctgagctgcgccgccagtggcttcaccttctctaattacgctatgagctgggtcagacaggcccctggaaagcggttggagtgggtggccaccatcagcaaccggggaagctacacctactacccagatagcgtgaaaggcaggtttaccatcagcagagataacgccaagaactcactgtacctgcagatgaacagcctgagagccgaggacaccgccctgtactactgcgccagagagagacctatggactactggggccaaggcacattagtcaccgtgtcctctgccagtaccaagggccctagcgtgttccctctggccccttgctccagaagcaccagcgagagcacagccgcacttggatgtctggttaaagattatttccccgagcccgtgacagtgtcttggaacagcggggccctgaccagcggtgttcataccttccctgctgtgctccagagctccggcctgtattccctgagttcagtagtgaccgtgcctagcagcagcctgggaaccaagacctacacatgcaacgtggaccacaagcctagcaataccaaggtggacaagcgggtgtga

SEQ ID NO: 41 - protein, 977 aa, synthetic construct
  REGION 1..977: note = Synthetic: [alpha-C1s GT2A Fab(G4S)3 alpha-Bb scFab] amino acid sequence (construct #18, FIG. 2F)
MEAPAQLLFLLLLWLPDTTGDIVLTQSPDSLAVSLGERATISCKASQSVDYDGDSYMNWYQQKPGQPPKILIYDASNLESGIPARFSGSGSGTDFTLTISSLEPEDFAIYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECRRKRGSGEGRGSLLTCGDVEENPGPMEAPAQLLFLLLLWLPDTTGQVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSGGGGSDIQMTQSPSTLSASVGDRVTITCKASQDVGTAVAWYQQKPGKAPKLLIYWASTRHTGVPDRFSGSGSGTDFTLTISSLQAEDFAVYFCHQHSSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSEVQLVESGGGLVKPGGSLRLSCAASGFTFSNYAMSWVRQAPGKRLEWVATISNRGSYTYYPDSVKGRFTISRDNAKNSLYLQMNSLRAEDTALYYCARERPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 42 - DNA, 2955 bp, synthetic construct
  misc_feature 1..2955: note = Synthetic: [alpha-C1s scFab(G4S)3 alpha-Bb F2A Fab] nucleic acid sequence (construct #19, FIG. 2F)
  source 1..2955: mol_type = other DNA
atggaagccccagcccagctgctgttcctgctgctgctgtggctgcccgataccaccggcgacatcgtgctgacacagagtcctgatagcctggccgtgtctctgggggaaagagccacaatctcttgcaaggcctcccagagtgtagactacgacggcgatagttacatgaactggtatcagcagaaacctggacaacctccaaagatcctgatctacgacgccagcaacctggagagcggcattcctgcccggttcagcggcagcggcagcggcaccgacttcaccctgacaatcagcagcctggagcccgaggactttgccatctactactgtcagcaaagcaacgaggacccctggacatttggcggcggcacgaaagtggaaatcaagcggaccgtcgccgcccccagcgtgttcatcttccctccttctgatgagcagctcaagagcggcacagccagcgtggtgtgcctgctgaacaatttctaccctagggaagccaaggtgcagtggaaggtggacaatgccctgcaaagcggcaactctcaggagtccgttaccgagcaagatagcaaggactctacatattctctgtctagcaccctgaccttgagcaaggccgactatgaaaagcacaaggtctacgcatgcgaggtgactcatcagggcctcagctccccagtgaccaaatccttcaaccggggcgagtgcggcggaggcggcagcgggggcggaggcagcggaggaggcggctcaggcggaggaggcagcggcggcggcggctcgggcggaggcggaagcggcggcggcggcagccaagtgcagctggtgcagagcggcgctgaagtgaaaaagcctggcgccagcgtgaagctgtcctgcaccgccagcggcttcaatatcaaggatgattacatccactgggtgaaacaggcccctggccagggccttgagtggatcggaaggatcgaccctgccgatggccacaccaagtacgctcccaagttccaggtgaaggtgaccatcaccgccgataccagcacgagcacagcctacctggaactgtcttccctgagaagcgaagataccgccgtgtactactgcgccagatacggatatggcagagaggtattcgactactggggacagggcaccaccgtgaccgtgtcctctgcctccaccaagggcccctctgtgtttcctctggccccctgctctagaagcaccagcgagagcacagccgccctgggctgtctggtgaaagactatttccctgagcccgtgaccgtgtcctggaacagcggcgccctgacaagtggcgtgcacacctttcctgctgttctgcagagtagcggcctgtacagcctgtcgagcgtggtcacagtgcctagcagcagtctgggcacaaagacctacacttgtaacgtggatcacaagccctctaataccaaggtggacaagcgggtgggaggcggcggaagcggaggcggcggctctgggggaggtggctcagaagtgcagctggtggaaagcggcggcgggctggtgaagcctggcggctctctccggctgagctgtgccgccagcggttttaccttctccaattacgccatgagctgggtcagacaggccccaggcaagagacttgagtgggttgctacaatcagcaacagaggcagctacacctactaccctgacagcgtgaagggcagattcacaatcagccgggacaacgccaagaacagcctgtacctgcagatgaacagcctgagagccgaggatacagccctttactactgtgccagagagagacctatggactactggggccagggcactctggtgaccgtttccagcgccagcaccaaaggcccaagcgtgttccctctggctccctgcagcagaagcaccagcgaaagcacagctgcgctgggctgcctggtgaaggattacttccccgagcctgtgaccgtgtcttggaactccggcgctctgacatccggcgttcacacattccccgctgtcctgcagtcaagtggcctgtacagcctgagcagtgtggtgaccgttccaagctcttctctgggaacaaaaacatacacctgcaacgtggaccacaagcctagcaacaccaaagtggataagcgggtgcggaagcgccggagcggaagcggcgcccctgtgaagcagaccctgaacttcgacctgctgaagctggctggcgacgtggaaagcaaccctggccctatggaagcccccgcacaactgctgttcctgctgctgctctggctgcctgacaccacaggcgacatccagatgacccaaagccctagcacactgagcgccagcgtcggcgacagagtgaccattacatgcaaggcctcccaggacgtcggcacagccgtggcctggtaccagcagaagcctggaaaggccccaaagctgctgatctactgggcctctacccggcataccggcgtgcctgacagattcagcggcagcggctctggtacagacttcaccctgaccattagcagcttacaggccgaggacttcgccgtgtacttctgccaccagcacagcagcaatcctctaaccttcggccagggaaccaagctggaaatcaaaagaaccgtggccgccccttctgtattcatatttcctccaagcgacgagcagctcaagagcggcacggcttctgtggtgtgtctgctgaacaacttttatcccagagaagccaaggtgcagtggaaggtggataacgccctgcaatccggaaactctcaggagtctgtcaccgagcaggactcaaaggactcgacgtacagcctgagcagcacactgaccctgagcaaggccgactacgagaagcacaaagtttacgcctgcgaggtgacacaccagggcctctctagccctgtgacaaagagcttcaacaggggcgagtgctga

SEQ ID NO: 43 - protein, 984 aa, synthetic construct
  REGION 1..984: note = Synthetic: [alpha-C1s scFab(G4S)3 alpha-Bb F2A Fab] amino acid sequence (construct #19, FIG. 2F)
MEAPAQLLFLLLLWLPDTTGDIVLTQSPDSLAVSLGERATISCKASQSVDYDGDSYMNWYQQKPGQPPKILIYDASNLESGIPARFSGSGSGTDFTLTISSLEPEDFAIYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSQVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSGGGGSEVQLVESGGGLVKPGGSLRLSCAASGFTFSNYAMSWVRQAPGKRLEWVATISNRGSYTYYPDSVKGRFTISRDNAKNSLYLQMNSLRAEDTALYYCARERPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVRKRRSGSGAPVKQTLNFDLLKLAGDVESNPGPMEAPAQLLFLLLLWLPDTTGDIQMTQSPSTLSASVGDRVTITCKASQDVGTAVAWYQQKPGKAPKLLIYWASTRHTGVPDRFSGSGSGTDFTLTISSLQAEDFAVYFCHQHSSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - DNA, 2934 bp, synthetic construct
  misc_feature 1..2934: note = Synthetic: [alpha-C1s scFab(G4S)3 alpha-Bb GT2A-Fab] nucleic acid sequence (construct #20, FIG. 2F)
  source 1..2934: mol_type = other DNA
atggaagcccctgcccagctgctgtttctgctgctgctgtggctgcctgacaccacaggcgacatcgttctgacccagagccctgacagcctggccgtgtccctaggcgaacgggccaccatcagctgcaaggccagccagagcgtggactatgatggcgacagctacatgaactggtatcagcaaaagcccggacagcctcctaagatcctgatctacgacgcctctaacctggaatctggcatccctgccagattttctggcagcggttctggcaccgatttcaccctgaccattagctctctggagcctgaggacttcgccatctactactgccagcagagcaacgaggatccttggacattcggcggcggtaccaaggtcgagattaaacggaccgtggctgctcccagcgtgttcatcttcccaccatctgatgagcagctgaaatctggcacggccagcgtcgtgtgcctgctgaacaacttctaccctagagaggccaaggtgcagtggaaggtggataacgccctgcagtccggcaatagccaggagagcgtgactgaacaggatagtaaagactctacctacagcctgtccagtacactgaccctgtctaaggccgattacgagaagcacaaagtgtacgcctgtgaagtgacacatcagggcctgagctcacctgtgactaagtccttcaaccggggcgagtgcggcggcggtggcagcggcggcggcggcagcggaggcggcggcagtggaggcggcgggtctggcggaggtggatctggtggcggcggtagcggcggcggcggcagccaggtgcaactggtgcagtctggagctgaggtgaagaaacctggggccagcgtgaagctgtcttgcaccgccagcggcttcaacatcaaggacgactacatccactgggtcaaacaggctcctggacagggcttggaatggatcggcagaatcgaccccgccgacggccacaccaagtacgccccaaaattccaggtgaaagtaacaatcaccgctgatacatctacttccacagcttatctggaactgagcagcctgaggtctgaggataccgccgtgtactactgcgcccggtacggctacggcagagaggtgttcgactactggggacagggcaccaccgtgaccgtgtcttccgccagcactaagggacctagcgtgttccccctggccccatgttcccggagcaccagcgagtctactgccgccctgggatgcctggtgaaggactactttcctgagcccgtgaccgtgtcttggaacagcggcgccctgaccagcggcgtgcacacattccctgccgtgctgcagagcagcggcctgtacagcctgtcctctgtggtgacagtgccctctagctctctcggcaccaaaacctacacctgcaacgtggaccataagcctagcaacaccaaggtcgacaagcgggtgggcggcggcgggagtggcggtggcggctctggcggaggggggagcgaagtgcagctggtcgaaagcggaggaggactagtgaagcctggcggcagcctgagactgagctgtgctgccagcggctttacattcagcaactacgccatgagctgggtgcgtcaggcccccggcaagcggctggaatgggtcgcaaccatcagcaatagaggcagctacacttactaccctgactccgtcaagggcagattcaccatctcccgcgacaacgccaaaaactccctgtacctgcaaatgaatagcctgagagccgaggacaccgccctgtactattgcgccagagagagacctatggactactggggccagggtaccctggtgaccgtgagctctgctagcacaaagggcccttccgtgttccctctggctccttgcagcagaagcacaagcgagagcacagccgccctgggctgcctggttaaggactattttcccgaacctgtgacagtctcctggaacagcggcgccctgacctctggggtgcacaccttccccgctgtcctgcagagcagcggcctgtactcgctgagctctgtggtgaccgtgcctagcagcagcctgggcaccaagacatacacatgtaatgtggaccacaagccctccaacaccaaggtcgataagagagtgcggagaaagagaggttccggcgagggcagaggcagcctgttaacatgcggcgacgtggaggaaaacccaggacctatggaggcccccgcccagctgctcttcctgctgctgctgtggctgcccgataccaccggcgatatccagatgacacagtccccttcaacccttagtgcctcggttggcgatagagtgacaattacatgtaaagctagccaggacgtgggcaccgccgtggcctggtaccagcagaagcccggcaaagccccaaagctgctcatctactgggcctcgacaagacacaccggcgtgccagatagattcagcggctctggctcaggcacagacttcaccctgactatcagctccctccaagccgaggatttcgccgtttacttctgccaccagcacagctccaatcccctgacattcggccaaggaaccaagctggaaatcaagcggaccgtggccgctcctagtgtcttcatcttccctccttccgacgagcagctgaagagcggcacagcctccgtggtgtgtctgctcaacaacttttaccccagagaggccaaggtgcagtggaaggtggacaatgccctgcagagcggaaacagccaggagtcggtgaccgagcaagacagcaaggactctacgtacagcctgtcaagcaccctgacgctgagcaaggccgattacgagaagcacaaagtgtacgcctgcgaggtgacccaccagggactgagcagccctgtgaccaagagctttaaccgtggagaatgctga

SEQ ID NO: 45 - protein, 977 aa, synthetic construct
  REGION 1..977: note = Synthetic: [alpha-C1s scFab(G4S)3 alpha-Bb GT2A-Fab] amino acid sequence (construct #20, FIG. 2F)
MEAPAQLLFLLLLWLPDTTGDIVLTQSPDSLAVSLGERATISCKASQSVDYDGDSYMNWYQQKPGQPPKILIYDASNLESGIPARFSGSGSGTDFTLTISSLEPEDFAIYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSQVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSGGGGSEVQLVESGGGLVKPGGSLRLSCAASGFTFSNYAMSWVRQAPGKRLEWVATISNRGSYTYYPDSVKGRFTISRDNAKNSLYLQMNSLRAEDTALYYCARERPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVRRKRGSGEGRGSLLTCGDVEENPGPMEAPAQLLFLLLLWLPDTTGDIQMTQSPSTLSASVGDRVTITCKASQDVGTAVAWYQQKPGKAPKLLIYWASTRHTGVPDRFSGSGSGTDFTLTISSLQAEDFAVYFCHQHSSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 50 aa, synthetic construct
  REGION 1..50: note = Synthetic: linker
  VARIANT 6..50: note = "GGGGS" may or may not be present
GGGGSGGGGSGGGGSGGGGSGGGGSGGGGSGGGGSGGGGSGGGGSGGGGS

SEQ ID NO: 47 - protein, 10 aa, synthetic construct
  REGION 1..10: note = Synthetic: linker
GGGGSGGGGS

SEQ ID NO: 48 - protein, 15 aa, synthetic construct
  REGION 1..15: note = Synthetic: linker
GGGGSGGGGSGGGGS

SEQ ID NO: 49 - protein, 35 aa, synthetic construct
  REGION 1..35: note = Synthetic: linker
GGGGSGGGGSGGGGSGGGGSGGGGSGGGGSGGGGS

SEQ ID NO: 50 - DNA, 4641 bp, synthetic construct
  misc_feature 1..4641: note = Synthetic: Nucleotide sequence of AAV2#9 (FIGs. 2B and 2I)
  source 1..4641: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacaattctagttccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacaggtttaaaccctgcagggagctctcacacccgcttatccaccttggtgttgctgggcttgtggtccacgttgcaggtgtaggtctttgtgcccaggctagagctaggcactgtcacgacagaggacagagagtacaggccgctgctctgcagcacggcggggaaggtgtgcaccccgcttgtcagggctccgctgttccaggacacggtcacaggctcagggaaataatccttgaccaggcagcccagagcagccgtgctctctgaggtacttctgctacaaggagccagtgggaacacgctagggccctttgtgctggcggacgacacggtcactgttgtgccctgtccccagtagtcgaacacttctctgccgtagccgtatctggcgcagtagtacacagcggtgtcctcggatctaaggctgctcagttccagataagctgtagaggtgctggtatcggcggtgatggtgactttcacctggaacttaggggcgtactttgtgtggccgtcggcagggtcgattctgccgatccactccagtccctggccgggggcctgcttcacccagtggatgtaatcgtccttgatattgaagccgctggcggtgcagctcagcttaacactagcgccaggctttttcacctcggctccgctctgcaccagctgcacctgggatccgccgccgccgctgccgcctccgccgctgccgcctccgccgcttccgcctcccccagagccgccgccaccgctgcctcctccgccggagccgccgccgccgcactcgccccggttgaagcttttggtcacaggagaggacaggccctgatgtgtcacttcacaggcgtacaccttgtgcttctcgtagtcggccttgctcaaggtcagggtgctggacaggctgtatgttgagtccttgctgtcctgctcggtcacgctctcttggctgttgccgctttgcagggcgttgtcaactttccattggacctttgcctctctggggtagaagttattcagcaggcacaccacagaggcggttccgctcttcagctgctcgtcgcttggagggaagataaagacagaaggggcggccacggtgcgcttgatttccaccttggtgccgcctccaaaggtccaggggtcctcgttgctctgctggcagtagtagatggcaaaatcctcgggttccagagaagaaattgtcagggtgaaatcagtgccagagccgctgccgctgaatctggcggggatgccgctttccagattgctggcgtcgtagatcaggatttttggaggctggccgggtttctgctggtaccagttcatgtagctgtcgccgtcatagtccacgctctgagaggctttacagctgattgtggcccgttcgccgaggctcacggccaggctatcagggctctgcgtcagcacgatatcgccggtggtgtcaggcagccacaggagcagcaggaacagcagctgggcaggggcttccatggtgggctctggcgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgccaactagcccaccatggaagcccccgctcagctgctgttcctgctgctgctgtggctgcctgacaccaccggcgacatccagatgacacagagccctagcaccctgagcgcctccgtgggggacagagtgacaatcacatgtaaagcctcccaggacgtgggcactgccgtggcctggtaccagcaaaaaccgggaaaagcccctaagctgctgatctactgggccagcaccagacacaccggcgtccccgatagattcagcggctctggcagcggaactgatttcaccctgaccatttcttctctgcaggccgaggacttcgccgtgtacttttgccaccagcacagcagcaaccctctgaccttcggacagggcacaaagctggaaatcaagcggacagtggctgctccttctgtgttcatctttccacctagcgacgagcagctgaagagcggcaccgcctctgtggtgtgcctgctgaacaacttctaccccagagaagccaaagtgcagtggaaggtggacaacgccctgcaatctggcaacagccaggagagcgtgacggaacaagatagcaaggacagcacctactccctgagcagcacactgaccttgtccaaggcagattacgagaagcacaaggtgtacgcctgcgaggtgacccaccagggactgagcagcccagtgaccaagagcttcaacagaggagagtgcggcggcggcggaagcggaggcggaggcagcggcggcggcggcagtggaggcggcggctctggcggagggggcagtggcggtggcggatccggcggcggcggcagcgaggtgcagcttgtggaatccggcggcggcctggtgaagcccggcggtagcctgagactgtcttgtgccgcctctggcttcacctttagcaattacgccatgagctgggtgcggcaggctcccggcaaaagactggaatgggtcgccaccatcagcaaccggggatcatatacctactaccctgatagcgtgaaaggcaggttcacaatcagccgggacaatgccaagaacagcctgtacctgcagatgaactcactgcgggccgaggacaccgccctgtattactgcgccagagagagacctatggactactggggccagggcaccctggtgaccgtttcctccgccagcaccaagggccctagcgtgttccctctggccccatgcagcagaagcacatctgagagcaccgccgctctgggctgcctggtgaaggactacttccccgagcctgtgacagtgagctggaactccggcgccctgaccagcggcgtgcacacatttccagctgtgctgcagtctagcggcctgtacagcctgagcagcgttgtgacagtgccttctagcagcctcggcaccaagacctacacctgtaacgtggatcataagccttctaataccaaggttgacaagagagtgtgagagctccctgcagggtttaaacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaactagaaattaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 51 - DNA, 4546 bp, synthetic construct
  misc_feature 1..4546: note = Synthetic: Nucleotide sequence of AAV2#12 (FIGs. 2C and 2J)
  source 1..4546: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttaccggtgcgggcctcttcgctattacgccagctggcgaaagggggatgtgctgcaaggcgattaagttgggtaacgccagggttttcccagtcacgacgttgtaaaacgacggccagtgaattcggaccgagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccggtacccaattgcccaccatggaagcccctgcccagctgctgttcctgctgctactgtggctgcctgataccaccggcgatatccagatgacgcagagtcccagcaccctgagcgcctctgtgggcgaccgggtgaccatcacctgtaaagcctcccaggacgtgggcacagctgttgcttggtatcagaaaaagcctggcaaggcccctaagctgctgatctactgggccagcacaagacacacaggagtgcctgacagattcagcggcagcggctctgggactgatttcaccttgacaatcagctctctgcaggccgaggactttgccgtgtacttctgccaccaacacagttctaaccccctgaccttcggccaaggaaccaagctggaaatcaagcggaccgtggccgctcctgccgtgttcatcttccctccaagcgatgagcagctgaaaagcggcaccgcgtccgtcgtgtgcctgctgaagaacttctacccgagagaagcgaaggtgcagtggaaagtcgacaacgccctgcagagcggaaatagccaggagagcgtgaccgaacaagactctaaggacagcacctactcgctgtcctccacgctgactctgtctaaggccgactatgagaagcacaaggtgtacgcctgcgaggtgacccaccagggcctgagcagccccgttaccaagagcttcaacagaggagaatgcggcggaggtggcagcggcggcggcgggagcggcggcggcggctcaggcggagggggaagtggcggcggcggcagcggcggcggaggcagcggcggtggcggctctgaggtgcaactggtggaatctgggggcggactggtgaagcctggcggcagtctgagactgagctgtgccgcttccggattcacctttagcaattacgccatgagctgggtgcgggaggcccctggaaagcggctggaatgggttgctacaatcagcaatagaggcagctacacatactaccccgacagtgtcaaaggccggtttacaatcagccgcgacaacgccaaaaacagcctgtacctgcagatgaactccctgcgggctgaggatacagccctctactactgtgccagagaacgtccaatggactattggggccaaggcacactggtgaccgtgagcagcgcgtctaccaagggcccttctgttttccctctggccccctgcagcagaagcacgagcgagagcaccgctgccctgggctgtctggtgaaggattatttccctgagcctgtgaccgtgtcttggaatagcggagccctgaccagcggagtgcatacattccctgctgtgctgcagtctagtgggctgtacagcctgtcttccgttgtggaagtccctagcagcagcctgggcaccaagacctacacctgcaacgtggatcataagccaagcaacaccaaggtggataagagagtgggcggtggcggaggctcgggcggcggcggcagcggcggcggcggcagcgacatcgtgctgacccagtctccagattctctggccgtgtcactgggagagagagccaccattagctgcaaggcctctcagagcgtagactacgacggcgactcctacatgaactggtaccaggaaaagcctggccagcctcctaagatcttgatctacgatgcctccaatctggagagcgggatccccgctagattcagcgggtctggaagtggaaccgacttcacactgaccatctctagcctggagcccgaggactttgccatctactactgccagcagagcaacgaggacccctggacattcggcggcggcacaaaggttgagatcaagagaaccgttgccgctcctagcgtgtttatcttccctccctctgacgagcagctgaagagcggcacagcctccgtggtgtgcctgctgaacaacttctaccccagagaggccaaggtccagtggaaggtcgacaatgcccttcagagcggcaacagccaggagtccgtgaccgagcaggatagcaaggactctacctacagcctgtcctctacgctgaccctgagcaaagccgattacgaaaagcacaaagtgtacgcctgtgaagtgacacaccagggcctgtctagccctgtgacaaagagctttaaccggggcgagtgcggcggcggtggaagcggaggtggaggttcaggaggcggcggaagcggaggcggaggcagtgggggcggcggctccggcggcggcggcagcggaggcggcggttcccaagtgcagctcgtgcagagcggcgccgaggtgaaaaagcccggagccagcgtgaagctgtcttgcaccgcctccggattcaacatcaaagacgactacatccactgggtcaagaaagccccagggcaggggctggagtggatcggcaggatcgaccctgctgatggccacaccaaatacgccccaaagttccaggtgaaagtgacaattaccgcagatacctccaccagcaccgcttatctggaactgagctctctgcggagcgaggacacagccgtgtactactgcgccagatacggctacggcagagaagtgttcgactactggggccagggcaccacagtgacagtgagctctgccagcacaaagggccccagcgtgtttcctctggccccttgcagcagaagcaccagcgagagcaccgccgccctgggctgcctggtgaaggactacttccctgaacccgtgaccgtctcctggaacagtggcgccttgacctctggcgtgcacaccttccccgccgtgctgcagagctccggcctgtacagcctgtctagcgtggtgaccgtgcctagctcgagcctgggcacaaagacatatacctgtaacgtggaccacaagcccagcaacacgaaggtggacaagcgagtgtgagtttaaacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaactagaaattaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 52 - DNA, 3723 bp, synthetic construct
  misc_feature 1..3723: note = Synthetic: Nucleotide sequence of AAV2#14 (FIGs. 2D and 2H)
  source 1..3723: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctaatttgatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccggtacccaattgccaccatggaagcccccgcccagctgctgttcctgctgctcctgtggctgcctgataccaccggcgatatcgtcctgacccagagccctgatagcctggccgtttcactgggcgagcgggccacaatctcctgcaaggcctctcagtctgttgactacgacggcgacagctacatgaactggtaccaggagaaacccggccaacctccaaagatcctgatctacgacgcctctaatctggagagcggcatccccgcccggttcagcgggtccggcagcggcaccgactttaccctgaccatctctagcctggagcctgaggacttcgccatctactactgtcagcagagcaacgaggatccttggacctttggcggcggcacaaaggtggaaatcaagcggaccgtcgccgctccatccgtgtttatcttccctccttccgacgagcagctcaagagcggtaccgccagcgtggtgtgcctgctgaacaacttctaccccagagaggccaaggtgcagtggaaggtagacaacgccttgcagagcggcaactctcaagagagcgtgacagagcaggactctaaggacagcacatacagcctaagctccaccctgaccctcagcaaggccgactacgagaagcacaaggtgtacgcctgtgaagttacacaccagggcctgagcagtccggtgaccaagtccttcaacagaggcgaatgcggcggaggaggctctggcggcggcggcagcggcggaggcggcagcggcggcggaggctctggcggcggtggcagcggaggcggcggaagcggcggaggtggcagccaggtgcagctggtgcagagcggtgctgaagtgaagaaacccggcgcttccgtgaaactgagctgcaccgccagcggatttaacatcaaggacgactacattcactgggtgaaaaaggcccctggccagggcctggaatggatcgggagaatcgaccccgccgatggccataccaagtacgctcctaagttccaggtgaaagtgaccatcaccgctgatacaagcacctctacagcctacctggagctgagctccctgcggtctgaggacaccgccgtgtactactgcgccagatacggctacggcagagaggtgttcgactactggggacagggcactacagtcaccgtgtctagtgctagcacgaagggccctagcgtgttccctctggctccatgtagcagaagcaccagcgaaagcacagctgctctgggctgcctggtgaaagactacttccccgagcctgtgaccgtcagctggaactccggcgccctgaccagcggagtgcacacctttcctgctgtgctgcaatcctctggcctgtactctctgagctctgttgtgacagtgccttctagcagcctgggaaccaagacctacacctgcaacgtggaccacaagcccagcaacaccaaggtggataagcgcgtgggcggcggcggatctggcggaggcggcagcgaggtgcagctggtggaaagcggcggcggcctggtgaagcctggcggctcactgagactgagctgtgccgccagcggcttcaccttctccaactacgccatgagctgggtgcgggaagccccaggaaagcgcctggagtgggtcgccaccatcagcaatagaggctcgtatacatattaccctgattccgtcaaaggcagattcaccatctctagagataatgccaagaacagcctgtacctgcagatgaactccctcagagccgaggatacagccctgtattactgcgccagagaacggcctatggactactggggccaaggcactctggtgacagtgagcagcggcggcggtggttccggcggcggaggctctggaggaggcggcagcgacatccagatgacccagagccctagcaccctgtccgccagcgtgggagatagagtgaccattacctgtaaagcgagccaggatgtgggcaccgccgtggcctggtatcagaagaagcctggcaaggcccctaagctgctgatctactgggcctctacccggcacacaggcgtgcccgacagattctccggctccggttctggaacagacttcacactgaccatcagctctcttcaggccgaggacttcgccgtgtacttctgccaccagcacagctctaatcctctgacattcggccaaggcacaaagctggaaatcaagtgagtttaaacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaactaggtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 53 - DNA, 921 bp, synthetic construct
  misc_feature 1..921: note = Synthetic: Bidirectional promoter and CMV enhancer
  source 1..921: mol_type = other DNA
cgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 54 - DNA, 1539 bp, synthetic construct
  misc_feature 1..1539: note = Synthetic: [alpha-C1s scFv(G4S)2 alpha-Bb scFv] nucleic acid sequence (construct #5, FIG. 2A)
  source 1..1539: mol_type = other DNA
atggaagccccagctcagctgctgttcctcctgctgctgtggctgcctgacacaaccggccaagtgcagctggtccagagcggcgccgaggtgaaaaagccaggagcctccgtcaaactgagctgtaccgccagcggctttaacatcaaggacgactacatccactgggtgaagcaggcccctggccaaggtctggaatggatcggcagaatcgaccccgctgacggccacaccaagtacgcccctaagttccaggtgaaggtgaccatcaccgccgacaccagcacaagcaccgcatacctggagctgtccagcctgagaagcgaggataccgctgtctactactgcgccagatacggctacggcagagaggtgttcgactactggggacaaggtaccaccgtgacggtgtctagcggcggtggcggcagcggaggaggcggctctggaggcggcggatctgatatcgtgctgacacagagtcctgacagcctggccgtgagcttgggggagcgggctacaatctcttgtaaagccagccagagcgtggactatgatggcgatagctacatgaactggtatcagcagaaacccggccagccccccaagatcctgatctacgacgccagcaatctggagagcggcatccccgcccggttcagcggcagcggctcgggcacagatttcaccctgaccattagctctctggaacctgaggacttcgctatctactactgccagcagagcaacgaggacccttggaccttcggcggaggtacaaaggtggaaatcaagggcggcggcggcagcggaggcggaggctctgaggtgcaactggtggagagcggcggcggactggtaaagcccggcggctcactgagactgtcctgcgctgccagcggcttcaccttttctaactacgccatgagctgggtgcggcaggctcctggaaagcgcctggaatgggtggccacaatcagcaaccggggctcttacacctactatcctgattctgtgaagggtaggttcaccatttcaagagataacgccaagaacagcctctacctgcagatgaacagcctgcgggccgaagacaccgccctgtactactgcgccagagaaagacctatggactactggggccagggcaccctggtgacagtttcctccggaggcggaggctccggcggcggcggctccggaggcggcggaagcgacatccagatgacccagagccctagcactctgtccgccagcgtgggcgacagagtgaccatcacatgcaaggcctctcaggacgtgggcaccgccgtggcctggtaccaacagaagcctggcaaggcccctaagctgctgatctactgggccagcacaagacatacaggcgtgcccgatagattcagcggctccggctctggcacagacttcacactgaccatcagcagcctccaggccgaggattttgccgtgtacttctgccaccagcacagcagcaatccactgacatttggccagggcaccaagctggagatcaaatga

SEQ ID NO: 55 - protein, 512 aa, synthetic construct
  REGION 1..512: note = Synthetic: [alpha-C1s scFv(G4S)2 alpha-Bb scFv] amino acid sequence (construct #5, FIG. 2A)
MEAPAQLLFLLLLWLPDTTGQVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSSGGGGSGGGGSGGGGSDIVLTQSPDSLAVSLGERATISCKASQSVDYDGDSYMNWYQQKPGQPPKILIYDASNLESGIPARFSGSGSGTDFTLTISSLEPEDFAIYYCQQSNEDPWTFGGGTKVEIKGGGGSGGGGSEVQLVESGGGLVKPGGSLRLSCAASGFTFSNYAMSWVRQAPGKRLEWVATISNRGSYTYYPDSVKGRFTISRDNAKNSLYLQMNSLRAEDTALYYCARERPMDYWGQGTLVTVSSGGGGSGGGGSGGGGSDIQMTQSPSTLSASVGDRVTITCKASQDVGTAVAWYQQKPGKAPKLLIYWASTRHTGVPDRFSGSGSGTDFTLTISSLQAEDFAVYFCHQHSSNPLTFGQGTKLEIK

SEQ ID NO: 56 - DNA, 1539 bp, synthetic construct
  misc_feature 1..1539: note = Synthetic: [alpha-Bb scFv(G4S)2 alpha-C1s scFv] nucleic acid sequence (construct #6, FIG. 2A)
  source 1..1539: mol_type = other DNA
atggaagcccctgcccagctgctgttcctgctgctgctgtggctacctgataccaccggcgaggtgcagctggtcgagagcggcgggggcctggtgaaaccaggaggaagcctgagactgagctgcgccgcctctggcttcaccttcagcaattacgctatgagctgggtcagacaggccccaggaaaaagactggaatgggtggccacaatttctaaccggggctcctacacctactatcctgacagcgtgaagggcagattcacaatcagccgggacaacgccaagaacagcctgtacctgcagatgaacagcctcagagccgaggacaccgccctgtactactgcgccagagagcggcctatggactactggggccaaggcacactggtcacagtttccagcggcggcggcggcagcggtggcggcggcagcggaggcggtggctctgatatccagatgacccagtcccctagcaccctgtctgcctctgtgggcgacagagtgaccattacatgcaaggcctctcaggacgtgggcaccgctgtggcctggtatcagcagaaacccggcaaggctcccaagctgctgatctactgggccagcacaagacacacaggcgtgcctgatagattcagcggcagcggtagcggcaccgacttcaccctgacaatcagctccctccaggctgaagattttgccgtgtacttctgccaccagcatagcagcaaccccctgacattcggccagggcacaaagctggaaatcaagggaggcggcggctctggaggcggcggaagccaagtgcagctggtgcaaagcggcgccgaggtgaaaaagcccggcgcatctgtgaagctgagttgtacagcttctggatttaacatcaaggacgactacatccactgggttaagcaggcccctggccagggcctggagtggatcggcagaatcgaccccgctgatggccacaccaagtacgcccctaagttccaggtgaaggtgaccatcacggccgacaccagcacaagcaccgcctacctggaactgagcagcctgcggagcgaggacaccgccgtgtactactgtgccagatacggctacggccgcgaggtgttcgactactggggacaaggaacaaccgtgaccgtgtccagcggcggcggcggcagcggcggaggaggctctggcggcggcggcagcgacatcgtgctgacccagagccccgattctctggccgtgagcctgggagagagagccaccatctcctgcaaggcttcccaatctgtggactatgatggagatagctacatgaactggtaccagcagaagcctggccagcctccaaagatcctgatctacgacgccagcaatctggaatccggcatccctgctcggtttagcggcagcggctccggaaccgacttcaccctgaccatcagctctctggagcctgaggatttcgccatctactactgccagcagtccaacgaagacccttggacctttggcggcggcaccaaggtcgaaatcaaatga

SEQ ID NO: 57 - protein, 512 aa, synthetic construct
  REGION 1..512: note = Synthetic: [alpha-Bb scFv(G4S)2 alpha-C1s scFv] amino acid sequence (construct #6, FIG. 2A)
MEAPAQLLFLLLLWLPDTTGEVQLVESGGGLVKPGGSLRLSCAASGFTFSNYAMSWVRQAPGKRLEWVATISNRGSYTYYPDSVKGRFTISRDNAKNSLYLQMNSLRAEDTALYYCARERPMDYWGQGTLVTVSSGGGGSGGGGSGGGGSDIQMTQSPSTLSASVGDRVTITCKASQDVGTAVAWYQQKPGKAPKLLIYWASTRHTGVPDRFSGSGSGTDFTLTISSLQAEDFAVYFCHQHSSNPLTFGQGTKLEIKGGGGSGGGGSQVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSSGGGGSGGGGSGGGGSDIVLTQSPDSLAVSLGERATISCKASQSVDYDGDSYMNWYQQKPGQPPKILIYDASNLESGIPARFSGSGSGTDFTLTISSLEPEDFAIYYCQQSNEDPWTFGGGTKVEIK

SEQ ID NO: 58 - DNA, 2230 bp, synthetic construct
  misc_feature 1..2230: note = Synthetic: [alpha-C1s scFab(G4S)3 - alpha-Bb scFv] nucleic acid sequence (construct #15, FIG. 2E)
  source 1..2230: mol_type = other DNA
atggaagctccagcccagctgctgttcctgctgctcctttggctgcctgacacaacaggcgatatcgtgctgacccagagccctgacagcctggccgtgtcactgggcgagcgggccacgatcagctgcaaggccagccagtccgtggattacgacggcgacagctacatgaactggtatcagcagaagcccggacagcctcccaagatcctgatctacgacgccagcaacctggaaagcggcatccctgccagattcagcgggtccggcagcggaacagacttcaccctgaccatctccagcctggaacctgaggatttcgccatctactactgtcagcagagcaacgaggatccttggaccttcggcggcggcaccaaggtcgagatcaagagaaccgtggccgctcctagcgtgttcatcttccctccttccgacgagcagctgaagagcggcaccgcctctgtggtgtgcctactgaacaacttctaccctagagaggctaaagtgcagtggaaggtggacaatgccctgcagagcggcaacagccaggagtctgtgaccgagcaggacagcaaggacagcacctacagcctgtcttccacactgaccctgtctaaggccgactacgagaagcacaaggtctacgcctgcgaggtgacacaccagggcctgagctcccccgtgaccaaaagcttcaacagaggagaatgcggcggaggcggaagcggcggcgggggctctggaggcggcggctccggcggcggaggcagcggaggtggcggctctggcggcggcggctccggaggcggcggctcacaggtgcagctggtgcaatctggtgctgaggtgaagaagccaggcgccagcgtgaagctaagctgcaccgcctccggtttcaacatcaaagacgactacatccactgggtgaaacaggccccaggccagggcctggagtggatcggcagaatcgaccctgccgatggccacaccaagtacgctcctaagttccaggtcaaggtgacaatcaccgcagataccagcacaagcaccgcctacctggagctgagctcgctgagaagcgaggacacagccgtgtactactgcgccagatacggctacggaagagaggtgtttgattactggggacagggcactaccgtgaccgtgagctccgccagcaccaagggccctagcgtgttccccctggccccatgttctagatctacatctgaaagcaccgctgctctgggctgcctggtaaaggactacttccccgagcccgtgaccgtgtcctggaacagcggcgccctgacctctggcgtgcatacatttcctgccgtgctgcagagctcaggcctgtactccctgagctctgtcgttacagtgcccagcagctccctgggaacaaagacctacacctgcaacgtggaccacaagcctagcaataccaaggtggacaagcgggtggggggcggtggatccggcggaggcgggagcggcggcggaggatccgaggtgcagctggtcgaatccggcgggggcctggtgaaacccggcggctctctgaggctgtcctgcgccgctagcggctttacctttagcaactacgctatgagctgggttagacaggcccctggcaagcggctcgaatgggtcgcaacaatttctaatagaggcagttacacatactaccccgactctgtgaagggccggttcaccattagcagagataacgccaagaactctctgtacctgcagatgaattcactgcgggccgaggacaccgccctgtattattgtgctcgggaacgtcctatggactactggggccagggcaccctggtgacagtgtcctctggcggcggcggcagcggcggtggcggcagcggcggcggcggtagcgacatccagatgacccaaagccccagcaccctgtctgccagcgtgggtgacagagtgaccatcacctgtaaagcctcccaggatgtgggaacagccgttgcctggtaccagcaaaaacctggcaaggcccctaagctgctgatctactgggccagcacccgccacactggcgtgcctgatcggttcagcggaagcggcagcggaacagattttacactgactatcagctccctccaggccgaagatttcgccgtgtacttctgccaccagcacagcagcaaccctctgaccttcggacaagggacaaaactcgaaatcaagtgag

SEQ ID NO: 59 - protein, 742 aa, synthetic construct
  REGION 1..742: note = Synthetic: [alpha-C1s scFab(G4S)3 - alpha-Bb scFv] amino acid sequence (construct #15, FIG. 2E)
MEAPAQLLFLLLLWLPDTTGDIVLTQSPDSLAVSLGERATISCKASQSVDYDGDSYMNWYQQKPGQPPKILIYDASNLESGIPARFSGSGSGTDFTLTISSLEPEDFAIYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSQVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSGGGGSEVQLVESGGGLVKPGGSLRLSCAASGFTFSNYAMSWVRQAPGKRLEWVATISNRGSYTYYPDSVKGRFTISRDNAKNSLYLQMNSLRAEDTALYYCARERPMDYWGQGTLVTVSSGGGGSGGGGSGGGGSDIQMTQSPSTLSASVGDRVTITCKASQDVGTAVAWYQQKPGKAPKLLIYWASTRHTGVPDRFSGSGSGTDFTLTISSLQAEDFAVYFCHQHSSNPLTFGQGTKLEIK

SEQ ID NO: 60 - DNA, 2229 bp, synthetic construct
  misc_feature 1..2229: note = Synthetic: [alpha-C1s scFab(G4S)3 - alpha-Bb scFv-CM] nucleic acid sequence (construct #16, FIG. 2E)
  source 1..2229: mol_type = other DNA
atggaagcccctgcccagctgctgttcctgctgctgctgtggctgcctgacacaaccggcgacatcgtgctgacacagagccccgacagcctcgccgtttccctcggcgagcgggccacaatctcatgcaaggcctcacagtccgtggactatgacggcgatagctacatgaactggtaccaggagaagcctggccaacctccaaagatcctgatctacgacgccagcaatctggaatccggtattcctgccagattcagcggctctggatccggcaccgactttactctgaccatcagctctctggaacctgaggactttgctatctactactgccagcagagcaacgaggacccctggaccttcggcggcggcaccaaagtggaaatcaagcggaccgtggccgctccttcagtgttcatcttcccaccttccgacgagcagctgaagagcggcaccgccagcgtggtgtgcctgctgaacaacttctaccccagagaggctaaggtgcagtggaaggtggataacgctctgcaaagtggcaactctcaggagtctgtgacagagcaggactccaaggacagcacctacagcctgtcctctaccctgacactgtccaaggccgactacgagaagcacaaggtgtacgcctgtgaagtgacacaccaggggctgagctcccctgtgacaaaatctttcaaccggggcgagtgcggcggaggaggcagcggcggcggcggcagcgggggcggaggctccggcggcggcggtagcggtgggggcggatctggaggcgggggatcgggcggaggcggcagccaggtgcagctggtccagagcggcgccgaggtgaaaaagccaggcgcctctgtgaagctgtcttgcaccgcctctggttttaatatcaaggacgactacatccactgggtgaagaaggctccaggtcaaggactggaatggatcggccggatcgaccccgctgatggccacaccaaatacgctcctaagttccaggtgaaagttacaattacagccgataccagcacaagcaccgcctacctggagctgagctctctgagaagcgaagatacagccgtgtactactgcgcaagatacggctacggcagagaggtgttcgactattggggacagggcaccacagtgaccgtgtctagtgccagcaccaagggccccagcgtgttccctctggccccttgtagcagatctaccagcgagtccaccgctgctctgggctgcctggtcaaggattacttccccgagcctgtgaccgttagctggaacagcggagccctgaccagcggcgtgcacacctttccagccgtgctgcagagcagcggactgtatagcctgagcagcgtcgtgacagtgcccagcagcagcctgggcaccaagacctacacctgcaacgtggaccacaagcccagcaacaccaaggtggacaagagagtgggcggcggaggctctggcggcggcggctctgggggcggcggaagcgaggtgcagctggtggaatctggcggcggactggtgaagcctggcggcagcctgagactgagctgcgccgccagcggcttcaccttcagcaactacgccatgagctgggttagagaagcccctggaaaaagactggaatgggtggccaccatctctaatagaggatcttatacatactaccctgattctgtgaaaggacggttcacaatctcccgcgacaacgccaagaactcactgtacctgcagatgaactctctgagggccgaggataccgccctgtactactgtgcccgagaaagacctatggattactggggccagggcaccctcgtcacagtttcctctggcgggggcggtagcggcggcggcggatccggcggaggtggcagcgacatccagatgacccaaagcccttctacactgagcgccagcgtcggcgaccgggtgaccatcacctgtaaagccagccaagacgtgggcacggctgtggcttggtatcagaagaaacctggcaaggcccccaagctgcttatctactgggccagcacaagacacacaggcgttcctgatagattcagcggcagcggctccggcacagatttcaccctgaccatctcgagtctgcaggccgaggatttcgccgtgtacttctgccaccagcattcttctaaccctctgacctttggccagggaaccaagctggaaatcaagtga

SEQ ID NO: 61 - protein, 742 aa, synthetic construct
  REGION 1..742: note = Synthetic: [alpha-C1s scFab(G4S)3 - alpha-Bb scFv-CM] amino acid sequence (construct #16, FIG. 2E)
MEAPAQLLFLLLLWLPDTTGDIVLTQSPDSLAVSLGERATISCKASQSVDYDGDSYMNWYQEKPGQPPKILIYDASNLESGIPARFSGSGSGTDFTLTISSLEPEDFAIYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSQVQLVQSGAEVKKPGASVKLSCTASGFNIKDDYIHWVKKAPGQGLEWIGRIDPADGHTKYAPKFQVKVTITADTSTSTAYLELSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSGGGGSEVQLVESGGGLVKPGGSLRLSCAASGFTFSNYAMSWVREAPGKRLEWVATISNRGSYTYYPDSVKGRFTISRDNAKNSLYLQMNSLRAEDTALYYCARERPMDYWGQGTLVTVSSGGGGSGGGGSGGGGSDIQMTQSPSTLSASVGDRVTITCKASQDVGTAVAWYQKKPGKAPKLLIYWASTRHTGVPDRFSGSGSGTDFTLTISSLQAEDFAVYFCHQHSSNPLTFGQGTKLEIK

SEQ ID NO: 62 - DNA, 3869 bp, synthetic construct
  misc_feature 1..3869: note = Synthetic: [alpha-C1s scFab - BiDir - alpha-Bb scFab-CM] nucleic acid sequence (construct #21, FIG. 2G)
  source 1..3869: mol_type = other DNA
tcacacccgcttatccaccttggtgttgctgggcttgtggtccacgttgcaggtgtaggtctttgtgcccaggctagagctaggcactgtcacgacagaggacagagagtacaggccgctgctctgcagcacggcggggaaggtgtgcaccccgcttgtcagggctccgctgttccaggacacggtcacaggctcagggaaataatccttgaccaggcagcccagagcagccgtgctctctgaggtacttctgctacaaggagccagtgggaacacgctagggccctttgtgctggcggacgacacggtcactgttgtgccctgtccccagtagtcgaacacttctctgccgtagccgtatctggcgcagtagtacacagcggtgtcctcggatctaaggctgctcagttccagataagctgtagaggtgctggtatcggcggtgatggtgactttcacctggaacttaggggcgtactttgtgtggccgtcggcagggtcgattctgccgatccactccagtccctggccgggggccttcttcacccagtggatgtaatcgtccttgatattgaagccgctggcggtgcagctcagcttaacactagcgccaggctttttcacctcggctccgctctgcaccagctgcacctgggatccgccgccgccgctgccgcctccgccgctgccgcctccgccgcttccgcctcccccagagccgccgccaccgctgcctcctccgccggagccgccgccgccgcactcgccccggttgaagcttttggtcacaggagaggacaggccctgatgtgtcacttcacaggcgtacaccttgtgcttctcgtagtcggccttgctcaaggtcagggtgctggacaggctgtatgttgagtccttgctgtcctgctcggtcacgctctcttggctgttgccgctttgcagggcgttgtcaactttccattggacctttgcctctctggggtagaagttattcagcaggcacaccacagaggcggttccgctcttcagctgctcgtcgcttggagggaagataaagacagaaggggcggccacggtgcgcttgatttccaccttggtgccgcctccaaaggtccaggggtcctcgttgctctgctggcagtagtagatggcaaaatcctcgggttccagagaagaaattgtcagggtgaaatcagtgccagagccgctgccgctgaatctggcggggatgccgctttccagattgctggcgtcgtagatcaggatttttggaggctggccgggtttctcctggtaccagttcatgtagctgtcgccgtcatagtccacgctctgagaggctttacagctgattgtggcccgttcgccgaggctcacggccaggctatcagggctctgcgtcagcacgatatcgccggtggtgtcaggcagccacaggagcagcaggaacagcagctgggcaggggcttccatggtgggctctggcgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgccaactagcccaccatggaagcccccgctcagctgctgttcctgctgctgctgtggctgcctgacaccaccggcgacatccagatgacacagagccctagcaccctgagcgcctccgtgggggacagagtgacaatcacatgtaaagcctcccaggacgtgggcactgccgtggcctggtaccagaaaaaaccgggaaaagcccctaagctgctgatctactgggccagcaccagacacaccggcgtccccgatagattcagcggctctggcagcggaactgatttcaccctgaccatttcttctctgcaggccgaggacttcgccgtgtacttttgccaccagcacagcagcaaccctctgaccttcggacagggcacaaagctggaaatcaagcggacagtggctgctccttctgtgttcatctttccacctagcgacgagcagctgaagagcggcaccgcctctgtggtgtgcctgctgaacaacttctaccccagagaagccaaagtgcagtggaaggtggacaacgccctgcaatctggcaacagccaggagagcgtgacggaacaagatagcaaggacagcacctactccctgagcagcacactgaccttgtccaaggcagattacgagaagcacaaggtgtacgcctgcgaggtgacccaccagggactgagcagcccagtgaccaagagcttcaacagaggagagtgcggcggcggcggaagcggaggcggaggcagcggcggcggcggcagtggaggcggcggctctggcggagggggcagtggcggtggcggatccggcggcggcggcagcgaggtgcagcttgtggaatccggcggcggcctggtgaagcccggcggtagcctgagactgtcttgtgccgcctctggcttcacctttagcaattacgccatgagctgggtgcgggaggctcccggcaaaagactggaatgggtcgccaccatcagcaaccggggatcatatacctactaccctgatagcgtgaaaggcaggttcacaatcagccgggacaatgccaagaacagcctgtacctgcagatgaactcactgcgggccgaggacaccgccctgtattactgcgccagagagagacctatggactactggggccagggcaccctggtgaccgtttcctccgccagcaccaagggccctagcgtgttccctctggccccatgcagcagaagcacatctgagagcaccgccgctctgggctgcctggtgaaggactacttccccgagcctgtgacagtgagctggaactccggcgccctgaccagcggcgtgcacacatttccagctgtgctgcagtctagcggcctgtacagcctgagcagcgttgtgacagtgccttctagcagcctcggcaccaagacctacacctgtaacgtggatcataagccttctaataccaaggttgacaagagagtgtga

SEQ ID NO: 63 - protein, 490 aa, synthetic construct
  REGION 1..490: note = Synthetic: alpha-C1s scFab-CM arm (construct #22, FIG.2G)
MEAPAQLLFLLLLWLPDTTGDIVLTQSPDSLAVSLGERATISCKASQSVDYDGDSYMNWYQEKPGQPPKILIYDASNLESGIPARFSGSGSGTDFTLTISSLEPEDFAIYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSQVQLVQSGAEVKKPGASVKLSCTASGFNIKDDYIHWVKKAPGQGLEWIGRIDPADGHTKYAPKFQVKVTITADTSTSTAYLELSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 64 - protein, 482 aa, synthetic construct
  REGION 1..482: note = Synthetic: alpha-Bb scFab-CM arm (construct #22, FIG.2G)
MEAPAQLLFLLLLWLPDTTGDIQMTQSPSTLSASVGDRVTITCKASQDVGTAVAWYQKKPGKAPKLLIYWASTRHTGVPDRFSGSGSGTDFTLTISSLQAEDFAVYFCHQHSSNPLTFGQGTKLEIKRTVAAPAVFIFPPSDEQLKSGTASVVCLLKNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSEVQLVESGGGLVKPGGSLRLSCAASGFTFSNYAMSWVREAPGKRLEWVATISNRGSYTYYPDSVKGRFTISRDNAKNSLYLQMNSLRAEDTALYYCARERPMDYWGQGTLVTVSSASTKGPSVFPLAPCSRSTSESTAALGCLVKDYFPEPVTVSWNSGALTSGVHTFPAVLQSSGLYSLSSVVEVPSSSLGTKTYTCNVDHKPSNTKVDKRV

SEQ ID NO: 65 - protein, 688 aa, synthetic construct
  REGION 1..688: note = Synthetic: Human complement C1s amino acid sequence prior to processing and activation
MWCIVLFSLLAWVYAEPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 66 - protein, 764 aa, synthetic construct
  REGION 1..764: note = Synthetic: Human complement factor B prior to processing and activation
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 67 - protein, 8 aa, synthetic construct
  REGION 1..8: note = Synthetic: HCDR1 of anti-C1s antibody (IMGT)
GFNIKDDY

SEQ ID NO: 68 - protein, 8 aa, synthetic construct
  REGION 1..8: note = Synthetic: HCDR2 of anti-C1s antibody (IMGT)
IDPADGHT

SEQ ID NO: 69 - protein, 12 aa, synthetic construct
  REGION 1..12: note = Synthetic: HCDR3 of anti-C1s antibody (IMGT)
ARYGYGREVFDY

SEQ ID NO: 70 - protein, 10 aa, synthetic construct
  REGION 1..10: note = Synthetic: LCDR1 of anti-C1s antibody (IMGT)
QSVDYDGDSY

SEQ ID NO: 71 - protein, 7 aa, synthetic construct
  REGION 1..7: note = Synthetic: HCDR1 of anti-C1s antibody (Chothia)
GFNIKDD

SEQ ID NO: 72 - protein, 6 aa, synthetic construct
  REGION 1..6: note = Synthetic: HCDR2 of anti-C1s antibody (Chothia)
DPADGH

SEQ ID NO: 73 - protein, 8 aa, synthetic construct
  REGION 1..8: note = Synthetic: HCDR1 of anti-Bb antibody (IMGR)
GFTFSNYA

SEQ ID NO: 74 - protein, 8 aa, synthetic construct
  REGION 1..8: note = Synthetic: HCDR2 of anti-Bb antibody (IMGR)
ISNRGSYT

SEQ ID NO: 75 - protein, 8 aa, synthetic construct
  REGION 1..8: note = Synthetic: HCDR3 of anti-Bb antibody (IMGR)
ARERPMDY

SEQ ID NO: 76 - protein, 6 aa, synthetic construct
  REGION 1..6: note = Synthetic: LCDR1 of anti-Bb antibody (IMGR)
QDVGTA

SEQ ID NO: 77 - protein, 7 aa, synthetic construct
  REGION 1..7: note = Synthetic: HCDR1 of anti-Bb antibody (Chothia)
GFTFSNY

SEQ ID NO: 78 - protein, 6 aa, synthetic construct
  REGION 1..6: note = Synthetic: HCDR2 of anti-Bb antibody (Chothia)
SNRGSY

SEQ ID NO: 79 - DNA, 3869 bp, synthetic construct
  misc_feature 1..3869: note = Synthetic: [alpha-Bb scFab - BiDir alpha-C1s scFab-CM] nucleic acid sequence (construct #22, FIG. 2G)
  source 1..3869: mol_type = other DNA
tcacactctcttgtcaaccttggtattagaaggcttatgatccacgttacaggtgtaggtcttggtgccgaggctgctagaaggcactgtcacaacgctgctcaggctgtacaggccgctagactgcagcacagctggaaatgtgtgcacgccgctggtcagggcgccggagttccagctcactgtcacaggctcggggaagtagtccttcaccaggcagcccagagcggcggtgctctcagatgtgcttctgctgcatggggccagagggaacacgctagggcccttggtgctggcggaggaaacggtcaccagggtgccctggccccagtagtccataggtctctctctggcgcagtaatacagggcggtgtcctcggcccgcagtgagttcatctgcaggtacaggctgttcttggcattgtcccggctgattgtgaacctgcctttcacgctatcagggtagtaggtatatgatccccggttgctgatggtggcgacccattccagtcttttgccgggagcctcccgcacccagctcatggcgtaattgctaaaggtgaagccagaggcggcacaagacagtctcaggctaccgccgggcttcaccaggccgccgccggattccacaagctgcacctcgctgccgccgccgccggatccgccaccgccactgccccctccgccagagccgccgcctccactgccgccgccgccgctgcctccgcctccgcttccgccgccgccgcactctcctctgttgaagctcttggtcactgggctgctcagtccctggtgggtcacctcgcaggcgtacaccttgtgcttctcgtaatctgccttggacaaggtcagtgtgctgctcagggagtaggtgctgtccttgctatcttgttccgtcacgctctcctggctgttgccagattgcagggcgttgtccaccttccactgcactttggcttctctggggtagaagttgttcagcaggcacaccacagaggcggtgccgctcttcagctgctcgtcgctaggtggaaagatgaacacagaaggagcagccactgtccgcttgatttccagctttgtgccctgtccgaaggtcagagggttgctgctgtgctggtggcaaaagtacacggcgaagtcctcggcctgcagagaagaaatggtcagggtgaaatcagttccgctgccagagccgctgaatctatcggggacgccggtgtgtctggtgctggcccagtagatcagcagcttaggggcttttcccggttttttctggtaccaggccacggcagtgcccacgtcctgggaggctttacatgtgattgtcactctgtcccccacggaggcgctcagggtgctagggctctgtgtcatctggatgtcgccggtggtgtcaggcagccacagcagcagcaggaacagcagctgagcgggggcttccatggtgggctagttggcgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgccagagcccaccatggaagcccctgcccagctgctgttcctgctgctcctgtggctgcctgacaccaccggcgatatcgtgctgacgcagagccctgatagcctggccgtgagcctcggcgaacgggccacaatcagctgtaaagcctctcagagcgtggactatgacggcgacagctacatgaactggtaccaggagaaacccggccagcctccaaaaatcctgatctacgacgccagcaatctggaaagcggcatccccgccagattcagcggcagcggctctggcactgatttcaccctgacaatttcttctctggaacccgaggattttgccatctactactgccagcagagcaacgaggacccctggacctttggaggcggcaccaaggtggaaatcaagcgcaccgtggccgccccttctgtctttatcttccctccaagcgacgagcagctgaagagcggaaccgcctctgtggtgtgcctgctgaataacttctaccccagagaggcaaaggtccaatggaaagttgacaacgccctgcaaagcggcaacagccaagagagcgtgaccgagcaggacagcaaggactcaacatacagcctgtccagcaccctgaccttgagcaaggccgactacgagaagcacaaggtgtacgcctgtgaagtgacacatcagggcctgtcctctcctgtgaccaaaagcttcaaccggggcgagtgcggcggcggcggctccggcggaggaggcagcggtggcggcggctctgggggaggcggaagcggcggaggcggcagcggcggaggcggcagcggcggcggcggatcccaggtgcagctggtgcagagcggagccgaggtgaaaaagcctggcgctagtgttaagctgagctgcaccgccagcggcttcaatatcaaggacgattacatccactgggtgaagaaggcccccggccagggactggagtggatcggcagaatcgaccctgccgacggccacacaaagtacgcccctaagttccaggtgaaagtcaccatcaccgccgataccagcacctctacagcttatctggaactgagcagccttagatccgaggacaccgctgtgtactactgcgccagatacggctacggcagagaagtgttcgactactggggacagggcacaacagtgaccgtgtcgtccgccagcacaaagggccctagcgtgttcccactggctccttgtagcagaagtacctcagagagcacggctgctctgggctgcctggtcaaggattatttccctgagcctgtgaccgtgtcctggaacagcggagccctgacaagcggggtgcacaccttccccgccgtgctgcagagcagcggcctgtactctctgtcctctgtcgtgacagtgcctagctctagcctgggcacaaagacctacacctgcaacgtggaccacaagcccagcaacaccaaggtggataagcgggtgtga

SEQ ID NO: 80 - DNA, 3869 bp, synthetic construct
  misc_feature 1..3869: note = Synthetic: [alpha-Bb scFab - BiDir alpha-C1s scFab] nucleic acid sequence (construct #10, FIG. 2B)
  source 1..3869: mol_type = other DNA
tcacactctcttgtcaaccttggtattagaaggcttatgatccacgttacaggtgtaggtcttggtgccgaggctgctagaaggcactgtcacaacgctgctcaggctgtacaggccgctagactgcagcacagctggaaatgtgtgcacgccgctggtcagggcgccggagttccagctcactgtcacaggctcggggaagtagtccttcaccaggcagcccagagcggcggtgctctcagatgtgcttctgctgcatggggccagagggaacacgctagggcccttggtgctggcggaggaaacggtcaccagggtgccctggccccagtagtccataggtctctctctggcgcagtaatacagggcggtgtcctcggcccgcagtgagttcatctgcaggtacaggctgttcttggcattgtcccggctgattgtgaacctgcctttcacgctatcagggtagtaggtatatgatccccggttgctgatggtggcgacccattccagtcttttgccgggagcctgccgcacccagctcatggcgtaattgctaaaggtgaagccagaggcggcacaagacagtctcaggctaccgccgggcttcaccaggccgccgccggattccacaagctgcacctcgctgccgccgccgccggatccgccaccgccactgccccctccgccagagccgccgcctccactgccgccgccgccgctgcctccgcctccgcttccgccgccgccgcactctcctctgttgaagctcttggtcactgggctgctcagtccctggtgggtcacctcgcaggcgtacaccttgtgcttctcgtaatctgccttggacaaggtcagtgtgctgctcagggagtaggtgctgtccttgctatcttgttccgtcacgctctcctggctgttgccagattgcagggcgttgtccaccttccactgcactttggcttctctggggtagaagttgttcagcaggcacaccacagaggcggtgccgctcttcagctgctcgtcgctaggtggaaagatgaacacagaaggagcagccactgtccgcttgatttccagctttgtgccctgtccgaaggtcagagggttgctgctgtgctggtggcaaaagtacacggcgaagtcctcggcctgcagagaagaaatggtcagggtgaaatcagttccgctgccagagccgctgaatctatcggggacgccggtgtgtctggtgctggcccagtagatcagcagcttaggggcttttcccggtttttgctggtaccaggccacggcagtgcccacgtcctgggaggctttacatgtgattgtcactctgtcccccacggaggcgctcagggtgctagggctctgtgtcatctggatgtcgccggtggtgtcaggcagccacagcagcagcaggaacagcagctgagcgggggcttccatggtgggctagttggcgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgccagagcccaccatggaagcccctgcccagctgctgttcctgctgctcctgtggctgcctgacaccaccggcgatatcgtgctgacgcagagccctgatagcctggccgtgagcctcggcgaacgggccacaatcagctgtaaagcctctcagagcgtggactatgacggcgacagctacatgaactggtaccagcagaaacccggccagcctccaaaaatcctgatctacgacgccagcaatctggaaagcggcatccccgccagattcagcggcagcggctctggcactgatttcaccctgacaatttcttctctggaacccgaggattttgccatctactactgccagcagagcaacgaggacccctggacctttggaggcggcaccaaggtggaaatcaagcgcaccgtggccgccccttctgtctttatcttccctccaagcgacgagcagctgaagagcggaaccgcctctgtggtgtgcctgctgaataacttctaccccagagaggcaaaggtccaatggaaagttgacaacgccctgcaaagcggcaacagccaagagagcgtgaccgagcaggacagcaaggactcaacatacagcctgtccagcaccctgaccttgagcaaggccgactacgagaagcacaaggtgtacgcctgtgaagtgacacatcagggcctgtcctctcctgtgaccaaaagcttcaaccggggcgagtgcggcggcggcggctccggcggaggaggcagcggtggcggcggctctgggggaggcggaagcggcggaggcggcagcggcggaggcggcagcggcggcggcggatcccaggtgcagctggtgcagagcggagccgaggtgaaaaagcctggcgctagtgttaagctgagctgcaccgccagcggcttcaatatcaaggacgattacatccactgggtgaagcaggcccccggccagggactggagtggatcggcagaatcgaccctgccgacggccacacaaagtacgcccctaagttccaggtgaaagtcaccatcaccgccgataccagcacctctacagcttatctggaactgagcagccttagatccgaggacaccgctgtgtactactgcgccagatacggctacggcagagaagtgttcgactactggggacagggcacaacagtgaccgtgtcgtccgccagcacaaagggccctagcgtgttcccactggctccttgtagcagaagtacctcagagagcacggctgctctgggctgcctggtcaaggattatttccctgagcctgtgaccgtgtcctggaacagcggagccctgacaagcggggtgcacaccttccccgccgtgctgcagagcagcggcctgtactctctgtcctctgtcgtgacagtgcctagctctagcctgggcacaaagacctacacctgcaacgtggaccacaagcccagcaacaccaaggtggataagcgggtgtga

SEQ ID NO: 81 - protein, 4 aa, synthetic construct
  REGION 1..4: note = Synthetic: Peptide linker
SGSG

SEQ ID NO: 82 - skipped

SEQ ID NO: 83 - DNA, 947 bp, synthetic construct
  misc_feature 1..947: note = Synthetic: minCBA promoter
  source 1..947: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcc

(19) United States
(12) Patent Application Publication
(10) Pub. No.: US 2024/0401082 A1
(43) Pub. Date: Dec. 5, 2024

(54) Treatment of Dry Age-Related Macular Degeneration

(71) Applicant: Genzyme Corporation, Cambridge, MA (US)

(72) Inventors: Matthew Adamowicz, Marlborough, MA (US); Hao Chen, Acton, MA (US); Amy Frederick, Hopkinton, MA (US); Christian Mueller, Concord, MA (US); Catherine O’Riordan, Newton, MA (US); Vaishnavi Rajagopal, Andover, MA (US); John C. Reed, Miami Beach, FL (US); Michael John Storek, Cambridge, MA (US)

(73) Assignee: Genzyme Corporation, Cambridge, MA (US)

(21) Appl. No.: 18/607,056

(22) Filed: Mar. 15, 2024

Related U.S. Application Data
(60) Provisional application No. 63/607,419, filed on Dec. 7, 2023.
(60) Provisional application No. 63/490,736, filed on Mar. 16, 2023.

(51) Int. Cl. C12N 15/864; A61K 9/00; A61K 31/711; A61P 27/02

(52) U.S. Cl. CPC C12N 15/8645; A61K 9/0048; A61K 31/711; A61P 27/02; C12N 2750/14143; C12N 2800/60

(57) ABSTRACT

The present disclosure provides gene therapy that targets complement pathways for treating dry age-related macular degeneration.

### CROSS REFERENCE TO RELATED APPLICATIONS

This application claims priority from U.S. Provisional Applications 63/490,736, filed on Mar. 16, 2023 and 63/607,419, filed on Dec. 7, 2023. The disclosures of the two priority applications are incorporated herein by reference in their entirety.

### SEQUENCE LISTING

The application contains a Sequence Listing which has been submitted electronically in .XML format and is hereby incorporated by reference in its entirety. Said .XML copy, created on Mar. 12, 2024, is named “122548.US006.xml” and is 189,331 bytes in size.

### BACKGROUND OF THE INVENTION

Age-related macular degeneration (AMD) is a leading cause of vision loss among the elderly. There are two types of AMD: dry and wet. Wet AMD, also called advanced neovascular AMD, is a less common type of AMD and usually causes faster vision loss. Dry AMD, on the other hand, accounts for 85 to 90% of AMD cases worldwide (Schultz et al., Clin Ther. (2021) 43 (10): 1792-818).

Dry AMD typically initiates with retinal pigment epithelium (RPE) dysfunction, initially in the macula of the eye, and progresses to advanced stages with RPE cell death, followed by photoreceptor death and eventual blindness. The hall mark of the disease is the accumulation of drusen in the RPE and activation of the complement pathway. This in turn results in a strong inflammatory response, geographic atrophy, and death of RPE cells and photoreceptors, leading to blindness.

Human genetic variants in multiple complement factors are associated with altered risk of AMD and implicate dysregulation of both the classical and alternative complement pathways as causal factors in disease pathogenesis. Cumulative damage to the retina by aging, environmental stress, and other factors triggers inflammation in multiple pathways, including the complement cascade. When regulatory components in these pathways are compromised, as with several geographic atrophy-linked genetic risk factors in the complement cascade, chronic inflammation can ultimately lead to retinal cell death characteristic of geographic atrophy/dry AMD. Levels of complement activity and inflammation are increased in patients with intermediate AMD and late dry AMD with geographic atrophy (GA). GA is a late-stage of dry AMD, and refers to regions of the retina where cells waste away and die, leading to significant bilateral central loss of vision.

Innate immunity via the complement cascade enables clearance of pathogens or damaged cells via phagocytosis. However, dysregulated complement cascade can also cause deleterious inflammation. There are three pathways of initiation of the complement cascade—the classical pathway, the lectin pathway, and the alternative pathway. The classical pathway is initiated by activation of the C1 complex (C1q, C1r, and C1s) upon binding to IgG or IgM immune complexes, leading to cleavage of C4 and C2, which assemble to form C4b2a, a C3 convertase. The lectin pathway is initiated, for example, by activation of the mannan-binding lectin (MBL)/MBL-associated serine protease (MASP) complex upon oligosaccharide binding, leading to cleavage of C4 and C2, which assemble to form C4b2a. The alternative pathway is constitutively active at a low level and is initiated by hydrolysis of C3 to C3 (H₂O), which binds factor B (FB), leading to the formation of the fluid-phase C3 proconvertase C3 (H₂O) B. This complex is recognized and cleaved by Factor D (FD) to form C3 (H₂O) Bb, the fluid-phase C3 convertase.

All C3 convertases cleave C3 into the anaphylatoxin C3a and the opsonin C3b. Covalently attached C3b mediates phagocytosis of the opsonin-tagged cell. In addition, opsonized C3b amplifies the complement response through the alternative pathway, regardless of the initiation pathway. This amplification triggers the activation of the terminal pathway through the formation of C5 convertases, which cleave C5 into C5a, a potent anaphylatoxin, and C5b, a component of C5b9 or the membrane attack complex (MAC), a large pore complex that can cause cell lysis.

To date, most management guidelines focus on risk factor reduction and use of dietary supplements (Schutz et al., ibid). The first treatment for GA, a C3 inhibitor (SYFOVRE™; pegcetacoplan injection), was recently approved, but it requires chronic, frequent intravitreal injection, which limits patient adherence and incurs an increased risk of developing neovascular AMD. In addition, C3 inhibition does not prevent complement effector functions that are mediated by upstream activation fragments. Another treatment for GA, a C5 inhibitor (IZERVAY™; avacincaptad pegol intravitreal solution) was approved by the FDA a few months after SYFOVRE™ was, but C5 inhibition has similar downsides to C3 inhibition. Thus, there remains an urgent need to develop effective, one-time therapies for dry AMD.

### SUMMARY OF THE INVENTION

The present disclosure provides an expression construct comprising a first nucleotide sequence encoding an inhibitor for activated complement subcomponent C1s and a second nucleotide sequence encoding an inhibitor for complement factor Bb; or a pair of expression constructs, one comprising the first nucleotide sequence and the other comprising the second nucleotide sequence. Unless otherwise specified herein, activated C1s is also referred to herein as “C1s.” Factor Bb is also referred to herein as “FBb” or simply “Bb.”

In some embodiments, the C1s inhibitor and the Bb inhibitor are each an antibody fragment, optionally wherein the antibody fragment is a single-chain Fv (scFv) or a single-chain Fab (scFab). In some embodiments, the C1s inhibitor is an anti-C1s antibody fragment comprising heavy chain CDR (HCDR) 1-3 in SEQ ID NO:7, optionally comprising SEQ ID NOs: 1-3, respectively, and light chain CDR (LCDR) 1-3 in SEQ ID NO:8, optionally comprising SEQ ID NOs: 4-6, respectively. In some embodiments, the Bb inhibitor is an anti-Bb antibody comprising HCDR1-3 in SEQ ID NO:19, optionally comprising SEQ ID NOs: 13-15, respectively, and LCDR1-3 in SEQ ID NO:20, optionally comprising SEQ ID NOs: 16-18, respectively.

In some embodiments, the C1s inhibitor comprises a heavy chain variable domain (V_{H}) comprising SEQ ID NO:7 or an amino acid sequence at least 95% (e.g., at least 96, 97, 98, or 99%) identical thereto, and a light chain variable domain (V_{L}) comprising SEQ ID NO: 8 or an amino acid sequence at least 95% (e.g., at least 96, 97, 98, or 99%) identical thereto. In some embodiments, the Bb inhibitor comprises a V_{H} comprising SEQ ID NO:19 or an amino acid sequence at least 95% (e.g., at least 96, 97, 98, or 99%) identical thereto, and a V_{L} comprising SEQ ID NO:20 or an amino acid sequence at least 95% (e.g., at least 96, 97, 98, or 99%) identical thereto.

In some embodiments, the C1s inhibitor comprises a heavy chain (HC) comprising SEQ ID NO: 10 or an amino acid sequence at least 95% (e.g., at least 96, 97, 98, or 99%) identical thereto, and a light chain (LC) comprising SEQ ID NO:11 or an amino acid sequence at least 95% (e.g., at least 96, 97, 98, or 99%) identical thereto. In some embodiments, the Bb inhibitor comprises an HC comprising SEQ ID NO:22 or an amino acid sequence at least 95% (e.g., at least 96, 97, 98, or 99%) identical thereto and an LC comprising SEQ ID NO:23 or an amino acid sequence at least 95% (e.g., at least 96, 97, 98, or 99%) identical thereto.

In some embodiments, the C1s inhibitor and the Bb inhibitor each comprise one or more charge mutations for promoting pairing between heavy and light chains of each inhibitor. In some embodiments, the charge mutations in the C1s inhibitor comprises Q42E and Q292K, wherein the numbering is in accordance with SEQ ID NO:12. In some embodiments, the charge mutations in the Bb inhibitor comprises Q38K and Q288E, optionally further comprising S114A, N137K, and T434E, wherein the numbering is in accordance with SEQ ID NO:24.

In some embodiments, the C1s inhibitor is an scFv or scFab in which the HC and the LC are linked by a peptide linker, optionally wherein the peptide linker comprises one or more, optionally 2, 3, 4, 5, 6, 7, 8, 9, or 10, G₄S (SEQ ID NO:46) repeats. In some embodiments, the Bb inhibitor is an scFv or scFab in which the HC and the LC are linked by a peptide linker, optionally wherein the peptide linker comprises one or more, optionally 2, 3, 4, 5, 6, 7, 8, 9, or 10, G₄S repeats.

In some embodiments, the expression construct herein comprises a transgene encoding a fusion protein comprising the C1s inhibitor and the Bb inhibitor linked by a peptide linker, optionally wherein the peptide linker comprises one or more, optionally 2, 3, 4, 5, 6, 7, 8, 9, or 10, G₄S repeats. In some embodiments, the transgene is linked operably to a minimal chicken β-actin (minCBA) promoter.

In some embodiments, the expression construct herein comprises a bidirectional promoter that directs expression of the C1s inhibitor and the Bb inhibitor as separate molecules, optionally wherein the bidirectional promoter is a pair of CBA promoters placed in opposite direction and separated by a CMV enhancer, further optionally wherein the bidirectional promoter comprises SEQ ID NO:53 or a nucleotide sequence at least 85% (e.g., at least 90, 91, 92, 93, 94, 95, 96, 97, 98, or 99%) identical thereto.

In some embodiments, the expression construct expresses a heterodimer comprising (i) a fusion protein comprising a single-chain anti-C1s antibody fragment fused to the HC or LC of an anti-Bb antibody fragment; and (ii) the LC or HC polypeptide of the anti-Bb antibody fragment, wherein the coding sequence for the fusion protein and the coding sequence of the LC or HC polypeptide of the anti-Bb antibody fragment are separated in frame by a coding sequence for a cleavable peptide, optionally wherein the cleavable peptide comprises a 2A sequence and/or a furin cleavage site, further optionally the expression construct comprises a minCBA promoter.

In some embodiments, the expression construct expresses a heterodimer comprising (i) a fusion protein comprising a single-chain anti-Bb antibody fragment fused to the HC or LC of an anti-C1s antibody fragment; and (ii) the LC or HC polypeptide of the anti-C1s antibody fragment, wherein the coding sequence for the fusion protein and the coding sequence of the LC or HC polypeptide of the anti-C1s antibody fragment are separated in frame by a coding sequence for a cleavable peptide, optionally wherein the cleavable peptide comprises a 2A sequence and/or a furin cleavage site, further optionally the expression construct comprises a minCBA promoter.

In some embodiments, the expression construct encodes a fusion protein comprises, from N-terminus to C-terminus, (i) an anti-C1s scFv, a (G₄S)₂ linker, and an anti-Bb scFv, optionally comprising SEQ ID NO:55 (with or without the signal peptide) or an amino acid sequence at least 95% (e.g., at least 96, 97, 98, or 99%) identical thereto; (ii) an anti-Bb scFv, a (G₄S)₂ linker, and an anti-C1s scFv, optionally comprising SEQ ID NO:57 (with or without the signal peptide) or an amino acid sequence at least 95% (e.g., at least 96, 97, 98, or 99%) identical thereto; (iii) an anti-C1s scFab, a (G₄S)₃ linker, and an anti-Bb scFab, optionally comprising SEQ ID NO:26 or 28 (with or without the signal peptide), or an amino acid sequence at least 95% (e.g., at least 96, 97, 98, or 99%) identical thereto; (iv) an anti-Bb scFab, a (G₄S)₃ linker, and an anti-C1s scFab, optionally comprising SEQ ID NO:30 or 32 (with or without the signal peptide), or an amino acid sequence at least 95% (e.g., at least 96, 97, 98, or 99%) identical thereto; (v) an anti-C1s scFab, a (G₄S)₂ linker, and an anti-Bb scFv, optionally comprising SEQ ID NO:34 or 36 (with or with the signal peptide), or an amino acid sequence at least 95% (e.g., at least 96, 97, 98, or 99%) identical thereto; or (vi) an anti-C1s scFab, a (G₄S)₃ linker, and an anti-Bb scFv, optionally comprising SEQ ID NO: 59 or 61 (with or without the signal peptide), or an amino acid sequence at least 95% (e.g., at least 96, 97, 98, or 99%) identical thereto.

In some embodiments, the expression construct(s) encodes an anti-C1s scFab, optionally comprising SEQ ID NO:12 or an amino acid sequence at least 95% (e.g., at least 96, 97, 98, or 99%) identical thereto, optionally wherein the amino acid sequence comprises Q42E and Q292K mutations relative to SEQ ID NO: 12; and an anti-Bb scFab, optionally comprising SEQ ID NO: 14 or an amino acid sequence at least 95% (e.g., at least 96, 97, 98, or 99%) identical thereto, optionally wherein the amino acid sequence comprises Q38K and Q288E, and optionally S114A, N137K, and T434E, mutations relative to SEQ ID NO: 14.

In some embodiments, the expression construct encodes a heterodimer comprised of (A) (i) an anti-C1s LC and (ii) a fusion protein comprising an anti-C1s HC fused to an αBb scFab, optionally wherein the expression construct comprises a coding sequence for SEQ ID NO:39, or an amino acid sequence at least 95% (e.g., at least 96, 97, 98, or 99%) identical thereto; (B) (i) an anti-C1s LC and (ii) a fusion protein comprising an anti-C1s HC fused to an anti-Bb scFab, optionally wherein the expression construct comprises a coding sequence for SEQ ID NO:41, or an amino acid sequence at least 95% (e.g., at least 96, 97, 98, or 99%) identical thereto; (C) (i) a fusion protein comprising an anti-C1s scFab fused to an anti-Bb HC and (ii) an anti-Bb LC, optionally wherein the expression construct comprises a coding sequence for SEQ ID NO:43, or an amino acid sequence at least 95% (e.g., at least 96, 97, 98, or 99%) identical thereto; or (D) (i) a fusion protein comprising an anti-C1s scFab fused to an anti-Bb HC and (ii) an anti-Bb LC, optionally wherein the expression construct comprises a coding sequence for SEQ ID NO:45, or an amino acid sequence at least 95% (e.g., at least 96, 97, 98, or 99%) identical thereto.

In another aspect, the present disclosure provides an isolated nucleic acid comprising a nucleotide sequence selected from SEQ ID NOs: 25, 27, 29, 31, 33, 35, 37, 38, 40, 42, 54, 56, 58, 60, 62, 79, or 80, or encodes the same amino acid sequence(s) as the selected nucleotide sequence docs.

In another aspect, the present disclosure provides one, two or more recombinant adeno-associated viruses (rAAV) comprising the expression construct(s) or isolated nucleic acid herein. In some embodiments, the genome of the rAAV herein comprises the expression construct flanked by AAV2 inverted terminal repeats (ITRs). In some embodiments, the genome comprises SEQ ID NO:50, 51, or 52; or encodes the same amino acid sequence(s) as SEQ ID NO:50, 51, or 52 does. In some embodiments, the rAAV herein comprises a capsid of AAV2, optionally wildtype AAV2.

In one aspect, the present disclosure provides a pharmaceutical composition comprising the rAAV herein and a pharmaceutically acceptable carrier.

In one aspect, the present disclosure provides a protein or proteins encoded by the expression construct(s) or rAAV(s) herein.

In one aspect, the present disclosure provides a host cell comprising the expression construct(s), the isolated nucleic acid, or the rAAV(s) herein.

In one aspect, the present disclosure provides a method for treating dry age-related macular degeneration (AMD) in a patient in need thereof, comprising administering an effective amount of the rAAV(s) or pharmaceutical composition herein. In some embodiments, the administering is by intravitreal injection. In some embodiments, the patient has geographic atrophy (GA) secondary to dry AMD. In some embodiments, the effective amount is 10⁷ to 10¹⁵, optionally 10⁸ to 10¹⁴, 10⁹ to 10¹³, further optionally 2×10⁹, 2×10¹⁰, or 2×10¹¹, vector genomes.

Also provided herein are recombinant AAVs or pharmaceutical compositions herein for use in treating dry age-related macular degeneration (AMD) in a patient in need thereof in a treatment method herein, as well as use of the recombinant AAVs or pharmaceutical compositions herein for the manufacture of a medicament for treating dry age-related macular degeneration (AMD) in a patient in need thereof in a treatment method herein.

In another aspect, the present disclosure provides a mammalian promoter comprising a sequence that is at least 85%, optionally at least 90, 91, 92, 93, 94, 95, 96, 97, 98, or 99%, or is 100%, identical to SED ID NO: 83.

In another aspect, the present disclosure provides a bidirectional mammalian promoter comprising a pair of chicken β-actin promoters placed in opposite orientation, separated by a CMV enhancer, optionally wherein the bidirectional mammalian promoter comprises a sequence that is at least 85%, optionally at least 90, 91, 92, 93, 94, 95, 96, 97, 98, or 99%, or is 100%, identical to SED ID NO: 53.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1A is a diagram illustrating an exemplary monocistronic construct for expressing linked (e.g., through a G₄S linker as shown) anti-C1s (αC1s) antibody fragment and anti-Bb (αBb) antibody fragment. minCBA: minimal chicken β-actin promoter. scFab: single-chain antibody fragment. scFv: single chain antibody variable domain. BGH: bovine growth hormone.

FIG. 1B is a diagram illustrating an exemplary bicistronic construct using a bidirectional promoter (a modified minCBA) that allows expression of two separate antibody fragments in opposite directions.

FIG. 1C is a diagram illustrating an exemplary recombinant AAV genome containing an expression cassette of FIG. 1A or FIG. 1B for expressing an anti-Bb antibody fragment and an anti-C1s antibody fragment. ITR: inverted terminal repeat.

FIG. 2A is a panel of diagrams illustrating linked anti-C1s/anti-Bb antibody fragments produced from four exemplary configurations (#5-#8) of a monocistronic construct. Heavy chain variable domain: V_{H}. Light chain variable domain: V_{L}. Heavy chain constant region: C_{H}. Light chain constant region: C_{L}.

FIG. 2B is a pair of diagrams illustrating two exemplary configurations (#9 and #10) of a construct harboring a bidirectional (“BiDir”) promoter driving expression of two independent antibody fragments.

FIG. 2C is a pair of diagrams illustrating exemplary linked anti-C1s/anti-Bb scFab antibody fragments (#11 and #12) with charge mutations (“CM”; Δ) that are intended to promote cognate heavy chain and light chain pairing. In the figures herein, “Δ” indicates the presence of a charge mutation and is not meant to illustrate the exact positions or numbers of the charge mutations in the antibody fragment.

FIG. 2D is a pair of diagrams illustrating exemplary linked anti-C1s/anti-Bb antibody fragments for αC1s scFab-(G₄S)₂-αBb scFv with (#14) or without (#13) charge mutations.

FIG. 2E is a pair of diagrams illustrating exemplary linked anti-C1s/anti-Bb antibody fragments for αC1s scFab-(G₄S)₃-αBb scFv with (#16) or without (#15) charge mutations.

FIG. 2F is a panel of diagrams illustrating exemplary linked anti-C1s/anti-Bb antibody fragments containing self-cleaving peptides, F2A or GT2A, between the heavy and light chains of the αC1s Fab fragment (#17: αC1s F2A Fab-(G₄S)₃-αBb scFab; and #18: αC1s GT2A Fab-(G₄S)₃-αBb scFab) or between the heavy and light chains of the αBb Fab fragment (#19: αC1s scFab-(G₄S)₃-αBb F2A Fab; and #20: αC1s scFab-(G₄S)₃-αBb GT2A Fab). F2A: a self-cleaving peptide comprising a furin cleavage site linked by a SGSG (SEQ ID NO:81) linker to a foot-and-mouth disease virus 2A peptide (Fuchs et al., PLOS One (2016) doi: 10.1371/journal.pone.0158009). GT2A: a self-cleaving peptide comprising a furin cleavage site linked by a GSG linker to a Thosea asigna virus 2A peptide.

FIG. 2G is a pair of diagrams illustrating exemplary configurations of a construct harboring a bidirectional promoter driving expression of two independent antibody fragments that differ from constructs #9 and #10 by having charge mutations (#21 and #22).

FIG. 2H is a diagram showing construct #14 of FIG. 2D («C1s scFab-(G₄S)₂-αBb scFv-CM) in the context of an AAV vector plasmid, including AAV2 ITRs. “αC1s”: αC1s. “αBb”: αBb.

FIG. 2I is a diagram showing construct «C1s scFab-BiDir-αBb scFab with (#21; FIG. 2G) or without (#9; FIG. 2B) charge mutations in the context of an AAV vector plasmid, including AAV2 ITRs. “aC1s”: αC1s. “aBb”: αBb.

FIG. 2J is a diagram showing construct αBb scFab-(G₄S)₃-αC1s scFab-CM (construct #12 of FIG. 2C) in the context of an AAV vector plasmid, including AAV2 ITRs. “αC1s”: αC1s. “αBb”: αBb.

FIG. 3 is a representative biolayer interferometry (BLI) sensorgram showing that the protein expressed from construct #19 of FIG. 2F can bind both C1s and Bb simultaneously.

FIG. 4A is a plot showing dose-dependent inhibition of complement activation by recombinant anti-C1s Fab and the purified protein expressed by construct #2 of FIG. 2A under conditions where both CP and AP are activated simultaneously in vitro.

FIG. 4B is a plot showing dose-dependent inhibition of complement activation by recombinant anti-Bb Fab and the purified protein expressed by construct #4 of FIG. 2A under conditions where both CP and AP are activated simultaneously in vitro.

FIG. 4C is a plot showing dose-dependent inhibition of complement activation by an equimolar mixture of recombinant anti-Bb Fab and anti-C1s Fab, tested alongside an equimolar mixture of purified proteins expressed by constructs #2 and #4 of FIG. 2A under conditions where both CP and AP are activated simultaneously in vitro.

FIG. 5 is a panel of photographs showing representative vector in situ hybridization of the mouse retina 3 weeks after administration of AAV2 #9. Vector-specific probes targeting the vector genome were used.

FIG. 6 is a panel of graphs showing combined inhibition of CP and AP on ARPE19 cells in a CRP-mediated complement activation model of dry AMD. The data shown are an average of twelve replicates along with standard deviation for each condition across two independent experiments. “NHS”: normal human serum. “CRP”: C-reactive protein. **** p<0.0001.

FIGS. 7A and 7B are graphs showing cell-ELISA data depicting complement deposition on induced pluripotent stem cell-derived retinal pigment epithelial cells (iPSC-RPE) in a cell model for AMD. Treatment with anti-Bb and anti-C1s scFabs significantly inhibited deposition of complement products C3d (FIG. 7A) and C5b9 (FIG. 7B) on iPSC-RPE relative to the CRP control. Error bars are standard deviation. **** p<0.0001.

FIGS. 8A and 8B show immunofluorescent staining of C5b9 on iPSC-RPE. FIG. 8A is a panel of confocal microscopy images showing C5b9 deposition (red) on iPSC-RPE. FIG. 8B is a graph showing the quantification analysis of the images in FIG. 8A.

FIG. 9 is a heat map showing ocular exam results based on the preclinical ocular toxicology scoring (SPOTS) system. The heat map shows the clinical indicators of ocular inflammation and irritation in controls before and after LPS treatment; it shows median severity scored during ocular exams using the SPOTS system.

### DETAILED DESCRIPTION OF THE INVENTION

The present disclosure is based on the discovery that dual targeting of the complement classical and alternative pathways can be used to treat eye diseases associated with a dysregulated or overactivated complement system in the eye. The present disclosure provides gene therapy that delivers to the eye(s) of a patient in need thereof both an inhibitor of activated complement component 1 subcomponents (aC1s or simply referred to as “C1s” herein) and an inhibitor of activated factor B (aka. Bb fragment, FBb, or Bb). The gene therapy can use a viral vector, such as recombinant adeno-associated virus (AAV, e.g., AAV2), as a vehicle to deliver transgenes that direct expression of the C1s and Bb inhibitors. In some embodiments, the C1s inhibitor and Bb inhibitor are antibody fragments such as single-chain Fab (scFab) or single-chain Fv (scFv). The C1s inhibitor and the Bb inhibitor can be expressed as a single protein, or as two separate proteins.

In some embodiments, the eye disease to be treated is dry AMD, including associated geographic atrophy. In some embodiments, the patient has a dysregulated/overactivated complement system in the RPE choroid interface. In some embodiments, the present therapy delivers (e.g., intravitreally or subretinally) the present recombinant expression constructs (e.g., recombinant AAV2) to the retinal ganglion cells (RGCs). Intravitreal delivery of rAAV2 transduces RGCs in the retina and facilitates secretion of the inhibitory proteins for distribution to the broader retina. For example, the rAAV2 may be delivered intravitreally to patients with geographic atrophy (GA) secondary to dry AMD to reduce the growth of retinal GA lesion size over a 12-month period and prevent inevitable vision loss. In addition to its benefit as a potential one-time treatment for GA, the presently disclosed gene therapy may have improved efficacy compared to therapeutic approaches that target downstream components in the complement pathway. This is because the present therapy broadly inhibits both proximal and terminal mediators of inflammation, phagocytosis, and membrane attack complex-mediated cell lysis.

Therapies that have been approved or currently under development involve repeat dosing (e.g., monthly or every other month) of complement inhibitors. A one-time treatment with an outpatient intravitreal delivery of a recombinant vector will provide a best-in-class approach. Further, in other therapies, the complement inhibitors block all complement pathways. By contrast, the present bifunctional complement inhibitors target upstream activation steps in the complement pathways implicated as drivers of dry AMD pathogenesis—the AP and CP—rather than targeting downstream convertases common to all three initiating pathways. This approach leaves C1q and the lectin pathway intact to maintain immune surveillance. Furthermore, this approach has a superior mode of action due to inhibition of not only the membrane attack complex (MAC) but also the complement amplification loop and terminal events that are mediated by upstream activation fragments, such as inflammation and opsonization and phagocytosis. The present approach may also reduce target-mediated drug disposition (TMDD) since the inhibitors target activated enzymes that are often present at much lower levels as compared to the intact pro-enzymes.

### I. C1s and Bb Inhibitors

The present gene therapy introduces both a C1s inhibitor and a Bb inhibitor, either linked or unlinked, to the diseased eye of a patient.

Prior to processing and activation, a human C1s polypeptide may have the amino acid sequence of SEQ ID NO:65 (UniProt. P09871), in which amino acids 1-15 constitute the signal peptide. Upon activation, the C1s polypeptide is cleaved and becomes a disulfide-linked heterodimer in which the heavy chain corresponds to amino acids 16-437 of SEQ ID NO: 65 and the light chain corresponds to amino acids 438-688 of SEQ ID NO:65. Unless otherwise indicated, the C1s inhibitor herein refers to an inhibitor of this activated form of C1s.

Prior to processing and activation, a human factor B polypeptide may have the amino acid sequence of SEQ ID NO:66 (UniProt. P00751), in which amino acids 1-25 constitute the signal peptide. Upon activation, the polypeptide is cleaved into two subcomponents, factor Ba, which corresponds to amino acids 26-259 of SEQ ID NO:66, and factor Bb, which corresponds to amino acids 260-764 of SEQ ID NO:66. Factor Bb is also simply referred to as “Bb” herein.

The C1s inhibitor and the Bb inhibitor herein may be linked recombinantly (e.g., expressed recombinantly as a fusion protein), with or without a peptide linker. Where these proteins are introduced into the cell through expression vectors, they may also be referred to as “vectorized” proteins (e.g., “vectorized” antibody fragments).

In some embodiments, the C1s inhibitor and the Bb inhibitor are antigen-binding fragments of full antibodies. A full “antibody” (Ab) or “immunoglobulin” (Ig) refers to a tetrameric protein comprising two heavy (H) chains (about 50-70 kDa) and two light (L) chains (about 25 kDa) inter-connected by disulfide bonds. Each heavy chain is comprised of a heavy chain variable domain (V_{H}) and a heavy chain constant region (C_{H}). Each light chain is composed of a light chain variable domain (V_{L}) and a light chain constant region (C_{L}). The V_{H} and V_{L} domains can be subdivided further into regions of hypervariability, called “complementarity-determining regions” (CDRs), interspersed with regions that are more conserved, called “framework regions” (FRs). Each V_{H} or V_{L} is composed of three CDRs and four FRs, arranged from amino-terminus to carboxyl-terminus in the following order: FR1, CDR1, FR2, CDR2, FR3, CDR3, and FR4. The assignment of amino acids to each region may be in accordance with IMGT® definitions (Lefranc et al., Dev Comp Immunol. (2003) 27 (1): 55-77; or the definitions of Kabat, Sequences of Proteins of Immunological Interest (National Institutes of Health, Bethesda, MD (1987 and 1991)); Chothia & Lesk, J. Mol. Biol. (1987) 196:901-917; or Chothia et al., Nature (1989) 342:878-83. Additional CDR definition systems include the AbM system and the Martin system (see, e.g., Abhinandan and Martin, Mol Immunol. (2008) 45 (14): 3832-9).

The term “antibody fragment,” “antigen-binding fragment” or a similar term refers to the portion of an intact antibody that comprises the amino acid residues that interact with an antigen and confer on the fragment its specificity and affinity for the antigen. The antibody fragment may be a single-chain variable fragment (scFv), which is a fusion protein of the V_{H} and the V_{L} of an antibody, connected with a short peptide linker; a diabody, which is a non-covalent dimer of scFv (Zapata et al., Protein Eng. (1995) 8 (10): 1057-62); or a Fab fragment, including a single-chain Fab (scFab) fragment. “Fab” fragments contain the constant domain of the light chain and the first constant domain (C_{H}1) of the heavy chain. Other nonlimiting examples of antigen-binding fragments of antibodies include Fd fragments, Fv fragments, dAb fragments and minimal recognition units consisting of the amino acid residues that mimic the hypervariable domain of the antibody. In particular embodiments, the antibody fragment is an scFv, a Fab, or an scFab.

A. Anti-C1s scFv and scFab

In some embodiments, the active C1s inhibitor is an antibody fragment such as an sc Fab or an scFv derived from anti-C1s antibody VH3/VK2 from WO 2018/071676. Antibody fragments derived from variants of this antibody as described in WO 2018/071676, or in WO 2016/164358, and U.S. Pat. Nos. 10,729,767 and 11,246,926, may also be used herein. In some embodiments, the anti-C1s (also termed herein “αC1s”) scFv or scFab herein comprises CDRs derived from the aforementioned VH3/VK2 antibody. The CDRs may be defined by any one of the well-known systems, including those described above. In some embodiments, the CDRs are defined by the Kabat system, the IMGT® system, or the Chothia system as shown in Table A below (SEQ ID NOs are shown in parenthesis).

TABLE A

| CDR | Kabat | IMGT ® | Chothia |
|---|---|---|---|
| HCDR1 | DDYIH | GFNIKDDY | GFNIKDD |
| | (1) | (67) | (71) |
| HCDR2 | RIDPADGHTKYAPKFQV | IDPADGHT | DPADGH |
| | (2) | (68) | (72) |
| HCDR3 | YGYGREVFDY | ARYGYGREVFDY | YGYGREVFDY |
| | (3) | (69) | (3) |
| LCDR1 | KASQSVDYDGDSYMN | QSVDYDGDSY | KASQSVDYDGDSYMN |
| | (4) | (70) | (4) |
| LCDR2 | DASNLES | DAS | DASNLES |
| | (5) | | (5) |
| LCDR3 | QQSNEDPWT | QQSNEDPWT | QQSNEDPWT |
| | (6) | (6) | (6) |

In some embodiments, the anti-C1s scFab or scFv comprises heavy chain CDR (HCDR) 1-3 comprising SEQ ID NOs: 1-3, respectively, and light chain CDR (LCDR) 1-3 comprising SEQ ID NOs: 4-6, respectively.

In particular embodiments, the anti-C1s scFv or scFab comprises a V_{H} comprising SEQ ID NO:7 or an amino acid sequence at least 95% (e.g., at least 96, 97, 98, or 99%) identical thereto; and a V_{L} comprising SEQ ID NO:8 or an amino acid sequence at least 95% (e.g., at least 96, 97, 98, or 99%) identical thereto. In certain embodiments, the anti-C1s scFv comprises a peptide linker, such as a flexible linker, e.g., a linker comprising (G₄S)ₙ (SEQ ID NO: 46), where n=1, 2, 3, 4, 5, 6, 7, 8, 9, or 10, linking the V_{H} and the V_{L}. In some embodiments, the linker comprises SEQ ID NO:48 (i.e., n=3). The V_{H} may be N-terminal, or C-terminal, to the V_{L}. In some embodiments, the anti-C1s scFv comprises SEQ ID NO:9 or an amino acid sequence at least 95% (e.g., at least 96, 97, 98, or 99%) identical thereto.

In certain embodiments, the anti-C1s scFab comprises a heavy chain (HC) comprising SEQ ID NO: 10 or an amino acid sequence at least 95% (e.g., at least 96, 97, 98, or 99%) identical thereto; and a light chain (LC) comprising SEQ ID NO:11 or an amino acid sequence at least 95% (e.g., at least 96, 97, 98, or 99%) identical thereto. In further embodiments, the HC and the LC are linked by a peptide linker, such as a flexible linker, e.g., a linker comprising (G₄S)ₙ (SEQ ID NO:46), where n=1, 2, 3, 4, 5, 6, 7, 8, 9, or 10, linking the HC and the LC. In some embodiments, the linker comprises SEQ ID NO:49 (i.e., n=7). The HC may be N-terminal, or C-terminal to the LC. In some embodiments, the αC1s scFab comprises SEQ ID NO: 12 or an amino acid sequence at least 95% (e.g., at least 96, 97, 98, or 99%) identical thereto.

In some embodiments, the C1s inhibitor is an antibody fragment such as an scFab or an scFv derived from anti-C1s antibody disclosed in US2022/0380483A1. For example, the C1s inhibitor may comprise the heavy and light chain CDRs, or V_{H} and V_{L}, of the parental anti-C1s antibody.

B. Anti-Bb scFv and scFab

In some embodiments, the Bb inhibitor is an antibody fragment such as an scFab or an scFv derived from anti-Bb antibody V_{H}6/Vκ7-IgG4v2 from U.S. Pat. No. 11,242,382 and WO 2021/216458. Antibody fragments derived from variants of this antibody as described in WO 2021/216458 may also be used herein. In some embodiments, the anti-Bb (also termed herein “αBb”) scFv or scFab herein comprises CDRs derived from the aforementioned V_{H}6/Vκ7-IgG4v2 antibody. The CDRs may be defined by any one of the well-known systems, including those described above. In some embodiments, the CDRs are defined by the Kabat system, the IMGT® system, or the Chothia system as shown in Table B below (SEQ ID NOs are shown in parenthesis).

TABLE B

| CDR | Kabat | IMGT ® | Chothia |
|---|---|---|---|
| HCDR1 | NYAMS | GFTFSNYA | GFTFSNY |
| | (13) | (73) | (77) |
| HCDR2 | TISNRGSYTYYPDSVKG | ISNRGSYT | SNRGSY |
| | (14) | (74) | (78) |
| HCDR3 | ERPMDY | ARERPMDY | ERPMDY |
| | (15) | (75) | (15) |
| LCDR1 | KASQDVGTAVA | QDVGTA | KASQDVGTAVA |
| | (16) | (76) | (16) |
| LCDR2 | WASTRHT | WAS | WASTRHT |
| | (17) | | (17) |
| LCDR3 | HQHSSNPLT | HQHSSNPLT | HQHSSNPLT |
| | (18) | (18) | (18) |

In some embodiments, the anti-Bb scFab or scFv comprises heavy chain CDR (HCDR) 1-3 comprising SEQ ID NOs: 13-15, respectively, and light chain CDR (LCDR) 1-3 comprising SEQ ID NOs: 16-18, respectively.

In particular embodiments, the anti-Bb scFv or scFab comprises a V_{H} comprising SEQ ID NO:19 or an amino acid sequence at least 95% (e.g., at least 96, 97, 98, or 99%) identical thereto; and a V_{L} comprising SEQ ID NO:20 or an amino acid sequence at least 95% (e.g., at least 96, 97, 98, or 99%) identical thereto. In certain embodiments, the anti-Bb scFv comprises a peptide linker, such as a flexible linker, e.g., a linker comprising (G₄S) n (SEQ ID NO:46), where n=1, 2, 3, 4, 5, 6, 7, 8, 9, or 10, linking the V_{H} and the V_{L}. In some embodiments, the linker comprises SEQ ID NO:48 (i.e., n=3). The V_{H} may be N-terminal, or C-terminal, to the V_{L}. In some embodiments, the anti-Bb scFv comprises SEQ ID NO:21 or an amino acid sequence at least 95% (e.g., at least 96, 97, 98, or 99%) identical thereto.

In certain embodiments, the anti-Bb scFab comprises a heavy chain (HC) comprising SEQ ID NO:22 or an amino acid sequence at least 95% (e.g., at least 96, 97, 98, or 99%) identical thereto; and a light chain (LC) comprising SEQ ID NO:23 or an amino acid sequence at least 95% (e.g., at least 96, 97, 98, or 99%) identical thereto. In further embodiments, the HC and the LC are linked by a peptide linker, such as a flexible linker, e.g., a linker comprising (G₄S)ₙ (SEQ ID NO:46), where n=1, 2, 3, 4, 5, 6, 7, 8, 9, or 10, linking the HC and the LC. In some embodiments, the linker comprises SEQ ID NO:49 (i.e., n=7). The HC may be N-terminal, or C-terminal to the LC. In some embodiments, the αBb scFab comprises SEQ ID NO:24 or an amino acid sequence at least 95% (e.g., at least 96, 97, 98, or 99%) identical thereto.

In some embodiments, the Bb inhibitor is an antibody fragment such as an scFab or an scFv derived from anti-Bb antibody disclosed in U.S. Pat. Nos. 10,131,706; 10,604,563; or 7,964,705. For example, the Bb inhibitor may comprise the heavy and light chain CDRs, or V_{H} and V_{L}, of the parental anti-Bb antibody.

### C. Anti-C1s/Bb Bispecific Fusion Proteins

In some embodiments, the C1s inhibitor (e.g., anti-C1s scFab or scFv) and the Bb inhibitor (e.g., anti-Bb scFab or scFv) are linked by a peptide linker, such as a flexible linker, e.g., a linker comprising (G₄S)ₙ (SEQ ID NO:46), where n=1, 2, 3, 4, 5, 6, 7, 8, 9, or 10, linking the two inhibitors. In some embodiments, the peptide linker is SEQ ID NO:47 (n=2) or 48 (n=3). The C1s inhibitor may be N-terminal, or C-terminal to the Bb inhibitor. The αC1s/αBb fusion protein may have the following exemplary, nonlimiting configurations (from N-terminus to C-terminus):
αC1s scFab-Linker-αBb scFab
αC1s scFv-Linker-αBb scFab
αC1s scFab-Linker-αBb scFv
αC1s scFv-Linker-αBb scFv
αBb scFab-Linker-αC1 scFab
αBb scFv-Linker-αC1 scFab
αBb scFab-Linker-αC1 scFv
αBb scFv-Linker-αC1 scFv
   wherein the “Linker” may be one of the peptide linkers described herein (e.g., a flexible linker described herein), such as (G₄S)₂ (SEQ ID NO:47) and (G₄S)₃ (SEQ ID NO:48), and wherein within each configuration, the scFab and/scFv may have the heavy chain and the light chain in the order of N-heavy-light-C, or N-light-heavy-C.

To facilitate cognate pairing of heavy and light chains within each antigen-binding domain of the fusion protein, each antigen-binding domain may contain charge mutations. Charge mutations refer to substitution of a charge-neutral amino acid (e.g., Q) by a positively charged (e.g., K) or negatively charged (e.g., E) amino acid, and substitution of a charged amino acid to an amino acid of the opposite charge. To increase pairing of two polypeptide chains, the interactive residues on the two chains may be mutated to amino acid residues of opposite charges. Exemplary charge mutations that may contribute to cognate antibody chain pairing are described in, e.g., Tan et al., Biophys J (1998) 75:1473-82; US2014/0242076A1; and WO 2020/136566. In some embodiments,
the charge mutations in the αC1s scFv or scFab comprise Q42E (V_{L}) and Q292K (V_{H}) mutations (numbering according to SEQ ID NO:12);
the charge mutations in the αBb scFv comprises Q38K (V_{L}) and Q288E (V_{H}) (numbering according to SEQ ID NO:24); and
the charge mutations in the αBb scFab comprises Q38K (V_{L}) and Q288E (V_{H}), and optionally further comprises S114A (C_{L}), N137K (C_{L}), and T434E (C_{H}1) (numbering according to SEQ ID NO: 24).

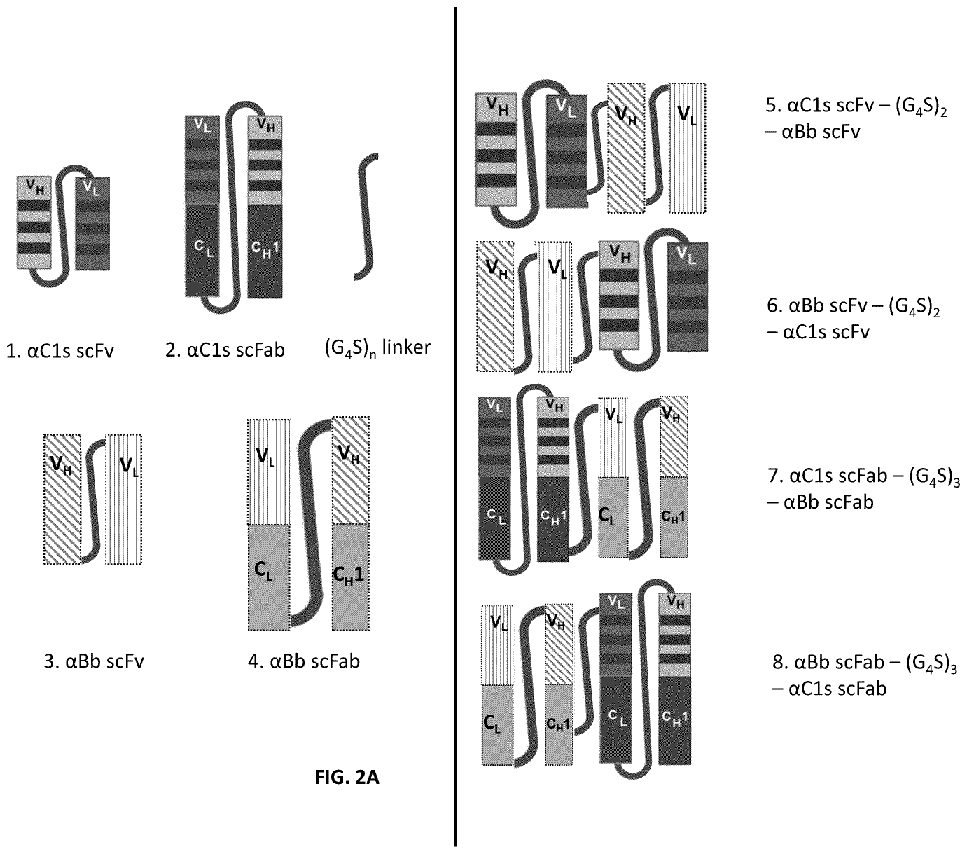

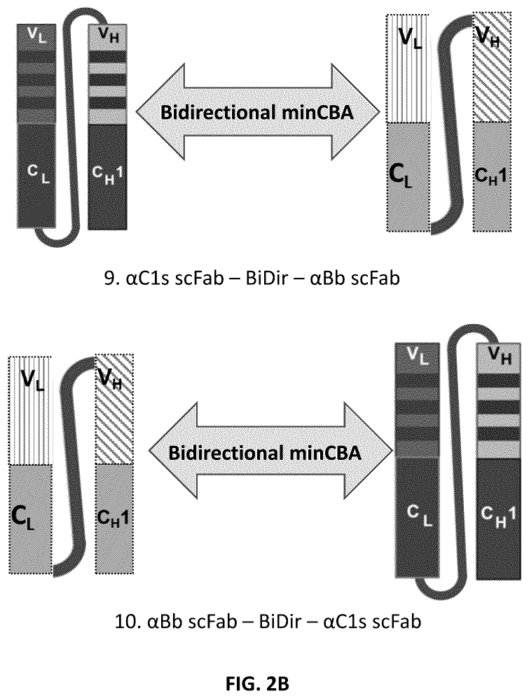

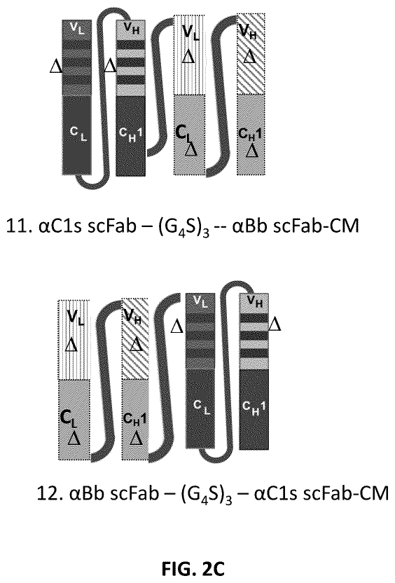

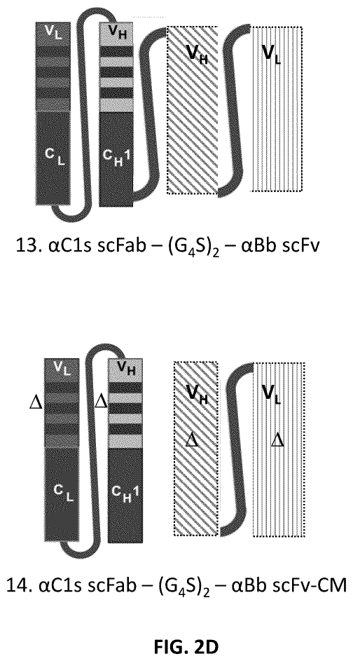

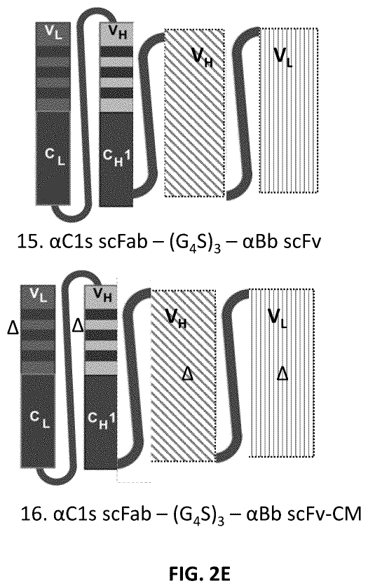

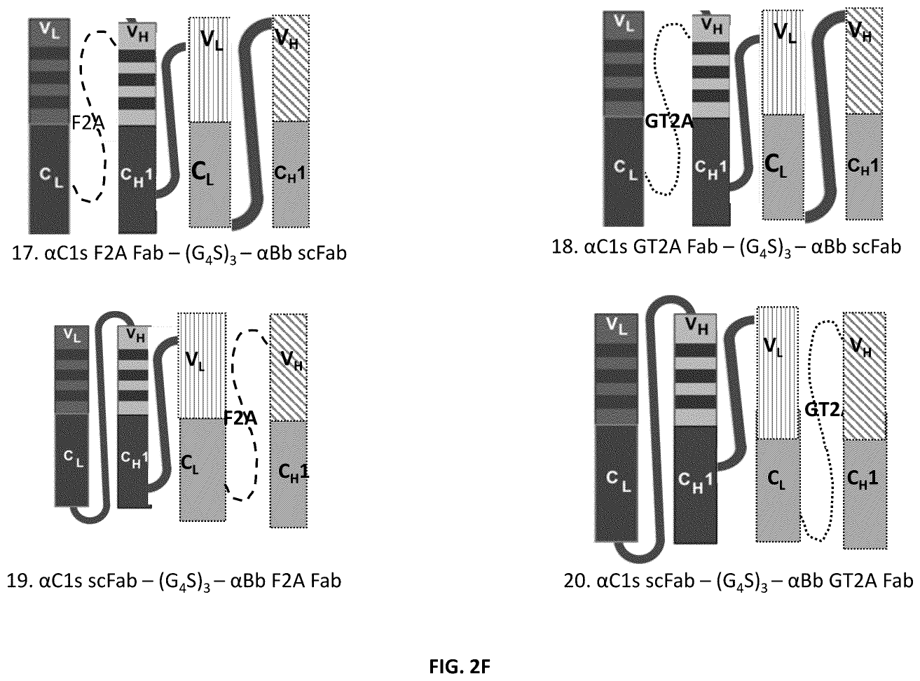

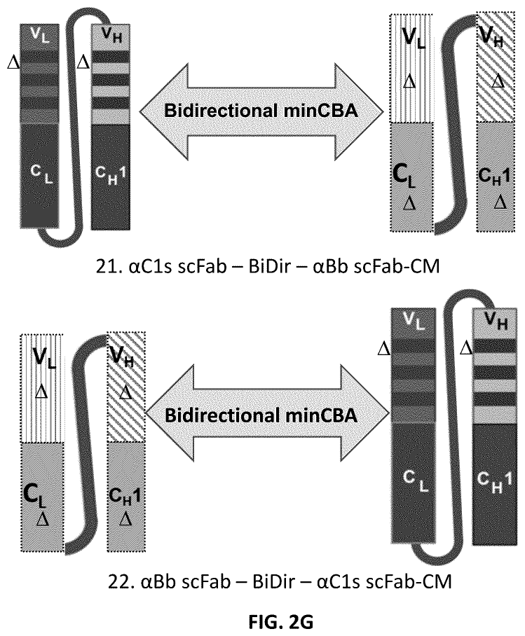

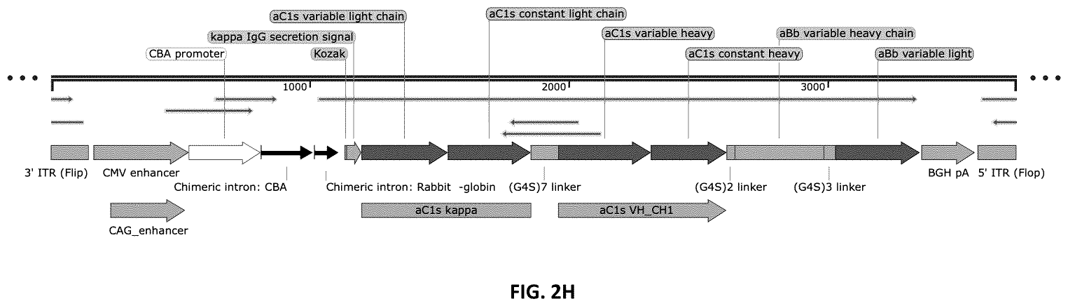

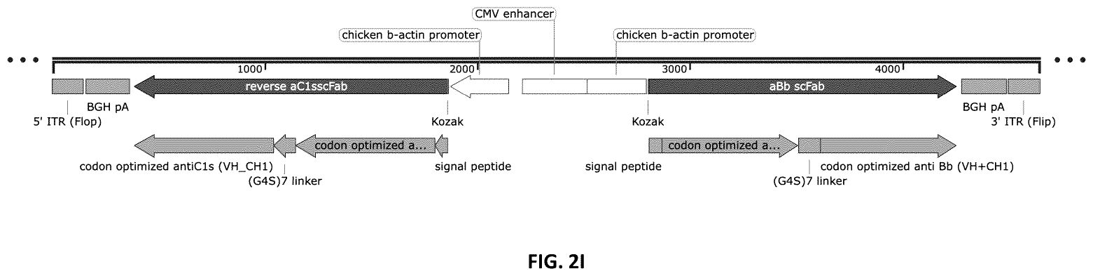

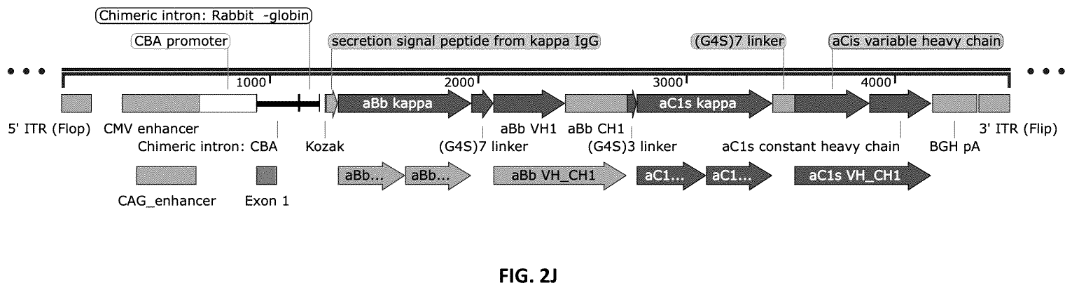

In some embodiments, the fusion protein has a structure shown in construct #5 (FIG. 2A), where components of the fusion protein are in the order of, from N-terminus to C-terminus, αC1s scFv-(G₄S)₂-αBb scFv. In particular embodiments, this fusion protein is encoded by SEQ ID NO:54, or comprises SEQ ID NO:55 (with or without the signal peptide) or an amino acid sequence at least 95% (e.g., at least 96, 97, 98, or 99%) identical thereto.

In some embodiments, the fusion protein has a structure shown in construct #6 (FIG. 2A), where components of the fusion protein are in the order of, from N-terminus to C-terminus, αBb scFv-(G₄S)₂-αC1s scFv. In particular embodiments, this fusion protein is encoded by SEQ ID NO:56, or comprises SEQ ID NO:57 (with or without the signal peptide) or an amino acid sequence at least 95% (e.g., at least 96, 97, 98, or 99%) identical thereto.

In some embodiments, the fusion protein has a structure shown in construct #7 (FIG. 2A), where components of the fusion protein are in the order of, from N-terminus to C-terminus, αC1s scFab-(G₄S)₃-αBb scFab. In particular embodiments, this fusion protein is encoded by SEQ ID NO:25, or comprises SEQ ID NO:26 (with or without the signal peptide) or an amino acid sequence at least 95% (e.g., at least 96, 97, 98, or 99%) identical thereto.

In some embodiments, the fusion protein has a structure shown in construct #8 (FIG. 2A), where components of the fusion protein are in the order of, from N-terminus to C-terminus, αBb scFab-(G₄S)₃-αC1s scFab. In particular embodiments, this fusion protein is encoded by SEQ ID NO:29, or comprises SEQ ID NO:30 (with or without the signal peptide) or an amino acid sequence at least 95% (e.g., at least 96, 97, 98, or 99%) identical thereto.

In some embodiments, the fusion protein has a structure shown in construct #11 (FIG. 2C), where components of the fusion protein are in the order of, from N-terminus to C-terminus, αC1s scFab-(G₄S)₃-αBb scFab (with CMs). In particular embodiments, this fusion protein is encoded by SEQ ID NO:27, or comprises SEQ ID NO:28 (with or with the signal peptide) or an amino acid sequence at least 95% (e.g., at least 96, 97, 98, or 99%) identical thereto.

In some embodiments, the fusion protein has a structure shown in construct #12 (FIG. 2C), where components of the fusion protein are in the order of, from N-terminus to C-terminus, αBb scFab-(G₄S)₃-αC1s scFab (with CMs). In particular embodiments, this fusion protein is encoded by SEQ ID NO:31, or comprises SEQ ID NO:32 (with or without the signal peptide) or an amino acid sequence at least 95% (e.g., at least 96, 97, 98, or 99%) identical thereto.

In some embodiments, the fusion protein has a structure shown in construct #13 (FIG. 2D), where components of the fusion protein are in the order of, from N-terminus to C-terminus, αC1s scFab-(G₄S)₂-αBb scFv. In particular embodiments, this fusion protein is encoded by SEQ ID NO:33, or comprises SEQ ID NO:34 (with or without the signal peptide) or an amino acid sequence at least 95% (e.g., at least 96, 97, 98, or 99%) identical thereto.

In some embodiments, the fusion protein has a structure shown in construct #14 (FIG. 2D), where components of the fusion protein are in the order of, from N-terminus to C-terminus, αC1s scFab-(G₄S)₂-αBb scFv-CM (#13 with CMs in both αC1s and αBb). In particular embodiments, this fusion protein is encoded by SEQ ID NO:35, or comprises SEQ ID NO: 36 (with or without the signal peptide) or an amino acid sequence at least 95% (e.g., at least 96, 97, 98, or 99%) identical thereto.

In some embodiments, the fusion protein has a structure shown in construct #15 (FIG. 2E), where components of the fusion protein are in the order of, from N-terminus to C-terminus, αC1s scFab-(G₄S)₃-αBb scFv. In particular embodiments, this fusion protein is encoded by SEQ ID NO:58, or comprises SEQ ID NO:59 (with or without the signal peptide) or an amino acid sequence at least 95% (e.g., at least 96, 97, 98, or 99%) identical thereto.

In some embodiments, the fusion protein has a structure shown in construct #16 (FIG. 2E), where components of the fusion protein are in the order of, from N-terminus to C-terminus, αC1s scFab-(G₄S)₃-αBb scFv-CM (with CMs). In particular embodiments, this fusion protein is encoded by SEQ ID NO:60, or comprises SEQ ID NO:61 (with or without the signal peptide) or an amino acid sequence at least 95% (e.g., at least 96, 97, 98, or 99%) identical thereto.

### D. Bispecific Heterodimers

In some embodiments, the dual-targeting complement inhibitors are anti-C1s/anti-Bb bispecific heterodimeric proteins. These proteins are encoded by one single open reading frame, but the HC and LC of one of the antibody fragments are cleaved upon translation and post-translational processing within the cell, yielding two separate polypeptides that are folded into two antigen-binding domains. FIG. 2F illustrates such configurations. In these illustrated configurations, the HC and the LC of one of the antibody fragments are linked by a cleavable peptide (e.g., a self-cleaving 2A peptide with or without a protease (e.g., furin) cleavage site). See also discussions in Section II (“Recombinant Expression Constructs”).

In some embodiments, the heterodimer has a structure shown in construct #17 (FIG. 2F), where the heterodimer is comprised of (i) an αC1s LC and (ii) a fusion protein comprising an αC1s HC fused to an αBb scFab. In particular embodiments, this heterodimer is encoded by SEQ ID NO:38, or comprise, pre-cleavage, SEQ ID NO:39 or an amino acid sequence at least 95% (e.g., at least 96, 97, 98, or 99%) identical thereto (including or not including the two signal peptide sequences).

In some embodiments, the heterodimer has a structure shown in construct #18 (FIG. 2F), where the heterodimer is comprised of (i) an αC1s LC and (ii) a fusion protein comprising an αC1s HC fused to an αBb scFab. In particular embodiments, this heterodimer is encoded by SEQ ID NO:40, or comprise, pre-cleavage, SEQ ID NO:41 or an amino acid sequence at least 95% (e.g., at least 96, 97, 98, or 99%) identical thereto (including or not including the two signal peptide sequences).

In some embodiments, the heterodimer has a structure shown in construct #19 (FIG. 2F), where the heterodimer is comprised of (i) a fusion protein comprising an αC1s scFab fused to an αBb HC and (ii) an αBb LC. In particular embodiments, this heterodimer is encoded by SEQ ID NO:42, or comprise, pre-cleavage, SEQ ID NO:43 or an amino acid sequence at least 95% (e.g., at least 96, 97, 98, or 99%) identical thereto (including or not including the two signal peptide sequences).

In some embodiments, the heterodimer has a structure shown in construct #20 (FIG. 2F), where the heterodimer is comprised of (i) a fusion protein comprising an αC1s scFab fused to an αBb HC and (ii) an αBb LC. In particular embodiments, this heterodimer is encoded by SEQ ID NO:44, or comprise, pre-cleavage, SEQ ID NO:45 or an amino acid sequence at least 95% (e.g., at least 96, 97, 98, or 99%) identical thereto (including or not including the two signal peptide sequences).

### E. Peptide Linkers

The peptide linkers linker the various domains of the present antibody fragments and fusion proteins may preferably be flexible linkers so as to allow for proper folding, movement, and interaction of the joined domains. In some embodiments, the flexible peptide linker herein largely comprises small amino acids (e.g., Gly, Ser, or Thr). In some embodiments, the peptide linker herein consists primarily (e.g., more than 50% of the residues) of Gly and Ser residues (“GS” linker). As described above, such a peptide linker may comprise (G₄S)ₙ (SEQ ID NO:46). By adjusting the copy number “n,” the length of the linker can be adjusted to achieve the desired distance of the joined functional domains. In some embodiments, the peptide linker may contain additional amino acids such as Thr and Ala to maintain flexibility, as well as polar amino acids such as Lys and Glu to improve solubility. See, e.g., Chen et al., Adv Drug Deliv Rev. (2013) 65 (10): 1357-69.

### II. Recombinant Expression Constructs

The present disclosure provides recombinant expression constructs for expressing the C1s/Bb inhibitors herein. The expression constructs have an expression cassette comprising coding sequences for the C1s/Bb inhibitors, linked operably to a promoter and a poly (A) signal sequence. The coding sequences may be human codon-optimized to improve expression in human cells. The coding sequences may encode a signal peptide (e.g., a signal peptide from IgG Kappa) to support secretion of the proteins. The expression cassette may also include additional transcription regulatory sequences, such as a Kozak sequence and a sequence that enhances gene expression or RNA stability (e.g., a WPRE element).

### A. Configurations of Expression Constructs

### 1. Expression Constructs Encoding a Single Fusion Protein

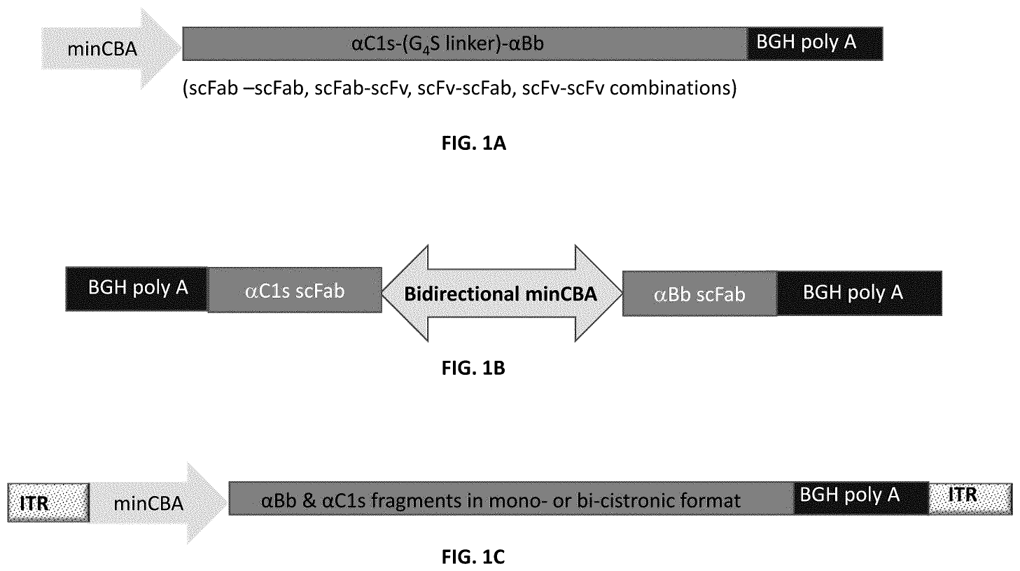

In some embodiments, the expression construct herein is monocistronic and comprises a coding sequence for an αC1s/αBb fusion protein. See, e.g., FIGS. 1A and 1C. By way of example, the expression construct may be one of the numbered constructs #5 through #8 and constructs #11 through #16, whose gene products are described in the section above.

### 2. Expression Constructs Encoding Two Separate Proteins

In some embodiments, the expression construct encodes the C1s inhibitor and the Bb inhibitor as two separate proteins. Independent target engagement may remove the possibility of steric hindrance.

For example, the expression construct has two separate expression cassettes, one for each of the C1s inhibitor (e.g., scFv or scFab) and the Bb inhibitor (e.g., scFv or scFab). Each expression cassette has its own transcriptional regulatory sequences such as promoters and enhancers.

In another configuration, the expression construct has a bicistronic expression cassette and a single promoter. The coding sequences for the C1s inhibitor and the Bb inhibitor are transcribed together under the single promoter, into one mRNA, and then the RNA sequence for each isoform is translated separately through the use of an internal ribosome entry site (IRES) in the mRNA. In another approach, the coding sequences of the C1s and Bb inhibitors are separated by the coding sequence for a self-cleaving peptide and/or a protease (e.g., furin) cleavage site, such that translation of the mRNA transcript and subsequent processing yield two separate gene products (C1s inhibitor and Bb inhibitor). Examples of self-cleaving peptides are 2A peptides, which are viral derived peptides with a typical length of 18-22 amino acids. 2A peptides include T2A, P2A, E2A, and F2A. Translation of the transgene can leave a few amino acid residues from the 2A peptide on one or both of the gene product. A furin cleavage site may be included to allow removal of the extra amino acid residues.

In yet another configuration, the bicistronic expression construct comprises a bidirectional promoter that allows for individual expression of each inhibitor. See, e.g., By way of example, the expression construct may be one of the numbered constructs #9, #10, #21, and #22 illustrated in FIGS. 2B and 2G and listed below (BiDir: bidirectional promoter)
#9: αC1s scFab-BiDir-αBb scFab, producing separate αC1s scFab and αBb sc Fab
#10: αBb scFab-BiDir-αC1s scFab, producing separate αC1s scFab and αBb scFab.
#21: αC1s scFab-BiDir-αBb scFab-CM, producing separate αC1s scFab-CM and αBb scFab-CM
#22: αBb scFab-BiDir-αC1s scFab-CM, producing separate αC1s scFab-CM and αBb scFab-CM

In constructs #21 and #22, both the anti-C1s and anti-Bb scFabs contain charge mutations (CMs) to promote cognate pairing of the heavy and light chains within each antibody fragment.

### 3. Expression Constructs Encoding Heterodimers

In some embodiments, the expression construct encodes a heterodimer comprised of a first single-chain antibody fragment (e.g., scFab or scFv) fused to one of the two chains of a second antibody fragment (e.g., Fab), where this fusion polypeptide complexes with the other chain of the second antibody fragment. The heterodimer is bispecific and binds both C1s and Bb.

Exemplary constructs that encode bispecific heterodimers configurations are illustrated in FIG. 2F and listed below:
#17: αC1s F2A Fab-(G₄S)₃-αBb scFab, producing a heterodimer comprised of (i) an αC1s LC and (ii) a fusion protein comprising an αC1s HC fused to an αBb scFab
#18: αC1s GT2A Fab-(G₄S)₃-αBb scFab, producing a heterodimer comprised of (i) an αC1s LC and (ii) a fusion protein comprising an αC1s HC fused to an αBb scFab
#19: αC1s scFab-(G₄S)₃-αBb F2A Fab, producing a heterodimer comprised of (i) a fusion protein comprising an αC1s scFab fused to an αBb HC and (ii) an αBb LC
#20: αC1s scFab-(G₄S)₃-αBb GT2A Fab, producing a heterodimer comprised of (i) a fusion protein comprising an αC1s scFab fused to an αBb HC and (ii) an αBb LC
   In the above constructs, inclusion of a coding sequence for a cleavable peptide, such as F2A and GT2A, lead to production of two separate polypeptides, which subsequently complex and fold into one single, bispecific heterodimer protein. The F2A and GT2A coding and amino acid sequences are shown in SEQ ID NOs: 38-45. Coding sequences for other cleavable peptides (e.g., those described above) may also be used.

### 4. Separate Expression Constructs for C1s Inhibitor and Bb Inhibitor

In some embodiments, the C1s inhibitor and the Bb inhibitor may be expressed from two separate constructions, e.g., two separate recombinant AAVs, as further described below. The two AAVs may be of the same or different serotypes.

### B. Transcriptional Regulatory Sequences

In the present expression constructs, the coding sequences for the C1s inhibitor and the Bb inhibitor are linked operably to transcription regulatory sequences such as a promoter and an enhancer, to allow expression of the encoded proteins in the intended target cells.

In some embodiments, the C1s and Bb inhibitors are produced in recombinant host cells. In such cases, the promoter and enhancer are those active in the host cells.

In some embodiments, the C1s and Bb inhibitors are delivered through gene therapy and are produced in vivo in the eye of a subject (e.g., a human, a nonhuman primate, or a mouse). In such cases, the promoter may be a constitutive promoter or an inducible promoter that functions in ocular or retina cells (e.g., RGCs and RPE cells of the inner and outer nuclear layers, Mueller cells, and photoreceptors).

In some embodiments, the promoter is a minCBA promoter comprising a CMV enhancer, a chicken β-actin promoter, and an intronic sequence. The minCBA promoter may have a sequence that is at least 85% (e.g., at least 90, 91, 92, 93, 94, 95, 96, 97, 98, or 99%), or completely, identical to SED ID NO: 83.

In some embodiments, the promoter is a bidirectional promoter. The bidirectional promoter may contain, for example, a pair of CBA promoters placed in opposite orientation, separated by a CMV enhancer. In particular embodiments, the bidirectional promoter comprises a sequence that is at least 85% (e.g., at least 90, 91, 92, 93, 94, 95, 96, 97, 98, or 99%), or completely, identical to SED ID NO: 53.

In some embodiments, the expression cassette has a poly (A) signal sequence derived from bovine growth hormone gene. In particular embodiments, the poly (A) signal sequence comprises a sequence that is at least 85% (e.g., at least 90, 91, 92, 93, 94, 95, 96, 97, 98, or 99%), or completely, identical to the sequence that is italicized and underlined in SED ID NO: 51 shown in the Sequences section below.

In some embodiments, the expression cassette contains an enhancer, such as a CMV enhancer. In particular embodiments, the CMV enhancer comprises a sequence that is at least 85% (e.g., at least 90, 91, 92, 93, 94, 95, 96, 97, 98, or 99%), or completely, identical to the sequence that is boldfaced and italicized in SED ID NO: 53 shown in the Sequence section below.

In some embodiments, the expression cassette contains an intron sequence such as a chimeric intron. The intron sequences may increase transgene expression levels by promoting transport of mRNA out of the nucleus and enhancing mRNA stability.

### C. Recombinant AAV Expression Vectors

In some embodiments, a viral vector is used to deliver vectorized antibody fragments to the eye of a patient. In some embodiments, the expression/delivery vector is a recombinant adeno-associated viral (rAAV) expression vector. The expression constructs herein may be rAAV genomes. In the case of rAAV genomes, an expression cassette herein may be flanked by a pair of AAV inverted terminal repeats (ITRs), such as AAV2 ITRs. A nonlimiting example of a unidirectional, monocistronic AAV2 recombinant genome is shown in FIG. 2H. A nonlimiting example of a bidirectional, bicistronic AAV2 recombinant genome is shown in FIG. 2I.

An exemplary rAAV genome harboring construct #9 may have an exemplary nucleotide sequence of SEQ ID NO:50, or a nucleotide sequence encoding the same amino acid sequences as does SEQ ID NO:50 and comprising a sequence that is at least 50% (e.g., at least 60, 65, 70, 75, 80, 85, 90, or 95%) identical to SEQ ID NO:50.

An exemplary rAAV genome harboring construct #12 may have an exemplary nucleotide sequence of SEQ ID NO:51, or a nucleotide sequence encoding the same amino acid sequences as does SEQ ID NO:51 and comprising a sequence that is at least 50% (e.g., at least 60, 65, 70, 75, 80, 85, 90, or 95%) identical to SEQ ID NO:51.

An exemplary rAAV genome harboring construct #14 may have an exemplary nucleotide sequence of SEQ ID NO:52, or a nucleotide sequence encoding the same amino acid sequences as does SEQ ID NO:52 and comprising a sequence that is at least 50% (e.g., at least 60, 65, 70, 75, 80, 85, 90, or 95%) identity to SEQ ID NO:52.

The rAAV genome can be constructed by inserting the expression cassettes herein into an rAAV genome that has had the major rAAV open reading frames excised therefrom. Other portions of the rAAV genome can also be deleted, so long as a sufficient portion of the ITRs remain to allow for replication and packaging functions.

Any suitable AAV serotype may be used. For example, the AAV may be AAV1, AAV2, AAV3, AAV3B, AAV4, AAV5, AAV6, AAV7, AAV8, AAV8.2, AAV9, or AAVrh10, or of a pseudotype or a serotype that is a mutant, variant or derivative of one of the AAV serotypes listed herein (i.e., AAV derived from multiple serotypes). The AAV may be engineered such that its capsid proteins have reduced immunogenicity or enhanced transduction ability in humans or nonhuman primates.

In some embodiments, the rAAV herein has an AAV2 capsid. In particular embodiments, the AAV2 capsid is a wildtype AAV2 capsid. In other embodiments, the AAV2 capsid contains mutations that improve the rAAV2's potency and production yield.

Viral vectors described herein may be produced using methods known in the art. Any suitable permissive or packaging cells may be employed to produce the viral particles. For example, mammalian (e.g., 293 or HeLa) or insect (e.g., Sf9) cells may be used as the packaging cell line. Recombinant AAV vectors can be replicated and packaged into infectious viral particles when introduced into host cells that have been infected with a suitable helper virus (or that is expressing suitable helper functions) and that is expressing AAV rep and cap gene products (i.e., AAV Rep and capsid proteins). See, e.g., U.S. Pat. No. 11,261,463.

### D. Transfection of Host Cells

Where the C1s and Bb inhibitors are delivered directly to patients, the inhibitors may be produced in recombinant mammalian host cells such as COS, NS0, 293, HeLa, or CHO cells. Once the vector has been incorporated into the appropriate host, the host is maintained under conditions suitable for high level expression of the nucleotide sequences, and the collection and purification of the inhibitors.

### III. Pharmaceutical Compositions and Use

The present disclosure provides pharmaceutical compositions comprising the dual targeting C1s/Bb inhibitors or recombinant viral vectors such as AAV vectors encoding the inhibitors. The pharmaceutical compositions may comprise pharmacologically, especially ophthalmologically, acceptable carriers, diluents, and/or excipients. For example, the composition may comprise a tonicity agent (e.g., sodium chloride, amino acids, sugars, or combinations thereof), a surfactant (e.g., polysorbate 20 or polysorbate 80), and/or a stabilizer (e.g., a methioninc).

The pharmaceutical compositions may be delivered by intraocular injection, e.g., injection into the anterior chamber via the temporal limbus, suprachoroidal injection, intracameral injection, intrastromal injection, subretinal injection, intravitreal injection (e.g., front, mid or back vitreous injection).

The present pharmaceutical compositions may be delivered in a therapeutically effective amount to treat dry AMD and geographic atrophy (GA) secondary to dry AMD. An “therapeutically effective amount” means a dosage sufficient to produce a desired result, e.g., amelioration of one or more symptoms (e.g., growth of GA lesions, retinal lesions, or destruction of retinal layer) of the disease to be treated, and/or slowing progression of the disease. A desired result may also include improvement in one or more functional symptoms; for example, the desired result may be reduction of visual distortions, improved central vision, improved vision in low light settings, and/or reduced blurriness. By “treat” is meant amelioration of one or more symptoms of the disease and/or slowing of the progress of the disease.

The present pharmaceutical compositions may be delivered in a prophylactically effective amount to prevent the onset of dry AMD or geographic atrophy (GA) secondary to dry AMD. An “prophylactically effective amount” means a dosage sufficient to produce a desired result, e.g., prevention or delay of the onset of dry AMD and/or GA, and/or prevention or delay of the onset of one or more symptoms of dry AMD and/or GA. Patients who are at high risk of developing dry AMD, such as those with genetic predisposition, may be administered with the present pharmaceutical compositions prophylactically.

In some embodiments, the dosage of recombinant AAV (rAAV) injected into the eye is 10⁷ to 10¹⁵ vector genomes (vg), for example, 10⁸ to 10¹⁴, 10⁹ to 10¹³, or 10⁹ to 10¹², vg. In some embodiments, the dosage of rAAV is 2×10⁹, 2×10¹⁰, or 2×10¹¹ vg.

In some embodiments, the patient is treated, before, during, and/or after the rAAV injection, with an anti-inflammatory agent (e.g., a steroid) to prevent or ameliorate potential immune response against the rAAV. In some embodiments, the patient may be pre-treated with an IgG-degrading enzyme, such as IdeS, to reduce pre-existing neutralizing antibodies to the AAV capsid. These immune modulators may be administered locally or systematically. In some embodiments, the modulators may be administered intraocularly (e.g., intravitreally), orally, intravenously, intramuscularly, or subcutaneously.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

As used herein, the percent identity of two amino acid sequences (or of two nucleic acid sequences) may be obtained by, e.g., BLAST® using default parameters (available at the U.S. National Library of Medicine's National Center for Biotechnology

Information website). In some embodiments, the length of a query sequence aligned for comparison purposes is at least 30% (e.g., at least 40, 50, 60, 70, 80, or 90%) of the length of the reference sequence.

According to the present disclosure, back-references in the dependent claims are meant as short-hand writing for a direct and unambiguous disclosure of each and every combination of claims that is indicated by the back-reference. Any compound disclosed herein can be used in any of the treatment methods disclosed herein, wherein the individual to be treated is as defined anywhere herein.

In order that this invention may be better understood, the following examples are set forth. These examples are for purposes of illustration only and are not to be construed as limiting the scope of the invention in any manner.

### Examples

### Example 1: Vectorized Antibodies and Expression Constructs Thereof

This Example describes the design of bifunctional expression constructs that express inhibitors to C1s and Bb, and the characterization of the recombinant proteins produced from these constructs. These constructs have the following features: (i) either a unidirectional or a bidirectional promoter (e.g., minCBA promoter) to drive constitutive transgene expression; (ii) a transgene (e.g., a transgene that contains human codon-optimized sequences); (iii) different combinations of antibody fragments (e.g., scFab-scFab and scFab-scFv) derived from parental anti-Bb IgG4 antibody (e.g., V_{H}6/Vκ7-IgG4v2 from U.S. Pat. No. 11,242,382 and WO 2021/216458), and parental anti-C1s IgG4 antibody (e.g., V_{H}3/Vκ2 from WO 2018/071676); (iv) peptide linkers (e.g., between antibody fragments and between heavy and light chains of each antibody fragment, containing G₄S repeats); (v) the presence or absence of rationally designed charge mutations (CM) that promote accurate heavy/light chain pairing; and (vi) a polyadenylation site (e.g., a bovine growth hormone (bGH) gene polyadenylation signal).

### A. Generation of Bifunctional Bicistronic or Monocistronic Constructs

The bifunctional monocistronic or bicistronic constructs generated herein contain DNA fragments expressing scFv or scFab of the constituent antibody fragments to active C1s and Bb, downstream of the ubiquitous minCBA promoter, and a poly (A) signal sequence from the bovine growth hormone gene. The entire expression cassette was cloned between wildtype inverted terminal repeat (ITR) sequences from AAV serotype 2 (FIGS. 1A-C). Glycine/serine-rich linkers (e.g., linkers with G₄S repeats) were inserted between the heavy and light chains of each single-chain αC1s and αBb antibody fragment (scFab or scFv) to facilitate proper folding of each antigen-binding domain formed by a pair of V_{H} and V_{L}. In the present studies, a linker with seven G₄S repeats was used to link the heavy and light chains of an scFab and a linker with three G₄S repeats was used to link the V_{H} and V_{L} of an scFv.

For monocistronic constructs, exemplary formats were scFab-scFab, scFv-scFab, scFab-scFv, and ScFv-ScFv (see, e.g., FIGS. 2A, 2D, and 2E). Glycine/serine-rich linkers (e.g., linkers with G₄S repeats such as two or three repeats) were inserted between the two single-chain fragments of a bifunctional fusion protein to allow for the flexibility of the bifunctional fusion protein.

For some monocistronic constructs, an additional feature was the inclusion of a canonical furin cleavage site (RX (R/K) R) (SEQ ID NO:82), e.g., in linkers F2A and GT2A (FIG. 2F). Linking the heavy chain (HC) and light chain (LC) genes on a single cassette using 2A peptides would allow improved control of LC and HC ratio. Insertion of a furin recognition site upstream of 2A would allow removal of 2A residues that would otherwise be attached to the HC and/or LC (see, e.g., FIG. 2F).

For bidirectional bifunctional constructs, a novel bidirectional promoter was designed based on the ubiquitous minimal chicken β-actin (minCBA) promoter. This promoter supports the concurrent expression of individual antibody fragments to factor C1s and factor Bb. MinCBA contains a CBA promoter and an CMV enhancer but with an abbreviated intronic sequence. The bidirectional promoter contains a pair of CBA promoters placed in opposite directions and separated by an CMV enhancer (SEQ ID NO:53). The bidirectional expression construct produces separate anti-C1s and anti-Bb antibody fragments for independent target engagement, which removes the possibility of steric hindrance.

The monocistronic or bicistronic expression cassette was cloned between AAV2 ITR sequences (see, e.g., FIGS. 1C, 2H, and 2I) for AAV delivery.

Some experiments used antibody fragments containing charge mutations that promote accurate pairing between heavy and light chains of each constituent antibody fragment. To generate charge mutants (CM), specific amino acids were substituted in the variable and/or constant domains of the αC1s and αBb antibody fragments. The following amino acid changes were introduced for the following mutated antibody fragments:
αC1s scFab-CM: Q42E and Q292K (numbering in accordance with SEQ ID NO: 12)
αBb scFab-CM: Q38K, S114A, N137K, Q288E, and T434E (numbering in accordance with SEQ ID NO:24)

Exemplary monodirectional construct configurations are illustrated in FIGS. 2A and 2C-F and listed below:
#5: αC1s scFv-(G₄S)₂-αBb scFv
#6: αBb scFv-(G₄S)₂-αC1s scFv
#7: αC1s scFab-(G₄S)₃-αBb scFab
#8: αBb scFab-(G₄S)₃-αC1s scFab
#11: αC1s scFab-(G₄S)₃-αBb scFab-CM (#7 with CMs in both αC1s and αBb).
#12: αBb scFab-(G₄S)₃-αC1s scFab-CM (#8 with CMs in both αC1s and αBb).
#13: αC1s scFab-(G₄S)₂-αBb scFv.
#14: αC1s scFab-(G₄S)₂-αBb scFv-CM (#13 with CMs in both αC1s and αBb).
#15: αC1s scFab-(G₄S)₃-αBb scFv.
#16: αC1s scFab-(G₄S)₃-αBb scFv-CM (#15 with CMs in both αC1s and αBb)
#17: αC1s F2A Fab-(G₄S)₃-αBb scFab, producing a heterodimer comprised of (i) an αC1s LC and (ii) a fusion protein comprising an αC1s HC fused to an αBb scFab
#18: αC1s GT2A Fab-(G₄S)₃-αBb scFab, producing a heterodimer comprised of (i) an αC1s LC and (ii) a fusion protein comprising an αC1s HC fused to an αBb scFab
#19: αC1s scFab-(G₄S)₃-αBb F2A Fab, producing a heterodimer comprised of (i) a fusion protein comprising an αC1s scFab fused to an αBb HC and (ii) an αBb LC
#20: αC1s scFab-(G₄S)₃-αBb GT2A Fab, producing a heterodimer comprised of (i) a fusion protein comprising an αC1s scFab fused to an αBb HC and (ii) an αBb LC

Exemplary bidirectional construct configurations are illustrated in FIG. 2B and listed below:
#9: αC1s scFab-BiDir-αBb scFab, producing separate αC1s scFab and αBb scFab
#10: αBb scFab-BiDir-αC1s scFab, producing separate αC1s scFab and αBb scFab
#21: αC1s scFab-BiDir-αBb scFab-CM, producing separate αC1s scFab-CM and αBb scFab-CM
#22: αBb scFab-BiDir-αC1s scFab-CM, producing separate αC1s scFab-CM and αBb scFab-CM

### B. Evaluation of Bb- and C1s-Binding

Each DNA construct was transfected into HEK293 cells. Supernatants containing the secreted recombinant proteins were harvested and purified over Protein L beads. More specifically, the supernatant was incubated with Protein L beads for 1 hour at room temperature. The beads were then washed three times with PBS containing polysorbate 20. The bead column was then eluted with 0.1 M glycine (pH 2.0) for ten minutes at room temperature. The eluate was neutralized with 15% v/v 1 M Tris (pH 8.5) and then desalted through buffer exchange into PBST.

Purity of the recombinant proteins was evaluated on SDS-PAGE (non-reduced and reduced) and on mass photometer (mass distribution). Concentrations of the proteins were measured on NanoDrop® (Thermo Fisher).

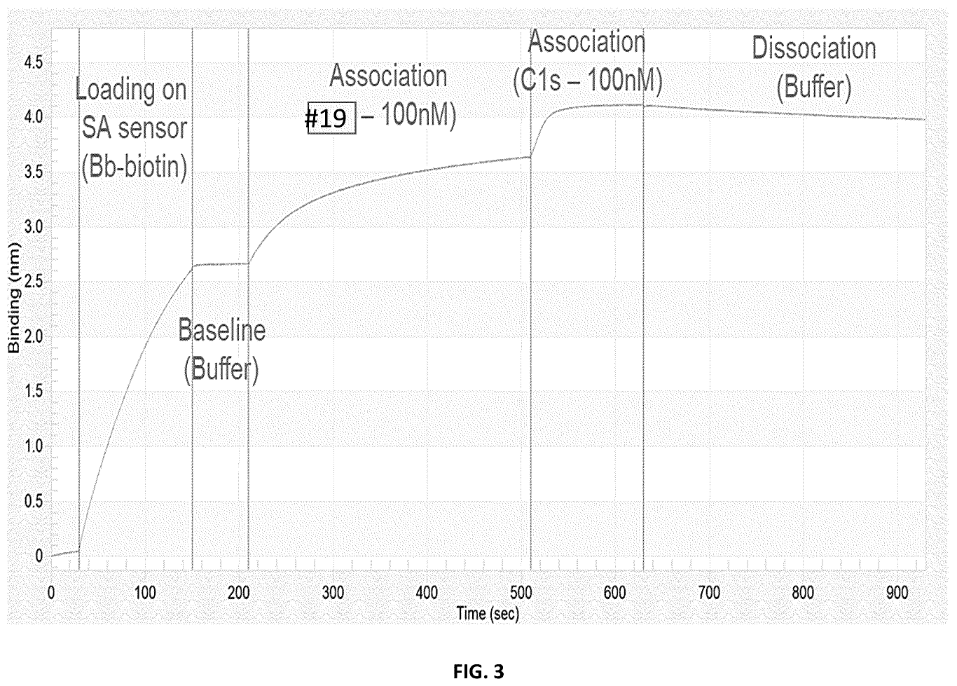

Bio-layer interferometry (BLI) was used to assess the recombinant proteins' target engagement to and binding affinity for complement C1s enzyme (active C1s or “C1s” herein) and factor Bb (Bb) (Complement Technology, Tyler, TX, USA). C1s and Bb were biotinylated with EZ-Link™ Sulfo-NHS-LC-LC-Biotin (Thermo Fisher, Waltham, MA, USA) according to manufacturer instructions. Biotinylated C1s or Bb was loaded on Octet® Streptavidin (SA) Biosensors (Sartorius, Göttingen, Germany), followed by a concentration range of purified proteins. To assess dual target engagement, biotinylated active C1s or Bb was loaded onto sensors, followed by purified proteins (“first association phase”), followed by the non-captured complement target (Bb or active C1s, non-biotinylated; “second association phase”). The assays were performed at 30° C. using PBS with 0.1% Tween 20 as a diluent (FIG. 3).

Additionally, inhibition of the classical and alternative complement pathways was evaluated by Wieslab® Complement System Classical Pathway and Wieslab® Complement System Alternative Pathway kits (Svar, Malmö, Sweden). Assays were performed according to manufacturer instructions. Serial dilutions of constructs were performed in the respective assay diluents for each assay.

### C. Results

To confirm vector-derived antibody fragments were expressed and secreted, supernatants were harvested from HEK293 cells transfected with plasmids encoding the transgenes and kappa light chain-containing antibody fragments were enriched from the supernatant by affinity purification using protein L beads. Western blot analyses of the enriched supernatants demonstrated that all transgenes produced antibody fragments.

Target engagement of antibody fragments from cell supernatants was evaluated using the Octet® binding assay. The data demonstrated that proteins produced from all expression constructs all exhibited dual target engagement for C1s and Bb. Across all tested constructs, the binding affinity of the partially purified bifunctional antibody fragments was within 2- to 10-fold of the purified parental anti-C1s and anti-Bb Fabs.

Exemplary data are shown in FIG. 3, which shows that when partially purified antibody fragments produced by construct #19 (FIG. 2F) were added in first “association” phase (for binding to Bb), an increase in signal was observed. When the second target «C1s was added in second “association” phase, an additional increase in signal was observed (FIG. 3). All antibody fragments produced by the tested bifunctional constructs, except construct #5 (FIG. 2A), exhibited similar levels of dual target engagement (see Example 2 below).

The parental monoclonal antibodies used to design the bifunctional complement inhibitors have been previously shown to inhibit either the complement classical (CP; see WO 2016/164358) or alternative (AP; see U.S. Pat. No. 11,242,382) pathway, with neither inhibiting the lectin pathway. The ability of the bifunctional antibody fragments or antibody fragment pairs to inhibit activity of both the CP and AP was assessed in vitro using Wieslab® assays. All tested bifunctional antibody constructs inhibited both IgM-stimulated activation of the CP and LPS-stimulated activation of the AP (see Example 2 below). The data show that for all tested constructs, the inhibitory activity was within 4-fold of the parental Fabs.

The results show that the vector expressed antibody fragments to complement factors Bb and C1s bind to target complement factors and inhibit activated complement with an efficiency that is similar to parental individual Fab proteins. These results were unexpected because parental antibody fragments are Fabs generated using recombinant mAB technology methods, i.e. expressed in a CHO cell and highly purified, in contrast the antibody fragments generated from the AAV pre-viral plasmids are scFab and scFV fragments and were tested as partially purified antibody fragments. Moreover, the plasmid derived antibody fragments, for some constructs, are monocistronic, and are therefore acting like bifunctional antibodies. Despite this difference in design/structure from the parental Fabs, inhibition of each target was largely preserved.

### Example 2: Functional Characterization of Anti-C1s and Anti-Bb scFabs

Constructs #2 and #4 were recombinantly expressed and purified to homogeneity as described above and tested in target binding assays as well as in serum-based and cell-based functional assays. Direct target binding was measured using surface plasmon resonance (SPR).

The inhibitory activity of the scFabs were tested in serum-based Wieslab® enzyme immune assays. In the commercial assay kits, the wells of the microtiter strips are coated with specific activators for each pathway of the complement system. Additionally, the buffers and reagents included in the kits prevent the cross-activation of multiple pathways, maintaining specificity of pathway activation. Test kits for the AP are coated with lipopolysaccharide, while test kits for the CP are coated with human IgM). The final readout is the detection of a neoepitope on the C5b9 complex generated due to the complement pathway activation, measured colorimetrically. The recombinant scFabs were also tested in a modified Wieslab® assay, where the microtiter plate was coated with both heat-aggregated (HAGG) IgG) and C3b to allow for simultaneous activation of CP and AP; in this assay, the C5b9 complex generated from the activation of both pathways was also measured colorimetrically.

Additionally, the recombinant scFabs were tested in an in vitro ARPE19 cell line-based model of dry AMD. In all the functional assays, the recombinant scFabs were tested individually as well as an equimolar mixture to be representative of the vector-derived product.

Table 1 below shows the characterization of recombinant scFabs and their comparison to parental scFabs (#2 and #4) and mAbs.

TABLE 1

| | | | | Wieslab ® assay IC₅₀ (nM) | | |
|---|---|---|---|---|---|---|
| | | K_{D} (nM) | | Alternative | Classical | Lectin |
| Sample | Const. | Bb | C1s | Pathway | Pathway | pathway |
| Const. | Purified anti-C1s | NA | 0.3 | NA | 6.7 | ND |
| #9 | scFab | | | | | |
| | Purified anti-Bb | 3.7 ± 0.4 | NA | 190.4 | NA | ND |
| | scFab | | | | | |
| | Equimolar mix of | ND | ND | 461.1 | 12.9 | ND |
| | anti-C1s scFab and | | | | | |
| | anti-Bb scFab | | | | | |
| Parental | Purified anti-C1s | NA | 0.3 | NA | 5.5 | ND |
| scFabs | Fab | | | | | |
| | Purified anti-Bb | 2.7 ± 0.4 | NA | 144.1 | NA | ND |
| | Fab | | | | | |
| | Equimolar mix of | ND | ND | 270.4 | 14.4 | ND |
| | anti-C1s Fab and | | | | | |
| | anti-Bb Fab | | | | | |
| Parental | anti-C1s mAb | NA | 1.5 | No | 22 | No |
| mAbs | | | | inhibition | | inhibition |
| | anti-Bb mAb | 7.3 ± 1.4 | NA | 189.67 | No | No |
| | | | | | inhibition | inhibition |

These data show that the recombinant scFabs against both C1s and Bb show similar binding and inhibitory properties as their corresponding parental scFabs.

### Example 3: Properties of Exemplary Complement Inhibitors with Charge Mutations

Three expression constructs were selected for further studies. The first one, construct #14 (FIGS. 2D and 2H), was composed of a unidirectional minCBA promoter driving expression of a single transcript encoding anti-C1s scFab connected to anti-Bb scFv by a flexible (G₄S)₂ linker [αC1s scFab-(G₄S)₂-αBb scFv] and followed by a bGH poly (A) signal. The sequences were human codon-optimized and contain charge mutations to promote accurate chain pairing.

The second expression construct, construct #12 (FIGS. 2C and 2J), was composed of a unidirectional minCBA promoter driving expression of a single transcript encoding anti-Bb scFab connected to anti-C1s scFab by a flexible (G₄S)₃ linker [αBb scFab-(G₄S)₃-αC1s scFab] and followed by a bGH poly (A) signal. The sequences were human codon-optimized and contain charge mutations to promote accurate chain pairing.

The third expression construct, construct #9 (FIGS. 2B and 21), is composed of a bidirectional minCBA promoter driving expression of separate transcripts encoding human codon-optimized αBb scFab or αC1s scFab [αC1s scFab+αBb scFab], each followed by a bGH poly (A) signal. In assays performed as described in Example 1, the complement-binding antibody fragments expressed from constructs #9 and #14 had a binding affinity for both C1s and Bb within 2- to 6-fold of the purified parental Fabs, while complement-binding antibody fragments expressed from construct #12 had a binding affinity for C1s and Bb within about 6- to 7-fold of the purified parental Fabs (Table 2).

TABLE 2

| Inhibitor | AP K_{D} (Bb, nM) | CP K_{D} (C1s, nM) |
|---|---|---|
| #14 | 2.5 | 1.4 |
| #12 | 6.6 | 3.6 |
| #9 | 0.9 | 1.8 |
| anti-Bb Fab | 0.7 | N/A |
| anti-C1s Fab | N/A | 0.3 |

In Wieslab® assays, the IC₅₀ values of construct #14-derived complement inhibitors were within about 6-fold of purified anti-C1s Fab (CP inhibition) and purified anti-Bb Fab (AP inhibition). The IC₅₀ values of #12-derived antibody fragments were within about 7-fold of purified anti-Bb Fab (AP inhibition) and 14-fold of purified anti-C1s (CP inhibition). The IC₅₀ values of #9-derived antibody fragments were within about 3-fold of purified anti-Bb Fab (AP inhibition) and 25-fold of purified anti-C1s Fab (CP inhibition) (Table 3).

TABLE 3

| Inhibitor | AP IC₅₀ (nM) | CP IC₅₀ (nM) |
|---|---|---|
| #14 | 551 | 38.6 |
| #12 | 702 | 84.4 |
| #9 | 224 | 157.3 |
| anti-Bb Fab | 99 | N/A |
| anti-C1s Fab | N/A | 6 |

Additionally, constructs #2, #4, #12, and #14 (FIGS. 2A, 2C, and 2D) were expressed and purified to >98% purity using chromatographic methods (referred to as recombinant constructs) and tested head-to-head with the parental anti-C1s and anti-Bb Fabs in the assays described in Example 1 to characterize the functional properties of these constructs. Constructs #2 and #4 were chosen to represent the two scFabs that would be expressed and secreted by the bidirectional vector construct #9.

The results of these experiments are summarized in Table 4 below (ND: not determined).

TABLE 4

| | | | IC₅₀ CP | IC₅₀ AP |
|---|---|---|---|---|
| | K_{D}, C1s | K_{D}, Bb | Wieslab ® | Wieslab ® |
| Construct No. | (nM) | (nM) | EIA (nM) | EIA (nM) |
| 2 | 0.3 | NA | 7.2 | NA |
| 4 | NA | 0.7 | NA | 192.9 |
| 12 | 5.4 | 1.5 | 10.4 | 428.1 |
| 14 | 1.3 | 4.2 | 5.9 | 346.6 |
| Equimolar mix of | ND | ND | 12.9 | 461.1 |
| #2 and #4 | | | | |
| Equimolar mix of | ND | ND | 14.4 | 270.4 |
| anti-C1s | | | | |
| Fab and anti-Bb Fab | | | | |

Table 5 below summarizes the in vitro binding and functional inhibition results of the proteins expressed by constructs #2, #4, #5, #6, #7, #8, #9, #11, #12, #13, #14, #15, #16, #17, #18, #19, and #20 from FIGS. 2A-2F) in comparison to the recombinant parental anti-anti-C1s Fab and anti-Bb Fab.

TABLE 5

| | Dual | | | | | | | | |
|---|---|---|---|---|---|---|---|---|---|
| | target | | | | | | | | |
| | binding | K_{D} (nM) | | kₐ (10⁵ M⁻¹s⁻¹) | | kₐ (10⁻⁴ s⁻¹) | | IC₅₀ (nM) | |
| Construct No. | (Y/N) | Bb | C1s | Bb | C1s | Bb | C1s | AP | CP |
| 2 | NA | NA | 0.3 | NA | 2.7 | NA | 7.6 | NA | 7.2 |
| 4 | NA | 0.7 | NA | 6.7 | NA | 4.9 | NA | 192.9 | NA |
| 5 | ND | ND | ND | ND | ND | ND | ND | 2826* | 216 |
| 6 | ND | ND | ND | ND | ND | ND | ND | 1258* | 118 |
| 7 | Y | 9.3 | 1.8 | 0.6 | 2.6 | 5.4 | 4.7 | 570 | 45.9 |
| 8 | Y | 1.8 | 2.1 | 1.2 | 3 | 2.1 | 6.3 | 420 | 87.7 |
| 9 | Y | 0.8 | 1.9 | 2.6 | 1.5 | 2 | 2.8 | 224 | 157 |
| 11 | Y | 8.8 | 2.7 | 0.6 | 2.1 | 5.1 | 5.8 | 1000* | 49.7 |
| 12 | Y | 6.6 | 3.6 | 0.8 | 1.8 | 5.5 | 6.5 | 702 | 84.4 |
| 13 | Y | 3 | 1 | 0.7 | 3 | 2.2 | 2.9 | 201 | 44.4 |
| 14 | Y | 2.5 | 1.4 | 0.7 | 3.6 | 1.7 | 4.9 | 551 | 38.6 |
| 15 | Y | 3.9 | 1.6 | 1.2 | 3.7 | 4.5 | 5.7 | 405 | 34.7 |
| 16 | Y | 2.5 | 1.4 | 0.9 | 3.6 | 2.3 | 5.1 | 586 | 35.4 |
| 17 | Y | ND | ND | ND | ND | ND | ND | ND | ND |
| 18 | Y | ND | ND | ND | ND | ND | ND | ND | ND |
| 19 | Y | ND | ND | ND | ND | ND | ND | 2093* | 34 |
| 20 | Y | ND | ND | ND | ND | ND | ND | ND | ND |
| Anti-C1s | NA | NA | 0.3 | NA | 2.9 | NA | 8.1 | NA | 5.4 |
| Fab | | | | | | | | | |
| Anti-Bb | NA | 0.73 | NA | 7 | NA | 5.1 | NA | 144.1 | NA |
| Fab | | | | | | | | | |

NA: not applicable.
ND: not determined.
*Curve not saturated; estimated IC₅₀.

In addition to direct target binding (BLI) and Wieslab® EIA assays, another functional assay was developed to assess the simultaneous inhibition of both CP and AP by these recombinant constructs. In this assay, ELISA plates were coated with both HAGG (heat-aggregated gamma globulin) and C3b and incubated with 12% C1s-depleted serum containing 380 ng/ml proenzyme C1s, to activate both CP and AP simultaneously. The conditions in the assays were optimized to achieve similar levels of CP and AP activation on the plate. Dose responses of constructs #2 and #4 were tested either individually or in an equimolar mix (to represent the expression condition from construct #9). An equimolar mix of the parental anti-C1s Fab and anti-Bb Fab was also tested alongside.

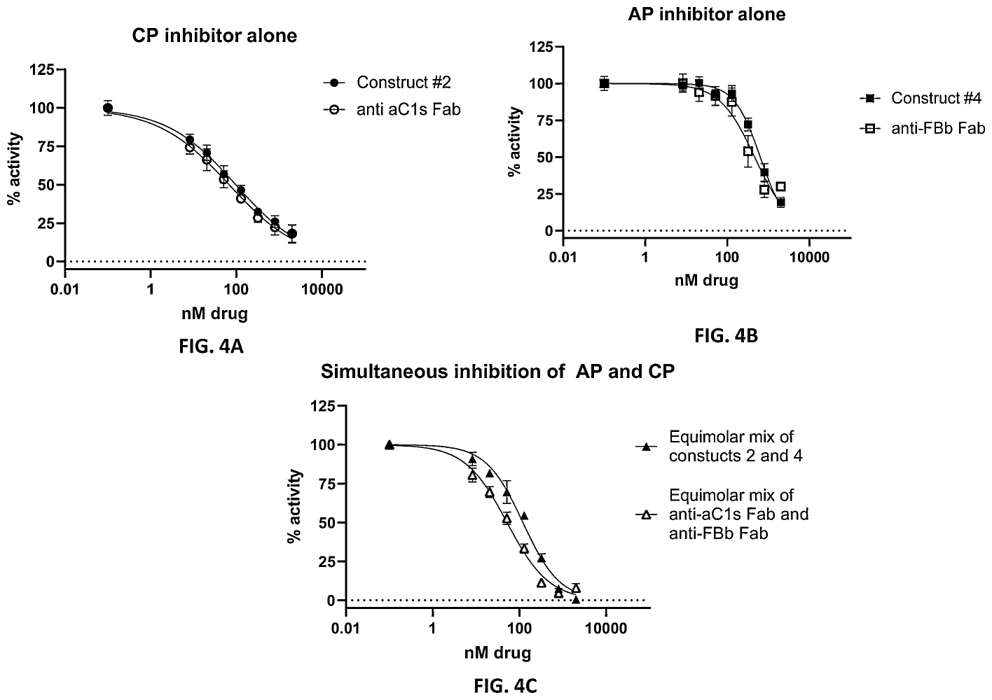

Under these conditions, constructs #2 and #4 achieved dose-dependent but partial inhibition (70-85%; FIGS. 4A and 4B). However, when these two constructs were mixed together in an equimolar ratio, it resulted in >99% inhibition of complement activation, similar to what was seen for the equimolar mix of the parental Fabs. The IC₅₀ observed was within 2- to 3-fold of what was observed for the equimolar mix of the parental Fabs (FIG. 4C). See also Table 6, which summarizes the half-maximal inhibitory concentrations of anti-C1s Fab, anti-Bb Fab, the protein expressed from construct #2, the protein expressed from construct #4, or an equimolar mixture of the two, as well as the maximum inhibition achieved, under conditions where both CP and AP were activated simultaneously in vitro.

TABLE 6

| Construct No. | IC₅₀ (nM) | Maximal inhibition |
|---|---|---|
| 2 | 97.3 | 75-80% |
| 4 | 633.4 | 80-85% |
| anti-C1s Fab | 68.2 | 75-85% |
| anti-Bb Fab | 462.6 | 70-75% |
| Equimolar mix of constructs | 119.8 | >99% |
| #2 and #4 | | |
| Equimolar mix of anti-C1s | 51.5 | 90-92% |
| Fab and anti-Bb Fab | | |

### Example 4: In Vivo Retina Studies in Mice

Based on the above in vitro results, constructs #9, #12, and #14 were selected for in vivo studies, and their ITR plasmid expression cassettes were packaged into AAV2 for delivery to target cells (see, e.g., FIGS. 2H, 2I, and 2J). This Example describes in vivo testing of these vectorized antibody constructs in wildtype mouse retina to confirm transduction of retinal ganglion cells (RGC) and secretion of the antibody fragments into the vitreous. Antibody fragments secreted into the mouse vitreous humor were evaluated in an in vitro assay to demonstrate target engagement with human complement factors C1s and Bb. Tolerability was assessed by optical coherence tomography (OCT).

### A. AAV Injection

More specifically, recombinant AAV2 expressing constructs #9, #12, and #14 flanked by AAV2 ITRs were produced. AAV2 #14, AAV2 #12, and AAV2 #9 were administered to C57BL/6J mice at three doses [10⁸, 10⁹, or 10¹⁰ vector genomes (vg) per eye] through intravitreal injection, and retinal transduction, transgene expression, antibody secretion, and tolerability were assessed after 3-4 weeks in-life exposure. A recombinant AAV2 encoding a secreted VEGF inhibitor was administered in parallel at 2×10⁹ vg per eye as a positive control. Un-injected, vector-naïve mice were used as a negative control.

### B. Vector Transduction

Vector transduction was quantified using a TaqMan® assay to detect the vector-derived bGH poly (A) in quantitative PCR analyses of DNA purified from the mouse retinas. The data show that all three vectors successfully transduced the retina, achieving about 10⁴-10⁵ vg per 500 ng DNA. The levels of transduction from the bifunctional antibody fragment vectors were comparable to what was achieved with the positive control. There was a vector dose-dependent increase in transduction of AAV2 #14 (10¹⁰ vs. 10⁸; p=0.01). Similar results are observed for AAV2 #12 (several mice administered 10⁹ vg had relatively low levels of transduction; this was likely due to a technical issue with the administration of that dose). AAV2 #9, which has two copies of the bGH poly (A), showed high levels of transduction at all doses.

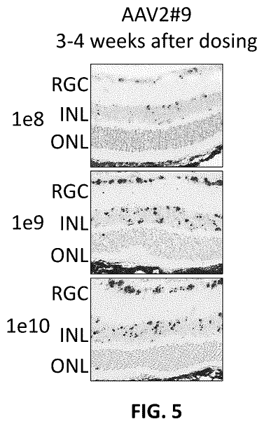

Vector transduction and cell targeting in the mouse retina were also assessed using vector-specific probe sets in in situ hybridization (ISH) analyses of sections from fixed, paraffin-embedded eyes. Each probe set included 40 pairs of probes of about 50 bases in length. In eyes administered each of the AAV2 vectors, vector transduction was detected primarily in retinal ganglion cells (RGC) and cells of the inner nuclear layer (INL), to a lesser degree in cells of the outer nuclear layer (ONL), and rarely in the cells of the retinal pigment epithelium (RPE) (FIG. 5).

Table 7A summarizes the levels of transduction (vector genomes/500 ng genomic DNA) achieved in the mouse retina at 3 weeks after intravitreal administration of AAV2 #9, AAV2 #12, and AAV2 #14 (median±MAD).

TABLE 7A

| | 3-4 weeks after dosing | | |
|---|---|---|---|
| | Dose | | |
| Construct | 1e8 vg | 1e9 vg | 1e10 vg |
| #14 | 1.23e4 ± 7.82e3 | 4.81e4 ± 4.11e4 | 8.49e4 ± 1.29e4 |
| #12 | 1.06e4 ± 6.07e3 | 2.23e3 ± 2.05e3 | 4.55e4 ± 1.66e4 |
| #9 | 3.12e4 ± 1.94e4 | 2.38e4 ± 1.51e4 | 4.18e4 ± 2.85e4 |

### C. Transgene Expression

Transgene expression in the retina was measured through quantitative RT-PCR analyses of RNA purified from the mouse retinas, using a TaqMan® assay to detect the vector-derived bGH poly (A) sequence. RNA quality was assessed and samples with an RNA integrity number (RIN) lower than 6 were not included in the analyses. The data show that all three AAV vectors produce high levels of transgene expression (˜10⁵ to 10⁶ transcripts per 500 ng RNA) in the retina after 3 weeks of in-life exposure. Table 7B summarizes the levels of transgene expression (bGH transcripts/500 ng RNA) achieved in the mouse retina at 3 weeks after intravitreal administration of AAV2 #9, AAV2 #12, and AAV2 #14 (median±MAD).

TABLE 7B

| | 3-4 weeks after dosing | | |
|---|---|---|---|
| | Dose | | |
| Construct | 1e8 vg | 1e9 vg | 1e10 vg |
| #14 | 3.16e5 ± 1.32e5 | 5.76e5 ± 2.37e5 | 1.39e6 ± 6.92e5 |
| #12 | 7.90e4 ± 2.96e4 | 5.34e2 ± 4.99e2 | 2.92e5 ± 2.11e5 |
| #9 | 9.42e5 ± 2.55e5 | 1.25e6 ± 1.91e5 | 3.02e6 ± 4.28e5 |

Across all samples, transcript levels correlate with levels of vector genomes (p=0.59), and expression levels were lower in poorly transduced AAV2 #12 retinas from the 10⁹ vg treatment group. AAV2 #9 showed a dose-dependent increase in transgene expression (10¹⁰ vs. 10⁸, with p=0.036; 10¹⁰ vs. 10⁹, with p=0.0495).

### D. Antibody Expression

Expression and distribution of the vector-derived complement inhibitors in the mouse retina was evaluated through immunohistochemistry (IHC) using an anti-human kappa light chain antibody to detect the vector-derived human antibody fragments. The data show that inhibitors produced by all three vectors were detected in RGCs (retinal ganglion cells) and cells of the INL., (inner nuclear layer.)

### E. Antibody Secretion and Target Engagement

To demonstrate that the viral vectors produced bifunctional complement inhibitors that were secreted, inhibitor levels in the vitreous humor from mice were assessed using an ELISA method. Vector-derived complement inhibitors present in mouse vitreous humor were quantified via target engagement capacity using C1s and Bb ELISA and purified anti-C1s and anti-Bb scFabs as standards. Tables 8A and 8B summarize the ex vivo dual target engagement results of secreted anti-C1s (Table 8A) and anti-Bb (Table 8B) antibody fragments present in mouse vitreous humor at 3 weeks following intravitreal administration of AAV2 #9, AAV2 #12, and AAV2 #14 (mean±SD; ng/ml).

TABLE 8A

| | 3-4 weeks after dosing | | |
|---|---|---|---|
| | Dose | | |
| Construct | 1e8 vg | 1e9 vg | 1e10 vg |
| #14 | 360 ± 170 | 159 ± 23 | 1022 ± 622 |
| #12 | 72 ± 11 | 107 ± 19 | 124 ± 28 |
| #9 | 145 ± 52 | 262 ± 194 | 1011 ± 578 |

TABLE 8B

| | 3-4 weeks after dosing | | |
|---|---|---|---|
| | Dose | | |
| Construct | 1e8 vg | 1e9 vg | 1e10 vg |
| #14 | 384 ± 225 | 144 ± 29 | 811 ± 413 |
| #12 | 84 ± 11 | 117 ± 17 | 149 ± 30 |
| #9 | 144 ± 60 | 339 ± 215 | 1159 ± 559 |

Overall, the C1s and Bb ELISAs demonstrate that all three rAAVs, when delivered intravitreally, led to expression and secretion from the mouse retinal ganglion cells. Proteins expressed by all three expression vectors could bind to C1s and Bb ex vivo. Overall, the data show that all three expression vectors produced comparable levels of anti-C1s and anti-Bb binding activity in mice. It was unexpected that retinal ganglion cells could support the in vivo production of vectorized antibody fragments that exhibit similar binding properties as parental antibodies generated in vitro using established recombinant antibody production methods.

AAV2 #14-treated mice have vitreous levels of bifunctional antibodies ranging from about 150 ng/ml to about 900 ng/ml. Vitreous levels of AAV2 #12-derived inhibitors show a slight dose-response across treatment groups, increasing from about 80 ng/mL to about 140 ng/ml. Levels of inhibitors in vitreous from AAV2 #9-treated mice increase in a dose-dependent manner, reaching about 1100 ng/ml at the highest dose. In addition to quantifying inhibitor levels in vitreous, these data demonstrate ex vivo dual target engagement of vector-derived antibody fragments.

Target engagement and efficacy of vector-derived complement inhibitors cannot be evaluated in vivo in mice because these inhibitors bind only human and nonhuman primate (NHP) C1s and Bb, and do not interact with murine complement factors.

In mice dosed with the AAV2 positive control (see above), secretion of the VEGF inhibitor into the vitreous was measured by ELISA. Vitreous levels of the VEGF inhibitor average about 57 ng/ml after 2 weeks in-life exposure. Therefore, AAV2 #14, AAV2 #12, and AAV2 #9 all generate higher levels of secreted proteins than the positive control.

### F. Tolerability

Photoreceptor damage can be detected as a thinning of the photoreceptors. Tolerability of the viral vectors was assessed by measuring the thickness of the photoreceptor (PR) layer [outer nuclear layer (ONL)+inner segment/outer segment (IS/OS)] in optical coherence tomography (OCT) images from vector-naïve and transduced mouse retinas. Photoreceptor thickness in AAV2 #14-, AAV2 #12-, and AAV2 #9-transduced retinas does not decrease at any dosage (10⁸, 10⁹, or 10¹⁰ vg) compared to vector-naïve retinas, suggesting no impact on photoreceptor tolerability for the doses and time points studied in mice.

### Example 5: Inhibition of Complement Activation in a Cell-Based Model of Dry AMD

C-reactive protein (CRP) is an acute phase reactive protein and an activator of the classical complement pathway (CP). CRP binds to dying cells and activates the CP, labeling those cells for clearance by phagocytes. CRP's levels are elevated under inflammatory conditions. It has been shown that elevated CRP levels is an independent risk factor for the pathogenesis of AMD, and high serum concentrations of CRP are linked to faster AMD progression to advanced disease and higher severity of vision loss in other retinal diseases like retinitis pigmentosa (Chen et al., Trans Vis Sci & Techno. (2021) 10 (7): 7; Molins et al., Front Immunol. (2018) 9:808; and Murakami et al., Acta Ophthalmol. (2018) 96 (2): e174-e179). Additionally, it has been shown that Bruch's membrane, drusen, and choroidal vessel walls stain for elevated levels of CRP in AMD patients' eyes, suggesting that complement activation during the disease is, at least in part, initiated by CRP (Bhutto et al., Br J Ophthalmol. (2011) 95 (9): 1323-30).

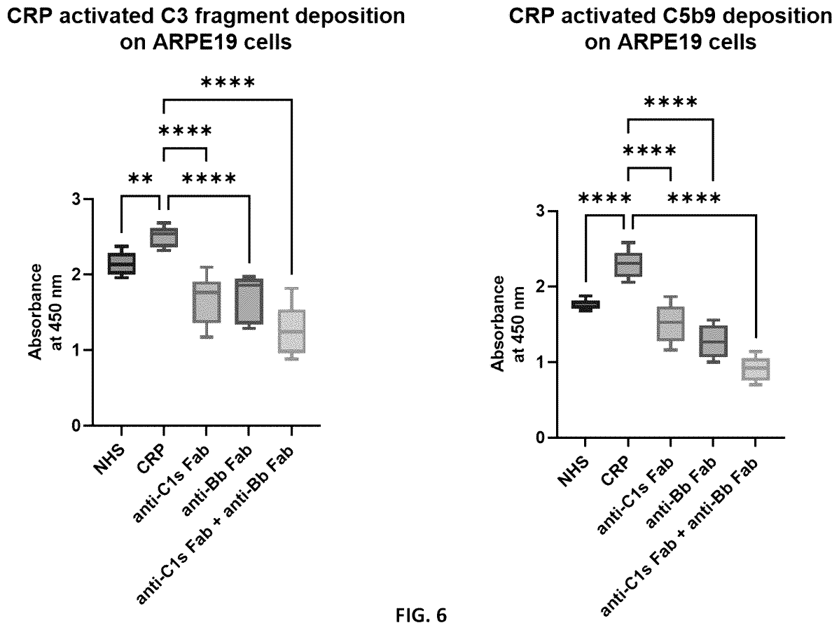

To recapitulate some of these patient characteristics in vitro in a cell-based model, ARPE19 cells (a retinal pigment epithelia (RPE) cell line) were treated with normal human serum (NHS) supplemented with CRP. The extent of complement activation was assessed by monitoring the levels of C3-fragment and C5b9 deposited on the cell surface using an on-cell ELISA protocol. The data show that treatment of ARPE19 cells with NHS supplemented with CRP resulted in elevated levels of both C3-fragments and C5b9 on the cells compared to treatment with NHS alone, indicating a stronger activation of the complement system in the presence of CRP (FIG. 6). When complement inhibitors were included in the treatment, combined inhibition of the CP and the AP (anti-C1s Fab+anti-Bb Fab) resulted in a stronger reduction of both C3-fragment and C5b9 levels as compared to the levels of inhibition achieved by either the anti-C1s Fab (CP) or the anti-Bb Fab (AP) individually (FIG. 6).

### Example 6: A New iPSC-Derived Cell Model for AMD

This Example describes a new cell model developed to demonstrate CRP-initiated complement activation in AMD. This model measures complement deposition on induced pluripotent stem cell-derived retinal pigment epithelial cells (iPSC-RPE). RPE have many vital roles in the eye and are responsible for the phagocytosis of photoreceptor outer segments and the transfer of nutrients from the choroid to the retina, in addition to many other essential functions. Complement activation on RPE may contribute to inflammation and cell death in AMD. iPSC-RPE were selected for this model because they maintain the morphology of native RPE and share similar cell markers. Measuring complement deposition on the surface of these cells can thus model how certain drug treatments limit complement activation in the retina during AMD disease course.

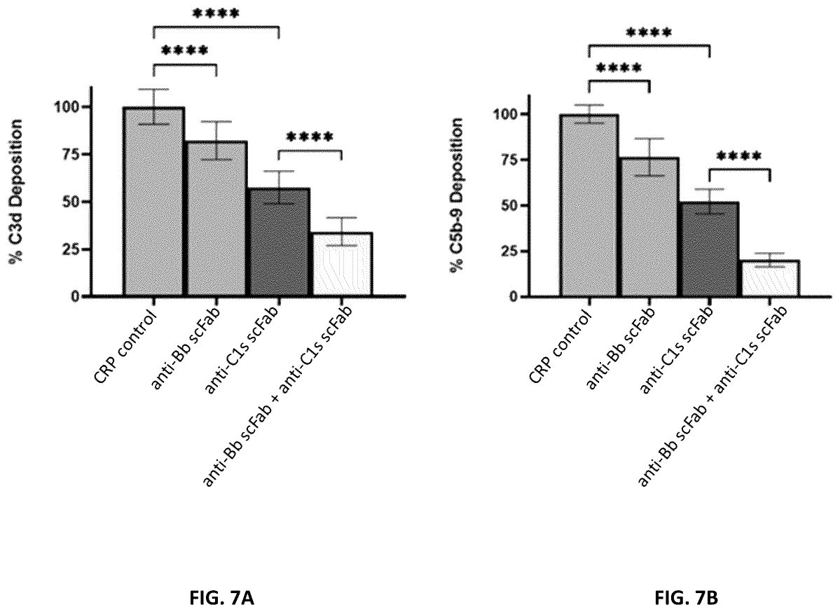

A cell-ELISA was used to measure complement deposition on the surface of iPSC-RPE. iPSC-RPE (FujiFilm Cellular Dynamics, Madison, WI) were grown in a fibronectin-coated black/clear bottom 96-well plate. CRP (100 μg/mL) (ImmunoPrecise Antibodies, Utrecht, The Netherlands), 10% normal human serum (Complement Technology, Tyler, TX) and complement inhibitors being tested were added to cell culture media and incubated with the iPSC-RPE overnight. The next day, the cells were washed and fixed with 4% paraformaldehyde. After blocking, the cells were incubated with an anti-C3d or anti-C5b9 HRP-conjugated antibody (Novus Biologicals, Centennial CO). QuantaRed™ Enhanced Chemifluorescent HRP Substrate (Thermo Fisher, Waltham, MA) was used to develop a fluorescent signal that was measured using a plate reader. The data show that individual treatment with either anti-C1s or anti-Bb scFabs led to a significant decrease in C3d and C5b9 deposition on iPSC-RPE; however, the combination of both scFabs decreased deposition of complement products to the greatest extent (FIGS. 7A and 7B).

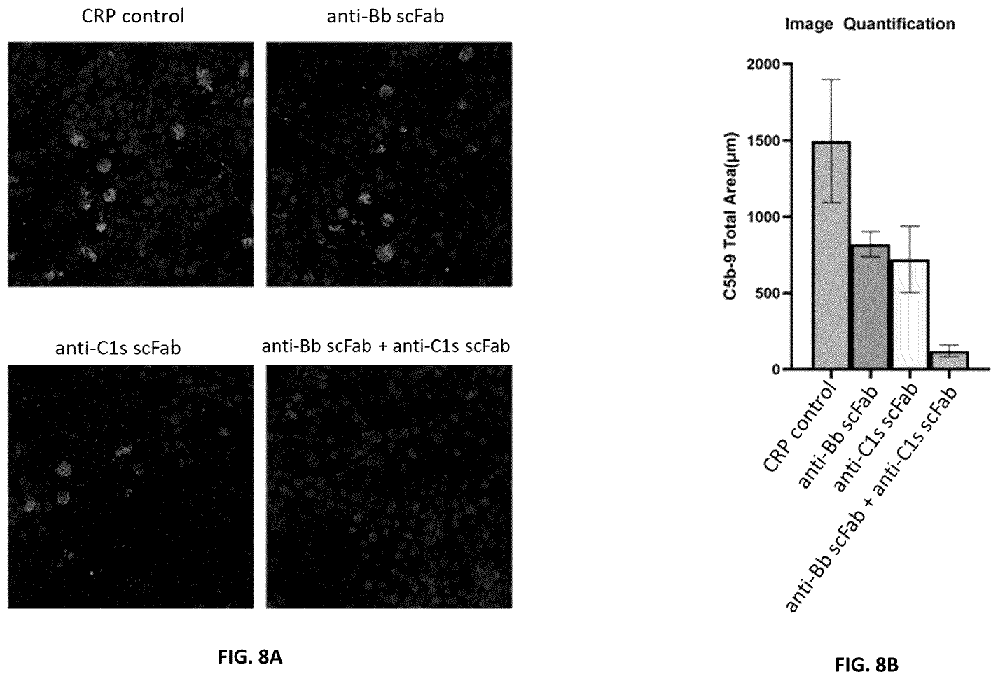

A similar method was used for fluorescent imaging of C5b9 deposition on iPSC-RPE. In this method, cells were grown on fibronectin-coated 24-well hanging cell culture inserts. Cells were treated with CRP, 10% normal human serum, and complement inhibitors overnight. Confocal microscopy was used to capture z-stack images at 40× magnification. For image quantification, three regions of interest (ROIs) were randomly imaged from each sample. Total areas of C5b9 were calculated within each ROI and were averaged for each sample. The average of three replicates was measured for each group and error bars were calculated from the average of standard deviations. The fluorescent imaging experiment was repeated three times with three different iPSC-RPE cell lines. The data similarly show that treatment with a combination of anti-C1s and anti-Bb scFabs led to a stark decrease in C5b9 (red) staining (FIGS. 8A and 8B).

In conclusion, the results from the iPSC-RPE model show that both the classical and alternative pathways likely play a role in AMD pathogenesis. Blocking each pathway separately led to a decrease in complement deposition on RPE cells. However, inhibiting both pathways simultaneously led to the greatest decrease in deposition, suggesting that concurrent classical and alternative pathway inhibition may be beneficial in AMD

### Example 7: In Vivo Retina Studies in Non-Human Primates

This Example describes in vivo testing of exemplary vectorized antibody constructs in non-human primates (NHPs) to confirm transduction and transgene expression in the retina. Activity of the viral vectors following intravitreal administration in NHPs was evaluated in two studies: (1) a 6-week dose-range study of AAV2 #14 and AAV2 #12 and (2) an 8-week single-dose study of AAV2 #14 and AAV2 #9. In each study, NHPs administered ocular formulation buffer were used as controls.

### A. Study 1

In the first study, NHPs were administered through intravitreal injection ocular formulation buffer (N=2 NHPs), or AAV2 #14 or AAV2 #12 at three doses (2×10⁹, 2×10¹⁰, or 2×10¹¹ vg per eye, based on vector titer determined using an assay that detects the BGH poly (A); N=3 NHPs per treatment group). The animals were assessed after six weeks of in-life exposure.

For evaluation of vector transduction, vector genome levels were quantified by using vector-specific TaqMan® assays in quantitative PCR analyses of DNA purified from the NHP retinas. Comparable DNA input across samples was confirmed using TUBB1 as a reference gene. The data show that both AAV2 #12 and AAV2 #14 successfully transduced the NHP retina, resulting in a dose-dependent increase in vector genome levels (dose-response AAV2 #14 p=0.0286, AAV2 #12 p=0.0095). Table 9 below summarizes the level of transduction achieved in the NHP retina at 6 weeks after intravitreal administration (median vector genomes/500 ng genomic DNA).

TABLE 9

| | vector genome levels 6 | | |
|---|---|---|---|
| | weeks after dosing | | |
| | Dose | | |
| Construct | 2 × 10⁹ vg | 2 × 10¹⁰ vg | 2 × 10¹¹ vg |
| AAV2#14 | 4.85 × 10³ | 2.8 × 10⁴ | 1.2 × 10⁵ |
| AAV2#12 | 2.3 × 10³ | 4.9 × 10⁴ | 9.2 × 10⁵ |

For evaluation of transgene expression, vector-derived transgene levels were quantified using transcript-specific TaqMan® assays in quantitative RT-PCR analyses of RNA purified from the NHP retinas. RNA quality was assessed, and all samples were shown to have an RNA integrity number (RIN) greater than 7.5. One sample was not included in RNA analyses due to low RNA input. Transcript levels were quantified relative to a double-stranded plasmid DNA standard curve. The data show that transduction of both AAV2 #12 and AAV2 #14 leads to dose-dependent levels of transgene expression in the NHP retina (dose-response AAV2 #14 p=0.0286, AAV2 #12 p=0.0286). Table 10 below summarizes transcript abundance in the NHP retina at 6 weeks after intravitreal administration (median transcripts/500 ng RNA).

TABLE 10

| | transcript abundance 6 | | |
|---|---|---|---|
| | weeks after dosing | | |
| | Dose | | |
| Construct | 2 × 10⁹ vg | 2 × 10¹⁰ vg | 2 × 10¹¹ vg |
| AAV2#14 | 1.8 × 10⁴ | 4.5 × 10⁴ | 6.3 × 10⁴ |
| AAV2#12 | 1.1 × 10⁴ | 2.5 × 10⁴ | 1.1 × 10⁵ |

### B. Study 2

In the second study, NHPs were administered through intravitreal injection ocular formulation buffer (N=2 NHPs), or AAV2 #14 or AAV2 #9 at 2×10¹¹ vg per eye (N=3 NHPs per vector treatment group). The vector titer was determined based on an assay that detects the BGH poly (A). The animals were assessed over 8 weeks of in-life exposure. Due to the presence of serum AAV2 neutralizing antibodies (Nab), all study 2 NHPs were administered an IgG degrading enzyme (IdeS) by intravitreal administration 2 days prior to vector dosing.

For evaluation of vector transduction, vector genome levels were quantified using vector-specific TaqMan® assays in quantitative PCR analyses of DNA purified from the NHP retinas. For AAV2 #9, vector genome levels were assessed using two different assays that detect the anti-Bb and anti-C1s arms. Comparable DNA input across samples was confirmed using TUBB as a reference gene. Despite potential hindrance by pre-existing AAV2 Nabs, the data show that both AAV2 #14 and AAV2 #9 successfully transduced the NHP retina, with AAV2 #14 achieving about 9.3×10³ vg and AAV2 #9 achieving levels between about 7.6×10⁴ and about 2.8×10⁵ vg at 8 weeks after intravitreal administration (median vector genomes/500 ng genomic DNA).

For evaluation of transgene expression, vector-derived transgene levels were quantified using transcript-specific TaqMan® assays in quantitative RT-PCR analyses of RNA purified from the NHP retinas. For AAV2 #9, the anti-Bb and anti-C1s transcripts are expressed independently and were therefore assessed separately. RNA quality was assessed, and all samples were shown to have a RNA integrity number (RIN) greater than 7.5. Transcript levels were quantified relative to a double-stranded plasmid DNA standard curve. After 8 weeks of in-life exposure, AAV2 #14 resulted in abundance levels of about 9.7×10⁴ transcripts and AAV2 #9 resulted in about 1.6×10⁶ anti-Bb transcripts and about 3.3×10⁵ anti-C1s transcripts (median transcripts per 500 ng retina RNA).

### C. Persistence Study in NHPs

The pharmacology and persistence across multiple dose levels of AAV2 #9 were evaluated in a study with a 16-week in-life assessment that included a 6-week interim necropsy.

NHPs (cynomolgus macaque) were administered through bilateral intravitreal injection the formulation buffer (180 mM NaCl, 5 mM sodium phosphate, 0.01% PS20, pH 7.4), or AAV2 #9 at multiple dose levels (based on vector titer determined by droplet digital PCR (ddPCR) analyses using a vector-specific assay targeting the anti-C1s region of AAV2 #9). All NHPs were given prophylactic steroids (1 mg/kg daily oral prednisolone) beginning two weeks prior to vector dosing and continuing throughout the entire study duration. Vector genome levels in the NHP retina were quantified using vector-specific C1s and Bb Taqman® assays in quantitative PCR analyses of DNA purified from the right eye.

The C1s and Bb assays detected comparable vector genome levels within each sample across the 6- and 16-week timepoints. At 6 weeks, AAV2 #9 transduction resulted in a dose-dependent increase in vector genome levels in the retina. A dose-dependent increase in retina transduction was also observed at 16 weeks.

Vector biodistribution in the NHP eye was assessed using an AAV2 #9 vector-specific probe set (containing 40 pairs of probes that each span about 50 bases, designed to detect the sense strand of the vector genome) in RNAscope™ ISH analyses. At 6- and 16-weeks, vector was detected in the retina and iris-ciliary body of eyes administered AAV2 #9. No vector was detected in the optic nerve. In the retina, vector was present in RGCs and in rare cells of the INL, often in the foveal and parafoveal region of the macula.

Levels of AAV2 #9-derived anti-C1s and anti-Bb transcripts in the NHP retina were quantified using C1s- and Bb-specific Taqman® assays in quantitative RT-PCR analyses of RNA purified from the right eye. Transcript levels were quantified relative to a double-stranded plasmid DNA standard curve. Transcript levels in both the 6- and 16-week cohorts were highly correlated with vector genome levels (Spearman r≥0.97). At 6 weeks, AAV2 #9 transduction resulted in a dose-dependent increase in transcript levels in the retina. A dose-dependent trend of increasing transcript levels was also observed at 16 weeks.

To assess the kinetics of peak scFab expression and persistence over time, aqueous humor was collected at baseline and during weeks 3, 6, 12, and 16. Vitreous humor was collected at necropsy. In the aqueous humor collected from some NHPs in the 16-week cohort, scFab levels peaked between 3-6 weeks and persisted through the end of the study at 4 months (day 113). Levels of scFabs in the vitreous humor at 4 months were similar to or higher than levels in the aqueous humor.

### D. Efficacy Study Evaluating AAV2 #9 Inhibition of LPS-Induced Complement Activation and Ocular Inflammation in NHPs

The ability of AAV2 #9-derived scFabs to inhibit complement pathway activation in vivo was assessed using an acute model of endotoxin-induced inflammation.

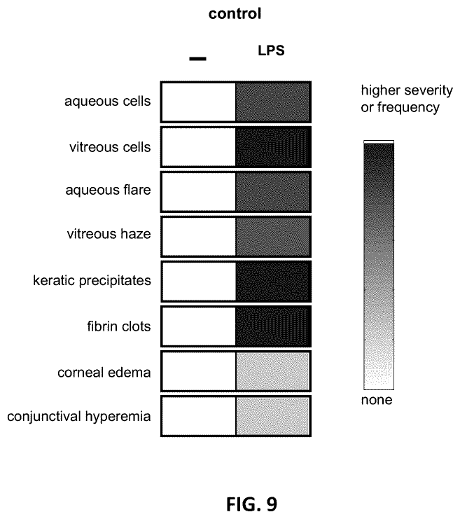

NHPs (cynomolgus macaque) were administered through bilateral intravitreal injection the formulation buffer (180 mM NaCl, 5 mM sodium phosphate, 0.01% PS20, pH 7.4) or AAV2 #9, followed by bilateral intravitreal lipo-polysaccharide (LPS) administration on day 41 [0.5 endotoxin units (EU) LPS per eye from Escherichia coli 0111: B4; Sigma-Aldrich L4391]. NHPs were given prophylactic steroids (1 mg/kg daily oral prednisolone) beginning two weeks prior to vector dosing and continuing daily for four weeks. NHPs were tapered off prednisolone prior to LPS administration on day 41. Study endpoints were assessed two days after LPS treatment (day 43), which induced high levels of ocular inflammation (FIG. 9).

Free drug levels of AAV2 #9-derived scFabs in the aqueous and vitreous humors were measured using Bb and C1s target-capture ELISAs. At the end of the study on day 43, aqueous humor and vitreous humor levels of free anti-C1s scFab averaged about 50-100 ng/ml (1-2 nM) and median levels of free anti-Bb scFab reached about 100-200 ng/mL (2-4 nM). The levels of the anti-C1s scFab in both the aqueous humor and vitreous humor were above the equilibrium dissociation constant of the anti-C1s scFab for both human and cynomolgus C1s (human K_{D}=0.34 nM; cynomolgus K_{D}=0.016 nM). The anti-Bb scFab had a lower affinity for cynomolgus Bb (K_{D}=14.8 nM) compared to human Bb (K_{D}=3.7 nM), and the levels of anti-Bb scFab reached in the aqueous humor and vitreous humor in this study were below the K_{D} of the anti-Bb scFab for cynomolgus Bb and were not sufficient for inhibition of Bb in NHP eye.

To assess complement pathway activation, we used a multiplexed ELISA from Quidel to measure activation fragment levels of C4a (classical pathway), Ba (alternative pathway) and sC5b9 (terminal pathway) in the aqueous humor. Compared to non-LPS treated control eyes, LPS-treated eyes had increased levels of Ba, C4a, and sC5b9 in the aqueous humor, demonstrating activation of the alternative, classical, and terminal pathways. LPS-treated eyes dosed with AAV2 #9 had reduced levels of C4a and sC5b9 compared to LPS-treated control eyes, demonstrating inhibition of the classical and terminal pathways. Inhibition of the alternative pathway (Ba) was not detected in AAV2 #9-treated eyes, likely due to the lower affinity of the anti-Bb scFab for the cynomolgus target.

Ocular exams performed two days after LPS dosing detected ocular inflammation in all treatment groups. However, eyes treated with AAV2 #9 had reduced severity and frequency of clinical indicators of inflammation scored using the SPOTS system.

SEQUENCES
SEQ ID NO: 1-HCDR1 of anti-C1s antibody
DDYIH
SEQ ID NO: 2-HCDR2 of anti-C1s antibody
RIDPADGHTK YAPKFQV
SEQ ID NO: 3-HCDR3 of anti-C1s antibody
YGYGREVEDY
SEQ ID NO: 4-LCDR1 of anti-C1s antibody
KASQSVDYDG DSYMN
SEQ ID NO: 5-LCDR2 of anti-C1s antibody
DASNLES
SEQ ID NO: 6-LCDR3 of anti-C1s antibody
QQSNEDPWT
SEQ ID NO: 7-V_{H} of anti-C1s antibody (Kabat CDRs underlined)
QVQLVQSGAE VKKPGASVKL SCTASGENIK DDYIHWVKQA PGQGLEWIGR IDPADGHTKY
APKFQVKVTI TADTSTSTAY LELSSLRSED TAVYYCARYG YGREVEDYWG QGTTVTVSS
SEQ ID NO: 8-V_{L} of anti-C1s antibody (Kabat CDRs underlined)
DIVLTQSPDS LAVSLGERAT ISCKASQSVD YDGDSYMNWY QQKPGOPPKI LIYDASNLES
GIPARESGSG SGTDFTLTIS SLEPEDFAIY YCQQSNEDPW TFGGGTKVEI K
SEQ ID NO: 9-αC1s scFv
QVOLVOSGAE VKKPGASVKL SCTASGENIK DDYIHWVKQA PGQGLEWIGR IDPADGHTKY
APKFQVKVTI TADTSTSTAY LELSSLRSED TAVYYCARYG YGREVEDYWG QGTTVTVSSG
GGGSGGGGSG GGGSDIVLTQ SPDSLAVSLG ERATISCKAS QSVDYDGDSY MNWYQQKPGQ
PPKILIYDAS NLESGIPARF SGSGSGTDET LTISSLEPED FAIYYCQQSN EDPWTFGGGT
KVEIK
SEQ ID NO: 10-heavy chain of anti-C1s Fab
QVOLVOSGAE VKKPGASVKL SCTASGENIK DDYIHWVKQA PGQGLEWIGR IDPADGHTKY
APKFQVKVTI TADTSTSTAY LELSSLRSED TAVYYCARYG YGREVFDYWG QGTTVTVSSA
STKGPSVFPL APCSRSTSES TAALGCLVKD YFPEPVTVSW NSGALTSGVH TFPAVLOSSG
LYSLSSVVTV PSSSLGTKTY TCNVDHKPSN TKVDKRV
SEQ ID NO: 11-light chain of anti-C1s Fab
DIVLTQSPDS LAVSLGERAT ISCKASQSVD YDGDSYMNWY QQKPGOPPKI LIYDASNLES
GIPARFSGSG SGTDETLTIS SLEPEDFAIY YCQQSNEDPW TFGGGTKVEI KRTVAAPSVF
IFPPSDEQLK SGTASVVCLL NNFYPREAKV QWKVDNALQS GNSQESVTEQ DSKDSTYSLS
STLTLSKADY EKHKVYACEV THQGLSSPVT KSENRGEC
SEQ ID NO: 12-αC1s scFab
DIVLTQSPDS LAVSLGERAT ISCKASQSVD YDGDSYMNWY QQKPGQPPKI LIYDASNLES
GIPARFSGSG SGTDFTLTIS SLEPEDFAIY YCQQSNEDPW TFGGGTKVEI KRTVAAPSVE
IFPPSDEQLK SGTASVVCLL NNFYPREAKV QWKVDNALQS GNSQESVTEQ DSKDSTYSLS
STLTLSKADY EKHKVYACEV THQGLSSPVT KSENRGECGG GGSGGGGSGG GGSGGGGSGG
GGSGGGGSGG GGSQVOLVOS GAEVKKPGAS VKLSCTASGF NIKDDYIHWV KQAPGOGLEW
IGRIDPADGH TKYAPKFQVK VTITADTSTS TAYLELSSLR SEDTAVYYCA RYGYGREVED
YWGQGTTVTV SSASTKGPSV FPLAPCSRST SESTAALGCL VKDYFPEPVT VSWNSGALTS
GVHTFPAVLQ SSGLYSLSSV VTVPSSSLGT KTYTCNVDHK PSNTKVDKRV
SEQ ID NO: 13-HCDR1 of anti-Bb antibody
NYAMS
SEQ ID NO: 14-HCDR2 of anti-Bb antibody
TISNRGSYTY YPDSVKG
SEQ ID NO: 15-HCDR3 of anti-Bb antibody
ERPMDY
SEQ ID NO: 16-LCDR1 of anti-Bb antibody
KASQDVGTAV A
SEQ ID NO:17-LCDR2 of anti-Bb antibody
WASTRHT
SEQ ID NO:18-LCDR3 of anti-Bb antibody
HQHSSNPLT
SEQ ID NO:19-V_{H} of anti-Bb antibody (Kabat CDRs boxed)

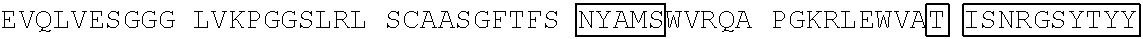

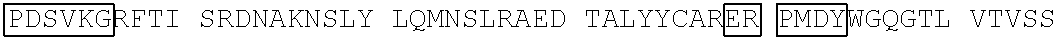

SEQ ID NO: 20-V_{L} of anti-Bb antibody (Kabat CDRs boxed)

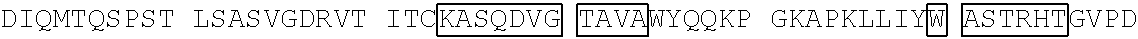

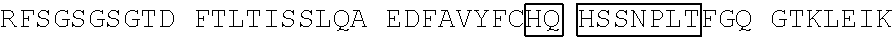

SEQ ID NO: 21-αBb scFv
EVOLVESGGG LVKPGGSLRL SCAASGFTES NYAMSWVRQA PGKRLEWVAT ISNRGSYTYY
PDSVKGRFTI SRDNAKNSLY LQMNSLRAED TALYYCARER PMDYWGQGTL VTVSSGGGGS
GGGGSGGGGS DIQMTQSPST LSASVGDRVT ITCKASQDVG TAVAWYQQKP GKAPKLLIYW
ASTRHTGVPD RESGSGSGTD FTLTISSLQA EDFAVYFCHQ HSSNPLTFGQ GTKLEIK
SEQ ID NO: 22-heavy chain of anti-Bb Fab
EVOLVESGGG LVKPGGSLRL SCAASGFTES NYAMSWVRQA PGKRLEWVAT ISNRGSYTYY
PDSVKGRFTI SRDNAKNSLY LOMNSLRAED TALYYCARER PMDYWGQGTL VTVSSASTKG
PSVEPLAPCS RSTSESTAAL GCLVKDYFPE PVTVSWNSGA LTSGVHTFPA VLOSSGLYSL
SSVVTVPSSS LGTKTYTCNV DHKPSNTKVD KRV
SEQ ID NO: 23-light chain of anti-Bb Fab
DIQMTQSPST LSASVGDRVT ITCKASQDVG TAVAWYQQKP GKAPKLLIYW ASTRHTGVPD
RFSGSGSGTD FTLTISSLQA EDFAVYFCHQ HSSNPLTFGQ GTKLEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSEN RGEC
SEQ ID NO: 24-αBb scFab
DIQMTQSPST LSASVGDRVT ITCKASQDVG TAVAWYQQKP GKAPKLLIYW ASTRHTGVPD
RFSGSGSGTD FTLTISSLQA EDFAVYFCHQ HSSNPLTFGQ GTKLEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSEN RGECGGGGSG GGGSGGGGSG GGGSGGGGSG
GGGSGGGGSE VOLVESGGGL VKPGGSLRLS CAASGFTFSN YAMSWVRQAP GKRLEWVATI
SNRGSYTYYP DSVKGRFTIS RDNAKNSLYL QMNSLRAEDT ALYYCARERP MDYWGQGTLV
TVSSASTKGP SVEPLAPCSR STSESTAALG CLVKDYFPEP VTVSWNSGAL TSGVHTEPAV
LOSSGLYSLS SVVTVPSSSL GTKTYTCNVD HKPSNTKVDK RV
SEQ ID NO: 25-[αC1s scFab-(G₄S)₃-αBb scFab] nucleic acid
sequence (construct #7, FIG. 2A)
ATGGAAGCCC CCGCCCAGCT GCTGTTCCTG CTGCTGCTGT GGCTGCCTGA CACCACTGGC
GATATCGTGC TGACACAGAG CCCTGATAGC CTGGCTGTTA GCCTGGGCGA ACGCGCCACA
ATCAGCTGCA AGGCCAGCCA GTCTGTGGAT TATGATGGTG ACAGCTACAT GAACTGGTAC
CAGCAGAAGC CCGGACAGCC TCCTAAGATC CTGATCTACG ACGCCAGCAA CCTGGAATCC
GGCATTCCTG CCCGGTTCAG CGGCTCCGGC AGCGGCACCG ACTTCACCCT GACCATCTCC
AGCCTGGAAC CCGAGGATTT CGCCATCTAC TACTGTCAGC AGAGCAATGA GGACCCATGG
ACCTTCGGCG GCGGTACCAA GGTCGAGATC AAGAGAACAG TGGCCGCTCC TAGCGTGTTC
ATCTTCCCTC CATCAGACGA GCAGCTGAAG AGCGGAACCG CTTCTGTGGT GTGTCTGCTC
AACAATTTCT ACCCTAGAGA AGCCAAGGTG CAGTGGAAGG TGGACAACGC TCTCCAGAGC
GGCAACAGCC AGGAGAGCGT GACCGAGCAA GATAGCAAGG ACAGCACCTA CTCTTTAAGC
TCTACACTGA CGCTGTCCAA GGCTGACTAC GAGAAGCACA AGGTGTACGC CTGTGAAGTG
ACCCACCAGG GCCTGAGCAG CCCTGTGACA AAGAGCTTCA ACAGAGGCGA GTGCGGCGGC
GGAGGCAGCG GCGGCGGAGG CTCTGGCGGT GGCGGAAGCG GCGGGGGAGG CTCTGGCGGC
GGCGGCAGTG GCGGCGGCGG CAGCGGAGGA GGAGGATCGC AAGTGCAACT GGTCCAGTCT
GGCGCCGAGG TGAAAAAGCC TGGAGCCAGC GTGAAACTGT CATGCACCGC CTCCGGGTTT
AACATCAAAG ATGACTACAT CCACTGGGTG AAACAGGCTC CAGGACAGGG CCTGGAGTGG
ATCGGCAGAA TCGACCCTGC GGATGGCCAC ACCAAGTACG CCCCAAAGTT CCAGGTGAAG
GTGACAATCA CAGCTGACAC CAGCACCAGC ACAGCCTACC TGGAACTGAG CAGCCTAAGA
AGCGAGGACA CCGCCGTGTA CTACTGCGCC CGGTACGGCT ACGGCCGGGA AGTGTTCGAC
TACTGGGGTC AGGGCACCAC CGTGACGGTG AGTAGCGCCT CTACAAAAGG CCCTTCCGTG
TTCCCCCTGG CCCCTTGCAG CCGGAGCACC AGCGAGAGCA CCGCCGCCTT GGGCTGTCTG
GTGAAAGACT ATTTCCCAGA GCCTGTCACA GTGTCTTGGA ACTCCGGAGC CCTCACCTCT
GGAGTGCACA CATTTCCCGC CGTGCTGCAG AGCAGCGGCT TGTACTCTCT GAGCAGCGTG
GTGACAGTGC CCTCTAGCAG CCTGGGCACA AAGACCTACA CCTGCAACGT GGACCACAAG
CCTTCTAACA CCAAGGTGGA TAAGAGAGTG GGTGGCGGAG GAAGCGGCGG CGGAGGAAGC
GGCGGCGGCG GGTCCGATAT TCAGATGACC CAGAGCCCTT CTACCCTTAG TGCCTCTGTT
GGAGACCGGG TGACCATCAC CTGTAAAGCC TCCCAGGACG TGGGAACAGC AGTTGCTTGG
TATCAGCAAA AGCCCGGCAA GGCCCCTAAG TTGCTGATCT ACTGGGCCTC CACAAGACAC
ACCGGCGTGC CTGATAGATT CAGCGGTAGC GGCAGCGGCA CCGATTTTAC CCTGACAATC
AGCTCTCTGC AGGCCGAGGA CTTTGCCGTG TACTTCTGCC ACCAGCATTC TAGCAATCCT
CTGACTTTTG GCCAGGGCAC CAAGCTGGAA ATCAAGCGGA CAGTAGCCGC TCCTTCTGTA
TTTATCTTCC CACCTTCTGA CGAGCAGCTG AAGTCTGGTA CCGCAAGCGT GGTGTGCCTG
CTGAACAACT TCTACCCCAG AGAGGCCAAA GTGCAATGGA AGGTGGACAA CGCCCTGCAG
AGTGGCAATA GCCAGGAGTC TGTCACTGAG CAGGACTCCA AGGATAGCAC CTACAGCCTG
TCTTCTACAC TCACCCTGTC CAAGGCCGAC TACGAGAAGC ACAAGGTGTA CGCCTGCGAG
GTGACACACC AGGGCCTGTC TTCCCCTGTG ACCAAAAGCT TCAACCGGGG CGAGTGCGGG
GGCGGCGGAA GCGGTGGCGG CGGGTCCGGC GGCGGCGGCA GCGGCGGCGG CGGCAGCGGA
GGCGGCGGCA GTGGTGGGGG CGGCTCGGGC GGCGGAGGCT CTGAGGTGCA GCTGGTGGAA
AGTGGCGGAG GCCTGGTGAA GCCCGGCGGC AGCCTGAGAC TGAGTTGCGC CGCGAGCGGA
TTCACTTTCT CCAACTACGC CATGTCTTGG GTGAGACAGG CCCCTGGCAA AAGACTGGAA
TGGGTCGCTA CCATCAGCAA CAGAGGTAGC TACACATACT ACCCTGATAG CGTGAAAGGC
AGGTTCACCA TCAGCAGGGA CAACGCCAAG AACAGCCTGT ATCTGCAGAT GAACAGCCTG
CGGGCCGAAG ATACAGCCCT TTATTACTGC GCGAGAGAGA GACCCATGGA CTACTGGGGC
CAGGGAACAC TGGTGACCGT TTCAAGCGCC TCTACCAAGG GCCCCTCTGT GTTTCCTCTG
GCCCCTTGTT CTCGGAGCAC CTCCGAGAGC ACCGCTGCTC TGGGATGCCT CGTGAAGGAC
TATTTCCCCG AACCCGTGAC CGTGTCCTGG AACAGCGGCG CCCTGACAAG CGGGGTCCAC
ACCTTCCCCG CCGTCCTGCA GAGTTCTGGA CTGTACAGCC TGAGCAGCGT CGTCACAGTG
CCTTCAAGCA GCCTGGGCAC CAAGACCTAC ACCTGCAACG TGGACCATAA GCCTTCCAAT
ACCAAGGTGG ACAAGAGAGT TTGA
SEQ ID NO: 26-[αC1s scFab-(G₄S)₃-αBb scFab] amino acid (signal
peptide boldfaced) (construct #7, FIG. 2A)
MEAPAQLLFL LLLWLPDTTG DIVLTOSPDS LAVSLGERAT ISCKASQSVD YDGDSYMNWY
QQKPGQPPKI LIYDASNLES GIPARFSGSG SGTDFTLTIS SLEPEDFAIY YCQQSNEDPW
TFGGGTKVEI KRTVAAPSVE IFPPSDEQLK SGTASVVCLL NNFYPREAKV QWKVDNALQS
GNSQESVTEQ DSKDSTYSLS STLTLSKADY EKHKVYACEV THQGLSSPVT KSENRGECGG
GGSGGGGSGG GGSGGGGSGG GGSGGGGSGG GGSQVQLVQS GAEVKKPGAS VKLSCTASGF
NIKDDYIHWV KQAPGOGLEW IGRIDPADGH TKYAPKFQVK VTITADTSTS TAYLELSSLR
SEDTAVYYCA RYGYGREVED YWGQGTTVTV SSASTKGPSV FPLAPCSRST SESTAALGCL
VKDYFPEPVT VSWNSGALTS GVHTFPAVLQ SSGLYSLSSV VTVPSSSLGT KTYTCNVDHK
PSNTKVDKRV GGGGSGGGGS GGGGSDIQMT QSPSTLSASV GDRVTITCKA SQDVGTAVAW
YQQKPGKAPK LLIYWASTRH TGVPDRESGS GSGTDFTLTI SSLQAEDFAV YFCHQHSSNP
LTFGQGTKLE IKRTVAAPSV FIFPPSDEQL KSGTASVVCL LNNFYPREAK VOWKVDNALQ
SGNSQESVTE QDSKDSTYSL SSTLTLSKAD YEKHKVYACE VTHQGLSSPV TKSENRGECG
GGGSGGGGSG GGGSGGGGSG GGGSGGGGSG GGGSEVQLVE SGGGLVKPGG SLRLSCAASG
FTESNYAMSW VRQAPGKRLE WVATISNRGS YTYYPDSVKG RFTISRDNAK NSLYLQMNSL
RAEDTALYYC ARERPMDYWG QGTLVTVSSA STKGPSVEPL APCSRSTSES TAALGCLVKD
YFPEPVTVSW NSGALTSGVH TFPAVLOSSG LYSLSSVVTV PSSSLGTKTY TCNVDHKPSN
TKVDKRV*
SEQ ID NO: 27-[αC1s scFab-(G₄S)₃-αBb scFab-CM] nucleic acid
sequence (construct #11, FIG. 2C)
ATGGAAGCCC CCGCCCAGCT GCTGTTCCTG CTGCTGCTGT GGCTGCCTGA CACCACTGGC
GATATCGTGC TGACACAGAG CCCTGATAGC CTGGCTGTTA GCCTGGGCGA ACGCGCCACA
ATCAGCTGCA AGGCCAGCCA GTCTGTGGAT TATGATGGTG ACAGCTACAT GAACTGGTAC
CAGGAGAAGC CCGGACAGCC TCCTAAGATC CTGATCTACG ACGCCAGCAA CCTGGAATCC
GGCATTCCTG CCCGGTTCAG CGGCTCCGGC AGCGGCACCG ACTTCACCCT GACCATCTCC
AGCCTGGAAC CCGAGGATTT CGCCATCTAC TACTGTCAGC AGAGCAATGA GGACCCATGG
ACCTTCGGCG GCGGTACCAA GGTCGAGATC AAGAGAACAG TGGCCGCTCC TAGCGTGTTC
ATCTTCCCTC CATCAGACGA GCAGCTGAAG AGCGGAACCG CTTCTGTGGT GTGTCTGCTC
AACAATTTCT ACCCTAGAGA AGCCAAGGTG CAGTGGAAGG TGGACAACGC TCTCCAGAGC
GGCAACAGCC AGGAGAGCGT GACCGAGCAA GATAGCAAGG ACAGCACCTA CTCTTTAAGC
TCTACACTGA CGCTGTCCAA GGCTGACTAC GAGAAGCACA AGGTGTACGC CTGTGAAGTG
ACCCACCAGG GCCTGAGCAG CCCTGTGACA AAGAGCTTCA ACAGAGGCGA GTGCGGCGGC
GGAGGCAGCG GCGGCGGAGG CTCTGGCGGT GGCGGAAGCG GCGGGGGAGG CTCTGGCGGC
GGCGGCAGTG GCGGCGGCGG CAGCGGAGGA GGAGGATCGC AAGTGCAACT GGTCCAGTCT
GGCGCCGAGG TGAAAAAGCC TGGAGCCAGC GTGAAACTGT CATGCACCGC CTCCGGGTTT
AACATCAAAG ATGACTACAT CCACTGGGTG AAAAAGGCTC CAGGACAGGG CCTGGAGTGG
ATCGGCAGAA TCGACCCTGC GGATGGCCAC ACCAAGTACG CCCCAAAGTT CCAGGTGAAG
GTGACAATCA CAGCTGACAC CAGCACCAGC ACAGCCTACC TGGAACTGAG CAGCCTAAGA
AGCGAGGACA CCGCCGTGTA CTACTGCGCC CGGTACGGCT ACGGCCGGGA AGTGTTCGAC
TACTGGGGTC AGGGCACCAC CGTGACGGTG AGTAGCGCCT CTACAAAAGG CCCTTCCGTG
TTCCCCCTGG CCCCTTGCAG CCGGAGCACC AGCGAGAGCA CCGCCGCCTT GGGCTGTCTG
GTGAAAGACT ATTTCCCAGA GCCTGTCACA GTGTCTTGGA ACTCCGGAGC CCTCACCTCT
GGAGTGCACA CATTTCCCGC CGTGCTGCAG AGCAGCGGCT TGTACTCTCT GAGCAGCGTG
GTGACAGTGC CCTCTAGCAG CCTGGGCACA AAGACCTACA CCTGCAACGT GGACCACAAG
CCTTCTAACA CCAAGGTGGA TAAGAGAGTG GGTGGCGGAG GAAGCGGCGG CGGAGGAAGC
GGCGGCGGCG GGTCCGATAT TCAGATGACC CAGAGCCCTT CTACCCTTAG TGCCTCTGTT
GGAGACCGGG TGACCATCAC CTGTAAAGCC TCCCAGGACG TGGGAACAGC AGTTGCTTGG
TATCAGAAAA AGCCCGGCAA GGCCCCTAAG TTGCTGATCT ACTGGGCCTC CACAAGACAC
ACCGGCGTGC CTGATAGATT CAGCGGTAGC GGCAGCGGCA CCGATTTTAC CCTGACAATC
AGCTCTCTGC AGGCCGAGGA CTTTGCCGTG TACTTCTGCC ACCAGCATTC TAGCAATCCT
CTGACTTTTG GCCAGGGCAC CAAGCTGGAA ATCAAGCGGA CAGTAGCCGC TCCTGCTGTA
TTTATCTTCC CACCTTCTGA CGAGCAGCTG AAGTCTGGTA CCGCAAGCGT GGTGTGCCTG
CTGAAGAACT TCTACCCCAG AGAGGCCAAA GTGCAATGGA AGGTGGACAA CGCCCTGCAG
AGTGGCAATA GCCAGGAGTC TGTCACTGAG CAGGACTCCA AGGATAGCAC CTACAGCCTG
TCTTCTACAC TCACCCTGTC CAAGGCCGAC TACGAGAAGC ACAAGGTGTA CGCCTGCGAG
GTGACACACC AGGGCCTGTC TTCCCCTGTG ACCAAAAGCT TCAACCGGGG CGAGTGCGGG
GGCGGCGGAA GCGGTGGCGG CGGGTCCGGC GGCGGCGGCA GCGGCGGCGG CGGCAGCGGA
GGCGGCGGCA GTGGTGGGGG CGGCTCGGGC GGCGGAGGCT CTGAGGTGCA GCTGGTGGAA
AGTGGCGGAG GCCTGGTGAA GCCCGGCGGC AGCCTGAGAC TGAGTTGCGC CGCGAGCGGA
TTCACTTTCT CCAACTACGC CATGTCTTGG GTGAGAGAGG CCCCTGGCAA AAGACTGGAA
TGGGTCGCTA CCATCAGCAA CAGAGGTAGC TACACATACT ACCCTGATAG CGTGAAAGGC
AGGTTCACCA TCAGCAGGGA CAACGCCAAG AACAGCCTGT ATCTGCAGAT GAACAGCCTG
CGGGCCGAAG ATACAGCCCT TTATTACTGC GCGAGAGAGA GACCCATGGA CTACTGGGGC
CAGGGAACAC TGGTGACCGT TTCAAGCGCC TCTACCAAGG GCCCCTCTGT GTTTCCTCTG
GCCCCTTGTT CTCGGAGCAC CTCCGAGAGC ACCGCTGCTC TGGGATGCCT CGTGAAGGAC
TATTTCCCCG AACCCGTGAC CGTGTCCTGG AACAGCGGCG CCCTGACAAG CGGGGTCCAC
ACCTTCCCCG CCGTCCTGCA GAGTTCTGGA CTGTACAGCC TGAGCAGCGT CGTCGAAGTG
CCTTCAAGCA GCCTGGGCAC CAAGACCTAC ACCTGCAACG TGGACCATAA GCCTTCCAAT
ACCAAGGTGG ACAAGAGAGT TTGA
SEQ ID NO: 28-[αCls scFab-(G₄S)₃-αBb scFab CM] amino acid
sequence (construct #11, FIG. 2C) (signal peptide boldfaced; charge
mutations boxed and italicized, numbering excluding signal peptide:
Q42E and Q292K in αCls scFab, and Q523K, S599A, N622K, Q773E and
T919E in αBb scFab)
MEAPAQLLFL LLLWLPDTTG DIVLTQSPDS LAVSLGERAT ISCKASQSVD YDGDSYMNWY

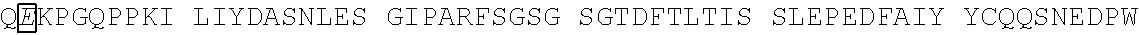

TFGGGTKVEI KRTVAAPSVF IFPPSDEQLK SGTASVVCLL NNFYPREAKV QWKVDNALQS
GNSQESVTEQ DSKDSTYSLS STLTLSKADY EKHKVYACEV THQGLSSPVT KSENRGECGG
GGSGGGGSGG GGSGGGGSGG GGSGGGGSGG GGSQVQLVQS GAEVKKPGAS VKLSCTASGF

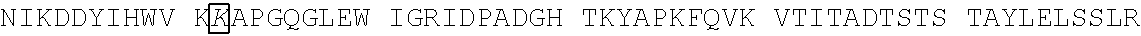

SEDTAVYYCA RYGYGREVFD YWGQGTTVTV SSASTKGPSV FPLAPCSRST SESTAALGCL
VKDYFPEPVT VSWNSGALTS GVHTFPAVLQ SSGLYSLSSV VTVPSSSLGT KTYTCNVDHK
PSNTKVDKRV GGGGSGGGGS GGGGSDIQMT QSPSTLSASV GDRVTITCKA SQDVGTAVAW

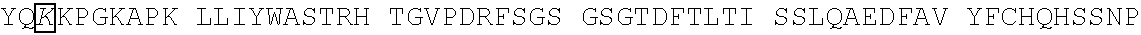

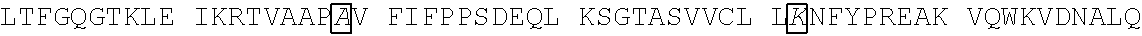

SGNSQESVTE QDSKDSTYSL SSTLTLSKAD YEKHKVYACE VTHQGLSSPV TKSENRGECG
GGGSGGGGSG GGGSGGGGSG GGGSGGGGSG GGGSEVQLVE SGGGLVKPGG SLRLSCAASG

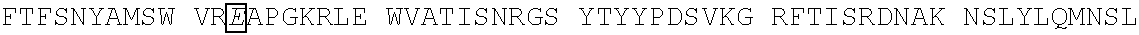

RAEDTALYYC ARERPMDYWG QGTLVTVSSA STKGPSVFPL APCSRSTSES TAALGCLVKD

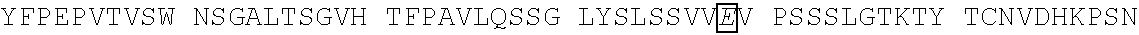

TKVDKRV*
SEQ ID NO: 29-[αBb scFab-(G₄S)₃-αC1s scFab] nucleic acid
sequence (construct #8, FIG. 2A)
ATGGAAGCCC CCGCCCAGCT GCTTTTCCTG CTGCTGCTGT GGCTGCCTGA TACCACCGGC
GATATCCAGA TGACCCAGAG CCCTAGCACC TTGAGCGCCT CTGTGGGCGA CAGAGTGACC
ATCACCTGCA AGGCCAGCCA GGACGTGGGC ACAGCCGTGG CTTGGTATCA GCAAAAACCT
GGCAAGGCCC CTAAGCTGCT GATTTACTGG GCCAGCACCA GACACACAGG CGTGCCTGAC
CGGTTTAGCG GCAGTGGCAG CGGGACAGAT TTTACCCTGA CCATCAGCTC TCTGCAGGCC
GAGGACTTCG CTGTGTACTT CTGCCACCAG CACAGCAGCA ACCCCCTGAC CTTTGGCCAG
GGCACCAAGC TGGAGATCAA GCGGACCGTG GCCGCACCCA GTGTGTTTAT CTTCCCCCCC
AGCGATGAGC AGCTGAAGAG CGGCACAGCC AGCGTGGTGT GTCTGCTGAA CAACTTCTAC
CCTAGAGAGG CTAAGGTGCA GTGGAAGGTG GATAATGCTC TGCAGAGCGG AAATAGCCAG
GAGTCTGTGA CCGAGCAGGA CAGCAAGGAC TCCACATACA GCCTCTCCTC CACCCTGACA
CTGTCCAAGG CCGATTACGA GAAGCACAAA GTGTACGCCT GCGAGGTGAC ACACCAGGGC
CTTAGCAGCC CTGTCACCAA ATCTTTCAAC AGAGGAGAGT GCGGCGGCGG CGGCTCCGGC
GGCGGCGGAT CTGGAGGCGG AGGCAGCGGA GGCGGGGGAA GCGGCGGAGG CGGCAGCGGC
GGCGGAGGTT CCGGCGGAGG CGGCTCAGAG GTGCAACTCG TGGAAAGCGG TGGCGGCCTG
GTTAAGCCCG GCGGCAGCCT GCGCCTGTCA TGCGCTGCAA GCGGCTTCAC CTTTTCAAAT
TACGCCATGA GCTGGGTGCG GCAGGCTCCT GGAAAACGGC TGGAATGGGT GGCTACAATC
TCTAACCGGG GCTCTTACAC CTACTACCCC GATAGCGTGA AAGGCAGATT CACAATCAGC
CGGGACAACG CCAAGAACTC ACTGTACCTG CAGATGAACT CCCTGCGGGC CGAGGACACA
GCTCTGTACT ACTGTGCCAG AGAAAGACCC ATGGACTACT GGGGACAGGG CACACTGGTT
ACAGTCTCCT CTGCCTCCAC GAAGGGCCCC AGCGTGTTCC CTCTGGCTCC TTGTAGCAGA
AGCACTTCTG AATCTACCGC TGCCCTGGGC TGCCTGGTGA AGGACTACTT CCCTGAGCCT
GTGACCGTTA GCTGGAACAG CGGAGCCCTG ACAAGCGGAG TGCATACATT CCCTGCCGTG
CTGCAGAGCA GCGGCCTCTA CAGCCTGTCC TCGGTGGTGA CCGTCCCCTC AAGCAGCCTG
GGCACCAAGA CCTACACTTG CAACGTGGAC CATAAGCCTA GCAACACAAA GGTGGACAAG
AGAGTCGGAG GCGGAGGTGG CTCCGGCGGC GGTGGCTCTG GCGGAGGCGG CAGCGACATC
GTGCTGACCC AAAGCCCTGA CAGCCTGGCC GTGTCCCTGG GAGAGCGGGC CACGATCTCC
TGCAAGGCCT CCCAATCCGT GGACTATGAT GGCGATAGCT ACATGAACTG GTACCAGCAG
AAGCCTGGCC AGCCTCCAAA GATCCTGATT TACGACGCCT CTAATCTGGA ATCCGGCATC
CCTGCTAGAT TCAGCGGAAG CGGTAGCGGC ACCGACTTCA CCCTGACAAT CAGCAGTCTG
GAGCCAGAGG ACTTCGCCAT CTACTACTGT CAGCAGTCTA ACGAGGATCC TTGGACCTTC
GGCGGCGGCA CCAAGGTGGA AATCAAGAGA ACCGTGGCCG CCCCTAGCGT CTTCATCTTC
CCTCCTAGTG ATGAGCAGCT GAAAAGCGGC ACAGCCAGCG TGGTGTGCCT CCTGAACAAC
TTCTACCCGC GCGAAGCCAA AGTGCAGTGG AAGGTGGACA ACGCCCTGCA GAGCGGCAAC
AGCCAGGAGT CCGTGACAGA GCAAGATAGC AAGGACAGCA CCTACTCCCT GTCGTCTACA
CTTACCCTGT CTAAAGCCGA CTATGAGAAG CACAAGGTAT ACGCCTGTGA AGTGACCCAC
CAGGGGCTGT CCTCTCCAGT AACCAAGTCC TTCAACAGAG GCGAATGCGG CGGAGGCGGA
TCTGGCGGCG GCGGCTCCGG CGGCGGCGGC AGCGGCGGCG GCGGCAGCGG GGGCGGAGGC
AGCGGCGGCG GAGGAAGCGG AGGCGGAGGC AGCCAGGTGC AGCTGGTGCA GTCAGGCGCT
GAGGTGAAAA AGCCTGGCGC CAGCGTCAAG CTGTCTTGCA CCGCTTCTGG CTTTAACATC
AAGGACGACT ACATCCACTG GGTCAAGCAG GCCCCCGGGC AAGGGCTGGA GTGGATCGGC
AGAATCGACC CTGCCGACGG CCACACCAAG TACGCCCCTA AGTTCCAGGT GAAGGTGACA
ATCACAGCTG ATACCAGCAC GAGCACCGCC TACCTGGAAC TGTCATCCCT CAGATCTGAA
GATACAGCCG TTTACTACTG CGCAAGGTAC GGGTACGGGC GGGAAGTGTT CGACTATTGG
GGCCAGGGCA CAACCGTGAC CGTGAGCAGC GCCTCTACCA AAGGCCCTAG CGTGTTCCCC
CTGGCTCCTT GCAGCAGATC TACAAGCGAG AGCACAGCCG CCCTGGGATG TCTGGTTAAA
GATTATTTCC CAGAACCTGT GACAGTGAGC TGGAACAGCG GCGCCCTGAC CAGCGGCGTG
CACACCTTCC CAGCCGTGCT GCAGTCATCC GGTCTGTATA GCCTGAGCAG CGTGGTTACC
GTGCCCAGCT CTAGCCTGGG CACCAAAACC TACACCTGCA ATGTGGACCA CAAGCCAAGC
AATACCAAGG TTGATAAGAG AGTCTGA
SEQ ID NO: 30-[αBb scFab-(G₄S)₃-αQC1s scFab] amino acid sequence
(construct #8, FIG. 2A) (signal peptide boldfaced)
MEAPAQLLFL LLLWLPDTTG DIQMTQSPST LSASVGDRVT ITCKASQDVG TAVAWYQQKP
GKAPKLLIYW ASTRHTGVPD RESGSGSGTD FTLTISSLQA EDFAVYFCHQ HSSNPLTEGO
GTKLEIKRTV AAPSVFIFPP SDEQLKSGTA SVVCLLNNFY PREAKVOWKV DNALQSGNSQ
ESVTEQDSKD STYSLSSTLT LSKADYEKHK VYACEVTHQG LSSPVTKSEN RGECGGGGSG
GGGSGGGGSG GGGSGGGGSG GGGSGGGGSE VOLVESGGGL VKPGGSLRLS CAASGFTESN
YAMSWVRQAP GKRLEWVATI SNRGSYTYYP DSVKGRFTIS RDNAKNSLYL QMNSLRAEDT
ALYYCARERP MDYWGQGTLV TVSSASTKGP SVEPLAPCSR STSESTAALG CLVKDYFPEP
VTVSWNSGAL TSGVHTFPAV LOSSGLYSLS SVVTVPSSSL GTKTYTCNVD HKPSNTKVDK
RVGGGGGSGG GGSGGGGSDI VLTQSPDSLA VSLGERATIS CKASQSVDYD GDSYMNWYQQ
KPGQPPKILI YDASNLESGI PARFSGSGSG TDFTLTISSL EPEDFAIYYC QQSNEDPWTF
GGGTKVEIKR TVAAPSVFIF PPSDEQLKSG TASVVCLLNN FYPREAKVQW KVDNALQSGN
SQESVTEQDS KDSTYSLSST LTLSKADYEK HKVYACEVTH QGLSSPVTKS FNRGECGGGG
SGGGGSGGGG SGGGGSGGGG SGGGGSGGGG SQVOLVOSGA EVKKPGASVK LSCTASGENI
KDDYIHWVKQ APGQGLEWIG RIDPADGHTK YAPKFQVKVT ITADTSTSTA YLELSSLRSE
DTAVYYCARY GYGREVEDYW GQGTTVTVSS ASTKGPSVFP LAPCSRSTSE STAALGCLVK
DYFPEPVTVS WNSGALTSGV HTFPAVLOSS GLYSLSSVVT VPSSSLGTKT YTCNVDHKPS
NTKVDKRV*
SEQ ID NO: 31-[αBb scFab-(g₄s)₃-αC1S scfAB-CM] nucleic acid
sequence (construct #12,FIG. 2C)
ATGGAAGCCC CTGCCCAGCT GCTGTTCCTG CTGCTACTGT GGCTGCCTGA TACCACCGGC
GATATCCAGA TGACGCAGAG TCCCAGCACC CTGAGCGCCT CTGTGGGCGA CCGGGTGACC
ATCACCTGTA AAGCCTCCCA GGACGTGGGC ACAGCTGTTG CTTGGTATCA GAAAAAGCCT
GGCAAGGCCC CTAAGCTGCT GATCTACTGG GCCAGCACAA GACACACAGG AGTGCCTGAC
AGATTCAGCG GCAGCGGCTC TGGGACTGAT TTCACCTTGA CAATCAGCTC TCTGCAGGCC
GAGGACTTTG CCGTGTACTT CTGCCACCAA CACAGTTCTA ACCCCCTGAC CTTCGGCCAA
GGAACCAAGC TGGAAATCAA GCGGACCGTG GCCGCTCCTG CCGTGTTCAT CTTCCCTCCA
AGCGATGAGC AGCTGAAAAG CGGCACCGCG TCCGTCGTGT GCCTGCTGAA GAACTTCTAC
CCGAGAGAAG CGAAGGTGCA GTGGAAAGTC GACAACGCCC TGCAGAGCGG AAATAGCCAG
GAGAGCGTGA CCGAACAAGA CTCTAAGGAC AGCACCTACT CGCTGTCCTC CACGCTGACT
CTGTCTAAGG CCGACTATGA GAAGCACAAG GTGTACGCCT GCGAGGTGAC CCACCAGGGC
CTGAGCAGCC CCGTTACCAA GAGCTTCAAC AGAGGAGAAT GCGGCGGAGG TGGCAGCGGC
GGCGGCGGGA GCGGCGGCGG CGGCTCAGGC GGAGGGGGAA GTGGCGGCGG CGGCAGCGGC
GGCGGAGGCA GCGGCGGTGG CGGCTCTGAG GTGCAACTGG TGGAATCTGG GGGCGGACTG
GTGAAGCCTG GCGGCAGTCT GAGACTGAGC TGTGCCGCTT CCGGATTCAC CTTTAGCAAT
TACGCCATGA GCTGGGTGCG GGAGGCCCCT GGAAAGCGGC TGGAATGGGT TGCTACAATC
AGCAATAGAG GCAGCTACAC ATACTACCCC GACAGTGTCA AAGGCCGGTT TACAATCAGC
CGCGACAACG CCAAAAACAG CCTGTACCTG CAGATGAACT CCCTGCGGGC TGAGGATACA
GCCCTCTACT ACTGTGCCAG AGAACGTCCA ATGGACTATT GGGGCCAAGG CACACTGGTG
ACCGTGAGCA GCGCGTCTAC CAAGGGCCCT TCTGTTTTCC CTCTGGCCCC CTGCAGCAGA
AGCACGAGCG AGAGCACCGC TGCCCTGGGC TGTCTGGTGA AGGATTATTT CCCTGAGCCT
GTGACCGTGT CTTGGAATAG CGGAGCCCTG ACCAGCGGAG TGCATACATT CCCTGCTGTG
CTGCAGTCTA GTGGGCTGTA CAGCCTGTCT TCCGTTGTGG AAGTCCCTAG CAGCAGCCTG
GGCACCAAGA CCTACACCTG CAACGTGGAT CATAAGCCAA GCAACACCAA GGTGGATAAG
AGAGTGGGCG GTGGCGGAGG CTCGGGCGGC GGCGGCAGCG GCGGCGGCGG CAGCGACATC
GTGCTGACCC AGTCTCCAGA TTCTCTGGCC GTGTCACTGG GAGAGAGAGC CACCATTAGC
TGCAAGGCCT CTCAGAGCGT AGACTACGAC GGCGACTCCT ACATGAACTG GTACCAGGAA
AAGCCTGGCC AGCCTCCTAA GATCTTGATC TACGATGCCT CCAATCTGGA GAGCGGGATC
CCCGCTAGAT TCAGCGGGTC TGGAAGTGGA ACCGACTTCA CACTGACCAT CTCTAGCCTG
GAGCCCGAGG ACTTTGCCAT CTACTACTGC CAGCAGAGCA ACGAGGACCC CTGGACATTC
GGCGGCGGCA CAAAGGTTGA GATCAAGAGA ACCGTTGCCG CTCCTAGCGT GTTTATCTTC
CCTCCCTCTG ACGAGCAGCT GAAGAGCGGC ACAGCCTCCG TGGTGTGCCT GCTGAACAAC
TTCTACCCCA GAGAGGCCAA GGTCCAGTGG AAGGTCGACA ATGCCCTTCA GAGCGGCAAC
AGCCAGGAGT CCGTGACCGA GCAGGATAGC AAGGACTCTA CCTACAGCCT GTCCTCTACG
CTGACCCTGA GCAAAGCCGA TTACGAAAAG CACAAAGTGT ACGCCTGTGA AGTGACACAC
CAGGGCCTGT CTAGCCCTGT GACAAAGAGC TTTAACCGGG GCGAGTGCGG CGGCGGTGGA
AGCGGAGGTG GAGGTTCAGG AGGCGGCGGA AGCGGAGGCG GAGGCAGTGG GGGCGGCGGC
TCCGGCGGCG GCGGCAGCGG AGGCGGCGGT TCCCAAGTGC AGCTCGTGCA GAGCGGCGCC
GAGGTGAAAA AGCCCGGAGC CAGCGTGAAG CTGTCTTGCA CCGCCTCCGG ATTCAACATC
AAAGACGACT ACATCCACTG GGTCAAGAAA GCCCCAGGGC AGGGGCTGGA GTGGATCGGC
AGGATCGACC CTGCTGATGG CCACACCAAA TACGCCCCAA AGTTCCAGGT GAAAGTGACA
ATTACCGCAG ATACCTCCAC CAGCACCGCT TATCTGGAAC TGAGCTCTCT GCGGAGCGAG
GACACAGCCG TGTACTACTG CGCCAGATAC GGCTACGGCA GAGAAGTGTT CGACTACTGG
GGCCAGGGCA CCACAGTGAC AGTGAGCTCT GCCAGCACAA AGGGCCCCAG CGTGTTTCCT
CTGGCCCCTT GCAGCAGAAG CACCAGCGAG AGCACCGCCG CCCTGGGCTG CCTGGTGAAG
GACTACTTCC CTGAACCCGT GACCGTCTCC TGGAACAGTG GCGCCTTGAC CTCTGGCGTG
CACACCTTCC CCGCCGTGCT GCAGAGCTCC GGCCTGTACA GCCTGTCTAG CGTGGTGACC
GTGCCTAGCT CGAGCCTGGG CACAAAGACA TATACCTGTA ACGTGGACCA CAAGCCCAGC
AACACGAAGG TGGACAAGCG AGTGTGA
SEQ ID NO: 32 [αBb scFab-(G₄S)₃-αC1s scFab-CM] amino acid sequence
(construct #12, FIG. 2C) (signal peptide boldfaced; charge mutations
boxed and italicized, numbering excluding signal peptide: 038K,
S114A, N137K, Q288E and T434E in αBb scFab, and Q520E and Q770K in
αC1s scFab)

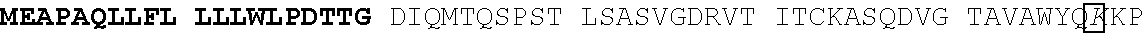

GKAPKLLIYW ASTRHTGVPD RESGSGSGTD FTLTISSLQA EDFAVYFCHQ HSSNPLTFGQ

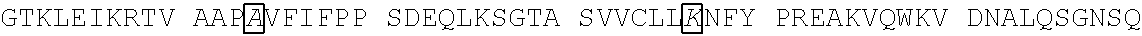

ESVTEQDSKD STYSLSSTLT LSKADYEKHK VYACEVTHQG LSSPVTKSEN RGECGGGGSG
GGGSGGGGSG GGGSGGGGSG GGGSGGGGSE VQLVESGGGL VKPGGSLRLS CAASGFTFSN

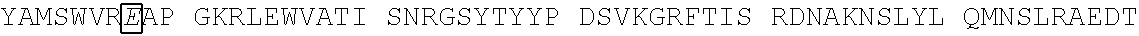

ALYYCARERP MDYWGQGTLV TVSSASTKGP SVEPLAPCSR STSESTAALG CLVKDYFPEP

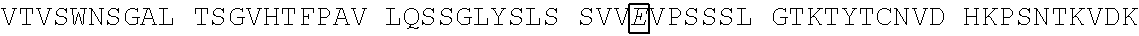

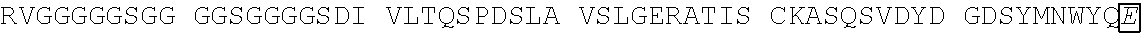

KPGQPPKILI YDASNLESGI PARFSGSGSG TDFTLTISSL EPEDFAIYYC QQSNEDPWTF
GGGTKVEIKR TVAAPSVFIF PPSDEQLKSG TASVVCLLNN FYPREAKVOW KVDNALQSGN
SQESVTEQDS KDSTYSLSST LTLSKADYEK HKVYACEVTH QGLSSPVTKS ENRGECGGGG
SGGGGSGGGG SGGGGSGGGG SGGGGSGGGG SQVOLVOSGA EVKKPGASVK LSCTASGENI

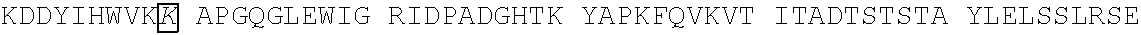

DTAVYYCARY GYGREVEDYW GQGTTVTVSS ASTKGPSVFP LAPCSRSTSE STAALGCLVK
DYFPEPVTVS WNSGALTSGV HTFPAVLOSS GLYSLSSVVT VPSSSLGTKT YTCNVDHKPS
NTKVDKRV*
SEQ ID NO: 33-[αC1S scFAB-(G₄S)₂-αBb scFV] nucleic acid sequence
(construct #13, FIG. 2D)
ATGGAAGCCC CAGCCCAGCT GCTGTTCCTG CTGCTGTTGT GGCTGCCCGA TACAACAGGC
GACATCGTGC TGACCCAGAG CCCCGACTCT CTGGCCGTGT CCCTGGGAGA AAGAGCCACA
ATCTCCTGTA AAGCCTCTCA GAGCGTGGAC TACGACGGCG ATTCTTACAT GAACTGGTAC
CAACAGAAAC CTGGACAGCC TCCTAAAATC CTGATCTACG ACGCCTCAAA CCTGGAAAGC
GGCATCCCTG CCAGATTCTC AGGCTCCGGT AGCGGCACCG ACTTCACACT GACCATCAGC
AGCCTGGAAC CTGAGGACTT CGCCATCTAC TATTGTCAGC AAAGCAACGA GGACCCTTGG
ACCTTCGGAG GCGGCACAAA GGTGGAAATC AAGCGGACCG TGGCAGCACC TTCTGTCTTC
ATCTTCCCCC CATCCGATGA GCAGCTGAAG AGCGGCACAG CTAGTGTGGT GTGCCTGCTG
AACAACTTCT ACCCAAGAGA AGCCAAGGTG CAGTGGAAGG TGGATAACGC CCTGCAGTCT
GGTAATAGCC AGGAGAGCGT GACCGAGCAG GATTCTAAGG ACAGCACATA CAGTCTGTCT
AGCACACTCA CCCTGAGCAA GGCCGACTAC GAGAAGCACA AGGTGTACGC CTGCGAGGTG
ACCCACCAGG GCCTGTCTTC TCCGGTGACC AAGTCTTTCA ACCGGGGCGA GTGCGGCGGC
GGCGGAAGCG GCGGCGGCGG CAGCGGCGGC GGGGGCAGCG GCGGCGGTGG GTCTGGCGGC
GGCGGATCAG GCGGAGGCGG CAGCGGCGGA GGCGGATCCC AAGTGCAGTT AGTTCAAAGC
GGCGCTGAGG TGAAAAAGCC TGGCGCTTCT GTGAAGCTGA GCTGCACCGC CAGCGGTTTT
AACATCAAGG ACGACTACAT CCACTGGGTG AAGCAGGCCC CTGGCCAGGG ACTGGAGTGG
ATCGGCAGAA TCGACCCCGC TGACGGCCAC ACCAAATACG CCCCTAAGTT CCAGGTGAAA
GTGACCATCA CCGCTGATAC CTCCACAAGC ACCGCCTACC TGGAACTGTC CAGCCTGAGA
AGCGAGGATA CCGCCGTCTA CTACTGTGCC AGATACGGCT ACGGCAGAGA GGTGTTCGAC
TACTGGGGAC AAGGCACCAC CGTGACAGTG TCTTCTGCTA GCACGAAAGG CCCTAGCGTG
TTTCCTCTGG CTCCATGTAG CAGAAGCACC AGCGAAAGCA CCGCCGCCCT GGGCTGCCTG
GTGAAAGACT ACTTTCCTGA GCCAGTGACC GTGTCCTGGA ACTCCGGAGC CCTCACGTCC
GGCGTGCACA CATTCCCCGC CGTGCTGCAG TCATCCGGCC TGTACAGCCT GAGCTCCGTT
GTGACCGTGC CTTCTTCCAG CCTGGGCACA AAGACCTACA CATGCAACGT GGACCACAAG
CCCAGCAATA CCAAGGTGGA CAAGAGAGTG GGCGGCGGCG GAAGCGGCGG CGGCGGCAGC
GAGGTGCAGC TGGTGGAATC TGGCGGTGGC CTTGTGAAGC CTGGAGGCAG CCTACGGCTG
AGCTGCGCCG CTAGCGGCTT CACCTTTAGC AATTACGCCA TGAGCTGGGT GCGGCAGGCT
CCTGGAAAGC GGCTGGAGTG GGTTGCAACA ATCAGCAATA GAGGCAGCTA CACCTACTAC
CCTGACTCTG TTAAGGGCAG ATTTACAATC AGCCGCGACA ACGCCAAGAA CAGCCTGTAT
CTGCAAATGA ACAGCCTGAG GGCCGAGGAC ACCGCCCTGT ACTACTGCGC CAGAGAGCGG
CCTATGGACT ATTGGGGACA GGGCACCCTG GTCACCGTCA GCAGCGGAGG GGGCGGTAGC
GGCGGTGGAG GCTCTGGCGG AGGAGGCAGC GACATACAGA TGACCCAGAG CCCTAGCACA
CTGAGCGCCT CCGTTGGCGA CCGGGTGACA ATTACCTGCA AGGCCAGCCA GGATGTGGGC
ACAGCCGTGG CCTGGTATCA GCAGAAGCCC GGCAAGGCCC CTAAGCTGCT GATCTACTGG
GCCAGCACCA GACATACAGG CGTCCCCGAC AGATTCTCTG GATCAGGCAG CGGCACCGAT
TTCACCCTGA CTATCAGCAG CCTGCAGGCC GAAGATTTCG CCGTGTACTT CTGCCACCAG
CACAGCTCTA ACCCCCTGAC CTTCGGCCAG GGCACAAAGC TTGAAATCAA GTGA
SEQ ID NO: 34-[αC1s scFab-(G₄S)₂-αBb scFv] amino acid sequence
(construct #13, FIG. 2D) (signal peptide boldfaced)
MEAPAQLLFL LLLWLPDTTG DIVLTQSPDS LAVSLGERAT ISCKASQSVD YDGDSYMNWY
QQKPGQPPKI LIYDASNLES GIPARFSGSG SGTDFTLTIS SLEPEDFAIY YCQQSNEDPW
TFGGGTKVEI KRTVAAPSVE IFPPSDEQLK SGTASVVCLL NNFYPREAKV QWKVDNALOS
GNSQESVTEQ DSKDSTYSLS STLTLSKADY EKHKVYACEV THQGLSSPVT KSENRGECGG
GGSGGGGSGG GGSGGGGSGG GGSGGGGSGG GGSQVQLVQS GAEVKKPGAS VKLSCTASGE
NIKDDYIHWV KQAPGQGLEW IGRIDPADGH TKYAPKFQVK VTITADTSTS TAYLELSSLR
SEDTAVYYCA RYGYGREVED YWGQGTTVTV SSASTKGPSV FPLAPCSRST SESTAALGCL
VKDYFPEPVT VSWNSGALTS GVHTFPAVLQ SSGLYSLSSV VTVPSSSLGT KTYTCNVDHK
PSNTKVDKRV GGGGSGGGGS EVOLVESGGG LVKPGGSLRL SCAASGFTES NYAMSWVRQA
PGKRLEWVAT ISNRGSYTYY PDSVKGRETI SRDNAKNSLY LOMNSLRAED TALYYCARER
PMDYWGQGTL VTVSSGGGGS GGGGSGGGGS DIQMTQSPST LSASVGDRVT ITCKASQDVG
TAVAWYQQKP GKAPKLLIYW ASTRHTGVPD RESGSGSGTD FTLTISSLQA EDFAVYFCHQ
HSSNPLTFGQ GTKLEIK*
SEQ ID No: 35-[αC1s scFab-(G₄S)₂-αBb scFv] nucleic acid sequence
(construct #14, FIG. 2D)
ATGGAAGCCC CCGCCCAGCT GCTGTTCCTG CTGCTCCTGT GGCTGCCTGA TACCACCGGC
GATATCGTCC TGACCCAGAG CCCTGATAGC CTGGCCGTTT CACTGGGCGA GCGGGCCACA
ATCTCCTGCA AGGCCTCTCA GTCTGTTGAC TACGACGGCG ACAGCTACAT GAACTGGTAC
CAGGAGAAAC CCGGCCAACC TCCAAAGATC CTGATCTACG ACGCCTCTAA TCTGGAGAGC
GGCATCCCCG CCCGGTTCAG CGGGTCCGGC AGCGGCACCG ACTTTACCCT GACCATCTCT
AGCCTGGAGC CTGAGGACTT CGCCATCTAC TACTGTCAGC AGAGCAACGA GGATCCTTGG
ACCTTTGGCG GCGGCACAAA GGTGGAAATC AAGCGGACCG TCGCCGCTCC ATCCGTGTTT
ATCTTCCCTC CTTCCGACGA GCAGCTCAAG AGCGGTACCG CCAGCGTGGT GTGCCTGCTG
AACAACTTCT ACCCCAGAGA GGCCAAGGTG CAGTGGAAGG TAGACAACGC CTTGCAGAGC
GGCAACTCTC AAGAGAGCGT GACAGAGCAG GACTCTAAGG ACAGCACATA CAGCCTAAGC
TCCACCCTGA CCCTCAGCAA GGCCGACTAC GAGAAGCACA AGGTGTACGC CTGTGAAGTT
ACACACCAGG GCCTGAGCAG TCCGGTGACC AAGTCCTTCA ACAGAGGCGA ATGCGGCGGA
GGAGGCTCTG GCGGCGGCGG CAGCGGCGGA GGCGGCAGCG GCGGCGGAGG CTCTGGCGGC
GGTGGCAGCG GAGGCGGCGG AAGCGGCGGA GGTGGCAGCC AGGTGCAGCT GGTGCAGAGC
GGTGCTGAAG TGAAGAAACC CGGCGCTTCC GTGAAACTGA GCTGCACCGC CAGCGGATTT
AACATCAAGG ACGACTACAT TCACTGGGTG AAAAAGGCCC CTGGCCAGGG CCTGGAATGG
ATCGGGAGAA TCGACCCCGC CGATGGCCAT ACCAAGTACG CTCCTAAGTT CCAGGTGAAA
GTGACCATCA CCGCTGATAC AAGCACCTCT ACAGCCTACC TGGAGCTGAG CTCCCTGCGG
TCTGAGGACA CCGCCGTGTA CTACTGCGCC AGATACGGCT ACGGCAGAGA GGTGTTCGAC
TACTGGGGAC AGGGCACTAC AGTCACCGTG TCTAGTGCTA GCACGAAGGG CCCTAGCGTG
TTCCCTCTGG CTCCATGTAG CAGAAGCACC AGCGAAAGCA CAGCTGCTCT GGGCTGCCTG
GTGAAAGACT ACTTCCCCGA GCCTGTGACC GTCAGCTGGA ACTCCGGCGC CCTGACCAGC
GGAGTGCACA CCTTTCCTGC TGTGCTGCAA TCCTCTGGCC TGTACTCTCT GAGCTCTGTT
GTGACAGTGC CTTCTAGCAG CCTGGGAACC AAGACCTACA CCTGCAACGT GGACCACAAG
CCCAGCAACA CCAAGGTGGA TAAGCGCGTG GGCGGCGGCG GATCTGGCGG AGGCGGCAGC
GAGGTGCAGC TGGTGGAAAG CGGCGGCGGC CTGGTGAAGC CTGGCGGCTC ACTGAGACTG
AGCTGTGCCG CCAGCGGCTT CACCTTCTCC AACTACGCCA TGAGCTGGGT GCGGGAAGCC
CCAGGAAAGC GCCTGGAGTG GGTCGCCACC ATCAGCAATA GAGGCTCGTA TACATATTAC
CCTGATTCCG TCAAAGGCAG ATTCACCATC TCTAGAGATA ATGCCAAGAA CAGCCTGTAC
CTGCAGATGA ACTCCCTCAG AGCCGAGGAT ACAGCCCTGT ATTACTGCGC CAGAGAACGG
CCTATGGACT ACTGGGGCCA AGGCACTCTG GTGACAGTGA GCAGCGGCGG CGGTGGTTCC
GGCGGCGGAG GCTCTGGAGG AGGCGGCAGC GACATCCAGA TGACCCAGAG CCCTAGCACC
CTGTCCGCCA GCGTGGGAGA TAGAGTGACC ATTACCTGTA AAGCGAGCCA GGATGTGGGC
ACCGCCGTGG CCTGGTATCA GAAGAAGCCT GGCAAGGCCC CTAAGCTGCT GATCTACTGG
GCCTCTACCC GGCACACAGG CGTGCCCGAC AGATTCTCCG GCTCCGGTTC TGGAACAGAC
TTCACACTGA CCATCAGCTC TCTTCAGGCC GAGGACTTCG CCGTGTACTT CTGCCACCAG
CACAGCTCTA ATCCTCTGAC ATTCGGCCAA GGCACAAAGC TGGAAATCAA GTGA
SEQ ID No: 36-[αC1s scFab-(G₄S)₂-αBb scFv-CM] amino acid
sequence (construct #14, Fig. 2D) (signal peptide boldfaced; charge
mutations boxed and italicized, numbering excluding signal peptide:
Q42E and Q292K in αC1s scFab, and Q519E and Q648K in αBb scFv)
MEAPAQLLFL LLLWLPDTTG DIVLTQSPDS LAVSLGERAT ISCKASQSVD YDGDSYMNWY

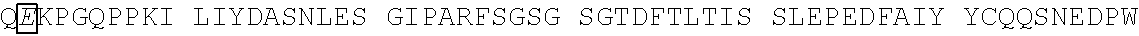

TFGGGTKVEI KRTVAAPSVF IFPPSDEQLK SGTASVVCLL NNFYPREAKV QWKVDNALQS
GNSQESVTEQ DSKDSTYSLS STLTLSKADY EKHKVYACEV THQGLSSPVT KSFNRGECGG
GGSGGGGSGG GGSGGGGSGG GGSGGGGSGG GGSQVQLVQS GAEVKKPGAS VKLSCTASGF

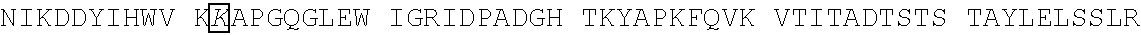

SEDTAVYYCA RYGYGREVFD YWGQGTTVTV SSASTKGPSV FPLAPCSRST SESTAALGCL
VKDYFPEPVT VSWNSGALTS GVHTFPAVLQ SSGLYSLSSV VTVPSSSLGT KTYTCNVDHK

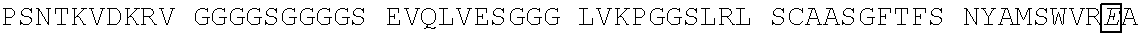

PGKRLEWVAT ISNRGSYTYY PDSVKGRFTI SRDNAKNSLY LQMNSLRAED TALYYCARER
PMDYWGQGTL VTVSSGGGGS GGGGSGGGGS DIQMTQSPST LSASVGDRVT ITCKASQDVG

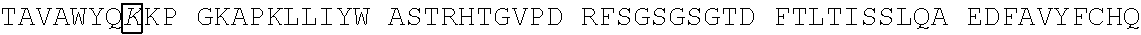

HSSNPLTFGQ GTKLEIK*
SEQ ID NO: 37-[αC1s scFab-bidirectional promoter-αBb scFab]
nucleic acid sequence (construct #9, FIG. 2B)
TCACACCCGC TTATCCACCT TGGTGTTGCT GGGCTTGTGG TCCACGTTGC AGGTGTAGGT
CTTTGTGCCC AGGCTAGAGC TAGGCACTGT CACGACAGAG GACAGAGAGT ACAGGCCGCT
GCTCTGCAGC ACGGCGGGGA AGGTGTGCAC CCCGCTTGTC AGGGCTCCGC TGTTCCAGGA
CACGGTCACA GGCTCAGGGA AATAATCCTT GACCAGGCAG CCCAGAGCAG CCGTGCTCTC
TGAGGTACTT CTGCTACAAG GAGCCAGTGG GAACACGCTA GGGCCCTTTG TGCTGGCGGA
CGACACGGTC ACTGTTGTGC CCTGTCCCCA GTAGTCGAAC ACTTCTCTGC CGTAGCCGTA
TCTGGCGCAG TAGTACACAG CGGTGTCCTC GGATCTAAGG CTGCTCAGTT CCAGATAAGC
TGTAGAGGTG CTGGTATCGG CGGTGATGGT GACTTTCACC TGGAACTTAG GGGCGTACTT
TGTGTGGCCG TCGGCAGGGT CGATTCTGCC GATCCACTCC AGTCCCTGGC CGGGGGCCTG
CTTCACCCAG TGGATGTAAT CGTCCTTGAT ATTGAAGCCG CTGGCGGTGC AGCTCAGCTT
AACACTAGCG CCAGGCTTTT TCACCTCGGC TCCGCTCTGC ACCAGCTGCA CCTGGGATCC
GCCGCCGCCG CTGCCGCCTC CGCCGCTGCC GCCTCCGCCG CTTCCGCCTC CCCCAGAGCC
GCCGCCACCG CTGCCTCCTC CGCCGGAGCC GCCGCCGCCG CACTCGCCCC GGTTGAAGCT
TTTGGTCACA GGAGAGGACA GGCCCTGATG TGTCACTTCA CAGGCGTACA CCTTGTGCTT
CTCGTAGTCG GCCTTGCTCA AGGTCAGGGT GCTGGACAGG CTGTATGTTG AGTCCTTGCT
GTCCTGCTCG GTCACGCTCT CTTGGCTGTT GCCGCTTTGC AGGGCGTTGT CAACTTTCCA
TTGGACCTTT GCCTCTCTGG GGTAGAAGTT ATTCAGCAGG CACACCACAG AGGCGGTTCC
GCTCTTCAGC TGCTCGTCGC TTGGAGGGAA GATAAAGACA GAAGGGGCGG CCACGGTGCG
CTTGATTTCC ACCTTGGTGC CGCCTCCAAA GGTCCAGGGG TCCTCGTTGC TCTGCTGGCA
GTAGTAGATG GCAAAATCCT CGGGTTCCAG AGAAGAAATT GTCAGGGTGA AATCAGTGCC
AGAGCCGCTG CCGCTGAATC TGGCGGGGAT GCCGCTTTCC AGATTGCTGG CGTCGTAGAT
CAGGATTTTT GGAGGCTGGC CGGGTTTCTG CTGGTACCAG TTCATGTAGC TGTCGCCGTC
ATAGTCCACG CTCTGAGAGG CTTTACAGCT GATTGTGGCC CGTTCGCCGA GGCTCACGGC
CAGGCTATCA GGGCTCTGCG TCAGCACGAT ATCGCCGGTG GTGTCAGGCA GCCACAGGAG
CAGCAGGAAC AGCAGCTGGG CAGGGGCTTC CATGGTGGGC TCTGGCGCCC GCCGCGCGCT
TCGCTTTTTA TAGGGCCGCC GCCGCCGCCG CCTCGCCATA AAAGGAAACT TTCGGAGCGC
GCCGCTCTGA TTGGCTGCCG CCGCACCTCT CCGCCTCGCC CCGCCCCGCC CCTCGCCCCG
CCCCGCCCCG CCTGGCGCGC GCCCCCCCCC CCCCCCCGCC CCCATCGCTG CACAAAATAA
TTAAAAAATA AATAAATACA AAATTGGGGG TGGGGAGGGG GGGGAGATGG GGAGAGTGAA
GCAGAACGTG GGGCTCACCT CGCTAGTTAT TAATAGTAAT CAATTACGGG GTCATTAGTT
CATAGCCCAT ATATGGAGTT CCGCGTTACA TAACTTACGG TAAATGGCCC GCCTGGCTGA
CCGCCCAACG ACCCCCGCCC ATTGACGTCA ATAATGACGT ATGTTCCCAT AGTAACGCCA
ATAGGGACTT TCCATTGACG TCAATGGGTG GAGTATTTAC GGTAAACTGC CCACTTGGCA
GTACATCAAG TGTATCATAT GCCAAGTACG CCCCCTATTG ACGTCAATGA CGGTAAATGG
CCCGCCTGGC ATTATGCCCA GTACATGACC TTATGGGACT TTCCTACTTG GCAGTACATC
TACGTATTAG TCATCGCTAT TACCATGGTC GAGGTGAGCC CCACGTTCTG CTTCACTCTC
CCCATCTCCC CCCCCTCCCC ACCCCCAATT TTGTATTTAT TTATTTTTTA ATTATTTTGT
GCAGCGATGG GGGCGGGGGG GGGGGGGGGG CGCGCGCCAG GCGGGGGGGG GCGGGGCGAG
GGGCGGGGCG GGGCGAGGCG GAGAGGTGCG GCGGCAGCCA ATCAGAGCGG CGCGCTCCGA
AAGTTTCCTT TTATGGCGAG GCGGCGGCGG CGGCGGCCCT ATAAAAAGCG AAGCGCGCGG
CGGGCGCCAA CTAGCCCACC ATGGAAGCCC CCGCTCAGCT GCTGTTCCTG CTGCTGCTGT
GGCTGCCTGA CACCACCGGC GACATCCAGA TGACACAGAG CCCTAGCACC CTGAGCGCCT
CCGTGGGGGA CAGAGTGACA ATCACATGTA AAGCCTCCCA GGACGTGGGC ACTGCCGTGG
CCTGGTACCA GCAAAAACCG GGAAAAGCCC CTAAGCTGCT GATCTACTGG GCCAGCACCA
GACACACCGG CGTCCCCGAT AGATTCAGCG GCTCTGGCAG CGGAACTGAT TTCACCCTGA
CCATTTCTTC TCTGCAGGCC GAGGACTTCG CCGTGTACTT TTGCCACCAG CACAGCAGCA
ACCCTCTGAC CTTCGGACAG GGCACAAAGC TGGAAATCAA GCGGACAGTG GCTGCTCCTT
CTGTGTTCAT CTTTCCACCT AGCGACGAGC AGCTGAAGAG CGGCACCGCC TCTGTGGTGT
GCCTGCTGAA CAACTTCTAC CCCAGAGAAG CCAAAGTGCA GTGGAAGGTG GACAACGCCC
TGCAATCTGG CAACAGCCAG GAGAGCGTGA CGGAACAAGA TAGCAAGGAC AGCACCTACT
CCCTGAGCAG CACACTGACC TTGTCCAAGG CAGATTACGA GAAGCACAAG GTGTACGCCT
GCGAGGTGAC CCACCAGGGA CTGAGCAGCC CAGTGACCAA GAGCTTCAAC AGAGGAGAGT
GCGGCGGCGG CGGAAGCGGA GGCGGAGGCA GCGGCGGCGG CGGCAGTGGA GGCGGCGGCT
CTGGCGGAGG GGGCAGTGGC GGTGGCGGAT CCGGCGGCGG CGGCAGCGAG GTGCAGCTTG
TGGAATCCGG CGGCGGCCTG GTGAAGCCCG GCGGTAGCCT GAGACTGTCT TGTGCCGCCT
CTGGCTTCAC CTTTAGCAAT TACGCCATGA GCTGGGTGCG GCAGGCTCCC GGCAAAAGAC
TGGAATGGGT CGCCACCATC AGCAACCGGG GATCATATAC CTACTACCCT GATAGCGTGA
AAGGCAGGTT CACAATCAGC CGGGACAATG CCAAGAACAG CCTGTACCTG CAGATGAACT
CACTGCGGGC CGAGGACACC GCCCTGTATT ACTGCGCCAG AGAGAGACCT ATGGACTACT
GGGGCCAGGG CACCCTGGTG ACCGTTTCCT CCGCCAGCAC CAAGGGCCCT AGCGTGTTCC
CTCTGGCCCC ATGCAGCAGA AGCACATCTG AGAGCACCGC CGCTCTGGGC TGCCTGGTGA
AGGACTACTT CCCCGAGCCT GTGACAGTGA GCTGGAACTC CGGCGCCCTG ACCAGCGGCG
TGCACACATT TCCAGCTGTG CTGCAGTCTA GCGGCCTGTA CAGCCTGAGC AGCGTTGTGA
CAGTGCCTTC TAGCAGCCTC GGCACCAAGA CCTACACCTG TAACGTGGAT CATAAGCCTT
CTAATACCAA GGTTGACAAG AGAGTGTGA
SEQ ID NO: 38-[αC1s F2A Fab-(G₄S)₃ αBb scFab] nucleic acid
sequence (construct #17, FIG. 2F)
ATGGAAGCCC CAGCTCAGCT GCTGTTCCTG CTGCTGCTGT GGCTGCCCGA CACCACCGGC
GACATCGTGC TGACCCAGAG CCCTGATAGC CTGGCCGTTT CTCTGGGAGA ACGGGCAACC
ATTAGCTGCA AGGCCAGCCA GTCTGTGGAC TACGACGGCG ACAGCTACAT GAATTGGTAT
CAGCAGAAGC CTGGCCAACC TCCCAAGATC CTGATCTACG ATGCCAGCAA CCTGGAATCC
GGAATCCCCG CCCGCTTCAG CGGCAGCGGC TCAGGCACCG ACTTCACCCT GACAATCTCC
TCGCTGGAAC CCGAGGATTT CGCTATCTAC TACTGTCAGC AGTCTAACGA GGATCCTTGG
ACCTTCGGCG GCGGCACAAA GGTCGAGATC AAGAGAACAG TTGCCGCCCC TTCTGTGTTT
ATCTTCCCTC CCTCTGACGA GCAGCTGAAG AGCGGCACTG CCAGCGTCGT GTGCCTGCTG
AACAACTTCT ACCCACGTGA GGCCAAAGTC CAATGGAAAG TGGATAACGC CCTGCAGAGC
GGCAACTCTC AGGAGTCTGT GACAGAGCAG GACAGCAAAG ATAGCACCTA CTCTCTGTCT
AGCACCCTGA CCCTGAGCAA GGCCGATTAC GAGAAGCACA AAGTGTACGC CTGCGAGGTG
ACCCACCAGG GCCTGAGCAG CCCCGTGACA AAGTCCTTCA ACAGGGGCGA GTGTCGGAAG
AGACGGAGCG GCAGCGGCGC CCCAGTCAAG CAGACCCTGA ACTTCGACCT GCTTAAGCTG
GCCGGCGATG TAGAAAGCAA TCCTGGCCCC ATGGAAGCCC CTGCCCAGCT GCTGTTCCTG
CTGCTGCTGT GGCTGCCTGA CACCACAGGA CAAGTGCAAC TAGTGCAGTC AGGCGCCGAG
GTAAAAAAGC CTGGCGCCAG CGTGAAACTG TCTTGCACCG CCTCCGGCTT CAATATCAAG
GACGACTACA TACACTGGGT GAAGCAGGCT CCCGGCCAGG GCCTGGAATG GATCGGCCGC
ATCGACCCTG CTGACGGCCA CACCAAGTAT GCCCCTAAGT TCCAGGTCAA AGTGACCATC
ACCGCTGATA CCAGCACAAG TACAGCCTAC CTGGAACTGA GCAGCCTGCG GAGCGAGGAC
ACAGCCGTGT ACTACTGCGC CCGGTACGGC TATGGCAGAG AGGTGTTCGA CTACTGGGGA
CAGGGCACCA CCGTGACAGT GTCTAGCGCC TCTACAAAGG GCCCTAGCGT GTTCCCGCTG
GCCCCCTGCA GCAGAAGCAC ATCTGAAAGC ACAGCAGCTC TCGGCTGCCT CGTCAAGGAC
TACTTTCCTG AGCCGGTGAC AGTTAGCTGG AACAGCGGCG CCCTGACTAG CGGCGTGCAT
ACATTCCCTG CCGTGCTGCA GTCCTCCGGC CTCTACAGCC TGTCCAGCGT GGTGACAGTC
CCTTCTTCCA GTCTGGGTAC GAAAACCTAC ACCTGCAACG TGGACCACAA GCCCTCCAAT
ACGAAAGTGG ACAAGAGAGT GGGCGGGGGA GGCTCTGGCG GAGGTGGCTC TGGCGGGGGC
GGAAGCGACA TCCAGATGAC ACAATCCCCT AGCACCCTGA GCGCCAGCGT GGGAGATAGA
GTGACGATCA CCTGTAAAGC CTCACAGGAC GTGGGCACCG CCGTGGCCTG GTACCAGCAG
AAACCTGGAA AGGCCCCTAA GCTGCTGATC TACTGGGCCT CCACCAGACA CACCGGCGTG
CCTGACAGAT TCAGCGGCTC TGGCAGCGGC ACAGACTTTA CCCTGACAAT CAGCAGCCTG
CAGGCTGAAG ATTTCGCCGT GTACTTCTGC CACCAACACA GCAGCAACCC CCTGACATTT
GGCCAAGGCA CCAAGCTGGA GATCAAGAGA ACCGTTGCTG CCCCTAGCGT GTTCATCTTC
CCGCCTAGCG ACGAGCAGCT GAAGAGCGGC ACCGCCTCTG TGGTTTGCCT GCTGAACAAC
TTCTACCCCA GAGAAGCCAA AGTGCAGTGG AAGGTGGACA ACGCCCTGCA GAGTGGAAAC
TCTCAAGAGA GCGTGACCGA ACAGGATAGC AAAGACAGCA CCTATAGCTT GTCTAGCACA
CTGACCCTGT CTAAGGCTGA CTACGAGAAG CACAAGGTGT ACGCATGCGA GGTCACCCAT
CAGGGACTGA GCAGCCCCGT GACCAAGTCT TTTAACCGGG GCGAGTGCGG CGGAGGAGGC
AGTGGCGGCG GGGGATCCGG CGGCGGCGGC AGCGGCGGAG GCGGATCCGG CGGCGGCGGT
AGCGGCGGTG GCGGCAGCGG TGGAGGGGGA AGCGAGGTGC AGCTCGTCGA GTCCGGAGGA
GGCCTTGTGA AGCCTGGCGG CAGCCTGAGA CTGAGCTGCG CCGCCAGCGG ATTCACCTTC
AGCAATTACG CCATGAGCTG GGTGCGGCAG GCCCCTGGCA AGAGACTGGA ATGGGTGGCC
ACCATCAGCA ACAGAGGCAG CTACACCTAC TACCCCGACT CCGTGAAGGG CAGATTTACC
ATCAGCCGGG ACAACGCCAA GAACAGCCTG TACCTGCAGA TGAACTCCCT GAGAGCCGAG
GACACCGCCC TGTACTACTG TGCCAGGGAA AGACCTATGG ACTACTGGGG CCAGGGAACA
CTGGTGACCG TATCTTCCGC CTCAACCAAA GGCCCCTCGG TGTTTCCACT GGCTCCTTGC
TCCAGATCCA CCTCCGAGAG CACCGCCGCC CTGGGCTGTC TGGTGAAGGA TTACTTCCCA
GAACCTGTGA CCGTGAGCTG GAATAGCGGC GCTCTCACCT CTGGAGTGCA CACCTTCCCT
GCCGTGCTGC AGAGCAGCGG CCTGTATAGC TTGTCCAGTG TGGTGACCGT GCCTAGCTCC
AGCCTGGGCA CTAAGACATA TACATGTAAC GTGGACCACA AGCCTAGCAA CACCAAGGTG
GATAAGAGAG TGTGA
SEQ ID NO: 39-[αC1s F2A Fab-(G₄S)₃-αBb scFab] amino acid
sequence (construct #17, FIG. 2F) (signal peptides boldfaced; furin
cleavage site underlined; F2A sequence italicized)
MEAPAQLLFL LLLWLPDTTG DIVLTOSPDS LAVSLGERAT ISCKASQSVD YDGDSYMNWY
QQKPGOPPKI LIYDASNLES GIPARESGSG SGTDETLTIS SLEPEDFAIY YCQQSNEDPW
TFGGGTKVEI KRTVAAPSVE IFPPSDEQLK SGTASVVCLL NNFYPREAKV QWKVDNALQS
GNSQESVTEQ DSKDSTYSLS STLTLSKADY EKHKVYACEV THQGLSSPVT KSENRGECRK
RRSGSGAPVK QTLNEDLLKL AGDVESNPGP MEAPAQLLFL LLLWLPDTTG QVOLVOSGAE
VKKPGASVKL SCTASGENIK DDYIHWVKQA PGQGLEWIGR IDPADGHTKY APKFQVKVTI
TADTSTSTAY LELSSLRSED TAVYYCARYG YGREVEDYWG QGTTVTVSSA STKGPSVEPL
APCSRSTSES TAALGCLVKD YFPEPVTVSW NSGALTSGVH TFPAVLQSSG LYSLSSVVTV
PSSSLGTKTY TCNVDHKPSN TKVDKRVGGG GSGGGGSGGG GSDIQMTQSP STLSASVGDR
VTITCKASQD VGTAVAWYQQ KPGKAPKLLI YWASTRHTGV PDRFSGSGSG TDFTLTISSL
QAEDFAVYFC HQHSSNPLTF GQGTKLEIKR TVAAPSVFIF PPSDEQLKSG TASVVCLLNN
FYPREAKVOW KVDNALQSGN SQESVTEQDS KDSTYSLSST LTLSKADYEK HKVYACEVTH
QGLSSPVTKS FNRGECGGGG SGGGGSGGGG SGGGGSGGGG SGGGGSGGGG SEVOLVESGG
GLVKPGGSLR LSCAASGFTF SNYAMSWVRQ APGKRLEWVA TISNRGSYTY YPDSVKGRFT
ISRDNAKNSL YLQMNSLRAE DTALYYCARE RPMDYWGQGT LVTVSSASTK GPSVFPLAPC
SRSTSESTAA LGCLVKDYFP EPVTVSWNSG ALTSGVHTEP AVLOSSGLYS LSSVVTVPSS
SLGTKTYTCN VDHKPSNTKV DKRV*
SEQ ID NO: 40-[αC1s GT2A Fab-(G₄S)₃-αBb scFab] nucleic acid
sequence (construct #18, FIG. 2F)
ATGGAAGCCC CAGCCCAGCT GCTGTTTCTG CTGCTGTTGT GGCTGCCCGA TACTACCGGC
GATATCGTGC TGACCCAGAG CCCTGATAGC CTGGCTGTGT CTCTGGGGGA GCGGGCTACC
ATCTCTTGTA AAGCCAGCCA AAGCGTGGAC TACGACGGCG ACTCCTACAT GAACTGGTAC
CAGCAGAAAC CTGGCCAGCC TCCAAAGATC CTGATCTACG ACGCCAGCAA CCTGGAAAGC
GGCATCCCTG CTCGGTTCAG CGGATCAGGC TCGGGCACAG ACTTTACACT GACAATTAGC
TCTCTGGAAC CTGAAGATTT TGCTATCTAC TATTGCCAGC AGAGCAACGA GGATCCTTGG
ACCTTTGGCG GCGGAACAAA GGTGGAAATC AAGCGGACAG TCGCTGCCCC TAGTGTGTTC
ATCTTCCCAC CTTCCGATGA GCAGCTCAAG TCTGGAACAG CCTCTGTGGT CTGCCTGCTG
AACAACTTCT ACCCCCGGGA GGCTAAAGTG CAGTGGAAGG TGGATAACGC CCTGCAGTCT
GGCAACTCGC AGGAGAGCGT TACAGAGCAG GACTCTAAGG ACAGTACCTA CAGCCTGTCA
TCAACCCTGA CCCTGAGCAA GGCCGACTAT GAAAAGCACA AGGTCTACGC CTGCGAGGTG
ACACACCAGG GCCTGAGCTC TCCTGTGACT AAGTCCTTCA ATAGAGGAGA GTGCAGACGG
AAGCGCGGCA GCGGAGAAGG CAGAGGCTCC CTGCTAACCT GTGGAGACGT GGAGGAAAAC
CCCGGCCCCA TGGAAGCCCC TGCTCAGCTG CTGTTCCTGC TGCTGCTGTG GCTGCCGGAT
ACAACCGGAC AAGTGCAGCT GGTGCAATCT GGCGCCGAAG TGAAAAAGCC CGGCGCTTCT
GTGAAGCTGT CTTGCACCGC CTCTGGATTC AACATCAAGG ACGACTACAT CCACTGGGTG
AAGCAGGCCC CTGGCCAGGG CCTGGAGTGG ATCGGCAGAA TCGACCCCGC TGATGGCCAC
ACAAAATACG CCCCTAAGTT CCAGGTGAAG GTGACCATCA CCGCTGACAC CTCGACAAGT
ACCGCCTACC TGGAGCTGAG CTCTCTGAGA TCCGAGGACA CAGCAGTGTA CTACTGCGCC
AGATACGGCT ACGGCAGAGA GGTTTTCGAC TACTGGGGCC AGGGCACCAC CGTGACCGTG
TCCAGCGCCA GCACAAAGGG CCCTTCTGTC TTCCCTCTGG CGCCTTGTAG CCGGAGCACA
AGCGAGAGCA CTGCCGCTCT TGGCTGCCTG GTGAAGGACT ACTTTCCTGA ACCTGTTACA
GTGAGCTGGA ACAGCGGCGC CCTGACATCT GGCGTGCACA CCTTTCCAGC CGTGCTGCAG
TCCTCCGGCC TGTACAGTCT GAGCAGCGTG GTGACCGTGC CTAGCAGCTC TCTGGGCACC
AAGACATATA CCTGCAATGT GGACCACAAA CCTAGCAACA CCAAGGTGGA CAAGAGAGTG
GGCGGCGGCG GGAGTGGAGG TGGAGGCAGC GGAGGTGGTG GCAGCGACAT CCAGATGACA
CAGAGCCCTA GCACTCTGAG CGCCAGCGTG GGCGATAGAG TGACCATTAC CTGCAAGGCC
TCCCAGGACG TGGGAACCGC CGTGGCCTGG TATCAGCAAA AGCCAGGCAA GGCCCCCAAG
CTTCTGATCT ACTGGGCCAG CACAAGACAC ACCGGCGTCC CCGACAGGTT CAGCGGCAGT
GGCTCAGGCA CCGACTTCAC CCTAACTATC AGCTCTCTGC AAGCTGAAGA CTTCGCCGTG
TACTTCTGCC ACCAGCACAG CTCCAACCCC TTGACCTTCG GCCAAGGCAC AAAGCTGGAA
ATCAAACGGA CAGTCGCCGC ACCTAGCGTG TTCATCTTCC CACCTTCTGA CGAGCAGCTG
AAGAGCGGCA CCGCGTCCGT GGTGTGTCTG CTCAACAACT TCTACCCAAG AGAGGCCAAG
GTGCAGTGGA AGGTTGACAA TGCCCTGCAG AGCGGGAATA GCCAGGAGAG CGTGACCGAG
CAGGACAGCA AGGACTCTAC CTACAGCCTC AGTTCTACCC TGACCCTGTC CAAGGCCGAT
TACGAGAAGC ACAAGGTGTA CGCCTGTGAA GTGACCCATC AGGGCCTGAG CAGTCCTGTG
ACTAAAAGCT TCAACAGAGG CGAATGCGGC GGCGGAGGCT CCGGCGGAGG CGGCAGCGGC
GGAGGCGGAT CTGGCGGCGG TGGCTCCGGA GGCGGCGGCA GCGGCGGCGG CGGCTCTGGC
GGCGGCGGCT CTGAGGTGCA ACTGGTTGAA AGCGGAGGCG GCCTGGTGAA GCCCGGAGGC
TCCCTGCGGC TGAGCTGCGC CGCCAGTGGC TTCACCTTCT CTAATTACGC TATGAGCTGG
GTCAGACAGG CCCCTGGAAA GCGGTTGGAG TGGGTGGCCA CCATCAGCAA CCGGGGAAGC
TACACCTACT ACCCAGATAG CGTGAAAGGC AGGTTTACCA TCAGCAGAGA TAACGCCAAG
AACTCACTGT ACCTGCAGAT GAACAGCCTG AGAGCCGAGG ACACCGCCCT GTACTACTGC
GCCAGAGAGA GACCTATGGA CTACTGGGGC CAAGGCACAT TAGTCACCGT GTCCTCTGCC
AGTACCAAGG GCCCTAGCGT GTTCCCTCTG GCCCCTTGCT CCAGAAGCAC CAGCGAGAGC
ACAGCCGCAC TTGGATGTCT GGTTAAAGAT TATTTCCCCG AGCCCGTGAC AGTGTCTTGG
AACAGCGGGG CCCTGACCAG CGGTGTTCAT ACCTTCCCTG CTGTGCTCCA GAGCTCCGGC
CTGTATTCCC TGAGTTCAGT AGTGACCGTG CCTAGCAGCA GCCTGGGAAC CAAGACCTAC
ACATGCAACG TGGACCACAA GCCTAGCAAT ACCAAGGTGG ACAAGCGGGT GTGA
SEQ ID NO: 41 [αC1s GT2A Fab-(G₄S)₃-αBb scFab] amino acid sequence
construct #18, FIG. 2F) (signal peptides boldfaced; furin cleavage
site underlined; GT2A sequence italicized)
MEAPAQLLFL LLLWLPDTTG DIVLTQSPDS LAVSLGERAT ISCKASQSVD YDGDSYMNWY
QQKPGQPPKI LIYDASNLES GIPARFSGSG SGTDFTLTIS SLEPEDFAIY YCQQSNEDPW
TFGGGTKVEI KRTVAAPSVF IFPPSDEQLK SGTASVVCLL NNFYPREAKV QWKVDNALQS
GNSQESVTEQ DSKDSTYSLS STLTLSKADY EKHKVYACEV THQGLSSPVT KSFNRGECRR
KRGSGEGRGS LLTCGDVEEN PGPMEAPAQL LFLLLLWLPD TTGQVQLVQS GAEVKKPGAS
VKLSCTASGF NIKDDYIHWV KQAPGQGLEW IGRIDPADGH TKYAPKFQVK VTITADTSTS
TAYLELSSLR SEDTAVYYCA RYGYGREVFD YWGQGTTVTV SSASTKGPSV FPLAPCSRST
SESTAALGCL VKDYFPEPVT VSWNSGALTS GVHTFPAVLQ SSGLYSLSSV VTVPSSSLGT
KTYTCNVDHK PSNTKVDKRV GGGGSGGGGS GGGGSDIQMT QSPSTLSASV GDRVTITCKA
SQDVGTAVAW YQQKPGKAPK LLIYWASTRH TGVPDRFSGS GSGTDFTLTI SSLQAEDFAV
YFCHQHSSNP LTFGQGTKLE IKRTVAAPSV FIFPPSDEQL KSGTASVVCL LNNFYPREAK
VQWKVDNALQ SGNSQESVTE QDSKDSTYSL SSTLTLSKAD YEKHKVYACE VTHQGLSSPV
TKSFNRGECG GGGSGGGGSG GGGSGGGGSG GGGSGGGGSG GGGSEVQLVE SGGGLVKPGG
SLRLSCAASG FTFSNYAMSW VRQAPGKRLE WVATISNRGS YTYYPDSVKG RFTISRDNAK
NSLYLQMNSL RAEDTALYYC ARERPMDYWG QGTLVTVSSA STKGPSVFPL APCSRSTSES
TAALGCLVKD YFPEPVTVSW NSGALTSGVH TFPAVLQSSG LYSLSSVVTV PSSSLGTKTY
TCNVDHKPSN TKVDKRV*
SEQ ID NO: 42-[αC1s scFab-(G₄S)₃-αBb F2A Fab] nucleic acid
sequence (construct #19, FIG. 2F)
ATGGAAGCCC CAGCCCAGCT GCTGTTCCTG CTGCTGCTGT GGCTGCCCGA TACCACCGGC
GACATCGTGC TGACACAGAG TCCTGATAGC CTGGCCGTGT CTCTGGGGGA AAGAGCCACA
ATCTCTTGCA AGGCCTCCCA GAGTGTAGAC TACGACGGCG ATAGTTACAT GAACTGGTAT
CAGCAGAAAC CTGGACAACC TCCAAAGATC CTGATCTACG ACGCCAGCAA CCTGGAGAGC
GGCATTCCTG CCCGGTTCAG CGGCAGCGGC AGCGGCACCG ACTTCACCCT GACAATCAGC
AGCCTGGAGC CCGAGGACTT TGCCATCTAC TACTGTCAGC AAAGCAACGA GGACCCCTGG
ACATTTGGCG GCGGCACGAA AGTGGAAATC AAGCGGACCG TCGCCGCCCC CAGCGTGTTC
ATCTTCCCTC CTTCTGATGA GCAGCTCAAG AGCGGCACAG CCAGCGTGGT GTGCCTGCTG
AACAATTTCT ACCCTAGGGA AGCCAAGGTG CAGTGGAAGG TGGACAATGC CCTGCAAAGC
GGCAACTCTC AGGAGTCCGT TACCGAGCAA GATAGCAAGG ACTCTACATA TTCTCTGTCT
AGCACCCTGA CCTTGAGCAA GGCCGACTAT GAAAAGCACA AGGTCTACGC ATGCGAGGTG
ACTCATCAGG GCCTCAGCTC CCCAGTGACC AAATCCTTCA ACCGGGGCGA GTGCGGCGGA
GGCGGCAGCG GGGGCGGAGG CAGCGGAGGA GGCGGCTCAG GCGGAGGAGG CAGCGGCGGC
GGCGGCTCGG GCGGAGGCGG AAGCGGCGGC GGCGGCAGCC AAGTGCAGCT GGTGCAGAGC
GGCGCTGAAG TGAAAAAGCC TGGCGCCAGC GTGAAGCTGT CCTGCACCGC CAGCGGCTTC
AATATCAAGG ATGATTACAT CCACTGGGTG AAACAGGCCC CTGGCCAGGG CCTTGAGTGG
ATCGGAAGGA TCGACCCTGC CGATGGCCAC ACCAAGTACG CTCCCAAGTT CCAGGTGAAG
GTGACCATCA CCGCCGATAC CAGCACGAGC ACAGCCTACC TGGAACTGTC TTCCCTGAGA
AGCGAAGATA CCGCCGTGTA CTACTGCGCC AGATACGGAT ATGGCAGAGA GGTATTCGAC
TACTGGGGAC AGGGCACCAC CGTGACCGTG TCCTCTGCCT CCACCAAGGG CCCCTCTGTG
TTTCCTCTGG CCCCCTGCTC TAGAAGCACC AGCGAGAGCA CAGCCGCCCT GGGCTGTCTG
GTGAAAGACT ATTTCCCTGA GCCCGTGACC GTGTCCTGGA ACAGCGGCGC CCTGACAAGT
GGCGTGCACA CCTTTCCTGC TGTTCTGCAG AGTAGCGGCC TGTACAGCCT GTCGAGCGTG
GTCACAGTGC CTAGCAGCAG TCTGGGCACA AAGACCTACA CTTGTAACGT GGATCACAAG
CCCTCTAATA CCAAGGTGGA CAAGCGGGTG GGAGGCGGCG GAAGCGGAGG CGGCGGCTCT
GGGGGAGGTG GCTCAGAAGT GCAGCTGGTG GAAAGCGGCG GCGGGCTGGT GAAGCCTGGC
GGCTCTCTCC GGCTGAGCTG TGCCGCCAGC GGTTTTACCT TCTCCAATTA CGCCATGAGC
TGGGTCAGAC AGGCCCCAGG CAAGAGACTT GAGTGGGTTG CTACAATCAG CAACAGAGGC
AGCTACACCT ACTACCCTGA CAGCGTGAAG GGCAGATTCA CAATCAGCCG GGACAACGCC
AAGAACAGCC TGTACCTGCA GATGAACAGC CTGAGAGCCG AGGATACAGC CCTTTACTAC
TGTGCCAGAG AGAGACCTAT GGACTACTGG GGCCAGGGCA CTCTGGTGAC CGTTTCCAGC
GCCAGCACCA AAGGCCCAAG CGTGTTCCCT CTGGCTCCCT GCAGCAGAAG CACCAGCGAA
AGCACAGCTG CGCTGGGCTG CCTGGTGAAG GATTACTTCC CCGAGCCTGT GACCGTGTCT
TGGAACTCCG GCGCTCTGAC ATCCGGCGTT CACACATTCC CCGCTGTCCT GCAGTCAAGT
GGCCTGTACA GCCTGAGCAG TGTGGTGACC GTTCCAAGCT CTTCTCTGGG AACAAAAACA
TACACCTGCA ACGTGGACCA CAAGCCTAGC AACACCAAAG TGGATAAGCG GGTGCGGAAG
CGCCGGAGCG GAAGCGGCGC CCCTGTGAAG CAGACCCTGA ACTTCGACCT GCTGAAGCTG
GCTGGCGACG TGGAAAGCAA CCCTGGCCCT ATGGAAGCCC CCGCACAACT GCTGTTCCTG
CTGCTGCTCT GGCTGCCTGA CACCACAGGC GACATCCAGA TGACCCAAAG CCCTAGCACA
CTGAGCGCCA GCGTCGGCGA CAGAGTGACC ATTACATGCA AGGCCTCCCA GGACGTCGGC
ACAGCCGTGG CCTGGTACCA GCAGAAGCCT GGAAAGGCCC CAAAGCTGCT GATCTACTGG
GCCTCTACCC GGCATACCGG CGTGCCTGAC AGATTCAGCG GCAGCGGCTC TGGTACAGAC
TTCACCCTGA CCATTAGCAG CTTACAGGCC GAGGACTTCG CCGTGTACTT CTGCCACCAG
CACAGCAGCA ATCCTCTAAC CTTCGGCCAG GGAACCAAGC TGGAAATCAA AAGAACCGTG
GCCGCCCCTT CTGTATTCAT ATTTCCTCCA AGCGACGAGC AGCTCAAGAG CGGCACGGCT
TCTGTGGTGT GTCTGCTGAA CAACTTTTAT CCCAGAGAAG CCAAGGTGCA GTGGAAGGTG
GATAACGCCC TGCAATCCGG AAACTCTCAG GAGTCTGTCA CCGAGCAGGA CTCAAAGGAC
TCGACGTACA GCCTGAGCAG CACACTGACC CTGAGCAAGG CCGACTACGA GAAGCACAAA
GTTTACGCCT GCGAGGTGAC ACACCAGGGC CTCTCTAGCC CTGTGACAAA GAGCTTCAAC
AGGGGCGAGT GCTGA
SEQ ID NO: 43-[αC1s scFab-(G₄S)₃-αBb F2A Fab] amino acid
sequence (construct #19, FIG. 2F) (signal peptides boldfaced; furin
cleavage site underlined; F2A sequence italicized)
MEAPAQLLFL LLLWLPDTTG DIVLTOSPDS LAVSLGERAT ISCKASQSVD YDGDSYMNWY
QQKPGQPPKI LIYDASNLES GIPARESGSG SGTDFTLTIS SLEPEDFAIY YCQQSNEDPW
TFGGGTKVEI KRTVAAPSVE IFPPSDEQLK SGTASVVCLL NNFYPREAKV QWKVDNALOS
GNSQESVTEQ DSKDSTYSLS STLTLSKADY EKHKVYACEV THQGLSSPVT KSENRGECGG
GGSGGGGSGG GGSGGGGSGG GGSGGGGSGG GGSQVOLVOS GAEVKKPGAS VKLSCTASGE
NIKDDYIHWV KQAPGQGLEW IGRIDPADGH TKYAPKFQVK VTITADTSTS TAYLELSSLR
SEDTAVYYCA RYGYGREVED YWGQGTTVTV SSASTKGPSV FPLAPCSRST SESTAALGCL
VKDYFPEPVT VSWNSGALTS GVHTFPAVLQ SSGLYSLSSV VTVPSSSLGT KTYTCNVDHK
PSNTKVDKRV GGGGSGGGGS GGGGSEVQLV ESGGGLVKPG GSLRLSCAAS GFTFSNYAMS
WVRQAPGKRL EWVATISNRG SYTYYPDSVK GRFTISRDNA KNSLYLOMNS LRAEDTALYY
CARERPMDYW GQGTLVTVSS ASTKGPSVEP LAPCSRSTSE STAALGCLVK DYFPEPVTVS
WNSGALTSGV HTFPAVLOSS GLYSLSSVVT VPSSSLGTKT YTCNVDHKPS NTKVDKRVRK
RRSGSGAPVK QTLNEDLLKL AGDVESNPGP MEAPAQLLFL LLLWLPDTTG DIQMTOSPST
LSASVGDRVT ITCKASQDVG TAVAWYQQKP GKAPKLLIYW ASTRHTGVPD RESGSGSGTD
FTLTISSLQA EDFAVYFCHQ HSSNPLTFGQ GTKLEIKRTV AAPSVFIFPP SDEQLKSGTA
SVVCLLNNFY PREAKVOWKV DNALQSGNSQ ESVTEQDSKD STYSLSSTLT LSKADYEKHK
VYACEVTHQG LSSPVTKSEN RGEC*
SEQ ID NO: 44-[αC1s scFab-(G₄S)₃-αBb GT2A-Fab] nucleic acid
sequence (construct #20, FIG. 2F)
ATGGAAGCCC CTGCCCAGCT GCTGTTTCTG CTGCTGCTGT GGCTGCCTGA CACCACAGGC
GACATCGTTC TGACCCAGAG CCCTGACAGC CTGGCCGTGT CCCTAGGCGA ACGGGCCACC
ATCAGCTGCA AGGCCAGCCA GAGCGTGGAC TATGATGGCG ACAGCTACAT GAACTGGTAT
CAGCAAAAGC CCGGACAGCC TCCTAAGATC CTGATCTACG ACGCCTCTAA CCTGGAATCT
GGCATCCCTG CCAGATTTTC TGGCAGCGGT TCTGGCACCG ATTTCACCCT GACCATTAGC
TCTCTGGAGC CTGAGGACTT CGCCATCTAC TACTGCCAGC AGAGCAACGA GGATCCTTGG
ACATTCGGCG GCGGTACCAA GGTCGAGATT AAACGGACCG TGGCTGCTCC CAGCGTGTTC
ATCTTCCCAC CATCTGATGA GCAGCTGAAA TCTGGCACGG CCAGCGTCGT GTGCCTGCTG
AACAACTTCT ACCCTAGAGA GGCCAAGGTG CAGTGGAAGG TGGATAACGC CCTGCAGTCC
GGCAATAGCC AGGAGAGCGT GACTGAACAG GATAGTAAAG ACTCTACCTA CAGCCTGTCC
AGTACACTGA CCCTGTCTAA GGCCGATTAC GAGAAGCACA AAGTGTACGC CTGTGAAGTG
ACACATCAGG GCCTGAGCTC ACCTGTGACT AAGTCCTTCA ACCGGGGCGA GTGCGGCGGC
GGTGGCAGCG GCGGCGGCGG CAGCGGAGGC GGCGGCAGTG GAGGCGGCGG GTCTGGCGGA
GGTGGATCTG GTGGCGGCGG TAGCGGCGGC GGCGGCAGCC AGGTGCAACT GGTGCAGTCT
GGAGCTGAGG TGAAGAAACC TGGGGCCAGC GTGAAGCTGT CTTGCACCGC CAGCGGCTTC
AACATCAAGG ACGACTACAT CCACTGGGTC AAACAGGCTC CTGGACAGGG CTTGGAATGG
ATCGGCAGAA TCGACCCCGC CGACGGCCAC ACCAAGTACG CCCCAAAATT CCAGGTGAAA
GTAACAATCA CCGCTGATAC ATCTACTTCC ACAGCTTATC TGGAACTGAG CAGCCTGAGG
TCTGAGGATA CCGCCGTGTA CTACTGCGCC CGGTACGGCT ACGGCAGAGA GGTGTTCGAC
TACTGGGGAC AGGGCACCAC CGTGACCGTG TCTTCCGCCA GCACTAAGGG ACCTAGCGTG
TTCCCCCTGG CCCCATGTTC CCGGAGCACC AGCGAGTCTA CTGCCGCCCT GGGATGCCTG
GTGAAGGACT ACTTTCCTGA GCCCGTGACC GTGTCTTGGA ACAGCGGCGC CCTGACCAGC
GGCGTGCACA CATTCCCTGC CGTGCTGCAG AGCAGCGGCC TGTACAGCCT GTCCTCTGTG
GTGACAGTGC CCTCTAGCTC TCTCGGCACC AAAACCTACA CCTGCAACGT GGACCATAAG
CCTAGCAACA CCAAGGTCGA CAAGCGGGTG GGCGGCGGCG GGAGTGGCGG TGGCGGCTCT
GGCGGAGGGG GGAGCGAAGT GCAGCTGGTC GAAAGCGGAG GAGGACTAGT GAAGCCTGGC
GGCAGCCTGA GACTGAGCTG TGCTGCCAGC GGCTTTACAT TCAGCAACTA CGCCATGAGC
TGGGTGCGTC AGGCCCCCGG CAAGCGGCTG GAATGGGTCG CAACCATCAG CAATAGAGGC
AGCTACACTT ACTACCCTGA CTCCGTCAAG GGCAGATTCA CCATCTCCCG CGACAACGCC
AAAAACTCCC TGTACCTGCA AATGAATAGC CTGAGAGCCG AGGACACCGC CCTGTACTAT
TGCGCCAGAG AGAGACCTAT GGACTACTGG GGCCAGGGTA CCCTGGTGAC CGTGAGCTCT
GCTAGCACAA AGGGCCCTTC CGTGTTCCCT CTGGCTCCTT GCAGCAGAAG CACAAGCGAG
AGCACAGCCG CCCTGGGCTG CCTGGTTAAG GACTATTTTC CCGAACCTGT GACAGTCTCC
TGGAACAGCG GCGCCCTGAC CTCTGGGGTG CACACCTTCC CCGCTGTCCT GCAGAGCAGC
GGCCTGTACT CGCTGAGCTC TGTGGTGACC GTGCCTAGCA GCAGCCTGGG CACCAAGACA
TACACATGTA ATGTGGACCA CAAGCCCTCC AACACCAAGG TCGATAAGAG AGTGCGGAGA
AAGAGAGGTT CCGGCGAGGG CAGAGGCAGC CTGTTAACAT GCGGCGACGT GGAGGAAAAC
CCAGGACCTA TGGAGGCCCC CGCCCAGCTG CTCTTCCTGC TGCTGCTGTG GCTGCCCGAT
ACCACCGGCG ATATCCAGAT GACACAGTCC CCTTCAACCC TTAGTGCCTC GGTTGGCGAT
AGAGTGACAA TTACATGTAA AGCTAGCCAG GACGTGGGCA CCGCCGTGGC CTGGTACCAG
CAGAAGCCCG GCAAAGCCCC AAAGCTGCTC ATCTACTGGG CCTCGACAAG ACACACCGGC
GTGCCAGATA GATTCAGCGG CTCTGGCTCA GGCACAGACT TCACCCTGAC TATCAGCTCC
CTCCAAGCCG AGGATTTCGC CGTTTACTTC TGCCACCAGC ACAGCTCCAA TCCCCTGACA
TTCGGCCAAG GAACCAAGCT GGAAATCAAG CGGACCGTGG CCGCTCCTAG TGTCTTCATC
TTCCCTCCTT CCGACGAGCA GCTGAAGAGC GGCACAGCCT CCGTGGTGTG TCTGCTCAAC
AACTTTTACC CCAGAGAGGC CAAGGTGCAG TGGAAGGTGG ACAATGCCCT GCAGAGCGGA
AACAGCCAGG AGTCGGTGAC CGAGCAAGAC AGCAAGGACT CTACGTACAG CCTGTCAAGC
ACCCTGACGC TGAGCAAGGC CGATTACGAG AAGCACAAAG TGTACGCCTG CGAGGTGACC
CACCAGGGAC TGAGCAGCCC TGTGACCAAG AGCTTTAACC GTGGAGAATG CTGA
SEQ ID NO: 45-[αC1s scFab-(G₄S)₃-αBb GT2A-Fab] amino acid
sequence (construct #20, FIG. 2F) (signal peptides boldfaced; furin
cleavage site underlined; GT2A sequence italicized)
MEAPAQLLFL LLLWLPDTTG DIVLTOSPDS LAVSLGERAT ISCKASQSVD YDGDSYMNWY
QQKPGOPPKI LIYDASNLES GIPARFSGSG SGTDETLTIS SLEPEDFAIY YCQQSNEDPW
TFGGGTKVEI KRTVAAPSVE IFPPSDEQLK SGTASVVCLL NNFYPREAKV QWKVDNALQS
GNSQESVTEQ DSKDSTYSLS STLTLSKADY EKHKVYACEV THQGLSSPVT KSENRGECGG
GGSGGGGSGG GGSGGGGSGG GGSGGGGSGG GGSQVQLVQS GAEVKKPGAS VKLSCTASGF
NIKDDYIHWV KQAPGQGLEW IGRIDPADGH TKYAPKFQVK VTITADTSTS TAYLELSSLR
SEDTAVYYCA RYGYGREVED YWGQGTTVTV SSASTKGPSV FPLAPCSRST SESTAALGCL
VKDYFPEPVT VSWNSGALTS GVHTFPAVLQ SSGLYSLSSV VTVPSSSLGT KTYTCNVDHK
PSNTKVDKRV GGGGSGGGGS GGGGSEVQLV ESGGGLVKPG GSLRLSCAAS GFTFSNYAMS
WVRQAPGKRL EWVATISNRG SYTYYPDSVK GRFTISRDNA KNSLYLQMNS LRAEDTALYY
CARERPMDYW GQGTLVTVSS ASTKGPSVEP LAPCSRSTSE STAALGCLVK DYFPEPVTVS
WNSGALTSGV HTFPAVLOSS GLYSLSSVVT VPSSSLGTKT YTCNVDHKPS NTKVDKRVRR
KRGSGEGRGS LLTCGDVEEN PGPMEAPAQL LFLLLLWLPD TTGDIQMTOS PSTLSASVGD
RVTITCKASQ DVGTAVAWYQ QKPGKAPKLL IYWASTRHTG VPDRESGSGS GTDETLTISS
LQAEDFAVYF CHQHSSNPLT FGQGTKLEIK RTVAAPSVFI FPPSDEQLKS GTASVVCLLN
NFYPREAKVQ WKVDNALQSG NSQESVTEQD SKDSTYSLSS TLTLSKADYE KHKVYACEVT
HQGLSSPVTK SENRGEC*
SEQ ID NO: 46
GGGGS
SEQ ID NO: 47
GGGGSGGGGS
SEQ ID NO: 48
GGGGSGGGGS GGGGS
SEQ ID NO: 49
GGGGSGGGGS GGGGGGGGS GGGGSGGGGS GGGGS
SEQ ID NO: 50-Nucleotide sequence of AAV2#9 (FIGs. 2B and 2I) (5′
ITR boldfaced; bGH polyA signal underlined; reverse complement of
αC1s scFab coding sequence italicized; IgG kappa signal coding
sequence italicized and underlined; Kozak sequence boxed; CBA
promoter (reverse) bolded and underlined; CBA promoter boxed and
underlined; CMV enhancer boldfaced and italicized; αBb scFab
boldfaced, italicized, and underlined; and 3′ ITR boxed and
italicized)
TTGGCCACTC CCTCTCTGCG CGCTCGCTCG CTCACTGAGG CCGCCCGGGC AAAGCCCGGG
CGTCGGGCGA CCTTTGGTCG CCCGGCCTCA GTGAGCGAGC GAGCGCGCAG AGAGGGAGTG
GCCAACTCCA TCACTAGGGG TTCCTTACAA TTCTAGTTCC CCAGCATGCC TGCTATTGTC
TTCCCAATCC TCCCCCTTGC TGTCCTGCCC CACCCCACCC CCCAGAATAG AATGACACCT
ACTCAGACAA TGCGATGCAA TTTCCTCATT TTATTAGGAA AGGACAGTGG GAGTGGCACC
TTCCAGGGTC AAGGAAGGCA CGGGGGAGGG GCAAACAACA GATGGCTGGC AACTAGAAGG
CACAGGTTTA AACCCTGCAG GGAGCTCTCA CACCCGCTTA TCCACCTTGG TGTTGCTGGG
CTTGTGGTCC ACGTTGCAGG TGTAGGTCTT TGTGCCCAGG CTAGAGCTAG GCACTGTCAC
GACAGAGGAC AGAGAGTACA GGCCGCTGCT CTGCAGCACG GCGGGGAAGG TGTGCACCCC
GCTTGTCAGG GCTCCGCTGT TCCAGGACAC GGTCACAGGC TCAGGGAAAT AATCCTTGAC
CAGGCAGCCC AGAGCAGCCG TGCTCTCTGA GGTACTTCTG CTACAAGGAG CCAGTGGGAA
CACGCTAGGG CCCTTTGTGC TGGCGGACGA CACGGTCACT GTTGTGCCCT GTCCCCAGTA
GTCGAACACT TCTCTGCCGT AGCCGTATCT GGCGCAGTAG TACACAGCGG TGTCCTCGGA
TCTAAGGCTG CTCAGTTCCA GATAAGCTGT AGAGGTGCTG GTATCGGCGG TGATGGTGAC
TTTCACCTGG AACTTAGGGG CGTACTTTGT GTGGCCGTCG GCAGGGTCGA TTCTGCCGAT
CCACTCCAGT CCCTGGCCGG GGGCCTGCTT CACCCAGTGG ATGTAATCGT CCTTGATATT
GAAGCCGCTG GCGGTGCAGC TCAGCTTAAC ACTAGCGCCA GGCTTTTTCA CCTCGGCTCC
GCTCTGCACC AGCTGCACCT GGGATCCGCC GCCGCCGCTG CCGCCTCCGC CGCTGCCGCC
TCCGCCGCTT CCGCCTCCCC CAGAGCCGCC GCCACCGCTG CCTCCTCCGC CGGAGCCGCC
GCCGCCGCAC TCGCCCCGGT TGAAGCTTTT GGTCACAGGA GAGGACAGGC CCTGATGTGT
CACTTCACAG GCGTACACCT TGTGCTTCTC GTAGTCGGCC TTGCTCAAGG TCAGGGTGCT
GGACAGGCTG TATGTTGAGT CCTTGCTGTC CTGCTCGGTC ACGCTCTCTT GGCTGTTGCC
GCTTTGCAGG GCGTTGTCAA CTTTCCATTG GACCTTTGCC TCTCTGGGGT AGAAGTTATT
CAGCAGGCAC ACCACAGAGG CGGTTCCGCT CTTCAGCTGC TCGTCGCTTG GAGGGAAGAT
AAAGACAGAA GGGGCGGCCA CGGTGCGCTT GATTTCCACC TTGGTGCCGC CTCCAAAGGT
CCAGGGGTCC TCGTTGCTCT GCTGGCAGTA GTAGATGGCA AAATCCTCGG GTTCCAGAGA
AGAAATTGTC AGGGTGAAAT CAGTGCCAGA GCCGCTGCCG CTGAATCTGG CGGGGATGCC
GCTTTCCAGA TTGCTGGCGT CGTAGATCAG GATTTTTGGA GGCTGGCCGG GTTTCTGCTG
GTACCAGTTC ATGTAGCTGT CGCCGTCATA GTCCACGCTC TGAGAGGCTT TACAGCTGAT
TGTGGCCCGT TCGCCGAGGC TCACGGCCAG GCTATCAGGG CTCTGCGTCA GCACGATATC

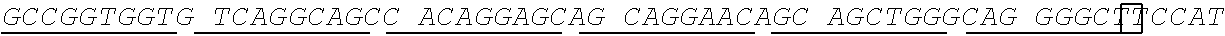

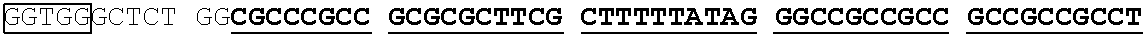

CGCCATAAAA GGAAACTTTC GGAGCGCGCC GCTCTGATTG GCTGCCGCCG CACCTCTCCG
CCTCGCCCCG CCCCGCCCCT CGCCCCGCCC CGCCCCGCCT GGCGCGCGCC CCCCCCCCCC
CCCCGCCCCC ATCGCTGCAC AAAATAATTA AAAAATAAAT AAATACAAAA TTGGGGGTGG
GGAGGGGGGG GAGATGGGGA GAGTGAAGCA GAACGTGGGG CTCACCTCGC TAGTTATTAA
TAGTAATCAA TTACGGGGTC ATTAGTTCAT AGCCCATATA TGGAGTTCCG CGTTACATAA
CTTACGGTAA ATGGCCCGCC TGGCTGACCG CCCAACGACC CCCGCCCATT GACGTCAATA
ATGACGTATG TTCCCATAGT AACGCCAATA GGGACTTTCC ATTGACGTCA ATGGGTGGAG
TATTTACGGT AAACTGCCCA CTTGGCAGTA CATCAAGTGT ATCATATGCC AAGTACGCCC
CCTATTGACG TCAATGACGG TAAATGGCCC GCCTGGCATT ATGCCCAGTA CATGACCTTA

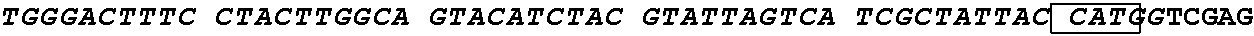

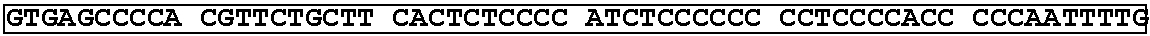

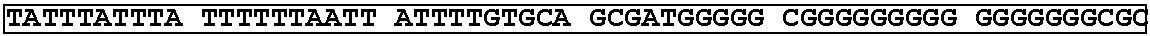

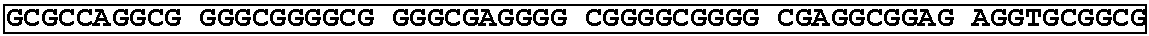

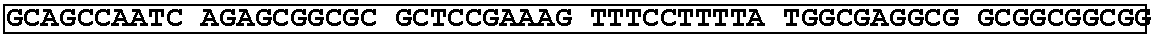

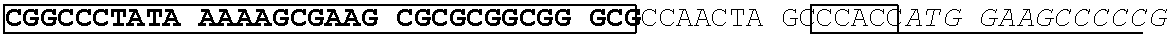

CTCAGCTGCT GTTCCTGCTG CTGCTGTGGC TGCCTGACAC CACCGGCGAC ATCCAGATGA
CACAGAGCCC TAGCACCCTG AGCGCCTCCG TGGGGGACAG AGTGACAATC ACATGTAAAG
CCTCCCAGGA CGTGGGCACT GCCGTGGCCT GGTACCAGCA AAAACCGGGA AAAGCCCCTA
AGCTGCTGAT CTACTGGGCC AGCACCAGAC ACACCGGCGT CCCCGATAGA TTCAGCGGCT
CTGGCAGCGG AACTGATTTC ACCCTGACCA TTTCTTCTCT GCAGGCCGAG GACTTCGCCG
TGTACTTTTG CCACCAGCAC AGCAGCAACC CTCTGACCTT CGGACAGGGC ACAAAGCTGG
AAATCAAGCG GACAGTGGCT GCTCCTTCTG TGTTCATCTT TCCACCTAGC GACGAGCAGC
TGAAGAGCGG CACCGCCTCT GTGGTGTGCC TGCTGAACAA CTTCTACCCC AGAGAAGCCA
AAGTGCAGTG GAAGGTGGAC AACGCCCTGC AATCTGGCAA CAGCCAGGAG AGCGTGACGG
AACAAGATAG CAAGGACAGC ACCTACTCCC TGAGCAGCAC ACTGACCTTG TCCAAGGCAG
ATTACGAGAA GCACAAGGTG TACGCCTGCG AGGTGACCCA CCAGGGACTG AGCAGCCCAG
TGACCAAGAG CTTCAACAGA GGAGAGTGCG GCGGCGGCGG AAGCGGAGGC GGAGGCAGCG
GCGGCGGCGG CAGTGGAGGC GGCGGCTCTG GCGGAGGGGG CAGTGGCGGT GGCGGATCCG
GCGGCGGCGG CAGCGAGGTG CAGCTTGTGG AATCCGGCGG CGGCCTGGTG AAGCCCGGCG
GTAGCCTGAG ACTGTCTTGT GCCGCCTCTG GCTTCACCTT TAGCAATTAC GCCATGAGCT
GGGTGCGGCA GGCTCCCGGC AAAAGACTGG AATGGGTCGC CACCATCAGC AACCGGGGAT
CATATACCTA CTACCCTGAT AGCGTGAAAG GCAGGTTCAC AATCAGCCGG GACAATGCCA
AGAACAGCCT GTACCTGCAG ATGAACTCAC TGCGGGCCGA GGACACCGCC CTGTATTACT
GCGCCAGAGA GAGACCTATG GACTACTGGG GCCAGGGCAC CCTGGTGACC GTTTCCTCCG
CCAGCACCAA GGGCCCTAGC GTGTTCCCTC TGGCCCCATG CAGCAGAAGC ACATCTGAGA
GCACCGCCGC TCTGGGCTGC CTGGTGAAGG ACTACTTCCC CGAGCCTGTG ACAGTGAGCT
GGAACTCCGG CGCCCTGACC AGCGGCGTGC ACACATTTCC AGCTGTGCTG CAGTCTAGCG
GCCTGTACAG CCTGAGCAGC GTTGTGACAG TGCCTTCTAG CAGCCTCGGC ACCAAGACCT
ACACCTGTAA CGTGGATCAT AAGCCTTCTA ATACCAAGGT TGACAAGAGA GTGTGAGAGC
TCCCTGCAGG GTTTAAACCT GTGCCTTCTA GTTGCCAGCC ATCTGTTGTT TGCCCCTCCC
CCGTGCCTTC CTTGACCCTG GAAGGTGCCA CTCCCACTGT CCTTTCCTAA TAAAATGAGG
AAATTGCATC GCATTGTCTG AGTAGGTGTC ATTCTATTCT GGGGGGTGGG GTGGGGCAGG

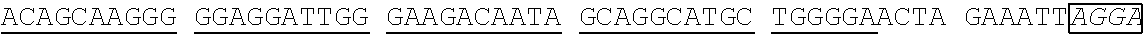

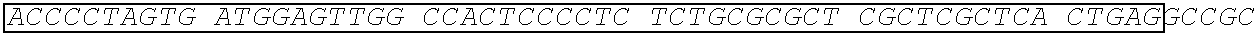

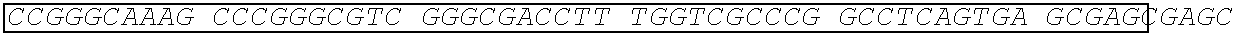

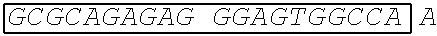

SEQ ID NO: 51-Nucleotide sequence of AAV2#12 (FIGs. 2C and 2J) (5′
ITR boldfaced; minCBA promoter (which comprises a CMV enhancer, a
CBA promoter, and a truncated chimeric intron) underlined; Kozak
sequence boxed; IgG kappa signal coding sequence italicized; αBb
scFab coding sequence bolded and underlined; (G₄S)₇, linker coding
sequence in lower case, boldfaced and italicized; (G₄S)₃ linker
coding sequence boxed and italicized; αC1s scFab coding sequence
boldfaced and italicized; bGH polyA italicized and underlined; and
3′ ITR boxed and boldfaced)
TTGGCCACTC CCTCTCTGCG CGCTCGCTCG CTCACTGAGG CCGCCCGGGC AAAGCCCGGG
CGTCGGGCGA CCTTTGGTCG CCCGGCCTCA GTGAGCGAGC GAGCGCGCAG AGAGGGAGTG
GCCAACTCCA TCACTAGGGG TTCCTTACCG GTGCGGGCCT CTTCGCTATT ACGCCAGCTG
GCGAAAGGGG GATGTGCTGC AAGGCGATTA AGTTGGGTAA CGCCAGGGTT TTCCCAGTCA
CGACGTTGTA AAACGACGGC CAGTGAATTC GGACCGAGAT CTGAATTCGG TACCTAGTTA
TTAATAGTAA TCAATTACGG GGTCATTAGT TCATAGCCCA TATATGGAGT TCCGCGTTAC
ATAACTTACG GTAAATGGCC CGCCTGGCTG ACCGCCCAAC GACCCCCGCC CATTGACGTC
AATAATGACG TATGTTCCCA TAGTAACGCC AATAGGGACT TTCCATTGAC GTCAATGGGT
GGAGTATTTA CGGTAAACTG CCCACTTGGC AGTACATCAA GTGTATCATA TGCCAAGTAC
GCCCCCTATT GACGTCAATG ACGGTAAATG GCCCGCCTGG CATTATGCCC AGTACATGAC
CTTATGGGAC TTTCCTACTT GGCAGTACAT CTACGTATTA GTCATCGCTA TTACCATGGT
CGAGGTGAGC CCCACGTTCT GCTTCACTCT CCCCATCTCC CCCCCCTCCC CACCCCCAAT
TTTGTATTTA TTTATTTTTT AATTATTTTG TGCAGCGATG GGGGCGGGGG GGGGGGGGGG
GCGCGCGCCA GGCGGGGCGG GGCGGGGCGA GGGGCGGGGC GGGGCGAGGC GGAGAGGTGC
GGCGGCAGCC AATCAGAGCG GCGCGCTCCG AAAGTTTCCT TTTATGGCGA GGCGGCGGCG
GCGGCGGCCC TATAAAAAGC GAAGCGCGCG GCGGGCGGGA GTCGCTGCGC GCTGCCTTCG
CCCCGTGCCC CGCTCCGCCG CCGCCTCGCG CCGCCCGCCC CGGCTCTGAC TGACCGCGTT
ACTCCCACAG GTGAGCGGGC GGGACGGCCC TTCTCCTCCG GGCTGTAATT AGCGCTTGGT
TTAATGACGG CTTGTTTCTT TTCTGTGGCT GCGTGAAAGC CTTGAGGGGC TCCGGGAGCT
AGAGCCTCTG CTAACCATGT TCATGCCTTC TTCTTTTTCC TACAGCTCCT GGGCAACGTG
CTGGTTATTG TGCTGTCTCA TCATTTTGGC AAAGAATTCC TCGAAGATCC GGTACCCAAT

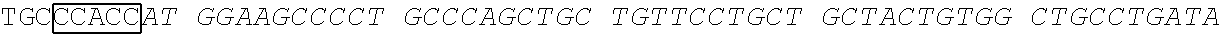

CCACCGGCGA TATCCAGATG ACGCAGAGTC CCAGCACCCT GAGCGCCTCT GTGGGCGACC
GGGTGACCAT CACCTGTAAA GCCTCCCAGG ACGTGGGCAC AGCTGTTGCT TGGTATCAGA
AAAAGCCTGG CAAGGCCCCT AAGCTGCTGA TCTACTGGGC CAGCACAAGA CACACAGGAG
TGCCTGACAG ATTCAGCGGC AGCGGCTCTG GGACTGATTT CACCTTGACA ATCAGCTCTC
TGCAGGCCGA GGACTTTGCC GTGTACTTCT GCCACCAACA CAGTTCTAAC CCCCTGACCT
TCGGCCAAGG AACCAAGCTG GAAATCAAGC GGACCGTGGC CGCTCCTGCC GTGTTCATCT
TCCCTCCAAG CGATGAGCAG CTGAAAAGCG GCACCGCGTC CGTCGTGTGC CTGCTGAAGA
ACTTCTACCC GAGAGAAGCG AAGGTGCAGT GGAAAGTCGA CAACGCCCTG CAGAGCGGAA
ATAGCCAGGA GAGCGTGACC GAACAAGACT CTAAGGACAG CACCTACTCG CTGTCCTCCA
CGCTGACTCT GTCTAAGGCC GACTATGAGA AGCACAAGGT GTACGCCTGC GAGGTGACCC
ACCAGGGCCT GAGCAGCCCC GTTACCAAGA GCTTCAACAG AGGAGAATGC ggcggaggtg
gcagcggcgg cggcgggago ggcggcggcg gctcaggcgg agggggaagt ggcggcggcg
gcagcggcgg cggaggcagc ggcggtggcg gctctGAGGT GCAACTGGTG GAATCTGGGG
GCGGACTGGT GAAGCCTGGC GGCAGTCTGA GACTGAGCTG TGCCGCTTCC GGATTCACCT
TTAGCAATTA CGCCATGAGC TGGGTGCGGG AGGCCCCTGG AAAGCGGCTG GAATGGGTTG
CTACAATCAG CAATAGAGGC AGCTACACAT ACTACCCCGA CAGTGTCAAA GGCCGGTTTA
CAATCAGCCG CGACAACGCC AAAAACAGCC TGTACCTGCA GATGAACTCC CTGCGGGCTG
AGGATACAGC CCTCTACTAC TGTGCCAGAG AACGTCCAAT GGACTATTGG GGCCAAGGCA
CACTGGTGAC CGTGAGCAGC GCGTCTACCA AGGGCCCTTC TGTTTTCCCT CTGGCCCCCT
GCAGCAGAAG CACGAGCGAG AGCACCGCTG CCCTGGGCTG TCTGGTGAAG GATTATTTCC
CTGAGCCTGT GACCGTGTCT TGGAATAGCG GAGCOCTGAC CAGCGGAGTG CATACATTCC
CTGCTGTGCT GCAGTCTAGT GGGCTGTACA GCCTGTCTTC CGTTGTGGAA GTCCCTAGCA
GCAGCCTGGG CACCAAGACC TACACCTGCA ACGTGGATCA TAAGCCAAGC AACACCAAGG

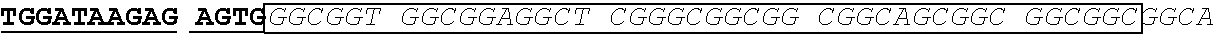

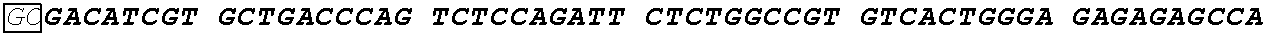

CCATTAGCTG CAAGGCCTCT CAGAGCGTAG ACTACGACGG CGACTCCTAC ATGAACTGGT
ACCAGGAAAA GCCTGGCCAG CCTCCTAAGA TCTTGATCTA CGATGCCTCC AATCTGGAGA
GCGGGATCCC CGCTAGATTC AGCGGGTCTG GAAGTGGAAC CGACTTCACA CTGACCATCT
CTAGCCTGGA GCCCGAGGAC TTTGCCATCT ACTACTGCCA GCAGAGCAAC GAGGACCCCT
GGACATTCGG CGGCGGCACA AAGGTTGAGA TCAAGAGAAC CGTTGCCGCT CCTAGCGTGT
TTATCTTCCC TCCCTCTGAC GAGCAGCTGA AGAGCGGCAC AGCCTCCGTG GTGTGCCTGC
TGAACAACTT CTACCCCAGA GAGGCCAAGG TCCAGTGGAA GGTCGACAAT GCCCTTCAGA
GCGGCAACAG CCAGGAGTCC GTGACCGAGC AGGATAGCAA GGACTCTACC TACAGCCTGT
CCTCTACGCT GACCCTGAGC AAAGCCGATT ACGAAAAGCA CAAAGTGTAC GCCTGTGAAG
TGACACACCA GGGCCTGTCT AGCCCTGTGA CAAAGAGCTT TAACCGGGGC GAGTGCggcg
gcggtggaag cggaggtgga ggttcaggag geggeggaag cggaggcgga ggcagtgggg
geggeggctc cggaggcagc ggcageggag geggeggttc ccAAGTGCAG CTCGTGCAGA
GCGGCGCCGA GGTGAAAAAG CCCGGAGCCA GCGTGAAGCT GTCTTGCACC GCCTCCGGAT
TCAACATCAA AGACGACTAC ATCCACTGGG TCAAGAAAGC CCCAGGGCAG GGGCTGGAGT
GGATCGGCAG GATCGACCCT GCTGATGGCC ACACCAAATA CGCCCCAAAG TTCCAGGTGA
AAGTGACAAT TACCGCAGAT ACCTCCACCA GCACCGCTTA TCTGGAACTG AGCTCTCTGC
GGAGCGAGGA CACAGCCGTG TACTACTGCG CCAGATACGG CTACGGCAGA GAAGTGTTCG
ACTACTGGGG CCAGGGCACC ACAGTGACAG TGAGCTCTGC CAGCACAAAG GGCCCCAGCG
TGTTTCCTCT GGCCCCTTGC AGCAGAAGCA CCAGCGAGAG CACCGCCGCC CTGGGCTGCC
TGGTGAAGGA CTACTTCCCT GAACCCGTGA CCGTCTCCTG GAACAGTGGC GCCTTGACCT
CTGGCGTGCA CACCTTCCCC GCCGTGCTGC AGAGCTCCGG CCTGTACAGC CTGTCTAGCG
TGGTGACCGT GCCTAGCTCG AGCCTGGGCA CAAAGACATA TACCTGTAAC GTGGACCACA
AGCCCAGCAA CACGAAGGTG GACAAGCGAG TGTGAGTTTA AACCTGTGCC TTCTAGTTGC
CAGCCATCTG TTGTTTGCCC CTCCCCCGTG CCTTCCTTGA CCCTGGAAGG TGCCACTCCC
ACTGTCCTTT CCTAATAAAA TGAGGAAATT GCATCGCATT GTCTGAGTAG GTGTCATTCT
ATTCTGGGGG GTGGGGTGGG GCAGGACAGC AAGGGGGAGG ATTGGGAAGA CAATAGCAGG

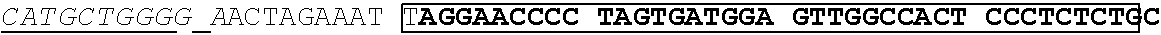

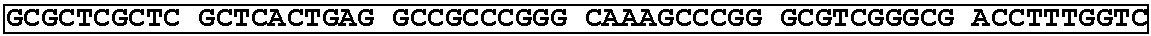

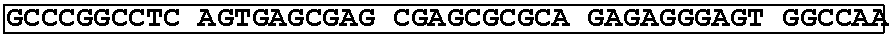

SEQ ID NO: 52-Nucleotide sequence of AAV2#14 (FIGs. 2D and 2H) (3′
ITR boldfaced; minCBA promoter underlined; Kozak sequence boxed;
IgG kappa signal sequence italicized; αC1s scFab coding sequence
boldfaced and underlined; (G₄S)₂ linker coding sequence boxed and
italicized; (G4S); coding sequence in lower case, boldfaced, and
italicized; αBb scFv coding sequence boldfaced and italicized;
bGH polyA signal italicized and underlined; and 5′ ITR boxed and
boldfaced)
TTGGCCACTC CCTCTCTGCG CGCTCGCTCG CTCACTGAGG CCGGGCGACC AAAGGTCGCC
CGACGCCCGG GCTTTGCCCG GGCGGCCTCA GTGAGCGAGC GAGCGCGCAG AGAGGGAGTG
GCCAACTCCA TCACTAGGGG TTCCTAATTT GATCTGAATT CGGTACCTAG TTATTAATAG
TAATCAATTA CGGGGTCATT AGTTCATAGC CCATATATGG AGTTCCGCGT TACATAACTT
ACGGTAAATG GCCCGCCTGG CTGACCGCCC AACGACCCCC GCCCATTGAC GTCAATAATG
ACGTATGTTC CCATAGTAAC GCCAATAGGG ACTTTCCATT GACGTCAATG GGTGGAGTAT
TTACGGTAAA CTGCCCACTT GGCAGTACAT CAAGTGTATC ATATGCCAAG TACGCCCCCT
ATTGACGTCA ATGACGGTAA ATGGCCCGCC TGGCATTATG CCCAGTACAT GACCTTATGG
GACTTTCCTA CTTGGCAGTA CATCTACGTA TTAGTCATCG CTATTACCAT GGTCGAGGTG
AGCCCCACGT TCTGCTTCAC TCTCCCCATC TCCCCCCCCT CCCCACCCCC AATTTTGTAT
TTATTTATTT TTTAATTATT TTGTGCAGCG ATGGGGGCGG GGGGGGGGGG GGGGCGCGCG
CCAGGCGGGG CGGGGCGGGG CGAGGGGCGG GGCGGGGCGA GGCGGAGAGG TGCGGCGGCA
GCCAATCAGA GCGGCGCGCT CCGAAAGTTT CCTTTTATGG CGAGGCGGCG GCGGCGGCGG
CCCTATAAAA AGCGAAGCGC GCGGCGGGCG GGAGTCGCTG CGCGCTGCCT TCGCCCCGTG
CCCCGCTCCG CCGCCGCCTC GCGCCGCCCG CCCCGGCTCT GACTGACCGC GTTACTCCCA
CAGGTGAGCG GGCGGGACGG CCCTTCTCCT CCGGGCTGTA ATTAGCGCTT GGTTTAATGA
CGGCTTGTTT CTTTTCTGTG GCTGCGTGAA AGCCTTGAGG GGCTCCGGGA GCTAGAGCCT
CTGCTAACCA TGTTCATGCC TTCTTCTTTT TCCTACAGCT CCTGGGCAAC GTGCTGGTTA

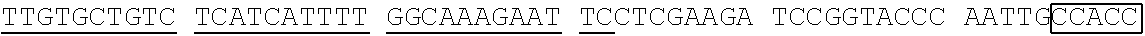

ATGGAAGCCC CCGCCCAGCT GCTGTTCCTG CTGCTCCTGT GGCTGCCTGA TACCACCGGC
GATATCGTCC TGACCCAGAG CCCTGATAGC CTGGCCGTTT CACTGGGCGA GCGGGCCACA
ATCTCCTGCA AGGCCTCTCA GTCTGTTGAC TACGACGGCG ACAGCTACAT GAACTGGTAC
CAGGAGAAAC CCGGCCAACC TCCAAAGATC CTGATCTACG ACGCCTCTAA TCTGGAGAGC
GGCATCCCCG CCCGGTTCAG CGGGTOCGGC AGCGGCACCG ACTTTACCCT GACCATCTCT
AGCCTGGAGC CTGAGGACTT CGCCATCTAC TACTGTCAGC AGAGCAACGA GGATCCTTGG
ACCTTTGGCG GCGGCACAAA GGTGGAAATC AAGCGGACCG TCGCCGCTCC ATCCGTGTTT
ATCTTCCCTC CTTCCGACGA GCAGCTCAAG AGCGGTACCG CCAGCGTGGT GTGCCTGCTG
AACAACTTCT ACCCCAGAGA GGCCAAGGTG CAGTGGAAGG TAGACAACGC CTTGCAGAGC
GGCAACTCTC AAGAGAGCGT GACAGAGCAG GACTCTAAGG ACAGCACATA CAGCCTAAGC
TCCACCCTGA CCCTCAGCAA GGCCGACTAC GAGAAGCACA AGGTGTACGC CTGTGAAGTT
ACACACCAGG GCCTGAGCAG TCCGGTGACC AAGTCCTTCA ACAGAGGCGA ATGCggcgga
ggaggctctg gcggcggcgg cagcggcgga ggcggcagcg gcggcggagg ctctggcggc
ggtggcagcg gaggcggcgg aagcggcgga ggtggcagcC AGGTGCAGCT GGTGCAGAGC
GGTGCTGAAG TGAAGAAACC CGGCGCTTCC GTGAAACTGA GCTGCACCGC CAGCGGATTT
AACATCAAGG ACGACTACAT TCACTGGGTG AAAAAGGCCC CTGGCCAGGG CCTGGAATGG
ATCGGGAGAA TCGACCCCGC CGATGGCCAT ACCAAGTACG CTCCTAAGTT CCAGGTGAAA
GTGACCATCA CCGCTGATAC AAGCACCTCT ACAGCCTACC TGGAGCTGAG CTCCCTGCGG
TCTGAGGACA CCGCCGTGTA CTACTGCGCC AGATACGGCT ACGGCAGAGA GGTGTTCGAC
TACTGGGGAC AGGGCACTAC AGTCACCGTG TCTAGTGCTA GCACGAAGGG CCCTAGCGTG
TTCCCTCTGG CTCCATGTAG CAGAAGCACC AGCGAAAGCA CAGCTGCTCT GGGCTGCCTG
GTGAAAGACT ACTTCCCCGA GCCTGTGACC GTCAGCTGGA ACTCCGGCGC CCTGACCAGC
GGAGTGCACA CCTTTCCTGC TGTGCTGCAA TCCTCTGGCC TGTACTCTCT GAGCTCTGTT
GTGACAGTGC CTTCTAGCAG CCTGGGAACC AAGACCTACA CCTGCAACGT GGACCACAAG

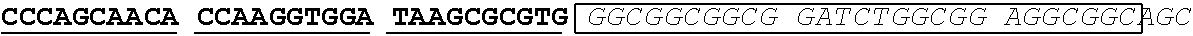

GAGGTGCAGC TGGTGGAAAG CGGCGGCGGC CTGGTGAAGC CTGGCGGCTC ACTGAGACTG
AGCTGTGCCG CCAGCGGCTT CACCTTCTCC AACTACGCCA TGAGCTGGGT GCGGGAAGCC
CCAGGAAAGC GCCTGGAGTG GGTCGCCACC ATCAGCAATA GAGGCTCGTA TACATATTAC
CCTGATTCCG TCAAAGGCAG ATTCACCATC TCTAGAGATA ATGCCAAGAA CAGCCTGTAC
CTGCAGATGA ACTCCCTCAG AGCCGAGGAT ACAGCCCTGT ATTACTGCGC CAGAGAACGG
CCTATGGACT ACTGGGGCCA AGGCACTCTG GTGACAGTGA GCAGCGGCGG CGGTGGTTCC
GGCGGCGGAG GCTCTGGAGG AGGCGGCAGC GACATCCAGA TGACCCAGAG CCCTAGCACC
CTGTCCGCCA GCGTGGGAGA TAGAGTGACC ATTACCTGTA AAGCGAGCCA GGATGTGGGC
ACCGCCGTGG CCTGGTATCA GAAGAAGCCT GGCAAGGCCC CTAAGCTGCT GATCTACTGG
GCCTCTACCC GGCACACAGG CGTGCCCGAC AGATTCTCCG GCTCCGGTTC TGGAACAGAC
TTCACACTGA CCATCAGCTC TCTTCAGGCC GAGGACTTCG CCGTGTACTT CTGCCACCAG
CACAGCTCTA ATCCTCTGAC ATTCGGCCAA GGCACAAAGC TGGAAATCAA GTGAGTTTAA
ACCTGTGCCT TCTAGTTGCC AGCCATCTGT TGTTTGCCCC TCCCCCGTGC CTTCCTTGAC
CCTGGAAGGT GCCACTCCCA CTGTCCTTTC CTAATAAAAT GAGGAAATTG CATCGCATTG
TCTGAGTAGG TGTCATTCTA TTCTGGGGGG TGGGGTGGGG CAGGACAGCA AGGGGGAGGA

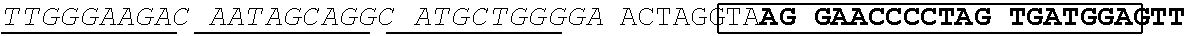

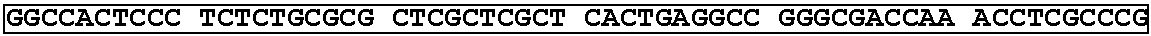

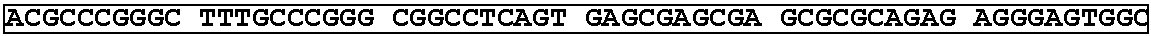

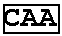

SEQ ID NO: 53-Bidirectional promoter and CMV enhancer (CBA
promoter (reverse) bolded and underlined; CMV enhancer boldfaced and
italicized; CBA promoter boxed and underlined)
CGCCCGCCGC GCGCTTCGCT TTTTATAGGG CCGCCGCCGC CGCCGCCTCG CCATAAAAGG
AAACTTTCGG AGCGCGCCGC TCTGATTGGC TGCCGCCGCA CCTCTCCGCC TCGCCCCGCC
CCGCCCCTCG CCCCGCCCCG CCCCGCCTGG CGCGCGCCCC CCCCCCCCCC CCGCCCCCAT
CGCTGCACAA AATAATTAAA AAATAAATAA ATACAAAATT GGGGGTGGGG AGGGGGGGGA
GATGGGGAGA GTGAAGCAGA ACGTGGGGCT CACCTCGCTA GTTATTAATA GTAATCAATT
ACGGGGTCAT TAGTTCATAG CCCATATATG GAGTTCCGCG TTACATAACT TACGGTAAAT
GGCCCGCCTG GCTGACCGCC CAACGACCCC CGCCCATTGA CGTCAATAAT GACGTATGTT
CCCATAGTAA CGCCAATAGG GACTTTCCAT TGACGTCAAT GGGTGGAGTA TTTACGGTAA
ACTGCCCACT TGGCAGTACA TCAAGTGTAT CATATGCCAA GTACGCCCCC TATTGACGTC
AATGACGGTA AATGGCCCGC CTGGCATTAT GCCCAGTACA TGACCTTATG GGACTTTCCT

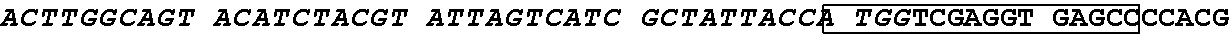

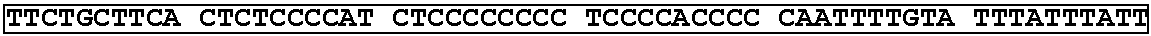

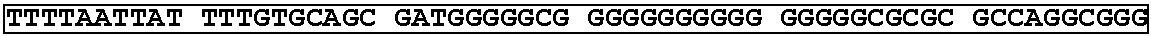

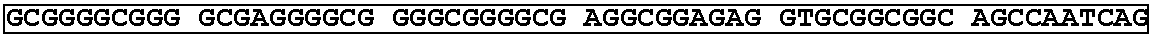

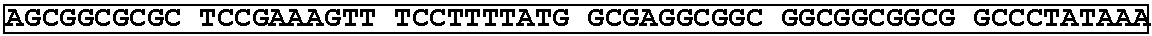

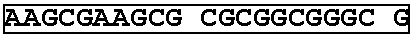

SEQ ID NO: 54-[αC1s scFv-(G₄S)₂-αBb scFv] nucleic acid sequence
(construct #5, FIG. 2A)
ATGGAAGCCC CAGCTCAGCT GCTGTTCCTC CTGCTGCTGT GGCTGCCTGA CACAACCGGC
CAAGTGCAGC TGGTCCAGAG CGGCGCCGAG GTGAAAAAGC CAGGAGCCTC CGTCAAACTG
AGCTGTACCG CCAGCGGCTT TAACATCAAG GACGACTACA TCCACTGGGT GAAGCAGGCC
CCTGGCCAAG GTCTGGAATG GATCGGCAGA ATCGACCCCG CTGACGGCCA CACCAAGTAC
GCCCCTAAGT TCCAGGTGAA GGTGACCATC ACCGCCGACA CCAGCACAAG CACCGCATAC
CTGGAGCTGT CCAGCCTGAG AAGCGAGGAT ACCGCTGTCT ACTACTGCGC CAGATACGGC
TACGGCAGAG AGGTGTTCGA CTACTGGGGA CAAGGTACCA CCGTGACGGT GTCTAGCGGC
GGTGGCGGCA GCGGAGGAGG CGGCTCTGGA GGCGGCGGAT CTGATATCGT GCTGACACAG
AGTCCTGACA GCCTGGCCGT GAGCTTGGGG GAGCGGGCTA CAATCTCTTG TAAAGCCAGC
CAGAGCGTGG ACTATGATGG CGATAGCTAC ATGAACTGGT ATCAGCAGAA ACCCGGCCAG
CCCCCCAAGA TCCTGATCTA CGACGCCAGC AATCTGGAGA GCGGCATCCC CGCCCGGTTC
AGCGGCAGCG GCTCGGGCAC AGATTTCACC CTGACCATTA GCTCTCTGGA ACCTGAGGAC
TTCGCTATCT ACTACTGCCA GCAGAGCAAC GAGGACCCTT GGACCTTCGG CGGAGGTACA
AAGGTGGAAA TCAAGGGCGG CGGCGGCAGC GGAGGCGGAG GCTCTGAGGT GCAACTGGTG
GAGAGCGGCG GCGGACTGGT AAAGCCCGGC GGCTCACTGA GACTGTCCTG CGCTGCCAGC
GGCTTCACCT TTTCTAACTA CGCCATGAGC TGGGTGCGGC AGGCTCCTGG AAAGCGCCTG
GAATGGGTGG CCACAATCAG CAACCGGGGC TCTTACACCT ACTATCCTGA TTCTGTGAAG
GGTAGGTTCA CCATTTCAAG AGATAACGCC AAGAACAGCC TCTACCTGCA GATGAACAGC
CTGCGGGCCG AAGACACCGC CCTGTACTAC TGCGCCAGAG AAAGACCTAT GGACTACTGG
GGCCAGGGCA CCCTGGTGAC AGTTTCCTCC GGAGGCGGAG GCTCCGGCGG CGGCGGCTCC
GGAGGCGGCG GAAGCGACAT CCAGATGACC CAGAGCCCTA GCACTCTGTC CGCCAGCGTG
GGCGACAGAG TGACCATCAC ATGCAAGGCC TCTCAGGACG TGGGCACCGC CGTGGCCTGG
TACCAACAGA AGCCTGGCAA GGCCCCTAAG CTGCTGATCT ACTGGGCCAG CACAAGACAT
ACAGGCGTGC CCGATAGATT CAGCGGCTCC GGCTCTGGCA CAGACTTCAC ACTGACCATC
AGCAGCCTCC AGGCCGAGGA TTTTGCCGTG TACTTCTGCC ACCAGCACAG CAGCAATCCA
CTGACATTTG GCCAGGGCAC CAAGCTGGAG ATCAAATGA
SEQ ID NO: 55-[αC1s scFv-(G₄S)₂-αBb scFv] amino acid sequence
(construct #5, FIG. 2A) (signal peptide boldfaced)
MEAPAQLLFL LLLWLPDTTG QVOLVOSGAE VKKPGASVKL SCTASGENIK DDYIHWVKQA
PGQGLEWIGR IDPADGHTKY APKFQVKVTI TADTSTSTAY LELSSLRSED TAVYYCARYG
YGREVEDYWG QGTTVTVSSG GGGSGGGGSG GGGSDIVLTQ SPDSLAVSLG ERATISCKAS
QSVDYDGDSY MNWYQQKPGQ PPKILIYDAS NLESGIPARF SGSGSGTDFT LTISSLEPED
FAIYYCQQSN EDPWTFGGGT KVEIKGGGGS GGGGSEVQLV ESGGGLVKPG GSLRLSCAAS
GFTFSNYAMS WVRQAPGKRL EWVATISNRG SYTYYPDSVK GRFTISRDNA KNSLYLQMNS
LRAEDTALYY CARERPMDYW GQGTLVTVSS GGGGSGGGGS GGGGSDIQMT QSPSTLSASV
GDRVTITCKA SQDVGTAVAW YQQKPGKAPK LLIYWASTRH TGVPDRESGS GSGTDETLTI
SSLQAEDFAV YFCHQHSSNP LTFGQGTKLE IK*
SEQ ID NO: 56-[αBb scFv-(G₄S)₂-αC1s scFv] nucleic acid sequence
(construct #6, FIG. 2A)
ATGGAAGCCC CTGCCCAGCT GCTGTTCCTG CTGCTGCTGT GGCTACCTGA TACCACCGGC
GAGGTGCAGC TGGTCGAGAG CGGCGGGGGC CTGGTGAAAC CAGGAGGAAG CCTGAGACTG
AGCTGCGCCG CCTCTGGCTT CACCTTCAGC AATTACGCTA TGAGCTGGGT CAGACAGGCC
CCAGGAAAAA GACTGGAATG GGTGGCCACA ATTTCTAACC GGGGCTCCTA CACCTACTAT
CCTGACAGCG TGAAGGGCAG ATTCACAATC AGCCGGGACA ACGCCAAGAA CAGCCTGTAC
CTGCAGATGA ACAGCCTCAG AGCCGAGGAC ACCGCCCTGT ACTACTGCGC CAGAGAGCGG
CCTATGGACT ACTGGGGCCA AGGCACACTG GTCACAGTTT CCAGCGGCGG CGGCGGCAGC
GGTGGCGGCG GCAGCGGAGG CGGTGGCTCT GATATCCAGA TGACCCAGTC CCCTAGCACC
CTGTCTGCCT CTGTGGGCGA CAGAGTGACC ATTACATGCA AGGCCTCTCA GGACGTGGGC
ACCGCTGTGG CCTGGTATCA GCAGAAACCC GGCAAGGCTC CCAAGCTGCT GATCTACTGG
GCCAGCACAA GACACACAGG CGTGCCTGAT AGATTCAGCG GCAGCGGTAG CGGCACCGAC
TTCACCCTGA CAATCAGCTC CCTCCAGGCT GAAGATTTTG CCGTGTACTT CTGCCACCAG
CATAGCAGCA ACCCCCTGAC ATTCGGCCAG GGCACAAAGC TGGAAATCAA GGGAGGCGGC
GGCTCTGGAG GCGGCGGAAG CCAAGTGCAG CTGGTGCAAA GCGGCGCCGA GGTGAAAAAG
CCCGGCGCAT CTGTGAAGCT GAGTTGTACA GCTTCTGGAT TTAACATCAA GGACGACTAC
ATCCACTGGG TTAAGCAGGC CCCTGGCCAG GGCCTGGAGT GGATCGGCAG AATCGACCCC
GCTGATGGCC ACACCAAGTA CGCCCCTAAG TTCCAGGTGA AGGTGACCAT CACGGCCGAC
ACCAGCACAA GCACCGCCTA CCTGGAACTG AGCAGCCTGC GGAGCGAGGA CACCGCCGTG
TACTACTGTG CCAGATACGG CTACGGCCGC GAGGTGTTCG ACTACTGGGG ACAAGGAACA
ACCGTGACCG TGTCCAGCGG CGGCGGCGGC AGCGGCGGAG GAGGCTCTGG CGGCGGCGGC
AGCGACATCG TGCTGACCCA GAGCCCCGAT TCTCTGGCCG TGAGCCTGGG AGAGAGAGCC
ACCATCTCCT GCAAGGCTTC CCAATCTGTG GACTATGATG GAGATAGCTA CATGAACTGG
TACCAGCAGA AGCCTGGCCA GCCTCCAAAG ATCCTGATCT ACGACGCCAG CAATCTGGAA
TCCGGCATCC CTGCTCGGTT TAGCGGCAGC GGCTCCGGAA CCGACTTCAC CCTGACCATC
AGCTCTCTGG AGCCTGAGGA TTTCGCCATC TACTACTGCC AGCAGTCCAA CGAAGACCCT
TGGACCTTTG GCGGCGGCAC CAAGGTCGAA ATCAAATGA
SEQ ID NO:57-[αBb scFv-(G₄S)₂-αC1s scFv] amino acid sequence
(construct #6, FIG. 2A) (signal peptide boldfaced)
MEAPAQLLFL LLLWLPDTTG EVOLVESGGG LVKPGGSLRL SCAASGFTES NYAMSWVRQA
PGKRLEWVAT ISNRGSYTYY PDSVKGRETI SRDNAKNSLY LQMNSLRAED TALYYCARER
PMDYWGQGTL VTVSSGGGGS GGGGSGGGGS DIQMTQSPST LSASVGDRVT ITCKASQDVG
TAVAWYQQKP GKAPKLLIYW ASTRHTGVPD RESGSGSGTD FTLTISSLQA EDFAVYFCHQ
HSSNPLTFGQ GTKLEIKGGG GSGGGGSQVQ LVOSGAEVKK PGASVKLSCT ASGENIKDDY
IHWVKQAPGQ GLEWIGRIDP ADGHTKYAPK FQVKVTITAD TSTSTAYLEL SSLRSEDTAV
YYCARYGYGR EVEDYWGQGT TVTVSSGGGG SGGGGSGGGG SDIVLTQSPD SLAVSLGERA
TISCKASQSV DYDGDSYMNW YQQKPGQPPK ILIYDASNLE SGIPARESGS GSGTDETLTI
SSLEPEDFAI YYCQQSNEDP WTFGGGTKVE IK*
SEQ ID NO: 58-[αC1s scFab-(G₄S)₃-αBb scFv] nucleic acid
sequence (construct #15, FIG. 2E)
ATGGAAGCTC CAGCCCAGCT GCTGTTCCTG CTGCTCCTTT GGCTGCCTGA CACAACAGGC
GATATCGTGC TGACCCAGAG CCCTGACAGC CTGGCCGTGT CACTGGGCGA GCGGGCCACG
ATCAGCTGCA AGGCCAGCCA GTCCGTGGAT TACGACGGCG ACAGCTACAT GAACTGGTAT
CAGCAGAAGC CCGGACAGCC TCCCAAGATC CTGATCTACG ACGCCAGCAA CCTGGAAAGC
GGCATCCCTG CCAGATTCAG CGGGTCCGGC AGCGGAACAG ACTTCACCCT GACCATCTCC
AGCCTGGAAC CTGAGGATTT CGCCATCTAC TACTGTCAGC AGAGCAACGA GGATCCTTGG
ACCTTCGGCG GCGGCACCAA GGTCGAGATC AAGAGAACCG TGGCCGCTCC TAGCGTGTTC
ATCTTCCCTC CTTCCGACGA GCAGCTGAAG AGCGGCACCG CCTCTGTGGT GTGCCTACTG
AACAACTTCT ACCCTAGAGA GGCTAAAGTG CAGTGGAAGG TGGACAATGC CCTGCAGAGC
GGCAACAGCC AGGAGTCTGT GACCGAGCAG GACAGCAAGG ACAGCACCTA CAGCCTGTCT
TCCACACTGA CCCTGTCTAA GGCCGACTAC GAGAAGCACA AGGTCTACGC CTGCGAGGTG
ACACACCAGG GCCTGAGCTC CCCCGTGACC AAAAGCTTCA ACAGAGGAGA ATGCGGCGGA
GGCGGAAGCG GCGGCGGGGG CTCTGGAGGC GGCGGCTCCG GCGGCGGAGG CAGCGGAGGT
GGCGGCTCTG GCGGCGGCGG CTCCGGAGGC GGCGGCTCAC AGGTGCAGCT GGTGCAATCT
GGTGCTGAGG TGAAGAAGCC AGGCGCCAGC GTGAAGCTAA GCTGCACCGC CTCCGGTTTC
AACATCAAAG ACGACTACAT CCACTGGGTG AAACAGGCCC CAGGCCAGGG CCTGGAGTGG
ATCGGCAGAA TCGACCCTGC CGATGGCCAC ACCAAGTACG CTCCTAAGTT CCAGGTCAAG
GTGACAATCA CCGCAGATAC CAGCACAAGC ACCGCCTACC TGGAGCTGAG CTCGCTGAGA
AGCGAGGACA CAGCCGTGTA CTACTGCGCC AGATACGGCT ACGGAAGAGA GGTGTTTGAT
TACTGGGGAC AGGGCACTAC CGTGACCGTG AGCTCCGCCA GCACCAAGGG CCCTAGCGTG
TTCCCCCTGG CCCCATGTTC TAGATCTACA TCTGAAAGCA CCGCTGCTCT GGGCTGCCTG
GTAAAGGACT ACTTCCCCGA GCCCGTGACC GTGTCCTGGA ACAGCGGCGC CCTGACCTCT
GGCGTGCATA CATTTCCTGC CGTGCTGCAG AGCTCAGGCC TGTACTCCCT GAGCTCTGTC
GTTACAGTGC CCAGCAGCTC CCTGGGAACA AAGACCTACA CCTGCAACGT GGACCACAAG
CCTAGCAATA CCAAGGTGGA CAAGCGGGTG GGGGGCGGTG GATCCGGCGG AGGCGGGAGC
GGCGGCGGAG GATCCGAGGT GCAGCTGGTC GAATCCGGCG GGGGCCTGGT GAAACCCGGC
GGCTCTCTGA GGCTGTCCTG CGCCGCTAGC GGCTTTACCT TTAGCAACTA CGCTATGAGC
TGGGTTAGAC AGGCCCCTGG CAAGCGGCTC GAATGGGTCG CAACAATTTC TAATAGAGGC
AGTTACACAT ACTACCCCGA CTCTGTGAAG GGCCGGTTCA CCATTAGCAG AGATAACGCC
AAGAACTCTC TGTACCTGCA GATGAATTCA CTGCGGGCCG AGGACACCGC CCTGTATTAT
TGTGCTCGGG AACGTCCTAT GGACTACTGG GGCCAGGGCA CCCTGGTGAC AGTGTCCTCT
GGCGGCGGCG GCAGCGGCGG TGGCGGCAGC GGCGGCGGCG GTAGCGACAT CCAGATGACC
CAAAGCCCCA GCACCCTGTC TGCCAGCGTG GGTGACAGAG TGACCATCAC CTGTAAAGCC
TCCCAGGATG TGGGAACAGC CGTTGCCTGG TACCAGCAAA AACCTGGCAA GGCCCCTAAG
CTGCTGATCT ACTGGGCCAG CACCCGCCAC ACTGGCGTGC CTGATCGGTT CAGCGGAAGC
GGCAGCGGAA CAGATTTTAC ACTGACTATC AGCTCCCTCC AGGCCGAAGA TTTCGCCGTG
TACTTCTGCC ACCAGCACAG CAGCAACCCT CTGACCTTCG GACAAGGGAC AAAACTCGAA
ATCAAGTGAG
SEQ ID NO: 59-[αC1s scFab-(G₄S)₃-αBb scFv] amino acid sequence
(construct #15, FIG. 2E) (signal Peptide boldfaced)
MEAPAQLLFL LLLWLPDTTG DIVLTQSPDS LAVSLGERAT ISCKASQSVD YDGDSYMNWY
QQKPGOPPKI LIYDASNLES GIPARESGSG SGTDFTLTIS SLEPEDFAIY YCQQSNEDPW
TFGGGTKVEI KRTVAAPSVE IFPPSDEQLK SGTASVVCLL NNFYPREAKV QWKVDNALQS
GNSQESVTEQ DSKDSTYSLS STLTLSKADY EKHKVYACEV THQGLSSPVT KSENRGECGG
GGSGGGGSGG GGSGGGGSGG GGSGGGGSGG GGSQVOLVQS GAEVKKPGAS VKLSCTASGE
NIKDDYIHWV KQAPGQGLEW IGRIDPADGH TKYAPKFQVK VTITADTSTS TAYLELSSLR
SEDTAVYYCA RYGYGREVED YWGQGTTVTV SSASTKGPSV FPLAPCSRST SESTAALGCL
VKDYFPEPVT VSWNSGALTS GVHTFPAVLQ SSGLYSLSSV VTVPSSSLGT KTYTCNVDHK
PSNTKVDKRV GGGGSGGGGS GGGGSEVQLV ESGGGLVKPG GSLRLSCAAS GFTFSNYAMS
WVRQAPGKRL EWVATISNRG SYTYYPDSVK GRFTISRDNA KNSLYLOMNS LRAEDTALYY
CARERPMDYW GQGTLVTVSS GGGGSGGGGS GGGGSDIQMT QSPSTLSASV GDRVTITCKA
SQDVGTAVAW YQQKPGKAPK LLIYWASTRH TGVPDRESGS GSGTDETLTI SSLQAEDFAV
YFCHQHSSNP LTFGQGTKLE IK*
SEQ ID NO: 60-[αC1s scFab-(G₄S)₃-αBb scFv-CM] nucleic acid
sequence (construct #16, FIG. 2E)
ATGGAAGCCC CTGCCCAGCT GCTGTTCCTG CTGCTGCTGT GGCTGCCTGA CACAACCGGC
GACATCGTGC TGACACAGAG CCCCGACAGC CTCGCCGTTT CCCTCGGCGA GCGGGCCACA
ATCTCATGCA AGGCCTCACA GTCCGTGGAC TATGACGGCG ATAGCTACAT GAACTGGTAC
CAGGAGAAGC CTGGCCAACC TCCAAAGATC CTGATCTACG ACGCCAGCAA TCTGGAATCC
GGTATTCCTG CCAGATTCAG CGGCTCTGGA TCCGGCACCG ACTTTACTCT GACCATCAGC
TCTCTGGAAC CTGAGGACTT TGCTATCTAC TACTGCCAGC AGAGCAACGA GGACCCCTGG
ACCTTCGGCG GCGGCACCAA AGTGGAAATC AAGCGGACCG TGGCCGCTCC TTCAGTGTTC
ATCTTCCCAC CTTCCGACGA GCAGCTGAAG AGCGGCACCG CCAGCGTGGT GTGCCTGCTG
AACAACTTCT ACCCCAGAGA GGCTAAGGTG CAGTGGAAGG TGGATAACGC TCTGCAAAGT
GGCAACTCTC AGGAGTCTGT GACAGAGCAG GACTCCAAGG ACAGCACCTA CAGCCTGTCC
TCTACCCTGA CACTGTCCAA GGCCGACTAC GAGAAGCACA AGGTGTACGC CTGTGAAGTG
ACACACCAGG GGCTGAGCTC CCCTGTGACA AAATCTTTCA ACCGGGGCGA GTGCGGCGGA
GGAGGCAGCG GCGGCGGCGG CAGCGGGGGC GGAGGCTCCG GCGGCGGCGG TAGCGGTGGG
GGCGGATCTG GAGGCGGGGG ATCGGGCGGA GGCGGCAGCC AGGTGCAGCT GGTCCAGAGC
GGCGCCGAGG TGAAAAAGCC AGGCGCCTCT GTGAAGCTGT CTTGCACCGC CTCTGGTTTT
AATATCAAGG ACGACTACAT CCACTGGGTG AAGAAGGCTC CAGGTCAAGG ACTGGAATGG
ATCGGCCGGA TCGACCCCGC TGATGGCCAC ACCAAATACG CTCCTAAGTT CCAGGTGAAA
GTTACAATTA CAGCCGATAC CAGCACAAGC ACCGCCTACC TGGAGCTGAG CTCTCTGAGA
AGCGAAGATA CAGCCGTGTA CTACTGCGCA AGATACGGCT ACGGCAGAGA GGTGTTCGAC
TATTGGGGAC AGGGCACCAC AGTGACCGTG TCTAGTGCCA GCACCAAGGG CCCCAGCGTG
TTCCCTCTGG CCCCTTGTAG CAGATCTACC AGCGAGTCCA CCGCTGCTCT GGGCTGCCTG
GTCAAGGATT ACTTCCCCGA GCCTGTGACC GTTAGCTGGA ACAGCGGAGC CCTGACCAGC
GGCGTGCACA CCTTTCCAGC CGTGCTGCAG AGCAGCGGAC TGTATAGCCT GAGCAGCGTC
GTGACAGTGC CCAGCAGCAG CCTGGGCACC AAGACCTACA CCTGCAACGT GGACCACAAG
CCCAGCAACA CCAAGGTGGA CAAGAGAGTG GGCGGCGGAG GCTCTGGCGG CGGCGGCTCT
GGGGGCGGCG GAAGCGAGGT GCAGCTGGTG GAATCTGGCG GCGGACTGGT GAAGCCTGGC
GGCAGCCTGA GACTGAGCTG CGCCGCCAGC GGCTTCACCT TCAGCAACTA CGCCATGAGC
TGGGTTAGAG AAGCCCCTGG AAAAAGACTG GAATGGGTGG CCACCATCTC TAATAGAGGA
TCTTATACAT ACTACCCTGA TTCTGTGAAA GGACGGTTCA CAATCTCCCG CGACAACGCC
AAGAACTCAC TGTACCTGCA GATGAACTCT CTGAGGGCCG AGGATACCGC CCTGTACTAC
TGTGCCCGAG AAAGACCTAT GGATTACTGG GGCCAGGGCA CCCTCGTCAC AGTTTCCTCT
GGCGGGGGCG GTAGCGGCGG CGGCGGATCC GGCGGAGGTG GCAGCGACAT CCAGATGACC
CAAAGCCCTT CTACACTGAG CGCCAGCGTC GGCGACCGGG TGACCATCAC CTGTAAAGCC
AGCCAAGACG TGGGCACGGC TGTGGCTTGG TATCAGAAGA AACCTGGCAA GGCCCCCAAG
CTGCTTATCT ACTGGGCCAG CACAAGACAC ACAGGCGTTC CTGATAGATT CAGCGGCAGC
GGCTCCGGCA CAGATTTCAC CCTGACCATC TCGAGTCTGC AGGCCGAGGA TTTCGCCGTG
TACTTCTGCC ACCAGCATTC TTCTAACCCT CTGACCTTTG GCCAGGGAAC CAAGCTGGAA
ATCAAGTGA
SEQ ID NO: 61-SEQ ID NO: 60-[αC1s scFab-(G₄S)₃-αBb scFv-CM]
amino acid sequence (construct #16, FIG. 2E) (signal peptide
boldfaced; charge mutations boxed and italicized, numbering
excluding signal peptide: Q42E and Q292K in αC1s scFab, and Q524E
and Q653K in aBb scFv)
MEAPAQLLFL LLLWLPDTTG DIVLTOSPDS LAVSLGERAT ISCKASQSVD YDGDSYMNWY

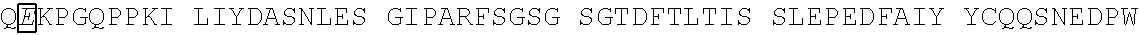

TFGGGTKVEI KRTVAAPSVE IFPPSDEQLK SGTASVVCLL NNFYPREAKV QWKVDNALOS
GNSQESVTEQ DSKDSTYSLS STLTLSKADY EKHKVYACEV THQGLSSPVT KSENRGECGG
GGSGGGGSGG GGSGGGGSGG GGSGGGGSGG GGSQVOLVOS GAEVKKPGAS VKLSCTASGF

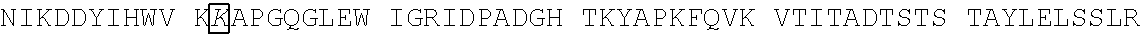

SEDTAVYYCA RYGYGREVED YWGQGTTVTV SSASTKGPSV FPLAPCSRST SESTAALGCL
VKDYFPEPVT VSWNSGALTS GVHTFPAVLQ SSGLYSLSSV VTVPSSSLGT KTYTCNVDHK
PSNTKVDKRV GGGGSGGGGS GGGGSEVQLV ESGGGLVKPG GSLRLSCAAS GFTESNYAMS

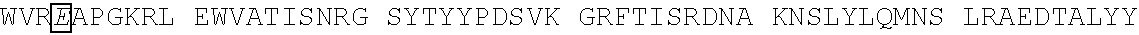

CARERPMDYW GQGTLVTVSS GGGGSGGGGS GGGGSDIQMT QSPSTLSASV GDRVTITCKA

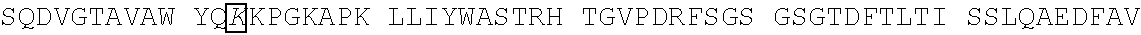

YFCHQHSSNP LTFGQGTKLE IK*
SEQ ID NO: 62-[αC1s scFab-BiDir-αBb scFab-CM] nucleic acid
sequence (construct #21, FIG. 2G)
TCACACCCGC TTATCCACCT TGGTGTTGCT GGGCTTGTGG TCCACGTTGC AGGTGTAGGT
CTTTGTGCCC AGGCTAGAGC TAGGCACTGT CACGACAGAG GACAGAGAGT ACAGGCCGCT
GCTCTGCAGC ACGGCGGGGA AGGTGTGCAC CCCGCTTGTC AGGGCTCCGC TGTTCCAGGA
CACGGTCACA GGCTCAGGGA AATAATCCTT GACCAGGCAG CCCAGAGCAG CCGTGCTCTC
TGAGGTACTT CTGCTACAAG GAGCCAGTGG GAACACGCTA GGGCCCTTTG TGCTGGCGGA
CGACACGGTC ACTGTTGTGC CCTGTCCCCA GTAGTCGAAC ACTTCTCTGC CGTAGCCGTA
TCTGGCGCAG TAGTACACAG CGGTGTCCTC GGATCTAAGG CTGCTCAGTT CCAGATAAGC
TGTAGAGGTG CTGGTATCGG CGGTGATGGT GACTTTCACC TGGAACTTAG GGGCGTACTT
TGTGTGGCCG TCGGCAGGGT CGATTCTGCC GATCCACTCC AGTCCCTGGC CGGGGGCCTT
CTTCACCCAG TGGATGTAAT CGTCCTTGAT ATTGAAGCCG CTGGCGGTGC AGCTCAGCTT
AACACTAGCG CCAGGCTTTT TCACCTCGGC TCCGCTCTGC ACCAGCTGCA CCTGGGATCC
GCCGCCGCCG CTGCCGCCTC CGCCGCTGCC GCCTCCGCCG CTTCCGCCTC CCCCAGAGCC
GCCGCCACCG CTGCCTCCTC CGCCGGAGCC GCCGCCGCCG CACTCGCCCC GGTTGAAGCT
TTTGGTCACA GGAGAGGACA GGCCCTGATG TGTCACTTCA CAGGCGTACA CCTTGTGCTT
CTCGTAGTCG GCCTTGCTCA AGGTCAGGGT GCTGGACAGG CTGTATGTTG AGTCCTTGCT
GTCCTGCTCG GTCACGCTCT CTTGGCTGTT GCCGCTTTGC AGGGCGTTGT CAACTTTCCA
TTGGACCTTT GCCTCTCTGG GGTAGAAGTT ATTCAGCAGG CACACCACAG AGGCGGTTCC
GCTCTTCAGC TGCTCGTCGC TTGGAGGGAA GATAAAGACA GAAGGGGCGG CCACGGTGCG
CTTGATTTCC ACCTTGGTGC CGCCTCCAAA GGTCCAGGGG TCCTCGTTGC TCTGCTGGCA
GTAGTAGATG GCAAAATCCT CGGGTTCCAG AGAAGAAATT GTCAGGGTGA AATCAGTGCC
AGAGCCGCTG CCGCTGAATC TGGCGGGGAT GCCGCTTTCC AGATTGCTGG CGTCGTAGAT
CAGGATTTTT GGAGGCTGGC CGGGTTTCTC CTGGTACCAG TTCATGTAGC TGTCGCCGTC
ATAGTCCACG CTCTGAGAGG CTTTACAGCT GATTGTGGCC CGTTCGCCGA GGCTCACGGC
CAGGCTATCA GGGCTCTGCG TCAGCACGAT ATCGCCGGTG GTGTCAGGCA GCCACAGGAG
CAGCAGGAAC AGCAGCTGGG CAGGGGCTTC CATGGTGGGC TCTGGCGCCC GCCGCGCGCT
TCGCTTTTTA TAGGGCCGCC GCCGCCGCCG CCTCGCCATA AAAGGAAACT TTCGGAGCGC
GCCGCTCTGA TTGGCTGCCG CCGCACCTCT CCGCCTCGCC CCGCCCCGCC CCTCGCCCCG
CCCCGCCCCG CCTGGCGCGC GCCCCCCCCC CCCCCCCGCC CCCATCGCTG CACAAAATAA
TTAAAAAATA AATAAATACA AAATTGGGGG TGGGGAGGGG GGGGAGATGG GGAGAGTGAA
GCAGAACGTG GGGCTCACCT CGCTAGTTAT TAATAGTAAT CAATTACGGG GTCATTAGTT
CATAGCCCAT ATATGGAGTT CCGCGTTACA TAACTTACGG TAAATGGCCC GCCTGGCTGA
CCGCCCAACG ACCCCCGCCC ATTGACGTCA ATAATGACGT ATGTTCCCAT AGTAACGCCA
ATAGGGACTT TCCATTGACG TCAATGGGTG GAGTATTTAC GGTAAACTGC CCACTTGGCA
GTACATCAAG TGTATCATAT GCCAAGTACG CCCCCTATTG ACGTCAATGA CGGTAAATGG
CCCGCCTGGC ATTATGCCCA GTACATGACC TTATGGGACT TTCCTACTTG GCAGTACATC
TACGTATTAG TCATCGCTAT TACCATGGTC GAGGTGAGCC CCACGTTCTG CTTCACTCTC
CCCATCTCCC CCCCCTCCCC ACCCCCAATT TTGTATTTAT TTATTTTTTA ATTATTTTGT
GCAGCGATGG GGGCGGGGGG GGGGGGGGGG CGCGCGCCAG GCGGGGGGGG GCGGGGCGAG
GGGCGGGGCG GGGCGAGGCG GAGAGGTGCG GCGGCAGCCA ATCAGAGCGG CGCGCTCCGA
AAGTTTCCTT TTATGGCGAG GCGGCGGCGG CGGCGGCCCT ATAAAAAGCG AAGCGCGCGG
CGGGCGCCAA CTAGCCCACC ATGGAAGCCC CCGCTCAGCT GCTGTTCCTG CTGCTGCTGT
GGCTGCCTGA CACCACCGGC GACATCCAGA TGACACAGAG CCCTAGCACC CTGAGCGCCT
CCGTGGGGGA CAGAGTGACA ATCACATGTA AAGCCTCCCA GGACGTGGGC ACTGCCGTGG
CCTGGTACCA GAAAAAACCG GGAAAAGCCC CTAAGCTGCT GATCTACTGG GCCAGCACCA
GACACACCGG CGTCCCCGAT AGATTCAGCG GCTCTGGCAG CGGAACTGAT TTCACCCTGA
CCATTTCTTC TCTGCAGGCC GAGGACTTCG CCGTGTACTT TTGCCACCAG CACAGCAGCA
ACCCTCTGAC CTTCGGACAG GGCACAAAGC TGGAAATCAA GCGGACAGTG GCTGCTCCTT
CTGTGTTCAT CTTTCCACCT AGCGACGAGC AGCTGAAGAG CGGCACCGCC TCTGTGGTGT
GCCTGCTGAA CAACTTCTAC CCCAGAGAAG CCAAAGTGCA GTGGAAGGTG GACAACGCCC
TGCAATCTGG CAACAGCCAG GAGAGCGTGA CGGAACAAGA TAGCAAGGAC AGCACCTACT
CCCTGAGCAG CACACTGACC TTGTCCAAGG CAGATTACGA GAAGCACAAG GTGTACGCCT
GCGAGGTGAC CCACCAGGGA CTGAGCAGCC CAGTGACCAA GAGCTTCAAC AGAGGAGAGT
GCGGCGGCGG CGGAAGCGGA GGCGGAGGCA GCGGCGGCGG CGGCAGTGGA GGCGGCGGCT
CTGGCGGAGG GGGCAGTGGC GGTGGCGGAT CCGGCGGCGG CGGCAGCGAG GTGCAGCTTG
TGGAATCCGG CGGCGGCCTG GTGAAGCCCG GCGGTAGCCT GAGACTGTCT TGTGCCGCCT
CTGGCTTCAC CTTTAGCAAT TACGCCATGA GCTGGGTGCG GGAGGCTCCC GGCAAAAGAC
TGGAATGGGT CGCCACCATC AGCAACCGGG GATCATATAC CTACTACCCT GATAGCGTGA
AAGGCAGGTT CACAATCAGC CGGGACAATG CCAAGAACAG CCTGTACCTG CAGATGAACT
CACTGCGGGC CGAGGACACC GCCCTGTATT ACTGCGCCAG AGAGAGACCT ATGGACTACT
GGGGCCAGGG CACCCTGGTG ACCGTTTCCT CCGCCAGCAC CAAGGGCCCT AGCGTGTTCC
CTCTGGCCCC ATGCAGCAGA AGCACATCTG AGAGCACCGC CGCTCTGGGC TGCCTGGTGA
AGGACTACTT CCCCGAGCCT GTGACAGTGA GCTGGAACTC CGGCGCCCTG ACCAGCGGCG
TGCACACATT TCCAGCTGTG CTGCAGTCTA GCGGCCTGTA CAGCCTGAGC AGCGTTGTGA
CAGTGCCTTC TAGCAGCCTC GGCACCAAGA CCTACACCTG TAACGTGGAT CATAAGCCTT
CTAATACCAA GGTTGACAAG AGAGTGTGA
SEQ ID NO: 63-αC1s scFab-CM arm (construct #22, FIG.2G) (signal
sequence boldfaced; charge mutations boxed and italicized, numbering
excluding signal peptide: Q42E and Q292K)
MEAPAQLLFL LLLWLPDTTG DIVLTQSPDS LAVSLGERAT ISCKASQSVD YDGDSYMNWY

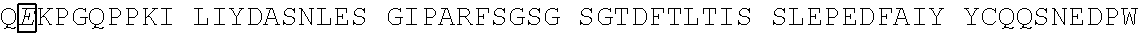

TFGGGTKVEI KRTVAAPSVE IFPPSDEQLK SGTASVVCLL NNFYPREAKV QWKVDNALQS
GNSQESVTEQ DSKDSTYSLS STLTLSKADY EKHKVYACEV THQGLSSPVT KSENRGECGG
GGSGGGGSGG GGSGGGGSGG GGSGGGGSGG GGSQVQLVQS GAEVKKPGAS VKLSCTASGF

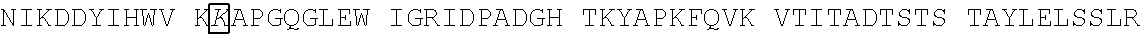

SEDTAVYYCA RYGYGREVED YWGQGTTVTV SSASTKGPSV FPLAPCSRST SESTAALGCL
VKDYFPEPVT VSWNSGALTS GVHTFPAVLQ SSGLYSLSSV VTVPSSSLGT KTYTCNVDHK
PSNTKVDKRV*
SEQ ID NO: 64-αBb scFab-CM arm (construct #22, FIG. 2G) (signal
sequence boldfaced; charge mutations boxed and italicized, numbering
excluding signal peptide: Q38K and Q288E, and S114A, N137K, and
T434E)

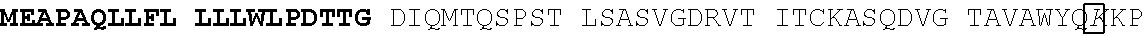

GKAPKLLIYW ASTRHTGVPD RFSGSGSGTD FTLTISSLQA EDFAVYFCHQ HSSNPLTFGQ

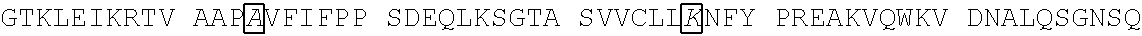

ESVTEQDSKD STYSLSSTLT LSKADYEKHK VYACEVTHQG LSSPVTKSEN RGECGGGGSG
GGGSGGGGSG GGGSGGGGSG GGGSGGGGSE VOLVESGGGL VKPGGSLRLS CAASGFTFSN

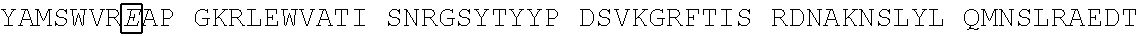

ALYYCARERP MDYWGQGTLV TVSSASTKGP SVEPLAPCSR STSESTAALG CLVKDYFPEP

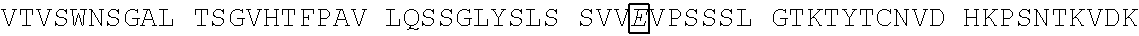

VTVSWNSGAL
SEQ ID NO: 65-Human complement C1s amino acid sequence prior to
processingand activation (signalsequence boldfaced)
MWCIVLFSLL AWVYAEPTMY GEILSPNYPQ AYPSEVEKSW DIEVPEGYGI HLYFTHLDIE
LSENCAYDSV QIISGDTEEG RLCGQRSSNN PHSPIVEEFQ VPYNKLQVIF KSDESNEERE
TGFAAYYVAT DINECTDEVD VPCSHFCNNF IGGYFCSCPP EYFLHDDMKN CGVNCSGDVE
TALIGEIASP NYPKPYPENS RCEYQIRLEK GFQVVVTLRR EDEDVEAADS AGNCLDSLVE
VAGDRQFGPY CGHGFPGPLN IETKSNALDI IFQTDLTGOK KGWKLRYHGD PMPCPKEDTP
NSVWEPAKAK YVERDVVOIT CLDGFEVVEG RVGATSFYST CQSNGKWSNS KLKCQPVDCG
IPESIENGKV EDPESTLEGS VIRYTCEEPY YYMENGGGGE YHCAGNGSWV NEVLGPELPK
CVPVCGVPRE PFEEKQRIIG GSDADIKNEP WQVFFDNPWA GGALINEYWV LTAAHVVEGN
REPTMYVGST SVQTSRLAKS KMLTPEHVFI HPGWKLLEVP EGRTNEDNDI ALVRLKDPVK
MGPTVSPICL PGTSSDYNLM DGDLGLISGW GRTEKRDRAV RLKAARLPVA PLRKCKEVKV
EKPTADAEAY VFTPNMICAG GEKGMDSCKG DSGGAFAVOD PNDKTKFYAA GLVSWGPQCG
TYGLYTRVKN YVDWIMKTMQ ENSTPRED
SEQ ID NO: 66-Human complement factor B prior to processing and
activation (signal peptide boldfaced)
MGSNLSPQLC LMPFILGLLS GGVTTTPWSL ARPQGSCSLE GVEIKGGSER LLQEGQALEY
VCPSGFYPYP VOTRTCRSTG SWSTLKTQDQ KTVRKAECRA IHCPRPHDFE NGEYWPRSPY
YNVSDEISFH CYDGYTLRGS ANRTCQVNGR WSGQTAICDN GAGYCSNPGI PIGTRKVGSQ
YRLEDSVTYH CSRGLTLRGS QRRTCQEGGS WSGTEPSCQD SFMYDTPQEV AEAFLSSLTE
TIEGVDAEDG HGPGEQQKRK IVLDPSGSMN IYLVLDGSDS IGASNETGAK KCLVNLIEKV
ASYGVKPRYG LVTYATYPKI WVKVSEADSS NADWVTKQLN EINYEDHKLK SGTNTKKALQ
AVYSMMSWPD DVPPEGWNRT RHVIILMTDG LHNMGGDPIT VIDEIRDLLY IGKDRKNPRE
DYLDVYVFGV GPLVNQVNIN ALASKKDNEQ HVFKVKDMEN LEDVFYQMID ESQSLSLCGM
VWEHRKGTDY HKQPWQAKIS VIRPSKGHES CMGAVVSEYF VLTAAHCFTV DDKEHSIKVS
VGGEKRDLEI EVVLFHPNYN INGKKEAGIP EFYDYDVALI KLKNKLKYGQ TIRPICLPCT
EGTTRALRLP PTTTCQQQKE ELLPAQDIKA LEVSEEEKKL TRKEVYIKNG DKKGSCERDA
QYAPGYDKVK DISEVVTPRF LCTGGVSPYA DPNTCRGDSG GPLIVHKRSR FIQVGVISWG
VVDVCKNQKR QKOVPAHARD FHINLFQVLP WLKEKLQDED LGEL
SEQ ID NO: 67-HCDR1 of anti-C1s antibody (IMGT ®)
GENIKDDY
SEQ ID NO: 68-HCDR2 of anti-C1s antibody (IMGT ®)
IDPADGHT
SEQ ID NO: 69-HCDR3 of anti-C1s antibody (IMGT ®)
ARYGYGREVEDY
SEQ ID NO:70-LCDR1 of anti-C1s antibody (IMGT ®)
QSVDYDGDSY
SEQ ID NO:71-HCDR1 of anti-C1s antibody (Chothia)
GFNIKDD
SEQ ID NO: 72-HCDR2 of anti-C1s antibody (Chothia)
DPADGH
SEQ ID NO: 73-HCDR1 of anti-Bb antibody (IMGR ®)
GFTFSNYA
SEQ ID NO:74-HCDR2 of anti-Bb antibody (IMGR ®)
ISNRGSYT
SEQ ID NO: 75-HCDR3 of anti-Bb antibody (IMGR ®)
ARERPMDY
SEQ ID NO: 76-LCDR1 of anti-Bb antibody (IMGR ®)
QDVGTA
SEQ ID NO: 77-HCDR1 of anti-Bb antibody (Chothia)
GFTFSNY
SEQ ID NO: 78-HCDR2 of anti-Bb antibody (Chothia)
SNRGSY
SEQ ID NO: 79-[αBb scFab-BiDir-αC1s scFab-CM] nucleic acid
sequence (construct #22, FIG. 2G)
TCACACTCTC TTGTCAACCT TGGTATTAGA AGGCTTATGA TCCACGTTAC AGGTGTAGGT
CTTGGTGCCG AGGCTGCTAG AAGGCACTGT CACAACGCTG CTCAGGCTGT ACAGGCCGCT
AGACTGCAGC ACAGCTGGAA ATGTGTGCAC GCCGCTGGTC AGGGCGCCGG AGTTCCAGCT
CACTGTCACA GGCTCGGGGA AGTAGTCCTT CACCAGGCAG CCCAGAGCGG CGGTGCTCTC
AGATGTGCTT CTGCTGCATG GGGCCAGAGG GAACACGCTA GGGCCCTTGG TGCTGGCGGA
GGAAACGGTC ACCAGGGTGC CCTGGCCCCA GTAGTCCATA GGTCTCTCTC TGGCGCAGTA
ATACAGGGCG GTGTCCTCGG CCCGCAGTGA GTTCATCTGC AGGTACAGGC TGTTCTTGGC
ATTGTCCCGG CTGATTGTGA ACCTGCCTTT CACGCTATCA GGGTAGTAGG TATATGATCC
CCGGTTGCTG ATGGTGGCGA CCCATTCCAG TCTTTTGCCG GGAGCCTCCC GCACCCAGCT
CATGGCGTAA TTGCTAAAGG TGAAGCCAGA GGCGGCACAA GACAGTCTCA GGCTACCGCC
GGGCTTCACC AGGCCGCCGC CGGATTCCAC AAGCTGCACC TCGCTGCCGC CGCCGCCGGA
TCCGCCACCG CCACTGCCCC CTCCGCCAGA GCCGCCGCCT CCACTGCCGC CGCCGCCGCT
GCCTCCGCCT CCGCTTCCGC CGCCGCCGCA CTCTCCTCTG TTGAAGCTCT TGGTCACTGG
GCTGCTCAGT CCCTGGTGGG TCACCTCGCA GGCGTACACC TTGTGCTTCT CGTAATCTGC
CTTGGACAAG GTCAGTGTGC TGCTCAGGGA GTAGGTGCTG TCCTTGCTAT CTTGTTCCGT
CACGCTCTCC TGGCTGTTGC CAGATTGCAG GGCGTTGTCC ACCTTCCACT GCACTTTGGC
TTCTCTGGGG TAGAAGTIGT TCAGCAGGCA CACCACAGAG GCGGTGCCGC TCTTCAGCTG
CTCGTCGCTA GGTGGAAAGA TGAACACAGA AGGAGCAGCC ACTGTCCGCT TGATTTCCAG
CTTTGTGCCC TGTCCGAAGG TCAGAGGGTT GCTGCTGTGC TGGTGGCAAA AGTACACGGC
GAAGTCCTCG GCCTGCAGAG AAGAAATGGT CAGGGTGAAA TCAGTTCCGC TGCCAGAGCC
GCTGAATCTA TCGGGGACGC CGGTGTGTCT GGTGCTGGCC CAGTAGATCA GCAGCTTAGG
GGCTTTTCCC GGTTTTTTCT GGTACCAGGC CACGGCAGTG CCCACGTCCT GGGAGGCTTT
ACATGTGATT GTCACTCTGT CCCCCACGGA GGCGCTCAGG GTGCTAGGGC TCTGTGTCAT
CTGGATGTCG CCGGTGGTGT CAGGCAGCCA CAGCAGCAGC AGGAACAGCA GCTGAGCGGG
GGCTTCCATG GTGGGCTAGT TGGCGCCCGC CGCGCGCTTC GCTTTTTATA GGGCCGCCGC
CGCCGCCGCC TCGCCATAAA AGGAAACTTT CGGAGCGCGC CGCTCTGATT GGCTGCCGCC
GCACCTCTCC GCCTCGCCCC GCCCCGCCCC TCGCCCCGCC CCGCCCCGCC TGGCGCGCGC
CCCCCCCCCC CCCCCGCCCC CATCGCTGCA CAAAATAATT AAAAAATAAA TAAATACAAA
ATTGGGGGTG GGGAGGGGGG GGAGATGGGG AGAGTGAAGC AGAACGTGGG GCTCACCTCG
ACCATGGTAA TAGCGATGAC TAATACGTAG ATGTACTGCC AAGTAGGAAA GTCCCATAAG
GTCATGTACT GGGCATAATG CCAGGCGGGC CATTTACCGT CATTGACGTC AATAGGGGGC
GTACTTGGCA TATGATACAC TTGATGTACT GCCAAGTGGG CAGTTTACCG TAAATACTCC
ACCCATTGAC GTCAATGGAA AGTCCCTATT GGCGTTACTA TGGGAACATA CGTCATTATT
GACGTCAATG GGCGGGGGTC GTTGGGCGGT CAGCCAGGCG GGCCATTTAC CGTAAGTTAT
GTAACGCGGA ACTCCATATA TGGGCTATGA ACTAATGACC CCGTAATTGA TTACTATTAA
TAACTAGCGA GGTGAGCCCC ACGTTCTGCT TCACTCTCCC CATCTCCCCC CCCTCCCCAC
CCCCAATTTT GTATTTATTT ATTTTTTAAT TATTTTGTGC AGCGATGGGG GCGGGGGGGG
GGGGGGGGCG CGCGCCAGGC GGGGGGGGGC GGGGCGAGGG GCGGGGGGGG GCGAGGCGGA
GAGGTGCGGC GGCAGCCAAT CAGAGCGGCG CGCTCCGAAA GTTTCCTTTT ATGGCGAGGC
GGCGGCGGCG GCGGCCCTAT AAAAAGCGAA GCGCGCGGCG GGCGCCAGAG CCCACCATGG
AAGCCCCTGC CCAGCTGCTG TTCCTGCTGC TCCTGTGGCT GCCTGACACC ACCGGCGATA
TCGTGCTGAC GCAGAGCCCT GATAGCCTGG CCGTGAGCCT CGGCGAACGG GCCACAATCA
GCTGTAAAGC CTCTCAGAGC GTGGACTATG ACGGCGACAG CTACATGAAC TGGTACCAGG
AGAAACCCGG CCAGCCTCCA AAAATCCTGA TCTACGACGC CAGCAATCTG GAAAGCGGCA
TCCCCGCCAG ATTCAGCGGC AGCGGCTCTG GCACTGATTT CACCCTGACA ATTTCTTCTC
TGGAACCCGA GGATTTTGCC ATCTACTACT GCCAGCAGAG CAACGAGGAC CCCTGGACCT
TTGGAGGCGG CACCAAGGTG GAAATCAAGC GCACCGTGGC CGCCCCTTCT GTCTTTATCT
TCCCTCCAAG CGACGAGCAG CTGAAGAGCG GAACCGCCTC TGTGGTGTGC CTGCTGAATA
ACTTCTACCC CAGAGAGGCA AAGGTCCAAT GGAAAGTTGA CAACGCCCTG CAAAGCGGCA
ACAGCCAAGA GAGCGTGACC GAGCAGGACA GCAAGGACTC AACATACAGC CTGTCCAGCA
CCCTGACCTT GAGCAAGGCC GACTACGAGA AGCACAAGGT GTACGCCTGT GAAGTGACAC
ATCAGGGCCT GTCCTCTCCT GTGACCAAAA GCTTCAACCG GGGCGAGTGC GGCGGCGGCG
GCTCCGGCGG AGGAGGCAGC GGTGGCGGCG GCTCTGGGGG AGGCGGAAGC GGCGGAGGCG
GCAGCGGCGG AGGCGGCAGC GGCGGCGGCG GATCCCAGGT GCAGCTGGTG CAGAGCGGAG
CCGAGGTGAA AAAGCCTGGC GCTAGTGTTA AGCTGAGCTG CACCGCCAGC GGCTTCAATA
TCAAGGACGA TTACATCCAC TGGGTGAAGA AGGCCCCCGG CCAGGGACTG GAGTGGATCG
GCAGAATCGA CCCTGCCGAC GGCCACACAA AGTACGCCCC TAAGTTCCAG GTGAAAGTCA
CCATCACCGC CGATACCAGC ACCTCTACAG CTTATCTGGA ACTGAGCAGC CTTAGATCCG
AGGACACCGC TGTGTACTAC TGCGCCAGAT ACGGCTACGG CAGAGAAGTG TTCGACTACT
GGGGACAGGG CACAACAGTG ACCGTGTCGT CCGCCAGCAC AAAGGGCCCT AGCGTGTTCC
CACTGGCTCC TTGTAGCAGA AGTACCTCAG AGAGCACGGC TGCTCTGGGC TGCCTGGTCA
AGGATTATTT CCCTGAGCCT GTGACCGTGT CCTGGAACAG CGGAGCCCTG ACAAGCGGGG
TGCACACCTT CCCCGCCGTG CTGCAGAGCA GCGGCCTGTA CTCTCTGTCC TCTGTCGTGA
CAGTGCCTAG CTCTAGCCTG GGCACAAAGA CCTACACCTG CAACGTGGAC CACAAGCCCA
GCAACACCAA GGTGGATAAG CGGGTGTGA
SEQ ID NO: 80 [αBb scFab-BiDir-αC1s scFab] nucleic acid sequence
(construct #10, FIG. 2B)
TCACACTCTC TTGTCAACCT TGGTATTAGA AGGCTTATGA TCCACGTTAC AGGTGTAGGT
CTTGGTGCCG AGGCTGCTAG AAGGCACTGT CACAACGCTG CTCAGGCTGT ACAGGCCGCT
AGACTGCAGC ACAGCTGGAA ATGTGTGCAC GCCGCTGGTC AGGGCGCCGG AGTTCCAGCT
CACTGTCACA GGCTCGGGGA AGTAGTCCTT CACCAGGCAG CCCAGAGCGG CGGTGCTCTC
AGATGTGCTT CTGCTGCATG GGGCCAGAGG GAACACGCTA GGGCCCTTGG TGCTGGCGGA
GGAAACGGTC ACCAGGGTGC CCTGGCCCCA GTAGTCCATA GGTCTCTCTC TGGCGCAGTA
ATACAGGGCG GTGTCCTCGG CCCGCAGTGA GTTCATCTGC AGGTACAGGC TGTTCTTGGC
ATTGTCCCGG CTGATTGTGA ACCTGCCTTT CACGCTATCA GGGTAGTAGG TATATGATCC
CCGGTTGCTG ATGGTGGCGA CCCATTCCAG TCTTTTGCCG GGAGCCTGCC GCACCCAGCT
CATGGCGTAA TTGCTAAAGG TGAAGCCAGA GGCGGCACAA GACAGTCTCA GGCTACCGCC
GGGCTTCACC AGGCCGCCGC CGGATTCCAC AAGCTGCACC TCGCTGCCGC CGCCGCCGGA
TCCGCCACCG CCACTGCCCC CTCCGCCAGA GCCGCCGCCT CCACTGCCGC CGCCGCCGCT
GCCTCCGCCT CCGCTTCCGC CGCCGCCGCA CTCTCCTCTG TTGAAGCTCT TGGTCACTGG
GCTGCTCAGT CCCTGGTGGG TCACCTCGCA GGCGTACACC TTGTGCTTCT CGTAATCTGC
CTTGGACAAG GTCAGTGTGC TGCTCAGGGA GTAGGTGCTG TCCTTGCTAT CTTGTTCCGT
CACGCTCTCC TGGCTGTTGC CAGATTGCAG GGCGTTGTCC ACCTTCCACT GCACTTTGGC
TTCTCTGGGG TAGAAGTTGT TCAGCAGGCA CACCACAGAG GCGGTGCCGC TCTTCAGCTG
CTCGTCGCTA GGTGGAAAGA TGAACACAGA AGGAGCAGCC ACTGTCCGCT TGATTTCCAG
CTTTGTGCCC TGTCCGAAGG TCAGAGGGTT GCTGCTGTGC TGGTGGCAAA AGTACACGGC
GAAGTCCTCG GCCTGCAGAG AAGAAATGGT CAGGGTGAAA TCAGTTCCGC TGCCAGAGCC
GCTGAATCTA TCGGGGACGC CGGTGTGTCT GGTGCTGGCC CAGTAGATCA GCAGCTTAGG
GGCTTTTCCC GGTTTTTGCT GGTACCAGGC CACGGCAGTG CCCACGTCCT GGGAGGCTTT
ACATGTGATT GTCACTCTGT CCCCCACGGA GGCGCTCAGG GTGCTAGGGC TCTGTGTCAT
CTGGATGTCG CCGGTGGTGT CAGGCAGCCA CAGCAGCAGC AGGAACAGCA GCTGAGCGGG
GGCTTCCATG GTGGGCTAGT TGGCGCCCGC CGCGCGCTTC GCTTTTTATA GGGCCGCCGC
CGCCGCCGCC TCGCCATAAA AGGAAACTTT CGGAGCGCGC CGCTCTGATT GGCTGCCGCC
GCACCTCTCC GCCTCGCCCC GCCCCGCCCC TCGCCCCGCC CCGCCCCGCC TGGCGCGCGC
CCCCCCCCCC CCCCCGCCCC CATCGCTGCA CAAAATAATT AAAAAATAAA TAAATACAAA
ATTGGGGGTG GGGAGGGGGG GGAGATGGGG AGAGTGAAGC AGAACGTGGG GCTCACCTCG
ACCATGGTAA TAGCGATGAC TAATACGTAG ATGTACTGCC AAGTAGGAAA GTCCCATAAG
GTCATGTACT GGGCATAATG CCAGGCGGGC CATTTACCGT CATTGACGTC AATAGGGGGC
GTACTTGGCA TATGATACAC TTGATGTACT GCCAAGTGGG CAGTTTACCG TAAATACTCC
ACCCATTGAC GTCAATGGAA AGTCCCTATT GGCGTTACTA TGGGAACATA CGTCATTATT
GACGTCAATG GGCGGGGGTC GTTGGGCGGT CAGCCAGGCG GGCCATTTAC CGTAAGTTAT
GTAACGCGGA ACTCCATATA TGGGCTATGA ACTAATGACC CCGTAATTGA TTACTATTAA
TAACTAGCGA GGTGAGCCCC ACGTTCTGCT TCACTCTCCC CATCTCCCCC CCCTCCCCAC
CCCCAATTTT GTATTTATTT ATTTTTTAAT TATTTTGTGC AGCGATGGGG GCGGGGGGGG
GGGGGGGGCG CGCGCCAGGC GGGGGGGGGC GGGGCGAGGG GCGGGGGGGG GCGAGGCGGA
GAGGTGCGGC GGCAGCCAAT CAGAGCGGCG CGCTCCGAAA GTTTCCTTTT ATGGCGAGGC
GGCGGCGGCG GCGGCCCTAT AAAAAGCGAA GCGCGCGGCG GGCGCCAGAG CCCACCATGG
AAGCCCCTGC CCAGCTGCTG TTCCTGCTGC TCCTGTGGCT GCCTGACACC ACCGGCGATA
TCGTGCTGAC GCAGAGCCCT GATAGCCTGG CCGTGAGCCT CGGCGAACGG GCCACAATCA
GCTGTAAAGC CTCTCAGAGC GTGGACTATG ACGGCGACAG CTACATGAAC TGGTACCAGC
AGAAACCCGG CCAGCCTCCA AAAATCCTGA TCTACGACGC CAGCAATCTG GAAAGCGGCA
TCCCCGCCAG ATTCAGCGGC AGCGGCTCTG GCACTGATTT CACCCTGACA ATTTCTTCTC
TGGAACCCGA GGATTTTGCC ATCTACTACT GCCAGCAGAG CAACGAGGAC CCCTGGACCT
TTGGAGGCGG CACCAAGGTG GAAATCAAGC GCACCGTGGC CGCCCCTTCT GTCTTTATCT
TCCCTCCAAG CGACGAGCAG CTGAAGAGCG GAACCGCCTC TGTGGTGTGC CTGCTGAATA
ACTTCTACCC CAGAGAGGCA AAGGTCCAAT GGAAAGTTGA CAACGCCCTG CAAAGCGGCA
ACAGCCAAGA GAGCGTGACC GAGCAGGACA GCAAGGACTC AACATACAGC CTGTCCAGCA
CCCTGACCTT GAGCAAGGCC GACTACGAGA AGCACAAGGT GTACGCCTGT GAAGTGACAC
ATCAGGGCCT GTCCTCTCCT GTGACCAAAA GCTTCAACCG GGGCGAGTGC GGCGGCGGCG
GCTCCGGCGG AGGAGGCAGC GGTGGCGGCG GCTCTGGGGG AGGCGGAAGC GGCGGAGGCG
GCAGCGGCGG AGGCGGCAGC GGCGGCGGCG GATCCCAGGT GCAGCTGGTG CAGAGCGGAG
CCGAGGTGAA AAAGCCTGGC GCTAGTGTTA AGCTGAGCTG CACCGCCAGC GGCTTCAATA
TCAAGGACGA TTACATCCAC TGGGTGAAGC AGGCCCCCGG CCAGGGACTG GAGTGGATCG
GCAGAATCGA CCCTGCCGAC GGCCACACAA AGTACGCCCC TAAGTTCCAG GTGAAAGTCA
CCATCACCGC CGATACCAGC ACCTCTACAG CTTATCTGGA ACTGAGCAGC CTTAGATCCG
AGGACACCGC TGTGTACTAC TGCGCCAGAT ACGGCTACGG CAGAGAAGTG TTCGACTACT
GGGGACAGGG CACAACAGTG ACCGTGTCGT CCGCCAGCAC AAAGGGCCCT AGCGTGTTCC
CACTGGCTCC TTGTAGCAGA AGTACCTCAG AGAGCACGGC TGCTCTGGGC TGCCTGGTCA
AGGATTATTT CCCTGAGCCT GTGACCGTGT CCTGGAACAG CGGAGCCCTG ACAAGCGGGG
TGCACACCTT CCCCGCCGTG CTGCAGAGCA GCGGCCTGTA CTCTCTGTCC TCTGTCGTGA
CAGTGCCTAG CTCTAGCCTG GGCACAAAGA CCTACACCTG CAACGTGGAC CACAAGCCCA
GCAACACCAA GGTGGATAAG CGGGTGTGA
SEQ ID NO: 81-Peptide linker
SGSG
SEQ ID NO: 82-furin cleavage site
RX₁X₂R, where X₁ = any naturally occurring amino acid, and X₂ =
R or K
SEQ ID NO: 83-minCBA promoter (CMV enhancer underlined; CBA
promoter boldfaced and italicized; truncated chimeric intron:
boldfaced and underlined)
GCGTTACATA ACTTACGGTA AATGGCCCGC CTGGCTGACC GCCCAACGAC CCCCGCCCAT
TGACGTCAAT AATGACGTAT GTTCCCATAG TAACGCCAAT AGGGACTTTC CATTGACGTC
AATGGGTGGA GTATTTACGG TAAACTGCCC ACTTGGCAGT ACATCAAGTG TATCATATGC
CAAGTACGCC CCCTATTGAC GTCAATGACG GTAAATGGCC CGCCTGGCAT TATGCCCAGT
ACATGACCTT ATGGGACTTT CCTACTTGGC AGTACATCTA CGTATTAGTC ATCGCTATTA
CCATGGTCGA GGTGAGCCCC ACGTTCTGCT TCACTCTCCC CATCTCCCCC CCCTCCCCAC
CCCCAATTTT GTATTTATTT ATTTTTTAAT TATTTTGTGC AGCGATGGGG GCGGGGGGGG
GGGGGGGGCG CGCGCCAGGC GGGGGGGGGC GGGGCGAGGG GCGGGGGGGG GCGAGGCGGA
GAGGTGCGGC GGCAGCCAAT CAGAGCGGCG CGCTCCGAAA GTTTCCTTTT ATGGCGAGGC
GGCGGCGGCG GCGGCCCTAT AAAAAGCGAA GCGCGCGGCG GGCGGGAGTC GCTGCGCGCT
GCCTTCGCCC CGTGCCCCGC TCCGCCGCCG CCTCGCGCCG CCCGCCCCGG CTCTGACTGA
CCGCGTTACT CCCACAGGTG AGCGGGCGGG ACGGCCCTTC TCCTCCGGGC TGTAATTAGC
GCTTGGTTTA ATGACGGCTT GTTTCTTTTC TGTGGCTGCG TGAAAGCCTT GAGGGGCTCC
GGGAGCTAGA GCCTCTGCTA ACCATGTTCA TGCCTTCTTC TTTTTCCTAC AGCTCCTGGG
CAACGTGCTG GTTATTGTGC TGTCTCATCA TTTTGGCAAA GAATTCC

1. A single expression construct comprising a first nucleotide sequence encoding an inhibitor for activated complement subcomponent C1s (C1s inhibitor) and a second nucleotide sequence encoding an inhibitor for complement factor Bb (Bb inhibitor); or
a pair of expression constructs, one comprising the first nucleotide sequence and the other comprising the second nucleotide sequence.

2. The expression construct(s) of claim 1, wherein the C1s inhibitor and the Bb inhibitor are each an antibody fragment, optionally wherein the antibody fragment is a single-chain Fv (scFv) or a single-chain Fab (scFab).

3. The expression construct(s) of claim 2, wherein
(a) the C1s inhibitor is an anti-C1s antibody fragment comprising
heavy chain CDR (HCDR) 1-3 in SEQ ID NO:7, optionally comprising SEQ ID NOs: 1-3, respectively, and
light chain CDR (LCDR) 1-3 in SEQ ID NO:8, optionally comprising SEQ ID NOs: 4-6, respectively; and/or
(b) the Bb inhibitor is an anti-Bb antibody comprising
HCDR1-3 in SEQ ID NO: 19, optionally comprising SEQ ID NOs: 13-15, respectively, and
LCDR1-3 in SEQ ID NO:20, optionally comprising SEQ ID NOs: 16-18, respectively.

4. The expression construct(s) of claim 3, wherein
(a) the C1s inhibitor comprises
a heavy chain variable domain (V_{H}) comprising SEQ ID NO:7 or an amino acid sequence at least 95% identical thereto, and
a light chain variable domain (V_{L}) comprising SEQ ID NO:8 or an amino acid sequence at least 95% identical thereto; and/or
(b) the Bb inhibitor comprises
a V_{H} comprising SEQ ID NO:19 or an amino acid sequence at least 95% identical thereto, and
a V_{L} comprising SEQ ID NO:20 or an amino acid sequence at least 95% identical thereto.

5. The expression construct(s) of claim 3, wherein
(a) the C1s inhibitor comprises
a heavy chain (HC) comprising SEQ ID NO:10 or an amino acid sequence at least 95% identical thereto, and
a light chain (LC) comprising SEQ ID NO:11 or an amino acid sequence at least 95% identical thereto; and/or
(b) the Bb inhibitor comprises
an HC comprising SEQ ID NO:22 or an amino acid sequence at least 95% identical thereto and
an LC comprising SEQ ID NO:23 or an amino acid sequence at least 95% identical thereto.

6. The expression construct(s) of claim 2,
wherein the C1s inhibitor and the Bb inhibitor each comprise one or more charge mutations for promoting pairing between heavy and light chains of each inhibitor.

7. The expression construct(s) of claim 6, wherein
(a) the charge mutations in the C1s inhibitor comprises Q42E and Q292K, wherein the numbering is in accordance with SEQ ID NO: 12; and
(b) the charge mutations in the Bb inhibitor comprises Q38K and Q288E, optionally further comprising S114A, N137K, and T434E, wherein the numbering is in accordance with SEQ ID NO:24.

8. The expression construct(s) of claim 2, wherein the C1s inhibitor is an scFv or scFab in which the HC and the LC are linked by a peptide linker, optionally wherein the peptide linker comprises one or more, optionally 2, 3, 4, 5, 6, 7, 8, 9, or 10, G₄S (SEQ ID NO:46) repeats.

9. The expression construct(s) of claim 2, wherein the Bb inhibitor is an scFv or scFab in which the HC and the LC are linked by a peptide linker, optionally wherein the peptide linker comprises one or more, optionally 2, 3, 4, 5, 6, 7, 8, 9, or 10, G₄S (SEQ ID NO:46) repeats.

10. The single expression construct of claim 1, comprising a transgene encoding a fusion protein comprising the C1s inhibitor and the Bb inhibitor linked by a peptide linker,
optionally wherein the peptide linker comprises one or more, optionally 2, 3, 4, 5, 6, 7, 8, 9, or 10, G₄S (SEQ ID NO:46) repeats,
further optionally wherein the transgene is linked operably to a minimal chicken β-actin (minCBA) promoter.

11. The single expression construct of claim 1, wherein the expression construct comprises a bidirectional promoter that directs expression of the C1s inhibitor and the Bb inhibitor as separate molecules,
optionally wherein the bidirectional promoter is a pair of chicken β-actin (CBA) promoters placed in opposite direction and separated by a CMV enhancer,
further optionally wherein the bidirectional promoter comprises SEQ ID NO:53 or a nucleotide sequence at least 85% identical thereto.

12. The single expression construct of claim 2, wherein the expression construct expresses a heterodimer comprising (i) a fusion protein comprising a single-chain anti-C1s antibody fragment fused to the HC or LC of an anti-Bb antibody fragment; and (ii) the LC or HC polypeptide of the anti-Bb antibody fragment, wherein the coding sequence for the fusion protein and the coding sequence of the LC or HC polypeptide of the anti-Bb antibody fragment are separated in frame by a coding sequence for a cleavable peptide,
optionally wherein the cleavable peptide comprises a 2A sequence and/or a furin cleavage site,
further optionally the expression construct comprises a minCBA promoter.

13. The single expression construct of claim 2, wherein the expression construct expresses a heterodimer comprising (i) a fusion protein comprising a single-chain anti-Bb antibody fragment fused to the HC or LC of an anti-C1s antibody fragment; and (ii) the LC or HC polypeptide of the anti-C1s antibody fragment, wherein the coding sequence for the fusion protein and the coding sequence of the LC or HC polypeptide of the anti-C1s antibody fragment are separated in frame by a coding sequence for a cleavable peptide,
optionally wherein the cleavable peptide comprises a 2A sequence and/or a furin cleavage site,
further optionally the expression construct comprises a minCBA promoter.

14. The single expression construct of claim 2, wherein the expression construct encodes a fusion protein comprises, from N-terminus to C-terminus,
(i) an anti-C1s scFv, a (G₄S)₂ linker, and an anti-Bb scFv, optionally comprising SEQ ID NO: 55 (with or without the signal peptide) or an amino acid sequence at least 95% identical thereto;
(ii) an anti-Bb scFv, a (G₄S)₂ linker, and an anti-C1s scFv, optionally comprising SEQ ID NO: 57 (with or without the signal peptide) or an amino acid sequence at least 95% identical thereto;
(iii) an anti-C1s scFab, a (G₄S)₃ linker, and an anti-Bb scFab, optionally comprising SEQ ID NO: 26 or 28 (with or without the signal peptide), or an amino acid sequence at least 95% identical thereto;
(iv) an anti-Bb scFab, a (G₄S)₃ linker, and an anti-C1s scFab, optionally comprising SEQ ID NO: 30 or 32 (with or without the signal peptide), or an amino acid sequence at least 95% identical thereto;
(v) an anti-C1s scFab, a (G₄S)₂ linker, and an anti-Bb scFv, optionally comprising SEQ ID NO: 34 or 36 (with or with the signal peptide), or an amino acid sequence at least 95% identical thereto; or
(vi) an anti-C1s scFab, a (G₄S)₃ linker, and an anti-Bb scFv, optionally comprising SEQ ID NO: 59 or 61 (with or without the signal peptide), or an amino acid sequence at least 95% identical thereto.

15. The expression construct(s) of claim 2, wherein the expression construct(s) encodes
an anti-C1s scFab, optionally comprising SEQ ID NO: 12 or an amino acid sequence at least 95% identical thereto, optionally wherein the amino acid sequence comprises Q42E and Q292K mutations relative to SEQ ID NO: 12; and
an anti-Bb scFab, optionally comprising SEQ ID NO: 14 or an amino acid sequence at least 95% identical thereto, optionally wherein the amino acid sequence comprises Q38K and Q288E, and optionally S114A, N137K, and T434E, mutations relative to SEQ ID NO:14.

16. The single expression construct of claim 2, wherein the expression construct encodes a heterodimer comprised of
(A) (i) an anti-C1s LC and (ii) a fusion protein comprising an anti-C1s HC fused to an αBb scFab, optionally wherein the expression construct comprises a coding sequence for SEQ ID NO: 39, or an amino acid sequence at least 95% identical thereto;
(B) (i) an anti-C1s LC and (ii) a fusion protein comprising an anti-C1s HC fused to an anti-Bb scFab, optionally wherein the expression construct comprises a coding sequence for SEQ ID NO: 41, or an amino acid sequence at least 95% identical thereto;
(C) (i) a fusion protein comprising an anti-C1s scFab fused to an anti-Bb HC and (ii) an anti-Bb LC, optionally wherein the expression construct comprises a coding sequence for SEQ ID NO: 43, or an amino acid sequence at least 95% identical thereto; or
(D) (i) a fusion protein comprising an anti-C1s scFab fused to an anti-Bb HC and (ii) an anti-Bb LC, optionally wherein the expression construct comprises a coding sequence for SEQ ID NO: 45, or an amino acid sequence at least 95% identical thereto.

17. An isolated nucleic acid comprising a nucleotide sequence selected from SEQ ID NO: 25, 27, 29, 31, 33, 35, 37, 38, 40, 42, 54, 56, 58, 60, 62, 79, or 80, or encodes the same amino acid sequence(s) as the selected nucleotide sequence does.

18. One, two or more recombinant adeno-associated viruses (rAAV) comprising the expression construct(s) of claim 1.

19. The rAAV(s) of claim 18, wherein the genome of the rAAV(s) comprises the expression construct flanked by AAV2 inverted terminal repeats (ITRs).

20. The rAAV(s) of claim 19, wherein the genome comprises SEQ ID NO:50, 51, or 52; or encodes the same amino acid sequence(s) as SEQ ID NO:50, 51, or 52 does.

21. The rAAV(s) of claim 18, comprising a capsid of AAV2, optionally wildtype AAV2.

22. A pharmaceutical composition comprising the rAAV(s) of claim 18 and a pharmaceutically acceptable carrier.

23. A protein or proteins encoded by the expression construct(s) of claim 1.

24. A host cell comprising the expression construct(s) of claim 1.

25. A method for treating dry age-related macular degeneration (AMD) in a patient in need thereof, comprising administering an effective amount of the pharmaceutical composition of claim 22.

26. The method of claim 25, wherein the administering is by intravitreal injection.

27. The method of claim 25, wherein the patient has geographic atrophy (GA) secondary to dry AMD.

28. The method of claim 25, wherein the effective amount is 10⁷ to 10¹⁵, optionally 10⁸ to 10¹⁴, 10⁹ to 10¹³, further optionally 2×10⁹, 2×10¹⁰, or 2×10¹¹, vector genomes.

29. A mammalian promoter comprising SEQ ID NO:83 or a sequence at least 85% identical thereto.

30. A bidirectional mammalian promoter comprising a pair of chicken β-actin promoters placed in opposite orientation, separated by a CMV enhancer, optionally wherein the bidirectional mammalian promoter comprises SEQ ID NO:53 or a sequence at least 85% identical thereto.

31. One, two or more recombinant adeno-associated viruses (rAAV) comprising the isolated nucleic acid of claim 17.
